(19) United States
(12) Patent Application Publication
(10) Pub. No.: US 2026/0345360 A1
(43) Pub. Date: Nov. 5, 2026

(54) SYSTEMS AND METHODS FOR OBSTRUCTING OR REDUCING BLOOD FLOW

(71) Applicant: Auro Medical, Inc., Mission, KS (US)

(72) Inventors: Stephen U. Stechschulte, Leawood, KS (US); Ivan Vesely, Mission, KS (US); Frank Nicholas Franano, Mission, KS (US); Mark J. Mendel, Seattle, WA (US); Nicholas James Franano, Mission, KS (US)

(21) Appl. No.: 19/701,545

(22) Filed: Jun. 8, 2026

Related U.S. Application Data
(63) Continuation of application No. PCT/US2025/055814, filed on Nov. 17, 2025.
(60) Provisional application No. 63/721,989, filed on Nov. 18, 2024.

(51) Int. Cl. A61F 2/958; A61F 2/06; A61F 2/24; A61M 25/10

(52) U.S. Cl. CPC A61F 2/958; A61F 2/2418; A61F 2/2475; A61F 2002/068; A61F 2250/0063; A61M 2025/1004; A61M 2025/1061

(57) ABSTRACT

An expandable implant can be used obstruct or reduce flow in arteries, veins, fusiform aneurysms, parent vessels of saccular aneurysms, paravalvular leak pathways, biological conduits, or other blood-containing, fluid-containing, or biological spaces. A balloon catheter delivery system can be used to deliver the implant. This can include an over-the-guidewire balloon catheter delivery system. The catheter portion of the system can deliver the expandable metallic implant to the treatment site, and the balloon portion of the system can expand the expandable metallic implant at the treatment site. The deliverability of the expandable implant devices can be optimized by reducing the overall diameter of the folded and pleated expandable metallic implant, and shortening the length of the rigid device segments. The risk of implant migration can be reduced by adding features to the expandable metallic implant implants that increase sliding friction in tissue.

### INCORPORATION BY REFERENCE TO ANY PRIORITY APPLICATIONS

This application is a continuation of PCT Application PCT/US2025/055814, filed Nov. 17, 2025, which claims priority to the U.S. Provisional Application 63/721989, filed Nov. 18, 2024, which is incorporated by reference herein in its entirety.

### BACKGROUND

### Field

Examples of this application are directed to systems, methods, and devices for obstructing or reducing flow in arteries, veins, fusiform aneurysms, parent vessels of saccular aneurysms, paravalvular leak pathways, biological conduits, or other blood-containing, fluid-containing, or biological spaces using an expandable implant.

### Description of the Related Art

In certain clinical situations, patients can benefit from an obstruction or reduction of flow in arteries, veins, aneurysms, parent vessels of saccular aneurysms, paravalvular leak pathways, biological conduits, or other blood-containing, fluid-containing, or biological spaces. Clinical settings where this is beneficial include reducing bleeding after vessel injury or rupture, reducing bleeding from esophageal varices, occluding pelvic venous varices, reducing blood flow to tumors, and rerouting the path of blood away from vascular abnormalities, anomalies, and malformations.

In certain clinical situations, devices comprising an expandable implant are used to reduce flow of blood or fluid. In some cases, expandable implants are configured as small, pre-formed metallic wire segments, which are often referred to as “coils”. Optionally, these coil expandable implants may also comprise coatings, coverings, threads, or fibers, including those comprised of polymers like polyesters, nylon, and polyvinyl alcohols or natural materials such as wool. In some cases, coil expandable implants are comprised of resilient materials. In other cases, coil expandable implants are not comprised of resilient materials. These coil expandable implants can obstruct flow directly and also induce the formation of occlusive thrombus locally through a process known as “embolization”. Coil expandable implants can be pushed through coiling or delivery catheters or other types of catheters using a delivery wire or another type of elongate body and deposited at the target location. There are benefits to using coil expandable implants for artery and vein embolization. They are flexible and can be elongated, making them easy to advance through catheters and to target locations, including locations accessible only through small diameter catheters or along tortuous paths. However, they have disadvantages. Precise placement of coil expandable implants is difficult, and misplacement and migration are common, sometimes leading to non-target embolization. Migration of coil expandable implants is especially common in veins, as the diameter of veins increases from distal to proximal. When a coil is placed in a vein, blood flow pushes the coil toward larger vessels, making it easier for a coil to come free and migrate long distances. Often, coil expandable implants that migrate after placement in veins can move to critical structures such as the right atrium, right ventricle, or pulmonary arteries. Because they present a porous barrier to blood flow, multiple coil expandable implants are often required for complete occlusion, leading to increased treatment time and cost. Late recanalization or re-opening of treated arteries and veins is also common.

In some cases, expandable implants are configured as structures comprising resilient metallic wires, which are often referred to as “vascular plugs”. Optionally, these vascular plug expandable implants may also comprise coatings, coverings, threads, or fibers, including those comprised of polymers like polyesters, nylon, and polyvinyl alcohols or natural materials such as wool. These vascular plug expandable implants can obstruct flow directly and also induce the formation of occlusive thrombus locally through a process known as “embolization”. Vascular plug expandable implants can be pushed through coiling or delivery catheters or other types of catheters using a delivery wire (or other type of elongate body such as a laser cut nitinol tube) and deposited at the target location. There are benefits to using vascular plug expandable implants for artery and vein embolization. Vascular plug expandable implants can be placed more precisely than coil expandable implants. Also, a single vascular plug expandable metallic implant is sometimes all that is needed for treatment, reducing the complexity of the treatment. However, as with coil expandable implants, there are disadvantages. Vascular plug expandable implants are often stiff, making them difficult to place in small, distal, and tortuous locations. Migration, and non-target embolization are common, especially in veins, for the reasons discussed above. Like coil expandable implants, vascular plug expandable implants also present an incomplete barrier to the flow of blood, and require the formation of thrombus, which can increase the time required for complete occlusion. Late recanalization or re-opening of treated arteries and veins is also common.

In some cases, other expandable implants are configured as structures comprising detachable balloon expandable implants, which are often referred to as “detachable balloons”.

Detachable balloon expandable implants, expand primarily by stretching or unfolding of the material of the wall.

In some cases, the detachable balloon (“a detachable balloon expandable implant”) is joined to an elongate body with an inflation lumen, a guidewire lumen, and two hubs (one for the inflation lumen and one for the guidewire lumen). The detachable balloon expandable implant is advanced to the target location in an artery or vein over a guidewire, inflated, and detached. These detachable balloon expandable implants obstruct flow directly and also induce the formation of occlusive thrombus locally through a process known as “embolization”. Detachable balloon expandable implants can be pushed through catheters or advanced over a guidewire using the elongate body and deposited at the target location. There are benefits to using detachable balloon expandable implants for artery and vein embolization. After inflation, the expanded, detachable balloon expandable implants generally conform well to the target artery or vein segment and provide a good seal, stopping flow. Detachable balloon expandable implants can be placed with precision and present a solid surface to the flow of blood, often providing immediate and complete occlusion. However, detachable balloon expandable implants have been mostly abandoned in favor of other devices. The balloon expandable implants are highly compliant and use valves to preserve a high internal pressure after detachment to maintain their expanded size and shape. There is a substantial rate of valve failure resulting in deflation of the balloon expandable implant and often leading to recanalization of the treated artery or vein segment and increasing the risk of migration. The latex and silicone walls resist tissue adhesion over time and can degrade in vivo, leading to deflation and migration of the detachable balloon expandable implants, and vessel recanalization.

There remains an unmet clinical need for devices and systems that can provide safe, effective, and reliable embolization of arteries and veins. Devices are needed which are low profile, flexible, and deliverable, easy to use, can be quickly placed with a high degree of precision, result in immediate and complete artery and vein occlusion with just one or a few devices, provide durable and permanent occlusion with low rates of device migration and recanalization, and are readily available in a wide range of sizes at a reasonable cost.

An aneurysm is an excessive localized enlargement of an artery or vein caused by a weakening of the artery or vein wall. Aneurysms can rupture without warning, leading to bleeding. In the brain, bleeding from a ruptured aneurysm can cause stroke and sometimes death. Outside the brain, bleeding from a ruptured aneurysm can cause hypotension and sometimes death. There are two main types of aneurysms, fusiform aneurysms and saccular aneurysms. A saccular aneurysm is a rounded or pouch-like localized enlargement that is attached by a neck to an artery or vein, or to a branching of an artery or vein. Saccular aneurysms may occur throughout the body but are most commonly found in the arteries of the brain. A fusiform aneurysm is an outpouching of an artery or vein wall that is expanded in all directions, without a distinct neck.

The rate of spontaneous rupture of aneurysms increases with increasing aneurysm size, and therefore large aneurysms that are found during a medical or surgical evaluation are usually treated. Ruptured aneurysms are almost always treated, if possible. Aneurysms can be treated with surgery, wherein a surgical clip is placed across the neck of the aneurysm to exclude it from blood flow (for saccular aneurysms) or wherein biological conduits such as a vein segment or a synthetic conduit) such as an expanded polytetrafluoroethylene (PTFE) or polyester graft) is used to surgically bypass the aneurysmal segment. Many aneurysms can also be treated with minimally invasive procedures. Embolic devices can be placed in the lumen or sac of fusiform aneurysms and saccular aneurysms. Embolic devices can be placed in the parent vessel of saccular aneurysms. Covered or flow-diverting stents can be placed in the parent vessel of saccular aneurysms or in the aneurysmal segment of fusiform aneurysms.

In some cases, coil expandable implants are used to treat fusiform aneurysms. To treat a fusiform aneurysm with coil expandable implants, a physician inserts a catheter into a lumen of the vascular system and maneuvers the catheter tip into the fusiform aneurysm. With the catheter tip in position, the physician pushes individual coil expandable implants into the lumen or sac of the fusiform aneurysm. During and after the treatment, thrombus forms in and around the coil expandable implants with the goal of reducing blood flow to the fusiform aneurysm and occluding the fusiform aneurysm. Over time, for successful treatments, the thrombus matures into fibrous tissue, which completely seals off the fusiform aneurysm from the adjacent vessel. Although effective, treatment of fusiform aneurysms with coil expandable implants has disadvantages. Coil placement is difficult to control, often resulting in coil misplacement or migration to non-target locations, sometimes resulting in occlusion of non-target vessels. In cases of coil migration, physicians may be compelled to attempt retrieval of the coil expandable implants from the non-target location. Numerous coil expandable implants are usually required during a treatment, resulting in high costs and long treatment times. Furthermore, coil expandable implants only partially fill the fusiform aneurysm. Thrombus and scar tissue can accumulate to seal the fusiform aneurysm completely, a process that can take months to years and is often incomplete. Slow fusiform aneurysm occlusion or sealing can reduce the effectiveness of coil expandable implants in the treatment of acute fusiform aneurysm rupture. Fusiform aneurysm occlusion or sealing after coiling is often incomplete, subjecting the patient to a persistent risk of fusiform aneurysm rupture and leading to unacceptably high rates of retreatment. Even when the use of coil expandable implants is initially effective, recanalization of the fusiform aneurysm may occur, resulting in a return of blood flow to the fusiform aneurysm and an increased risk of rupture. Incomplete filling, occluding, and sealing of fusiform aneurysms with coil expandable implants is especially common in the regions where coil density is low and blood flow rates are high. Coil expandable implants are susceptible to compaction, further exposing the fusiform aneurysm to persistent blood flow and contributing to the high rate of fusiform aneurysm recurrence and retreatment.

In some cases, coil expandable implants are used to treat saccular aneurysms by occluding the parent vessel of the saccular aneurysm. To treat a saccular aneurysm by occluding the parent vessel of the saccular aneurysm with coil expandable implants, a physician inserts a catheter into a lumen of the vascular system and maneuvers the catheter tip into the parent vessel of the saccular aneurysm. With the catheter tip in position, the physician pushes individual coil expandable implants into the lumen of the parent vessel. During and after the treatment, thrombus forms in and around the coil expandable implants with the goal of reducing blood flow to the saccular aneurysm and the parent vessel by occluding the saccular aneurysm and the parent vessel. Over time, for successful treatments, the thrombus matures into fibrous tissue, which seals off the saccular aneurysm and the parent vessel. Although effective, treatment of saccular aneurysms by occluding the parent vessel of the saccular aneurysm with coil expandable implants has disadvantages. Coil placement is difficult to control, often resulting in coil misplacement or migration to non-target locations, sometimes resulting in occlusion of non-target vessels. In cases of coil migration, physicians may be compelled to attempt retrieval of the coil expandable implants from the non-target location. Numerous coil expandable implants are usually required during a treatment, resulting in high costs and long treatment times. Furthermore, coil expandable implants only partially fill the parent vessel. Thrombus and scar tissue can accumulate to seal the parent vessel and saccular aneurysm completely, a process that can take months to years and is often incomplete. Slow parent vessel and saccular aneurysm occlusion or sealing can reduce the effectiveness of coil expandable implants in the treatment of acute saccular aneurysm rupture. Parent vessel and saccular aneurysm occlusion or sealing after coiling is often incomplete, subjecting the patient to a persistent risk of saccular aneurysm rupture and leading to unacceptably high rates of retreatment. Even when the use of coil expandable implants is initially effective, recanalization of the parent vessel and saccular aneurysm may occur, resulting in a return of blood flow to the saccular aneurysm and an increased risk of rupture. Incomplete filling, occluding, and sealing of parent vessels and aneurysms with coil expandable implants is especially common in the regions where coil density is low and blood flow rates are high. Coil expandable implants are susceptible to compaction, further exposing the saccular aneurysm to persistent blood flow and contributing to the high rate of saccular aneurysm recurrence and retreatment.

In some cases, detachable balloon expandable implant devices are used to treat fusiform aneurysms by occluding the sac of the fusiform aneurysm. In some cases, the detachable balloon expandable implant devices are advanced over a guidewire into the lumen or sac of the fusiform aneurysm, and the detachable balloon expandable implant is inflated, detached, and left in the lumen of the aneurysm while the elongate body is removed. The expanded detachable balloon expandable implant then blocks blood flow into the aneurysm and promotes aneurysm thrombosis. Detachable balloon expandable implant devices and their associated methods of use have several disadvantages. First, the wall of detachable balloon expandable implants are typically made of compliant polymers such as latex and silicone that generally resist tissue incorporation. This reduced fixation of the devices to the fusiform aneurysm wall increases the risk of migration of detachable balloon expandable implants and embolization of downstream artery segments. Second, the wall of detachable balloon expandable implants are elastic and use valves to preserve a high internal pressure after detachment that is needed to maintain their expanded size and shape. Unfortunately, there is a substantial rate of valve failure resulting in deflation of detachable balloon expandable implants leading to fusiform aneurysm recanalization and migration of detachable balloon expandable implants.

In some cases, detachable balloon expandable implant devices are used to treat saccular aneurysms by occluding the parent vessel of saccular aneurysms. In some cases, detachable balloon expandable implant devices are advanced over a guidewire into the lumen of the parent vessel of a saccular aneurysm, and the detachable balloon expandable implant is inflated, detached, and left in the lumen of the parent vessel while the elongate body is removed. The expanded detachable balloon expandable implant then blocks blood flow into the parent vessel and the saccular aneurysm and promotes aneurysm thrombosis. Detachable balloon expandable implant devices and their associated methods of use have several disadvantages. First, the wall of detachable balloon expandable implants are typically made of compliant polymers such as latex and silicone that generally resist tissue incorporation. This reduced fixation of the devices to the wall of the parent vessel increases the risk of migration of detachable balloon expandable implants and embolization of downstream artery segments.

Second, the wall of detachable balloon expandable implants are elastic and use valves to preserve a high internal pressure after detachment that is needed to maintain their expanded size and shape. Unfortunately, there is a substantial rate of valve failure resulting in deflation of detachable balloon expandable implants leading to parent vessel and saccular aneurysm recanalization and migration of detachable balloon expandable implants.

There remains an unmet clinical need for devices, systems, and methods for effectively and reliably treating fusiform aneurysms. Devices are needed which are low profile, flexible, and deliverable, easy to use, can be quickly placed with a high degree of precision, result in immediate and complete aneurysm occlusion, provide durable and permanent aneurysm occlusion with low rates of device migration and recanalization, and are readily available in a wide range of sizes and shapes at a reasonable cost.

The valves in the heart can become obstructed or develop leaks, resulting in either a reduction in cardiac output, an increase in the cardiac workload, or both. These faulty valves can be repaired or replaced, either during a surgery or a minimally invasive procedure. After valve repair and replacement, a new leak may form in the region adjacent to the valve, which can reduce cardiac output and increase cardiac workload. The development of these leaks is especially common when treating the aortic valve. These leaks can also lead to blood damage, including hemolysis of red blood cells, which can cause anemia and kidney damage. Patients who develop these “paravalvular leaks” can benefit from occlusion of the leak path, which can improve cardiac output, reduce cardiac workload, and reduce blood and kidney damage. There are currently no devices specifically approved for the treatment of paravalvular leaks in the U.S. Physicians sometimes use self-expanding vascular plug expandable implants that can be pushed through a catheter and deposited in the lumen of the paravalvular leak path in an attempt to occlude it. However, there are disadvantages with this approach. The devices are stiff, making them difficult to place. They also present a porous surface to the flow of blood resulting in a failure of some devices to fully occlude the leak path, which can be large and have a high rate of blood flow. By design, thrombus accumulates on device surfaces and in the high flow environment of a paravalvular leak, this thrombus can be released from the device surface and travel downstream, which can result in stroke, ischemia, and organ and tissue death. As with many porous devices, complete endothelialization of vascular plug expandable implants is usually slow and often incomplete, resulting in a persistent risk of thrombus formation and embolization of thrombi.

There remains an unmet clinical need for devices, systems, and methods for effectively and reliably occluding paravalvular leak paths. Devices are needed which are low profile, flexible, and deliverable, easy to use, can be quickly placed with a high degree of precision, result in immediate and complete occlusion of the leak path with just one or a few devices, have surfaces that are resistant to thrombus formation and are quickly covered with endothelium, provide durable and permanent occlusion with low rates of device migration and recanalization, and are readily available in a wide range of sizes at a reasonable cost.

In certain clinical situations, patients can benefit from the occlusion or sealing of biological conduits. Clinical settings where occlusion or sealing of biological conduits is beneficial include intentional blockage of fallopian tubes to prevent pregnancy. Examples of biological conduits includes thoracic ducts, lymphatic ducts, pancreatic ducts, biliary ducts, fallopian tubes, bronchi, ureters, urethras, esophagus, duodenum, jejunum, ileum, colon, vas deferens, salivary ducts, parotid ducts, lactiferous ducts, Schlemm's canals, tear and nasolacrimal ducts, cerebral aqueducts, peripheral nerve sheaths, and any other tubular structure or channel in a human that conveys biological fluid and suspensions, solid or semi-solid biological materials, gas, or air. Examples of biological fluids, solid or semi-solid biological materials, gas, or air includes lymph, cerebrospinal fluid, urine, bile, pancreatic juice, saliva, milk, tears, aqueous humor, eggs, semen, pulmonary secretions, food, water, feces, inspired air, or exhaled air.

There remains an unmet clinical need for devices, systems, and methods for effectively and reliably occluding or sealing biological conduits. Devices are needed which are low profile, flexible, and deliverable, easy to use, can be quickly placed with a high degree of precision, result in immediate and complete occlusion or sealing of biological conduits with just one or a few devices, provide durable and permanent occlusion or sealing with low rates of device migration and recanalization, and are readily available in a range of sizes at a reasonable cost.

The ability of a minimally invasive device to reach a particular location in vivo (a characteristic known as “deliverability”) is dependent upon several factors. First, the device can be long enough to reach from the location where it is inserted (the insertion site) to the location where the treatment is desired (the treatment site). Second, an ideal device should have an overall diameter or “profile” that is small enough to pass through arteries, veins, or other biological conduits and spaces that are located between device insertion site and the treatment site. Third, an ideal device should not have any large or abrupt changes in outer diameter (“step-offs”) that can cause friction during device advancement or retraction, especially when encountering edges associated catheter hubs, catheter tips, vessel side branches, atherosclerotic plaques, regions of vessel wall scarring or fibrosis, or intravascular thrombus, for example. Fourth, the device should be flexible enough to navigate a tortuous path from the insertion site to the treatment site. For an embolic device, an important component of deliverability involves the flexibility of the elongate body that is used to push the implant to the target location, as well as the flexibility of the implant itself. When optimizing device flexibility and deliverability, there is often a trade-off wherein increasing implant flexibility increases the risk of implant collapse, compression, or compaction in vivo leading to re-opening of the treated artery, vein, aneurysm, parent vessel of a saccular aneurysm, paravalvular leak pathway, biological conduit, or other blood-containing, fluid-containing, or biological space. Certain expandable implants comprising gold are rigid, which reduces the risk of implant collapse, compression, or compaction in vivo but also reduces deliverability. The deliverability of expandable implant devices can be optimized by: 1) shortening the length of the rigid device segments; 2) increasing the flexibility of the flexible device segments; and 3) placing flexible junctions between rigid device segments.

The safety and tolerability of devices can depend upon several factors, including the type and extent of the biological response to the expandable implant (a characteristic known as “biocompatibility”). A biocompatible device is one that is well-tolerated by the body and does not cause adverse reactions, excessive local or systemic host responses or tissue damage.

### SUMMARY

The present application describes expandable metallic implants and expandable metallic implant devices configured to obstruct or reduce flow in arteries, veins, aneurysms, parent vessels of saccular aneurysms, paravalvular leak pathways, biological conduits, or other blood-containing, fluid-containing, or biological spaces, and methods of their use. In some examples, the present application describes expandable metallic implant devices comprising an expandable metallic implant and a balloon catheter, wherein the balloon catheter comprises a balloon and an elongate body.

Expandable metallic implant devices are described comprising a hollow expandable metallic implant, including a hollow expandable metallic implant comprising gold, with a wall that is open on the proximal end and closed or obstructed on the distal end that can be joined to a balloon of a balloon catheter by the folding and pleating together of an expandable metallic implant and a balloon, delivered to a desired location in a human patient in a folded and pleated form using a balloon catheter, transitioned to an expanded configuration by inflation of a balloon, and separated from a balloon by deflation or collapse of a balloon, wherein the expanded expandable metallic implant remains in the patient while the balloon catheter is removed from the patient.

Various examples of expandable metallic implant devices are described, including devices comprising a compliant or semi-compliant balloon and an expanded expandable metallic implant with one or more flaps or barbs, including flaps or barbs with a pointed end, wherein the free end of a flap or barb can be extended radially from the surface of the expanded expandable metallic implant during balloon inflation as a means to reduce the risk of migration of the expanded expandable metallic implant after separation from the balloon. Examples of expandable metallic implant devices with a balloon retention region are also described, as a means to reduce the risk of separation of an expanded expandable metallic implant and an inflated balloon. Examples of expandable metallic implant devices are also described wherein deliverability is optimized by reducing the overall diameter of the folded and pleated portion of the expandable metallic implant, shortening the length of various rigid device segments, increasing the flexibility of various flexible device segments, and placing flexible junctions or segments between rigid device segments. Examples of expandable metallic implant devices are also described with various components for guiding folded and pleated expandable implants 4 into and through guide catheters or guide sheaths to desired locations in patients, including expandable metallic implants that incorporate a guidewire segment at the distal end of the expandable metallic implant.

Systems comprising an expandable metallic implant device and additional devices are described. The additional devices are used to assist in the delivery of folded and pleated expandable metallic implant or the separation of the expanded expandable metallic implant, and a balloon are described. Systems comprising an expandable metallic implant device and one or more additional devices comprising additional expandable implants are described, wherein the additional devices are configured to assist in reducing flow in arteries, veins, aneurysms, parent vessels of saccular aneurysms, paravalvular leak pathways, biological conduits, or other blood-containing, fluid-containing, or biological spaces, or to reduce the risk of migration of expanded expandable metallic implants in patients. Systems comprising an expandable metallic implant device and additional devices are described wherein the luminal diameter of a guide catheter or guide sheath designed to deliver expandable metallic implant devices are configured such that a coiling or delivery catheter can be advanced through the lumen of a guide catheter or guide sheath while the elongated body of a balloon catheter of an expandable metallic implant device is present in the lumen of a guide catheter or guide sheath, as a means of allowing the simultaneous placement of an expandable metallic implant and additional expandable implants, including one or more coil expandable implants.

Methods of use of expandable implant devices and systems comprising expandable implant devices and other devices to reduce flow in arteries, veins, aneurysms, parent vessels of saccular aneurysms, paravalvular leak pathways, biological conduits, or other blood-containing, fluid-containing, or biological spaces are described, including the simultaneous placement of an expandable metallic implant and one or more additional expandable implants, including one or more coil expandable implants.

Methods of manufacturing expandable metallic implants, balloons, balloon catheters 100, and expandable implant devices are described.

In some examples, a system for obstructing or reducing a flow of blood in an artery or vein in a patient can include: a balloon catheter including: an elongate body including a proximal portion and a distal portion; an inflation lumen extending from the proximal portion to the distal portion; and a balloon positioned at the distal portion of the elongate body in fluid communication with the inflation lumen; and an expandable metallic implant configured to be carried by the balloon catheter to an implantation site in an artery and vein in a patient and configured to expand from a unexpanded configuration to an expanded configuration, wherein the expandable metallic implant in the expanded configuration includes a wall defining a hollow shape having a proximally facing opening at a proximal end thereof and a closed or obstructed distal end; wherein the balloon and the expandable metallic implant are configured to be delivered in a unexpanded configuration to the implantation site, wherein the balloon is configured to be inflated at the implantation site through the inflation lumen to expand the expandable metallic implant to the expanded configuration, and wherein the balloon catheter is configured to be removed from the patient such that the expandable metallic implant remains in the artery or vein in an expanded configuration at the implantation site.

In some examples, the expandable metallic implant includes gold. In some examples, the expandable metallic implant includes a layer of gold with a thickness of between 5 microns and 100 microns. In some examples, the expandable metallic implant includes electroformed gold. In some examples, the expandable metallic implant further includes a neck portion on a distal end of the expandable metallic implant. In some examples, the system can include a nose cone configured to attach to the neck portion of the expandable metallic implant. A nose cone can be considered a form of “transitional body” that provides a profile transition from one device region with a larger diameter to another device region with a smaller diameter. In some examples, the system can include a guidewire segment extending from the neck portion of the expandable metallic implant or the nose cone. In some examples, the proximally facing opening is substantially circular. In some examples, the expandable metallic implant includes a balloon retention region, wherein in the expanded configuration, the balloon retention region has a smaller diameter than a portion of the expandable metallic implant distal to the balloon retention region. In some examples, the expandable metallic implant includes one or more flaps configured to extend radially outward from a surface of the expandable metallic implant in the expanded configuration during inflation of the balloon. In some examples, the one or more flaps include a pointed end, and wherein the one or more flaps are configured to embed into the wall of an artery or vein wall in the radially extended configuration and secure the expandable metallic implant to the artery or vein wall. In some examples, the one or more flaps are configured to protrude from one or more holes in a surface of the expandable metallic implant. In some examples, the one or more flaps are configured to protrude from a proximal end of the expandable metallic implant. In some examples, the balloon is semi-compliant. In some examples, the semi-compliant balloon includes a compliant polymer. In some examples, the semi-compliant balloon has a wall thickness between about 5 microns and about 30 microns. In some examples, the elongate body includes a single lumen. Another term that describes elongate bodies is “longitudinally extending bodies”. In some examples, a length of the inflated balloon is greater than a length of the expanded expandable metallic implant. In some examples, the expandable metallic implant has a folded and pleated configuration when in the unexpanded configuration. In some examples, the balloon and the expandable metallic implant are folded together into wings. In some examples, the proximal portion of the wings include two layers of balloon. In some examples, the two layers of balloon are external layers. In some examples, the distal portion of the wings include two layers of expandable metallic implant and two layers of balloon. In some examples, the two layers of balloon are internal layers, and the two layers of expandable metallic implant are outer layers. In some examples, the wings 121 of the balloon 110 are pleated around a central axis. For the purpose of definition, a central axis is located parallel to the first axis and central to the named structure, in this case the balloon 110.

In some examples, a method for obstructing or reducing a flow of blood in an artery or vein of a patient can include: delivering a balloon catheter carrying an expandable metallic implant in a unexpanded configuration to an implantation site in an artery or vein of a patient; inflating a balloon through an inflation lumen of the balloon catheter to cause the expandable metallic implant to expand to an expanded configuration that provides for an obstruction or reduction of flow of blood at the implantation site; and removing the balloon catheter from the patient while the expandable metallic implant remains at the implantation site in the patient in an expanded configuration.

In some examples, the expandable metallic implant has a folded and pleated configuration when in the unexpanded configuration. In some examples, the expandable metallic implant in the expanded configuration includes a wall defining a hollow shape having a proximally facing opening at a proximal end thereof and a closed or obstructed distal end. In some examples, the expandable metallic implant that engages with the artery or vein of the patient includes a layer of gold with a thickness of between 5 microns and 100 microns. In some examples, inflating the balloon includes delivering between about 1 atm and about 3 atm of pressure to the balloon. In some examples, the method can include deflating the balloon such that the expandable metallic implant remains in the expanded configuration and engaged with a wall of an artery or vein of the patient. In some examples, inflating the balloon causes a flap of the expandable metallic implant to extend radially outward from a surface of the expandable metallic implant. In some examples, the flap protrudes radially outward from a hole in the surface of the expandable metallic implant. In some examples, the flap protrudes from a proximal end of the expandable metallic implant. In some examples, the method can include delivering the balloon catheter and the expandable metallic implant to the implantation site through a guide catheter or guide sheath. In some examples, the method can include selecting the expandable metallic implant from a plurality of expandable metallic implants of different sizes based on a vessel size at the implantation site.

In a preferred example, the expandable metallic implant device 1 comprises a balloon catheter 100 including: an elongate body 197 comprising and inner shaft 172 and an outer shaft 171; an inflation lumen extending from the proximal portion to the distal portion and occupying space between the inner shaft 172 and the outer shaft 171; and a balloon 110 positioned at the distal portion of the elongate body 197 in fluid communication with the inflation lumen; and an expandable metallic implant 10 configured to be carried by the balloon catheter 100 to an implantation site in an artery and vein in a patient and configured to expand from a unexpanded configuration to an expanded configuration, wherein the expandable metallic implant 10 in the expanded configuration 11 includes a wall defining a hollow shape having a proximally facing opening at a proximal end thereof and a closed or obstructed distal end; wherein the balloon 110 and the expandable metallic implant 10 are configured to be delivered in a unexpanded configuration to the implantation site, wherein the balloon 110 is configured to be inflated at the implantation site through the inflation lumen to expand the expandable metallic implant 10 to the expanded configuration, and wherein the balloon catheter 100 is configured to be removed from the patient such that the expanded expandable metallic implant 11 remains in the artery or vein in an expanded configuration at the implantation site. In this preferred example, the expandable metallic implant 10 comprises a layer of electroformed gold with a thickness of between 5 microns and 100 microns and a neck portion 60 on a distal end of the expandable metallic implant 10 that further comprises a polymer valve 79 configured to be open when the balloon catheter 100 and the expandable metallic implant 10 are joined and configured to be closed when the balloon catheter 100 and the expandable metallic implant 10 are separated. In this preferred example, the balloon catheter 100 comprises a hub 176 with two ports, one port 178 for insertion of a guidewire and optionally for insertion of coil expandable bodies and a second port 177 for inflation of the balloon. Optionally, in this preferred example, the balloon catheter 100 comprises a semi-compliant balloon 110. Optionally, in this preferred example, the expandable metallic implant 10 includes a plurality of flaps 22 configured to extend radially outward from a surface of the expanded expandable metallic implant 11 during inflation of the balloon 110.

In some examples, a system for obstructing or reducing a flow of blood in an artery or vein in a patient can include: a balloon catheter including: an elongate body including a proximal portion and a distal portion; an inflation lumen extending from the proximal portion to the distal portion; a guidewire lumen extending from the proximal portion to the distal portion; and a balloon positioned at the distal portion of the elongate body in fluid communication with the inflation lumen; and an expandable metallic implant configured to be carried by the balloon catheter to an implantation site in an artery or a vein in a patient and configured to expand from an unexpanded configuration to an expanded configuration, wherein the expandable metallic implant in the expanded configuration includes a wall defining a hollow shape having a proximally facing opening at a proximal end thereof and an at least partially obstructed distal end, the at least partially obstructed distal end including an opening configured to allow for passage of the distal portion of the elongate body of the balloon catheter; wherein the balloon and the expandable metallic implant are configured to be delivered in an unexpanded configuration to the implantation site, wherein the balloon is configured to be inflated at the implantation site through the inflation lumen to expand the expandable metallic implant to the expanded configuration, wherein the balloon catheter is configured to be removed from the patient, wherein the expandable metallic implant is configured to remain in the artery or vein in the expanded configuration at the implantation site after removal of the balloon catheter, and wherein at least a portion of the opening of the expandable metallic implant is configured to be obstructed after separation of the balloon catheter and the expandable metallic implant in the expanded configuration.

In some examples, the expandable metallic implant includes gold. In some examples, the expandable metallic implant includes a layer of gold with a thickness of between 5 microns and 100 microns. In some examples, the expandable metallic implant includes electroformed gold. In some examples, the expandable metallic implant further includes a neck portion on a distal end of the expandable metallic implant, the neck portion including an opening configured to allow for passage of the distal portion of the elongate body of the balloon catheter. In some examples, the proximally facing opening is substantially circular. In some examples, the expandable metallic implant includes a balloon retention region, wherein in the expanded configuration, the balloon retention region has a smaller diameter than a portion of the expandable metallic implant distal to the balloon retention region. In some examples, the expandable metallic implant includes one or more flaps configured to extend radially outward from a surface of the expandable metallic implant in the expanded configuration during inflation of the balloon. In some examples, the opening of the neck portion includes a valve, the valve configured to allow for the distal portion of the elongate body to pass through the valve. In some examples, the valve is configured to obstruct at least a portion of the opening when the elongate body is withdrawn from the neck portion of the expandable metallic implant. In some examples, the valve includes at least one of uni-leaflet valve, a bi-leaflet valve, a tri-leaflet valve, a four-leaflet valve, a duckbill valve, a cross-slit valve, a dome valve, or a disc valve. In some examples, at least one of an inflow portion or an outflow portion of the valve is positioned to face the proximally facing opening of the expandable metallic implant. In some examples, the balloon is semi-compliant. In some examples, the semi-compliant balloon includes a compliant polymer. In some examples, one wall of the semi-compliant balloon has a wall thickness between about 3 microns and about 30 microns. In some examples, a length of the inflated balloon is greater than a length of the expandable metallic implant in the expanded configuration. In some examples, the guidewire lumen is configured to allow for passage of a guidewire and, when the guidewire is not within the guidewire lumen, the guidewire lumen is configured to allow for passage of one or more coil expandable implants. In some examples, the elongate body includes a radiopaque marker band near the distal end, and a more proximal marker band configured to assist in placing or detaching coil expandable implants. In some examples, the elongate body includes an inner shaft and an outer shaft, wherein a proximal tail of the balloon is joined to the outer shaft, a distal tail of the balloon is joined to the inner shaft, a fluid path of the inflation lumen passes between an outer surface of the inner shaft and an inner surface of the outer shaft, and the inner shaft passes through the neck of the expandable metallic implant. In some examples, a diameter of a lumen including at least one of the inflation lumen or the guidewire lumen is smaller at the distal end of the elongate body and larger at the proximal end of the elongate body, and wherein the lumen is configured to receive a guidewire to allow for balloon inflation. In some examples, the expandable metallic implant has a pleated and folded configuration when in the unexpanded configuration. In some examples, the balloon and the expandable metallic implant are pleated together into wings. In some examples, the proximal portion of the wings include two layers of balloon and do not include layers of expandable metallic implant. In some examples, the two layers of balloon are external layers. In some examples, the distal portion of the wings include two layers of expandable metallic implant and two layers of balloon. In some examples, the two layers of balloon are internal layers, and the two layers of expandable metallic implant are outer layers. In some examples, the wings are folded around a central axis.

In some examples, a system for obstructing or reducing flow of blood in an artery or vein in a patient can include: a catheter including: a body including a proximal portion and a distal portion; a lumen extending from the proximal portion to the distal portion of the body; and a balloon configured to be positioned at the distal portion of the body in fluid communication with the lumen; and an expandable implant configured to be carried by the catheter to an implantation site in an artery or vein in a patient and configured to expand from an unexpanded configuration to an expanded configuration that provides for an obstruction of flow of blood at the implantation site.

In some examples, the expandable implant includes an electroformed gold wall. In some examples, the electroformed gold wall is solid and lacks fenestrations. In some examples, the expandable implant includes flaps configured to extend radially outward from a wall of the expandable implant in the expanded configuration. In some examples, the flaps are at a proximal end of the expandable implant. In some examples, the expandable implant and the balloon are pleated and folded together in the unexpanded configuration. In some examples, the expandable implant includes a valve configured to prevent blood distal to the expandable implant from entering the expandable implant. In some examples, the valve is configured to allow a wire to pass through the expandable implant from a proximal side of the expandable implant.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1A is a side planar view of an example of an expanded expandable metallic implant having a seal zone, a transition zone and a neck with its overall geometric dimensions defined.

FIG. 1B is an end planar view of an example of an expanded expandable metallic implant having a seal zone, a transition zone and a neck with its overall geometric dimensions defined, wherein the observer is viewing from a distal to a proximal perspective.

FIG. 1C is an end planar view of an example of an expanded expandable metallic implant having a seal zone, a transition zone and a neck with its overall geometric dimensions defined, wherein the observer is viewing from a proximal to a distal perspective and can see into the central hollow region of an expandable metallic implant.

FIG. 1D is an end planar view of the example of an expanded expandable metallic implant shown in FIG. 1C showing the exterior surface of the core portion of the expandable metallic implant and the interior surface of the core portion of the expandable metallic implant.

FIG. 1E is a side view of an example of an expanded expandable metallic implant having a neck, nose cone, and guidewire segment, with overall geometric dimensions defined.

FIG. 2A is a side view of an example of a balloon catheter with an inflated balloon shown separated from an example of an expanded expandable metallic implant, with overall geometric dimensions defined.

FIG. 2B is a side view of the distal portion of an example of a balloon catheter with an inflated balloon, with overall geometric dimensions defined. There is a marker band present in the lumen of the distal neck of the balloon and a marker band present in the wall of the distal portion of the elongate body.

FIG. 3A is a side view of an example of an expandable metallic implant device with a folded and pleated expandable metallic implant and balloon assembly.

FIG. 3B is a perspective view of an example of an expandable metallic implant device with a folded and pleated expandable metallic implant and balloon assembly.

FIGS. 4A and 4B are side and perspective views of an example of expandable metallic implant device with an expanded expandable metallic implant and balloon assembly.

FIGS. 5A-G show an example of an expandable metallic implant device with a folded and pleated expandable metallic implant and balloon assembly, with cross-sectional views at locations B-G. The individual cross sections may not be shown equivalent in scale.

FIG. 6A is a cross-sectional view of a folded balloon of a balloon catheter with four balloon wings.

FIG. 6B is a cross-sectional view of the folded balloon of a balloon catheter with four balloon wings of FIG. 6A with a folded and pleated expandable metallic implant over the folded ballon. FIG. 6B also shows a straight portion of a wing of the expandable metallic implant and a curved portion of the wing of the expandable metallic implant.

FIG. 7A is a side view of an example of an expanded expandable metallic implant having a seal zone, a transition zone, and a neck, with its overall geometric dimensions defined.

FIG. 7B is side view of an example of an expanded expandable metallic implant having a proximal zone, a seal zone, a transition zone, and a neck, with its overall geometric dimensions defined. The proximal zone can also be a balloon retention region.

FIG. 7C is side view of an example of an expanded expandable metallic implant having a seal zone, a transition zone, and a neck, with its overall geometric dimensions defined. This example of expanded expandable metallic implant can have a balloon retention region between the seal zone and the transition zone.

FIG. 7D is side view of an example of an expanded expandable metallic implant having a seal zone, a transition zone, and a neck. This example of expanded expandable metallic implant can have a balloon retention region between the seal zone and the transition zone.

FIG. 7E is a side view of an example of an expanded expandable metallic implant having a seal zone, a transition zone, and a neck. This example of the expanded expandable metallic implant can have a balloon retention region within the seal zone.

FIG. 7F is a side view of an example of an expanded expandable metallic implant having a seal zone, a transition zone. The transition zone can be rounded.

FIG. 7G is a side view of an example of an expanded expandable metallic implant having a seal zone, a transition zone. This example can lack a neck. The transition zone can be flat.

FIG. 7H is a side view of an example of an expanded expandable metallic implant having a seal zone, a transition zone, and a neck. The transition zone can be flat.

FIG. 8A is a side view of an example of an expanded expandable metallic implant having a seal zone, a transition zone, and a neck.

FIG. 8B is a side view of an example of an expanded expandable metallic implant having a seal zone, a transition zone, and a neck. The proximal end of this expanded expandable metallic implant can have a fixation region with pointed flaps.

FIG. 9A is a side view of an example of an expanded expandable metallic implant having a seal zone, a transition zone, and a neck. The proximal end of this expanded expandable metallic implant can have a fixation region with pointed flaps.

FIG. 9B is a side view of an example of an expanded expandable metallic implant having a seal zone, a transition zone, and a neck, with its overall geometric dimensions defined. The proximal end of this expanded expandable metallic implant can have a fixation region with pointed flaps.

FIG. 9C is a side view of an example of an expanded expandable metallic implant having a seal zone, a transition zone, and a neck, with its overall geometric dimensions defined. The proximal end of this expanded expandable metallic implant can have a fixation region with rounded flaps.

FIG. 9D is a side view of an example of an expanded expandable metallic implant having a seal zone, a transition zone, and a neck, with its overall geometric dimensions defined. The proximal end of this expanded expandable metallic implant can have a fixation region with stalk-and-bud shaped flaps.

FIG. 10A-D show examples of an expanded expandable metallic implant having a seal zone, a transition zone, and a neck. Certain examples of expanded expandable metallic implant have a fixation region within the seal zone. In this example, the fixation region comprises pointed flaps with a proximal-distal alignment within a square flap window.

FIG. 11A is a side view of an example of an expanded expandable metallic implant having a seal zone, a transition zone, and a neck. This example of expanded expandable metallic implant can have a fixation region within the seal zone. The fixation region can have one row of pointed flaps with a proximal-distal alignment within a square flap window.

FIG. 11B is a side view of an example of an expanded expandable metallic implant having a seal zone, a transition zone, and a neck. This example of expanded expandable metallic implant can have a fixation region within the seal zone. The fixation region comprises one row of pointed flaps with a distal-proximal alignment within a square flap window.

FIG. 11C is a side view of an example of an expanded expandable metallic implant having a seal zone, a transition zone, and a neck. This example of expanded expandable metallic implant can have a fixation region within the seal zone. The fixation region can have two rows of pointed flaps, one with a distal-proximal alignment within a square flap window and the other with a proximal-distal alignment within a square flap window.

FIG. 11D is a side view of an example of an expanded expandable metallic implant can have a seal zone, a transition zone, and a neck. This example of expanded expandable metallic implant can have a fixation region within the seal zone. The fixation region can have one row of pointed flaps, wherein some flaps have a distal-proximal alignment within a square flap window, and other flaps have a proximal-distal alignment within a square flap window.

FIG. 12A is a planar view of a pointed flap of an expanded expandable metallic implant with a square flap window.

FIG. 12B is a planar view of a pointed flap of an expanded expandable metallic implant with a rectangular flap window that is wider than tall.

FIG. 12C is a planar view of a pointed flap of an expanded expandable metallic implant with a rectangular flap window that is taller than wide.

FIG. 12D is a planar view of a pointed flap of an expanded expandable metallic implant with a rectangular flap window that is taller than wide.

FIG. 12E is a planar view of a pointed flap of an expanded expandable metallic implant with a triangular flap window.

FIG. 13A is a side view of the distal end of one example of an expandable metallic implant device with folded and pleated expandable metallic implant and balloon assembly, with overall geometric dimensions defined.

FIG. 13B is a side view of the distal end of the example of the expandable metallic implant device shown in FIG. 13A after inflation of the balloon showing an inflated balloon and an expanded expandable metallic implant and an expanded expandable metallic implant and balloon assembly. This example of an expanded expandable metallic implant can have a proximal zone, a seal zone, a transition zone, and a neck. The proximal zone can have a balloon retention region and a fixation region. The fixation region can have one row of pointed flaps with a proximal-distal alignment within a rectangular flap window.

FIG. 13C is a side view of the distal end of the example of the expandable metallic implant device shown in FIG. 13B after over-inflation of the balloon showing an over-inflated balloon and flaps with free ends that are raised from the surface of the expanded expandable metallic implant.

FIG. 13D is a side view of the distal end of the example of the expandable metallic implant device shown in FIG. 13C after deflation or collapse of the balloon showing a deflated or collapsed balloon.

FIG. 13E is a side view of the distal end of the example of the expandable metallic implant device shown in FIG. 13D after separation of the deflated or collapsed balloon and the expanded expandable metallic implant.

FIG. 14 is a magnified, angled perspective view of the distal end of the example of the expandable metallic implant device shown in FIG. 13C.

FIG. 15A is a side view of the distal end of an alternative example of an expandable metallic implant device with a folded and pleated expandable metallic implant and balloon assembly.

FIG. 15B is a side view of the distal end of the example of the expandable metallic implant device shown in FIG. 15A after inflation of the balloon showing an expanded expandable metallic implant and balloon assembly. This example of an expanded expandable metallic implant has a proximal zone, a seal zone, a transition zone, and a neck. The proximal zone comprises a fixation region with one row of square flaps on the proximal end of the expanded expandable metallic implant wherein the flaps have a distal-proximal alignment. This example also comprises a seal zone with a balloon retention region.

FIG. 15C is a side view of the distal end of the example of the expandable metallic implant device shown in FIG. 15B after over-inflation of the balloon showing an over-inflated balloon and flaps with free ends that are raised from the surface of the expanded expandable metallic implant.

FIG. 15D is a side view of the distal end of the example of the expandable metallic implant device shown in FIG. 15C after deflation or collapse of the balloon showing a deflated or collapsed balloon.

FIG. 15E is a side view of the distal end of the example of the expandable metallic implant device shown in FIG. 15D after separation of the deflated or collapsed balloon and the expanded expandable metallic implant.

FIG. 16A is a planar view of a pointed flap of an expanded expandable metallic implant with a linear hinge region at the base of the flap and within a square flap window.

FIG. 16B is a planar view of a pointed flap of an expanded expandable metallic implant with a linear hinge region at the base of the flap and within a rectangular flap window with rounded edges.

FIG. 16C is a planar view of a pointed flap of an expanded expandable metallic implant with a linear hinge region at the base of the flap and within a rounded window.

FIG. 17A is an end planar view of an example of an expanded expandable metallic implant having a single structural metal layer.

FIG. 17B is an end planar view of an example of an expanded expandable metallic implant having a structural metal layer as an inner layer and a functional metal layer as an outer layer.

FIG. 17C is an end planar view of an example of an expanded expandable metallic implant having a structural metal layer as a middle layer, a functional metal layer as an outer layer, and a functional metal layer as an inner layer.

FIG. 17D is an end planar view of an example of an expanded expandable metallic implant having a structural metal layer as an inner layer and a polymer layer as an outer layer.

FIG. 17E is an end planar view of an example of an expanded expandable metallic implant having a structural metal layer as a middle layer, a polymer layer as an outer layer, and a polymer layer as in inner layer.

FIG. 17F is an end planar view of an example of an expanded expandable metallic implant having a structural metal layer as an outer layer, a polymer layer as an inner layer,

FIG. 18A is a planar view of a guide catheter or guide sheath and a Tuohy Borst adaptor, wherein the guide catheter or guide sheath and a Tuohy Borst adaptor are separated.

FIG. 18B is a planar view of a guide catheter or guide sheath and a Tuohy Borst adaptor, wherein the guide catheter or guide sheath and a Tuohy Borst adaptor are joined, wherein the Tuohy Borst adaptor is configured for flushing the lumen of the guide catheter or guide sheath.

FIG. 19A is a planar view of an assembly of a guide catheter or guide sheath, first Tuohy Borst adaptor, intermediate catheter, second Tuohy Borst adaptor, and obturator, wherein the first Tuohy Borst adaptor is configured for flushing the lumen of the guide catheter or guide sheath and the second Tuohy Borst adaptor is configured for flushing the lumen of the intermediate catheter.

FIG. 19B is a planar view of an assembly of a guide catheter or guide sheath, first Tuohy Borst adaptor, intermediate catheter, second Tuohy Borst adaptor, selective catheter, and guidewire, wherein the first Tuohy Borst adaptor is configured for flushing the lumen of the guide catheter or guide sheath and the second Tuohy Borst adaptor is configured for flushing the lumen of the intermediate catheter.

FIG. 19C is a planar view of an assembly of a guide catheter or guide sheath, Tuohy Borst adaptor, intermediate catheter, selective catheter, and guidewire, wherein the Tuohy Borst adaptor is configured for flushing the lumen of the guide catheter or guide sheath.

FIG. 20 is a planar view of an example of a system comprising a guide sheath, Tuohy first Borst adaptor, intermediate catheter, second Borst adaptor, selective catheter, and a guidewire.

FIG. 21 is a planar view of an example of a system comprising a guide sheath, Tuohy first Borst adaptor, expandable metallic implant device, a coiling or delivery catheter, and a coil expandable implant device.

FIG. 22A is a cross-sectional view of an example of an assembly of a guide catheter or guide sheath, intermediate catheter, obturator and guidewire.

FIG. 22B is a cross-sectional view of an example of an assembly of a guide catheter or guide sheath, intermediate catheter, selective catheter, and guidewire.

FIG. 23A is a cross-sectional view of an example of an assembly of a guide catheter or guide sheath, expandable metallic implant device, coiling or delivery catheter, and coil expandable implant device, shown with the coil expandable implant of the coil expandable implant device passing through the lumen of the coiling or delivery catheter.

FIG. 23B is a cross-sectional view of an example of an assembly of a guide catheter or guide sheath, expandable metallic implant device, coiling or delivery catheter, and coil expandable implant device, shown with the elongate body of the coil expandable implant device passing through the lumen of the coiling or delivery catheter.

FIG. 24A is a cross-sectional view of an example of a guidewire segment of an expandable metallic implant.

FIG. 24B is a cross-sectional view of an example of a guidewire.

FIG. 24C is a cross-sectional view of an example of a coil expandable implant device.

FIG. 25A is a planar view of an example of an intermediate catheter.

FIG. 25B is a planar view of an example of a selective catheter.

FIG. 26A is a planar side view of a preferred example of an expanded expandable metallic implant having a proximal zone for balloon retention, a seal zone, a transition zone, proximal flaps (which are angled or tilted) and a neck. In this example the flaps are present in the proximal zone. In this example the largest diameter of the neck is larger than the diameter of the neck transition region.

FIG. 26B is an end planar view of a preferred example of the expanded expandable metallic implant from FIG. 26A, wherein the observer is viewing from a proximal to a distal perspective and can see into the central hollow region of the expandable metallic implant, as well as the proximal flaps.

FIG. 27A is a perspective view of a preferred example of an expanded expandable metallic implant having a proximal zone for balloon retention, a seal zone, a transition zone, proximal flaps (which are angled or tilted), a neck, and a valve. In this example the flaps are present in the proximal zone. In this example the largest diameter of the neck is larger than the diameter of the neck transition region.

FIG. 27B is a perspective view of a preferred example of the expanded expandable metallic implant from FIG. 27A showing the proximal opening into the central hollow region of the expandable metallic implant, and the proximal angled or tilted flaps.

FIG. 27C is a perspective view of the expanded expandable metallic implant from FIG. 27A wherein the expanded expandable metallic implant is cut halfway through the longitudinal axis so as to provide an interior view of the expanded expandable metallic implant. In this view a valve metal ring/neck tubular segment is shown. In this view the duckbill valve is seen to be a duckbill valve oriented to reduce or obstruct blood from coming from proximal (from the proximal opening of the central hollow region of the expandable metallic implant) to distal (to the neck opening of the expandable metallic implant). In this view there is a valve metal ring interposed between the valve and the neck of the expanded expandable metallic implant. In this view, the single valve annular grove can be seen.

FIG. 28A is a perspective view of a preferred example of an expanded expandable metallic implant wherein the dimensions of a preferred example of the expanded expandable metallic implant are shown for this example wherein the outer diameter of the seal zone is 6 mm. This example of an expanded expandable metallic implant has a proximal zone for balloon retention, a seal zone, a transition zone, and a neck. In this example, the largest diameter of the neck is larger than the diameter of the neck transition region. In this example, the neck lumen is shown.

FIG. 28B is a perspective view of a portion of the expanded expandable metallic implant from FIG. 28A focused on the neck transition region wherein the expanded expandable metallic implant is cut halfway through the longitudinal axis so as to provide an interior view of the expanded expandable metallic implant. The minimum diameter of the expanded expandable metallic implant is shown in the region of the neck transition region.

FIG. 28C is an end view of the of the expanded expandable metallic implant from FIG. 28A wherein the observer is viewing from a distal to a proximal perspective and can see into the neck lumen.

FIG. 29A is a planar side view of a preferred example of an inflated balloon component of an expandable metallic implant device for expanding the expandable metallic implant shown in FIG. 23A-C wherein the wherein the outer diameter of the intermediate region of the balloon is 6 mm. In this view, the proximal neck and the distal neck of the balloon is shown. For this preferred example of an inflated balloon the distal region of the balloon is longer than the proximal region of the balloon.

FIG. 29B is an end view of a preferred example of an inflated balloon wherein the observer is viewing from a distal to a proximal perspective and can see into the distal neck opening of the balloon.

FIG. 30A is a perspective view of a preferred example of a hub of a balloon catheter of an over-the-guidewire version of an expandable metallic implant device showing the inflation port and the guidewire port.

FIG. 30B is a cutaway perspective view of a preferred example of a hub of the over-the-guidewire system from FIG. 30A showing the inflation lumen between the outer diameter of the inner shaft and the inner diameter of the outer shaft, which is in fluid connection with the inflation port of the hub. This preferred example shows three layers of strain relief, an inner layer, a middle layer, and an outer layer.

FIG. 31A is a planar cutaway of a preferred example of a hub of a balloon catheter of an over-the-guidewire version of an expandable metallic implant device configured to advance over a 0.014″ diameter guidewire showing the inflation port and the guidewire port.

This example also shows a narrowing of the lumen of the guidewire port from the proximal opening to the junction wherein the inner shaft is joined for guiding guidewires and coil expandable implant devices into the lumen of the inner shaft.

FIG. 31B is a face on view of a preferred example of the hub from FIG. 31A from a distal to a proximal perspective providing a view into the lumen of the cavity wherein the inner shaft and outer shafts are inserted and bonded.

FIG. 31C is a detail view of a preferred example of the hub from FIG. 31A focused on the geometry of the hub in the region between the guidewire port and where the inner shaft joins the hub.

FIG. 31D is a detail view of a preferred example of the hub from FIG. 31A focused on the Luer fitting of the guidewire port.

FIG. 32 is a side view of an over-the-guidewire example of a balloon catheter with an inflated balloon shown separated from an example of an expanded expandable metallic implant.

FIGS. 33A and 33B are side and perspective views of an over-the-guidewire example of expandable metallic implant device with an expanded expandable metallic implant and balloon assembly.

FIG. 34A is a side view of the over-the-guidewire example of an expandable metallic implant device with a folded and pleated expandable metallic implant and balloon assembly.

FIG. 34B is a perspective view of the over-the-guidewire example of an expandable metallic implant device with a folded and pleated expandable metallic implant and balloon assembly.

FIG. 35A is a side view of the over-the-guidewire example of an expandable metallic implant device with a folded and pleated expandable metallic implant and balloon assembly.

FIG. 35 B-I shows cross-sectional views of the over-the-guidewire example of FIG. 35A an expandable metallic implant device of at various locations, as shown. The individual cross sections may not be shown equivalent in scale.

FIG. 36A is a perspective view of an over-the-guidewire expandable metallic implant with the distal shaft of the balloon catheter positioned through the lumen of neck of the expandable metallic implant and through the valve.

FIG. 36B is a perspective view of the over-the-guidewire expandable metallic implant from FIG. 36A with the distal shaft of the balloon catheter removed from the lumen of the neck of the implant and from the valve.

FIG. 36C is a perspective view of an assembly of a duckbill valve, valve metal ring, and a distal portion of an inner shaft of a balloon catheter wherein a portion of the inner shaft positioned within the valve, displacing the valve leaflets open.

FIG. 36D is a perspective view of an assembly of the duckbill valve and valve metal ring from FIG. 36C, wherein distal portion of an inner shaft has been removed and showing the valve leaflets closed.

FIG. 37A is a perspective outflow view of an example of a valve that can be fitted into the neck of the expandable metallic implant, showing the two leaflets.

FIG. 37B is a perspective view of the valve from FIG. 37A wherein the perspective is from the inflow aspect

FIG. 37C is a cutaway through the midline of the valve from FIG. 37A.

FIG. 38A is a perspective view of a valve.

FIG. 38B is a perspective view of the valve of FIG. 38A.

FIG. 38C is a perspective view of the valve of FIG. 38A with a metal ring positioned over a portion of the valve.

FIG. 39A-C are perspective views one example of a valve fitted with a metal ring to add additional support to the valve. FIG. 39A shows the valve having an annular grove sized to fit a valve metal ring between two flanges. FIG. 39B shows the valve overmolded with a metallic ring, having several holes that enables the valve material to integrate with the metal ring. FIG. 39C shows a separate valve metal ring placed between two flanges and secured with glue.

FIG. 40A-D shows variations of valves that may be used, ranging from the monocusp (FIG. 40A), the two leaflet duckbill valve (FIG. 40B), a three-leaflet valve (FIG. 40C) and a four-leaflet cross-slit valve (FIG. 40D).

FIG. 41A-C is an alternative example of the expandable metallic implant with two discrete structural (outer) metal layer segments joined to a flexible inner polymer layer. (FIG. 41A) and (FIG. 41B) are perspective views of the example from the proximal and distal aspects and (FIG. 41C) is a partial cutaway showing the structural (outer) metal layer of the two segments and the flexible inner polymer layer. For this example, the structural (outer) metal layer of the distal segment is continuous with the metal layer of the neck.

FIG. 42A is a distal aspect perspective view of an example of the expandable metallic implant having flaps with holes in order to enable tissue ingrowth from the wall of the vessel.

FIG. 42B is a proximal aspect perspective view of an example of the expandable metallic having anchoring flaps with holes in order to enable better tissue ingrowth from the wall of the vessel.

FIG. 43A is a perspective proximal aspect view of an example of a secondary folded and pleated variable size expandable metallic implant wherein a portion of the wall is folded and is pleated over itself such that it can be expanded further after an initial expansion.

FIG. 43B is a perspective proximal aspect view of the mandrel that is used for the manufacture of the secondary folded and pleated variable size expandable metallic implant of FIG. 43A.

FIG. 43C is a cross-sectional view the secondary folded and pleated variable size expandable metallic implant of FIG. 43A.

FIG. 43D is a cross-sectional view of the mandrel that is used for the manufacture of the secondary folded and pleated variable size expandable metallic implant of FIG. 43C.

FIG. 43E is a perspective distal aspect view of an example of a secondary folded and pleated variable size expandable metallic implant wherein a portion of the wall is folded and is pleated over itself such that it can be expanded to a larger diameter after an initial expansion.

FIG. 44A is a perspective distal aspect view of an example of a slotted variable size expandable metallic implant showing a longitudinal discontinuity such that it can be expanded to a larger diameter further after an initial expansion.

FIG. 44B is a perspective proximal aspect view of a slotted variable size expandable metallic implant showing a longitudinal discontinuity such that it can be expanded to a larger diameter further after an initial expansion.

FIG. 45A is a perspective distal aspect view of an expandable metallic implant neck showing a valve and a valve metal ring.

FIG. 45B is a perspective proximal aspect view of the expandable metallic implant neck having an elongated distal section that is bent over the valve distally by pressing it into a concave form.

FIG. 45C is a perspective distal aspect view of the expandable metallic implant FIG. 45B wherein the elongated distal neck section has been bent over the valve distally by pressing it into a concave form, thus forming a curved or convex distal surface.

FIG. 46A-G shows a method of placing an expanded expandable metallic implant using an over-the-guidewire expandable metallic implant device. In FIG. 46A, a guidewire is placed in the target vessel segment. In FIG. 46B, a folded and pleated over-the-guidewire expandable metallic implant device is advanced until the folded and pleated expandable metallic implant is positioned in the target vessel segment. In FIG. 46C, the folded and pleated semi-compliant balloon of the expandable metallic implant device is inflated, expanding the expandable metallic implant device. In FIG. 46D, the semi-compliant balloon is overinflated, tilting or angling the flaps of the expanded expandable metallic implant. FIG. 46E shows deflation or collapse of the ballon and engagement of the flaps with the vessel wall. FIG. 46E shows removal of the balloon catheter from the expanded expandable metallic implant. FIG. 46E shows thrombus formation proximal and distal to the expanded expandable metallic implant and an obstruction or reduction of flow in the target vessel segment.

FIG. 47A is a cross-sectional illustration of a blood vessel with atheroma and an irregular lumen.

FIG. 47B is the cross-sectional illustration of FIG. 47A with a vascular plug expandable implant positioned adjacent to the atheroma and an incomplete fit between the vascular plug expandable implant and the vessel lumen

FIG. 47C is the cross-sectional illustration of FIG. 47A with an expanded expandable metallic implant positioned adjacent to atheroma with compression of the atheroma, circularization of the vessel lumen and a good fit between the expanded expandable metallic implant and the circularized vessel lumen.

FIG. 48A is a cross-sectional view of an expanded expandable metallic implant positioned over an inflated balloon of a balloon catheter. FIG. 48B is a cross-sectional view of

FIG. 48A wherein the expandable metallic implant and the balloon have been folded together into three wings. FIG. 48C is a cross-sectional view of FIG. 48B wherein the folds of the expandable metallic implant and the balloon have been pleated around the inner shaft of the expandable metallic implant device.

FIG. 49A shows a folded and pleated expandable metallic implant device. FIG. 49B shows inflation of the balloon in FIG. 49A and expansion the expandable metallic implant. FIG. 49C shows over-inflation of the balloon in FIG. 49B and tilting or angling of the proximal flaps. FIG. 49D shows the expandable metallic implant of FIG. 49C after deflation or collapse of the balloon and removal of the balloon catheter.

FIG. 50A is a perspective view of a photograph of the folded expandable metallic implant and balloon of a balloon catheter with three balloon wings, wherein the expandable metallic implant is external to the balloon. FIG. 50B is a perspective view of a photograph of the folded expandable metallic implant and balloon positioned inside the pleat head of the pleating machine.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes, or which is essential to practicing the systems, devices, and/or methods disclosed herein.

Parts, components, features, and/or elements of the systems and devices described herein that can function the same or similarly across various implementations are identified using similar reference numerals. Differences between the various implementations are discussed herein. Reference numerals are also provided for certain elements that are described for context or related to further examples. Although these elements may not be illustrated, reference numerals are provided for convenience to identify these elements.

Implementations of the present application relate to expandable metallic implant devices 1 for use in obstructing or reducing the flow of blood or other biological fluids in a human patient. In some examples, the devices include two separable components: 1) an expandable metallic implant 10 configured for permanent implantation in human patients; and 2) a balloon catheter 100.

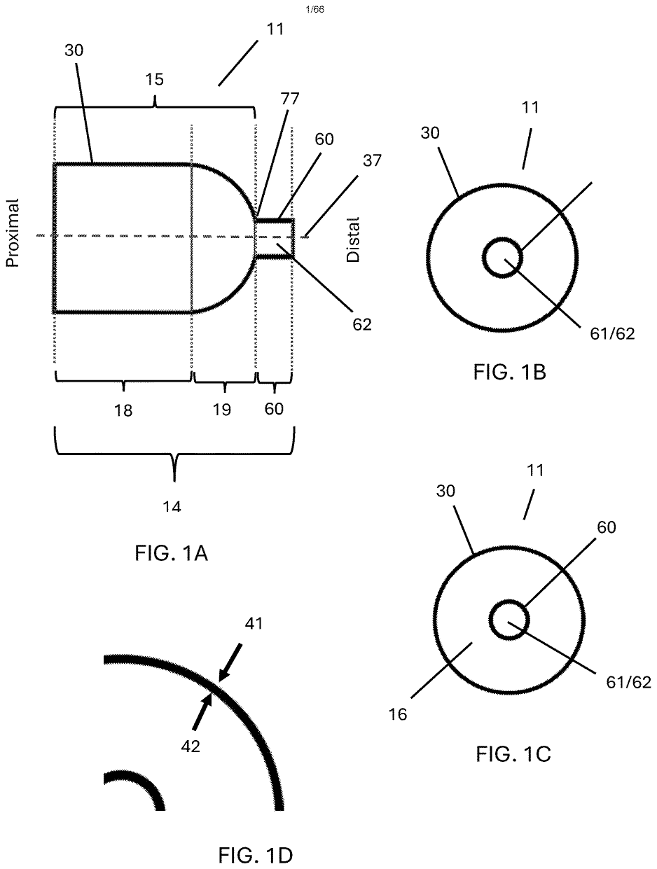

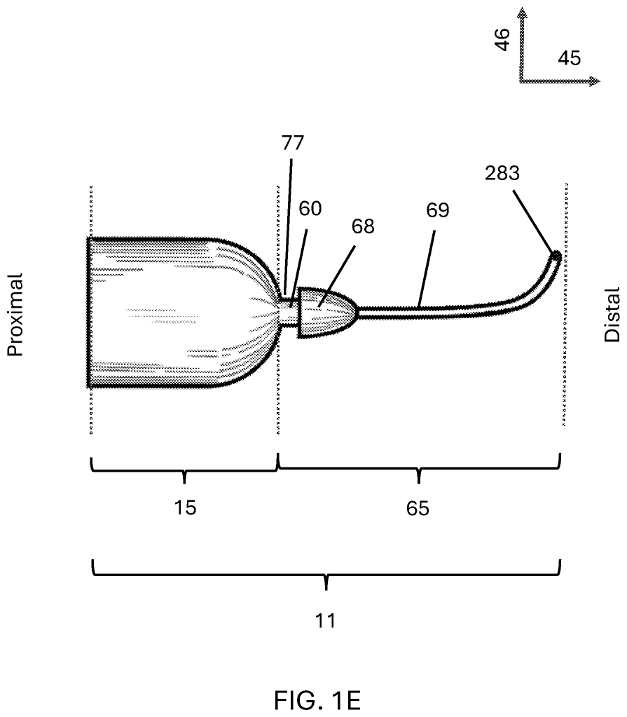

FIG. 1A is a side planar view of an example of an expanded expandable metallic implant 11 having a seal zone 18, a transition zone 19 and a neck 60. FIG. 1B is an end planar view of an example of an expanded expandable metallic implant 11 having a seal zone 18, a transition zone 19 and a neck 60, wherein the observer is viewing from a distal to a proximal perspective. FIG. 1C is an end planar view of an example of an expanded expandable metallic implant 11 having a seal zone 18, a transition zone 19 and a neck 60, wherein the observer is viewing from a proximal to a distal perspective and can see into the central hollow region 16 of an expanded expandable metallic implant 11. FIG. 1D is an end planar view of the example of an expanded expandable metallic implant 11 shown in FIG. 1C showing the exterior surface 41 of the core portion 14 of the expanded expandable metallic implant 11 and the interior surface 42 of the core portion 14 of the expanded expandable metallic implant 11. FIG. 1E is a side view of an example of an expanded expandable metallic implant 11 having a neck 60, nose cone 68, and guidewire segment 69.

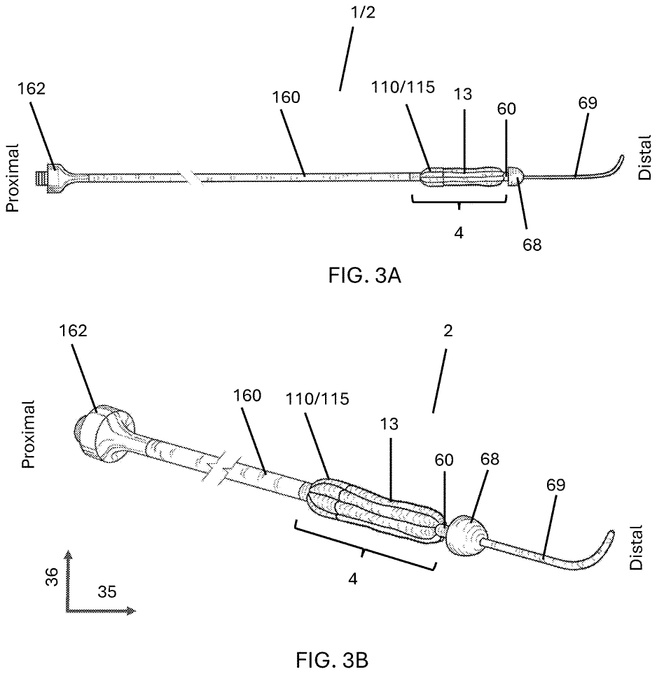

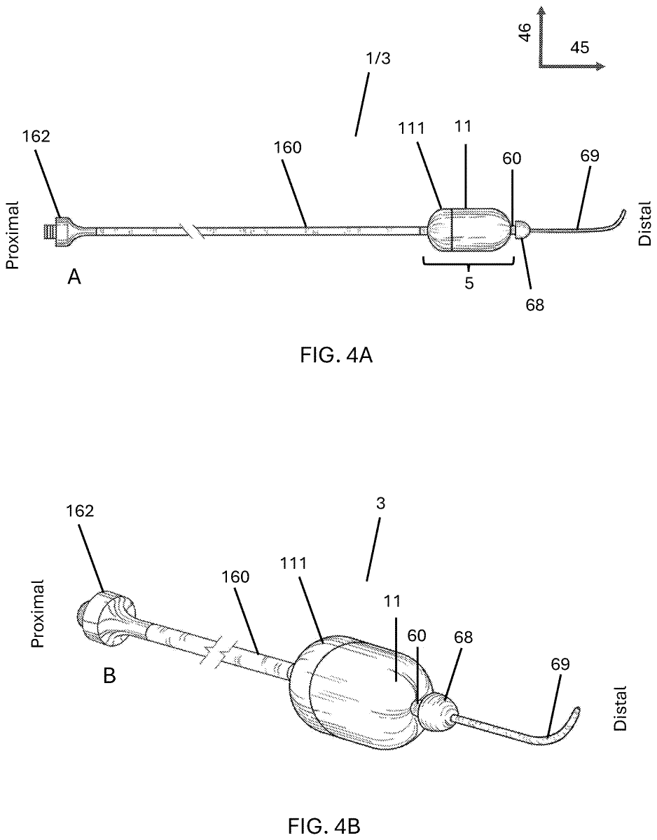

In some examples, the expandable metallic implant 10 can be configured for obstructing, occluding, embolizing, sealing, or reducing the flow of blood or other biological fluids in arteries, veins, aneurysms, parent vessels of saccular aneurysms, paravalvular leak pathways, biological conduits, or other blood-containing, fluid-containing, or biological spaces in human patients. In some examples, the expandable metallic implant 10 is configured to expand from a folded and pleated configuration, as shown in FIG. 3A-3B to an expanded configuration, as shown in FIG. 4A-4B. In some examples, the expandable metallic implant 10 is configured with a wall that is open on the proximal end and closed or obstructed on the distal end and wherein the wall continuously from the proximal end to the distal end. For example, the wall can lack fenestrations. Fenestrations can include grooves, cuts, or breaks.

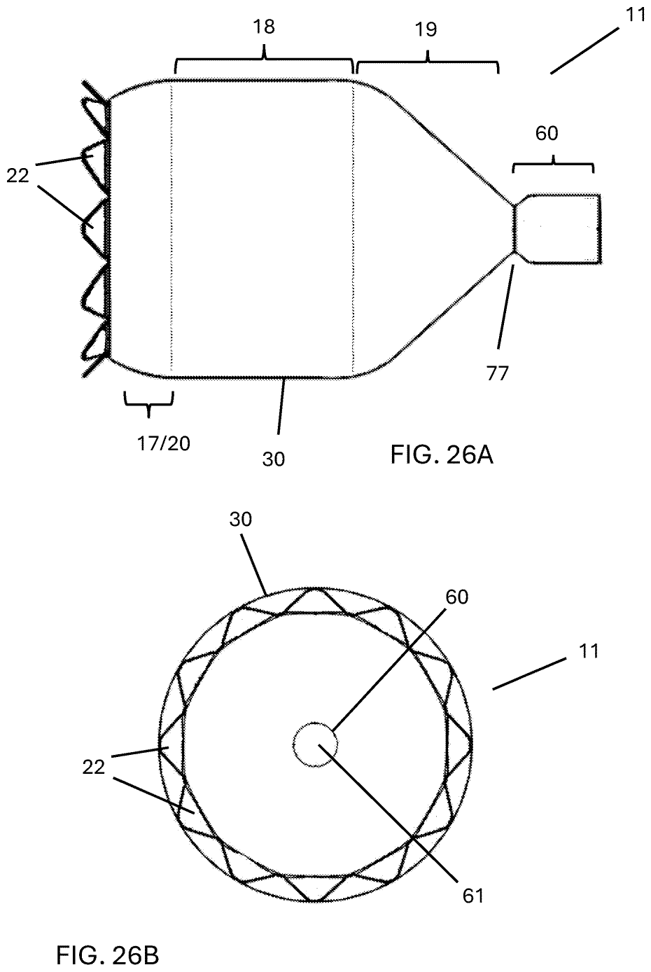

In some examples, the expandable metallic implant 10 can be comprised of: 1) a core portion 14; and optionally 2) additional neck components 66, and a neck transition zone 77 as shown in FIG. 26. In some examples, the core portion 14 is a unitary body comprises a seal zone 18 and a transition zone 19. In some examples, the core portion 14 further comprises a proximal zone 17, a neck 60, or both a proximal zone 17 and a neck 60. In some examples, the core portion 14 is formed by electroforming. In some examples, (FIG. 1E), the additional neck components 66 can comprise a neck tubular segment or structure 67, a nose cone 68, or a guidewire segment 69. In some examples, additional neck components 66 are formed by molding or machining. The proximal zone 17, seal zone 18, and transition zone 19 comprise the portion of the expandable metallic implant 10 that can be folded and pleated and combine to form an expandable portion 15 of the expandable metallic implant 10.

In some examples, the wall 30 of the expandable portion 15 of the expandable metallic implant 10 comprises at least one layer of metal with a thickness of at least 3 microns and wherein the wall 30 of the expandable portion 15 of the expandable metallic implant 10 can be folded and pleated with a balloon 110 for delivery, and expanded at the target location by injecting fluid into the interior space 120 of the balloon 110. In some examples, the thickness of the wall 30 of the expandable portion 15 of the expandable metallic implant 10 is configured to enable expansion in response to the injection of fluid into the interior space 120 of the balloon 110 at a pressure of 1, 2, 3, 4, 5, 6, 7, 8, 9, 10, 11, 12, 13, 14, 15, 16, 17, 18, 19, or 20 atmospheres. In some examples, the wall 30 of the core portion 14 of the expandable metallic implant 10 comprises a continuous layer of metal, excluding any openings in the neck 60. The proximal side of the expandable metallic implant 10 may be substantially circular. The proximal side of the expandable metallic implant 10 may be open or concave.

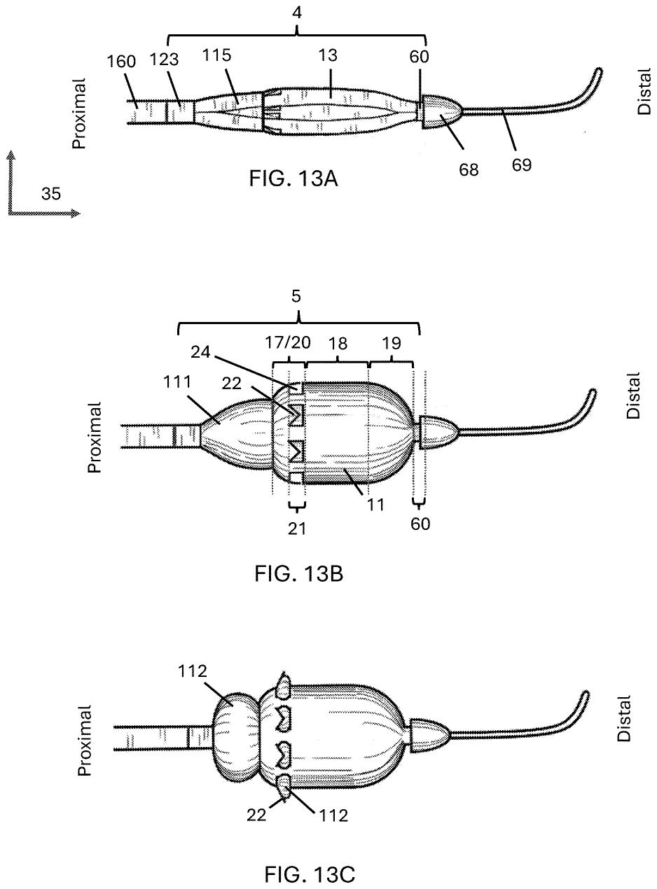

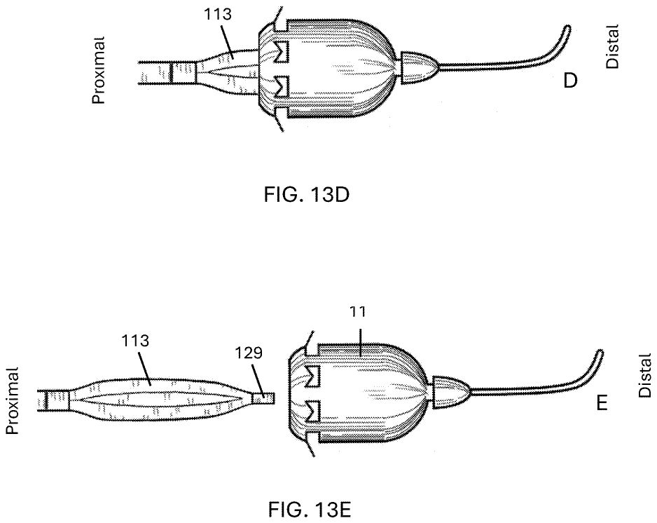

An expandable metallic implant 10 is a structure with an expandable portion 15 with a wall 30 comprising a material that can transition from a folded and pleated configuration to an expanded configuration, wherein the wall 30 of the expandable portion 15 of the expandable metallic implant 10 can be folded and pleated together with a balloon 110 and expanded by the inflation of the folded and pleated balloon 115 by the injection of a fluid into the interior space 120 of the balloon 110 under pressure, as shown in FIGS. 13A-C and 15A-C. This form of expandable metallic implant 10 can be delivered to a desired location in a human patient in a folded and pleated form using the balloon catheter 100, expanded, and separated from the balloon catheter 100 in a manner that allows the expanded expandable metallic implant 11 to remain in the patient while the balloon catheter 100 is removed from the patient.

The wall 30 of the expandable portion 15 of the expandable metallic implant 10, the wall 30 of the core portion 14 of an expandable metallic implant 10, or the wall 30 of the entire expandable metallic implant 10 may comprise at least one metal. The wall 30 of the expandable portion 15 of the expandable metallic implant 10, the wall 30 of the core portion 14 of an expandable metallic implant 10, or the wall 30 of the entire expandable metallic implant 10 may comprise gold, platinum, or combinations or alloys thereof. The wall 30 of the expandable portion 15 of the expandable metallic implant 10, the wall 30 of the core portion 14 of an expandable metallic implant 10, or the wall 30 of the entire expandable metallic implant 10 may comprise may further comprise additional metals, polymers, adhesives, and combinations thereof. Expandable metallic implants 10 possess sufficient strength to maintain an expanded or partially expanded configuration in vivo after separation from the balloon catheter 100, including sufficient strength to maintain an expanded or partially expanded configuration in vivo after separation from the balloon catheter 100 when no solid or semi-solid material, not derived from the patient, is present in the central hollow region 16 of the expanded expandable metallic implant 11.

In some examples, to expand a folded and pleated expandable metallic implant 13 of a folded and pleated expandable metallic implant device 2, fluid can be injected into the hub 162 of the balloon catheter 100, through the lumen 161 of the elongate body 160, through the proximal neck 123 of the balloon 110, and into the interior space 120 of the folded and pleated balloon 115. When fluid enters the interior space 120 of a folded and pleated balloon 115 under pressure (and the folded and pleated balloon 115 and folded and pleated expandable metallic implant 13 are not constrained by external forces) the folded and pleated balloon 115 can expand, which results in the expansion of the folded and pleated expandable metallic implant 13. Pleated and folded expandable metallic implants 15 can expand by an unfolding of the folded and pleated portion of the expandable portion 15 of the expandable metallic implant 10.

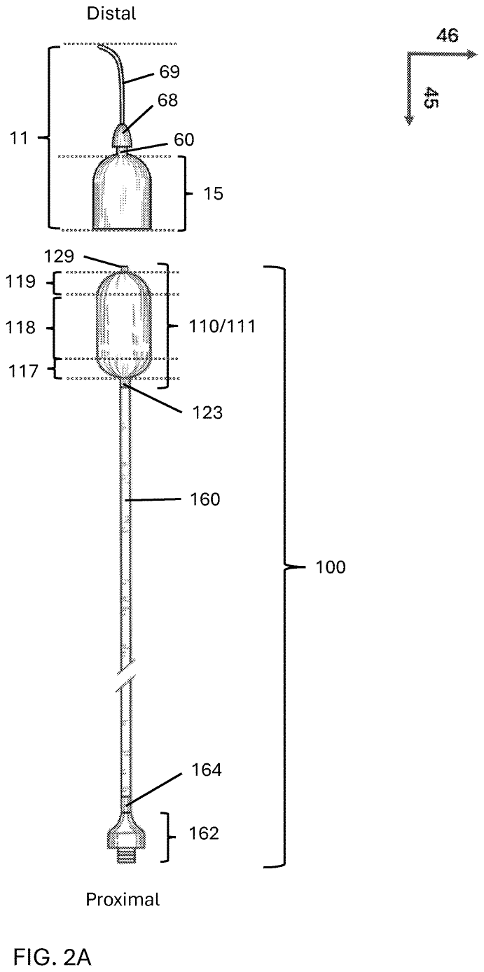

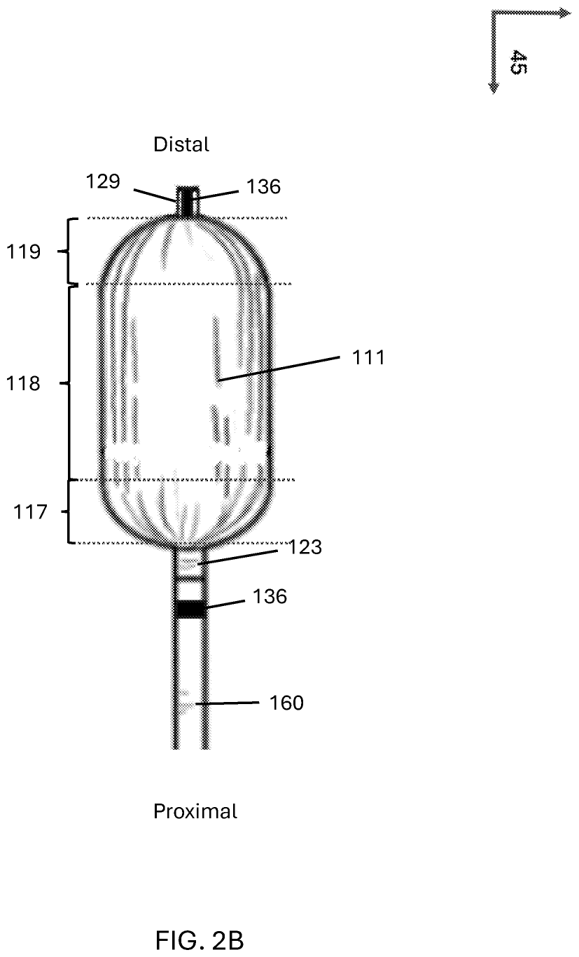

FIG. 2A is a perspective view of an example of a balloon catheter 100 with an inflated balloon 111 shown separated from an example of an expanded expandable metallic implant 11. FIG. 2B is a perspective view of the distal portion of an example of a balloon catheter 100 with an inflated balloon 111. There is a marker band 136 present in the lumen 131 of the distal neck 129 of the balloon 110 and a marker band 136 present in the wall 165 of the distal portion of the elongate body 160.

The balloon catheter 100 of expandable metallic implant devices are comprised of an elongate body 160 with a hub 162, and a balloon 110. In some examples, the balloon catheter 100 comprises a single marker band 163 that is radiopaque or conspicuous during fluoroscopy configured for identifying the tip region of the balloon catheter 100 during fluoroscopy. In some examples, the balloon catheter 100 comprises two marker bands 163 that are radiopaque or conspicuous during fluoroscopy configured for identifying the proximal 117 and distal 119 regions of the balloon 110 during fluoroscopy. In some examples, the balloon catheter 100 comprises a single marker band 163 that is radiopaque or conspicuous during fluoroscopy configured for identifying the tip region of the balloon catheter 100 during fluoroscopy and two marker bands 163 that are radiopaque or conspicuous during fluoroscopy configured for identifying the proximal 117 and distal 119 regions of the balloon 110 during fluoroscopy.

In some examples, (FIG. 4), the balloon catheter 100 can include an elongate body 160 comprising a hub 162. The hub 162 can be joined or bonded to the proximal end of the elongate body 160 and configured to couple with a syringe, inflation device, or other fluid source. The hub 162 can be configured to couple with a lumen 161 of elongate body 160 to allow for the passage of fluid to a balloon 110. The elongate body 160 may be joined or bonded to the inflatable portion 116 of the balloon 110, or to the proximal neck 123 and/or the distal neck 129 of the balloon 110. In another example, the elongate body 160 may be joined or bonded to one or more components of a proximal neck assembly and/or a distal neck assembly which are then joined or bonded to the balloon 110. In another example, the elongate body 160 may be joined or bonded to a neck bridging segment 135 which is joined or bonded to the balloon 110 or may be joined or bonded to a proximal neck joining structure and/or distal neck joining structure which is joined or bonded to the balloon 110, or combinations of joining or bonding thereof. In some examples, the balloon 110 comprises a proximal region 117, a distal region 119 generally opposite the proximal region 117, and an intermediate region 118 between the proximal 117 and distal region 119, and a first axis 45 extending proximal to distal between the proximal region 117 and the distal region 119.

In some examples, the lumen 161 of the elongate body 160 extends from the hub 162 to the distal end of the elongate body 160 and is configured to enable the passage of fluid from the hub 162, through the lumen 161 of the elongate body, optionally through a joining region comprising a proximal neck 123, proximal neck assembly, neck bridging segment 135, or proximal neck joining structure, or combination thereof), and into the interior space 120 of the balloon 110 to enable inflation and deflation or collapse of the balloon 110.

In some examples, the balloon catheter 100 is configured for: 1) delivering a folded and pleated expandable metallic implant 13 to the treatment site a patient; 2) expanding the folded and pleated expandable metallic implant 13 at the treatment site; and 3) detaching from the expanded expandable metallic implant 11 and being removed from the patient, wherein the expanded expandable metallic implant 11 remains in place in an expanded state after separation from the balloon catheter 100 and removal of the balloon catheter 100 from the patient's body. In some examples, the expanded expandable metallic implant 11 is hollow or comprises a central hollow region 16. In some examples, the inflated balloon 111 is configured to have a maximum diameter of between 2-50 mm when measured parallel to the second axis 46. In some examples, the inflatable portion 116 of the inflated balloon 111 is configured to have a maximum length of between 3-100 mm when measured parallel to the first axis 45. The volume of the inflated balloon 111 may range between 0.005 mL to 65 mL. In some examples, the overall length of the inflated balloon 111 is equal to or greater than the overall length of the expanded expandable metallic implant 11.

The balloon 110 is joined to the distal region of the elongate body 160 and configured for pleating and folding or compression, inflation or expansion, and deflation or collapse. In some examples, the balloon 110 comprises a proximal region 117, a distal region 119 generally opposite the proximal region 117, and an intermediate region 118 between the proximal region 117 and the distal region 119 configured, and a first axis 45 extending proximal to distal between the proximal region 117 and the distal region 119.

In some examples, the rated burst pressure of the balloon 110 is between 1-50 atmospheres, in the range of 3-20 atmospheres, or in the range of 3-10 atmospheres.

In some examples, the wall 137 of the balloon 110 comprises a polyethylene terephthalate, a nylon, or a material comprising block copolymers made of rigid polyamide blocks and soft polyether blocks, including Pebax, and combinations thereof. In some examples, after expansion to the nominal diameter, the balloon 110 is compliant or semi-compliant. In some examples, after expansion to the nominal diameter, the balloon 110 is semi-compliant or compliant when the pressure inside the balloon 110 is in a range between 1 and 20 atmospheres. In some examples, after expansion to the nominal diameter, the diameter of the inflated balloon 111 increases 5-10%, 11-15%, 16-20%, 21-25%, 26-30%, 31-35%, 36-40%, 41-45%, or 46-50% when inflated to a pressure of 5 atmospheres. In some examples, after expansion to the nominal diameter, the diameter of the inflated balloon 111 increases 5-10%, 11-15%, 16-20%, 21-25%, 26-30%, 31-35%, 36-40%, 41-45%, or 46-50% when inflated to a pressure of 10 atmospheres. In some examples, after expansion to the nominal diameter, the diameter of the inflated balloon 111 increases 5-10%, 11-15%, 16-20%, 21-25%, 26-30%, 31-35%, 36-40%, 41-45%, or 46-50% when inflated to a pressure of 15 atmospheres. In some examples, after expansion to the nominal diameter, the diameter of the inflated balloon 111 increases 5-10%, 11-15%, 16-20%, 21-25%, 26-30%, 31-35%, 36-40%, 41-45%, or 46-50% when inflated to a pressure of 20 atmospheres.

In some examples, the wall 137 of the balloon 110 may have a thickness of 3-100 microns, 5-30 microns, or 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47, 48, 49, or 50 microns. In some examples, the wall 137 of the balloon 110 comprises a layer of polyethylene terephthalate with a thickness of 3 microns, 4 microns, 5 microns, 6 microns, 7 microns, 8 microns, 9 microns, 10 microns, 11-15 microns, or 16-20 microns.

In some examples, the wall 137 of the balloon 110 comprises a single layer. For single layer examples of a balloon 110, the layer is a polymer layer 36. In some examples, the wall 137 of the balloon 110 comprises two or more layers. For examples of a multiple layer balloon 110, one layer is a polymer layer 36, and the one or more additional layers may be a layer of polymer, metal, or adhesive.

In some examples, a balloon 110 comprises a proximal neck 123 with a proximal neck opening 124 to enable fluid to pass from the elongate body 160 into the interior space 120 of the balloon 110. In some examples, the proximal neck 123 extends away from the interior space 120 of the balloon 110. In some examples, the proximal neck 123 extends into the interior space 120 of the balloon 110. The length of proximal neck 123 is between 1 mm and 20 mm, preferably with a length between 2 mm and 5 mm. The proximal neck opening 124 has a diameter between 0.5 mm and 5 mm. The thickness of the wall of the proximal neck 123 may be thicker, the same as the thickness, or thinner than the wall 137 of the inflatable portion 116 of the balloon 110. In some examples, wall of the proximal neck 123 may have a thickness between 3-2000 microns. In some examples, at least a portion of the proximal neck 123 comprises a layer of radiopaque metal that is visible under fluoroscopy, including a layer of platinum, iridium, gold, silver, or alloys or combinations thereof.

One or more ring structures, tubular structures, telescoping structures, tubular segments, catheter segments, telescoping catheter segments, or other structures may be joined to the proximal neck 123. These proximal neck ring structures, tubular structures, telescoping structures, tubular segments, catheter segments, telescoping catheter segments, or other structures may comprise metal, polymer and adhesive, and combinations thereof. These proximal neck ring structures, tubular structures, telescoping structures, tubular segments, catheter segments, telescoping catheter segments, or other structures may comprise a radiopaque metal that is visible during fluoroscopy, including platinum, iridium, gold, silver, or alloys or combinations thereof. These proximal neck ring structures, tubular structures, telescoping structures, tubular segments, catheter segments, telescoping catheter segments, or other structures may comprise one piece, while in other examples they may comprise two or more pieces that are bonded together, including bonded together with a glue or adhesive. In some examples, a proximal neck joining structure may be joined to the proximal neck 123 or proximal neck assembly of the balloon 110 and also joined to the elongate body 160, thereby forming a joining or sealing of the proximal neck 123 or proximal neck assembly and elongate body 160. In some examples, a distal neck joining structure may be joined to the distal neck 129 or distal neck assembly of the balloon 110 and also joined to the elongate body 160, thereby forming a joining or sealing of the distal neck 129 or distal neck assembly and elongate body 160.

The proximal neck assembly of a balloon 110 may comprise a structure, or a portion of a structure, joined to the proximal neck 123 of a balloon 110 that may be configured to provide a smooth transition from a portion of the expandable metallic implant device 1 that has a larger outer diameter to a portion of the expandable metallic implant device 1 that has a smaller outer diameter to reduce the risk of tissue injury or device damage when advancing or retracting the expandable metallic implant device 1 in vivo, (a “proximal neck transitional structure”). In some examples, the outer diameter of the proximal portion of a proximal neck transitional structure is within 0.01 inch of the outer diameter of the adjacent portion of the distal elongate body 160 and the outer diameter of the distal portion of the proximal neck transitional structure is within 0.01 inch of the outer diameter of the folded and pleated balloon 115. In some examples, the outer diameter of the proximal portion of a proximal neck transitional structure is within 0.01 inch of the outer diameter of the proximal neck 123 or proximal neck assembly and the outer diameter of the distal portion of the proximal neck transitional structure is within 0.01 inch of the outer diameter of the folded and pleated balloon 115. In some examples, the proximal neck transitional structure may be conical in shape. In some examples, the proximal neck transitional structure may comprise a radiopaque metal that is visible during fluoroscopy, including platinum, iridium, gold, silver, or alloys or combinations thereof. In some examples, the proximal neck transitional structure may comprise one or more polymers, including polyether ether ketone, polycarbonate, nylon, polyimide, polyethylene terephthalate, polytetrafluoroethylene, silicone, polyurethane, co-polyester polymer, thermoplastic rubber, silicone-polycarbonate copolymer, polyethylene ethyl-vinyl-acetate (PEVA)co-polymer, a biocompatible elastomer, biocompatible resilient material, or a biocompatible adhesive. In some examples, a proximal neck transitional structure may comprise one piece, while in other examples, a proximal neck transitional structure may comprise two or more pieces that are bonded together, including bonded together with a glue or adhesive. In some examples, a proximal neck transitional structure is bonded to a proximal neck 123. In some examples, a proximal neck transitional structure is bonded to a portion of a proximal neck assembly. In some examples, a proximal neck transitional structure is bonded to the proximal neck 123 and the proximal neck assembly. In some examples, a portion of the inner surface of a proximal neck transitional structure is bonded to a portion of the outer surface of a proximal neck 123 or a portion of a proximal neck assembly.

In some examples, a balloon 110 comprises a distal neck 129 with a distal neck opening 130. In some examples, the distal neck opening 130 is closed, obstructed, or sealed. In some examples, the distal neck 129 extends away from the interior space 120 of the balloon 110 and In some examples, the distal neck 129 extends into the interior space 120 of the balloon 110. The length of distal neck 129 is between 1 mm and 20 mm, preferably with a length between 2 mm and 5 mm. The distal neck opening 130 has a diameter between 0.5 mm and 5 mm. The thickness of the wall of the distal neck 129 may be thicker, the same as the thickness, or thinner than the wall 137 of the inflatable portion 116 of the balloon 110. In some examples, wall of the distal neck 129 may have a thickness between 3-2000 microns. In some examples, at least a portion of the distal neck 129 comprises a layer of radiopaque metal that is visible under fluoroscopy, including a layer of platinum, iridium, gold, silver, or alloys or combinations thereof.

One or more ring structures, tubular structures, telescoping structures, tubular segments, catheter segments, telescoping catheter segments, or other structures may be joined to the proximal neck 123, forming a distal neck assembly. These distal neck ring structures, tubular structures, telescoping structures, tubular segments, catheter segments, telescoping catheter segments, or other structures may comprise metal, polymer and adhesive, and combinations thereof. These distal neck ring structures, tubular structures, telescoping structures, tubular segments, catheter segments, telescoping catheter segments, or other structures may comprise a radiopaque metal that is visible during fluoroscopy, including platinum, iridium, gold, silver, or alloys or combinations thereof. These distal neck ring structures, tubular structures, telescoping structures, tubular segments, catheter segments, telescoping catheter segments, or other structures may comprise one piece, while in other examples they may comprise two or more pieces that are bonded together, including bonded together with a glue or adhesive.

In some examples, the proximal neck 123, proximal neck assembly, distal neck 129, or distal neck assembly of the balloon 110 may comprise a marker band 136 that is radiopaque or conspicuous during fluoroscopy and configured to help visualize the location of the balloon 110 and the separation of the balloon 110 or balloon catheter 100 from the expanded expandable metallic implant 11 during use. In some examples, there is a radiopaque marker band 136 present in the lumen 131 of the distal neck 129 of the balloon 110 that is radiopaque or conspicuous during fluoroscopy to help an operator see when the balloon 110 or balloon catheter 100 has separated from the expanded expandable metallic implant 11. In some examples, the radiopaque marker band 136 present in the lumen 131 of the distal neck 129 is cylindrical in shape. In some examples, the radiopaque marker band 136 comprises platinum, iridium, gold, silver, or alloys or combinations thereof.

The elongate body 160 has a lumen 161 configured to convey fluid and assist in the dilation of a folded and pleated balloon 115, including by assisting in the conveyance of fluid from the hub 162 to the interior space 120 of the balloon 110. In some examples, the elongate body 160 has a proximal end that is joined to a hub 162 a distal end configured for joining or fluidly coupling to the proximal neck 123 or proximal neck assembly of the balloon 110, or to a neck bridging segment 135. In some examples, the elongate body 160 is joined or bonded to the proximal neck 123 of balloon 110, the proximal neck assembly of the balloon 110, or a proximal neck joining structure. In some examples, a balloon catheter 100 is configured so that a fluid communication can be made between the hub 162 of the elongate body 160, the lumen 161 of the elongate body 160, and the interior space 120 of the balloon 110. In some examples, the elongate body 160 is joined or bonded to the distal neck 129 of balloon 110, the distal neck assembly of the balloon 110, or a distal neck joining structure.

In some examples, the length of the elongate body 160 is between 20-200 cm. In some examples, the total length of the folded and pleated expandable metallic implant device 2 is between 20.5-200.5 cm, excluding any guidewire segment 69. In some examples, the outer diameter of the elongate body 160 is between 0.027-0.085 inch. In some examples, the internal or luminal diameter of the elongate body 160 is between 0.016-0.072 inch.

In some examples, a layer of the distal end of the elongate body 160 comprises a material with a Shore durometer hardness of 20-60 D. In some examples, the distal portion of the elongate body 160 comprises an aliphatic polyether polyurethane or a polyether block amide. In some examples, the aliphatic polyether polyurethane is Tecoflex. In some examples, the middle portion of the elongate body 160 comprises a polyether block amide or a nylon. In some examples, the polyether block amide is Pebax. In some examples, the range of the durometer of the polyether block amide is between Pebax 7233 and Pebax 2522 distally. In some examples, the proximal portion of the elongate body 160 comprises a nylon. In some examples, the nylon is Grilamid. In some examples, a layer of the elongate body 160 comprises polyimide or polytetrafluoroethylene.

In some examples, the inner layer 166 of the wall 165 of the elongate body 160 comprises a lubricious polymer. In some examples, the lubricious polymer comprises polytetrafluoroethylene, polyimide, or a composite or mixture of polytetrafluoroethylene (PTFE) and polyimide. In some examples, a layer of the proximal end of the elongate body 160 comprises a material with a Shore durometer hardness of 40-90 D. In some examples, the wall 165 of the elongate body 160 further comprises a tie layer.

In some examples, the metal of the middle layer 167 of the elongate body 160 is configured as wire. In some examples, the wire is configured in a spiral, coil, braid, woven, or straight pattern, or combinations thereof. In some examples, at least some of the metal in the middle layer 167 of the elongate body 160 is configured as wire with a cross-sectional shape that is round, oval, square, or rectangular. In some examples, the wire comprises nitinol or stainless steel. In some examples, the wire is round and has a diameter of between 0.0005-0.0030 inch. In some examples, the wire is configured in a coil with a pitch of between 0.0010-0.0060 inch. In some examples, the wire is flat and has a thickness of between 0.0005-0.0060 inch and a width of between 0.001-0.030 inch. In some examples, the wire in the distal portion of the elongate body 160 is round, has a diameter of between 0.0005-0.0030 inch, and is configured in a coil pattern with a pitch of between 0.0010-0.0060 inch. In some examples, wherein the wire is configured in a braid, the braid has a picks per inch of length (PPI) of 50-300, In some examples, the metal of the middle layer 167 of the elongate body 160 is configured as a laser cut hypotube. In some examples, the laser cut hypotube comprises nitinol. In some examples, the metal or metal wire is absent from the distal portion of the elongate body 160.

In some examples, of an expandable metallic implant device 1, the wall 165 of the elongate body 160 comprises one or more liquid crystal polymer fibers. In some examples, of an expandable metallic implant device 1, the one or more liquid crystal polymer fibers are oriented parallel to the first axis 45 of the elongate body 160. In some examples, the one or more liquid crystal polymer fibers are coiled around the elongate body 160.

In some examples, the polymer comprises barium sulfate. In some examples, the polymer of a layer of the wall 165 of the elongate body 160 comprises barium sulfate at a concentration of 10-30% to help visualize elongate body 160 and to visualize the separation of the expanded expandable metallic implant 11 and the elongate body 160 during use during fluoroscopy, or to make it more radiopaque or conspicuous during fluoroscopy.

In some examples, the distal portion of the elongate body 160 comprises one or more marker bands 163 that are radiopaque or conspicuous during fluoroscopy and configured to help visualize the elongate body 160 and to visualize the separation of the expanded expandable metallic implant 11 and the elongate body 160 during use, or to make it more radiopaque or conspicuous during fluoroscopy. In some examples, the radiopaque marker band 136 comprises platinum, iridium, gold, silver, or alloys or combinations thereof. In some examples, the outer diameter of the distal end of the elongate body 160 has a radius.

In some examples, the elongate body 160 comprises a lubricious or hydrophilic coating layer 169. In some examples, the lubricious or hydrophilic coating layer 169 is present on the outer surface, the inner (luminal) surface, or both the outer and inner surface of the elongate body 160. In some examples, the lubricious or hydrophilic coating layer 169 is present on the outer surface of the distal portion of the elongate body 160, on the outer surface of the middle and distal portion of the elongate body 160, or on the entire outer surface of the elongate body 160. In some examples, the lubricious coating layer 169 is present only on the distal portion of the elongate body 160 and is absent from the proximal portion of the elongate body 160.

In some examples, the inner and outer diameter of the distal portion of the elongate body 160 of the expandable metallic implant device 1 is larger than the inner and outer diameter of the proximal or middle portions of the elongate body 160 of the expandable metallic implant device 1. In some examples, this larger diameter provides space in the lumen 161 of the distal portion of the elongate body 160 for components that are involved in the attachment of the elongate body 160 to: the proximal neck 123 of the balloon 110; components of the proximal neck assembly; or the neck bridging segment 135. In some examples, the inner and outer diameter of the distal portion of the elongate body 160 of the expandable metallic implant device 1 is smaller than the inner and outer diameter of the proximal or middle portions of the elongate body 160 of the expandable metallic implant device 1. In some examples, this smaller diameter provides space in the lumen 161 of the distal portion of the elongate body 160 for components that are involved in the attachment of the elongate body 160 to: the proximal neck 123 of the balloon 110; components of the proximal neck assembly; or the neck bridging segment 135. In some examples, the outer diameter of the distal portion of the elongate body 160 tapers from the larger diameter to the smaller diameter to reduce frictional forces when advancing or retracting the elongate body 160 in vivo.

The hub 162 of the elongate body 160 is configured for coupling with fluid-filled syringe or inflation device to enable the injection of fluid through the lumen 161 of the elongate body 160 and into the interior space 120 of the balloon 110 under pressure. The proximal portion of the elongate body 160 adjacent to the hub 162 may comprise a segment of polymer that provides strain relief 164 to the junction between the hub 162 and the elongate body 160.

In some examples, the elongate body 160 is straight or has no pre-formed shape or is straight or has no pre-formed shape when in an unconstrained configuration. In some examples, a portion of the elongate body 160 has a pre-formed shape or has a portion with a pre-formed shape when in an unconstrained configuration, including a distal portion. This pre-formed shape may assist operators in advancing an expandable metallic implant device 1 in vivo by enabling the operator to turn the tip of the expandable metallic implant device 1 and to direct the tip of the expandable metallic implant device 1 toward various locations in vivo. In some examples, the distal portion of the elongate body 160, when in an unconstrained configuration, is angled or has an angled shape. In some examples, the angle is 10-70 degrees.

FIGS. 3A and 3B are perspective views of an example of a folded and pleated expandable metallic implant device 2 with a folded and pleated expandable metallic implant 13 and folded and pleated balloon 115.

In some examples, the expandable metallic implant is folded and pleated together with the balloon 110 and configured for expansion when the folded and pleated balloon 115 is inflated and separation of the expandable metallic implant in vivo. During expansion of a folded and pleated balloon 115, the folded and pleated expandable metallic implant 13 transitions to an “expanded” configuration 11. After separation of an expanded expandable metallic implant 11 from the deflated or collapsed balloon 113, the expanded expandable metallic implant 11 is configured to maintain an expanded configuration and to obstruct, occlude, embolize, seal, or reduce the flow of blood or other biological fluids in arteries, veins, aneurysms, parent vessels of saccular aneurysms, paravalvular leak pathways, biological conduits, or other blood-containing, fluid-containing, or biological spaces in human patients. In some examples, the expandable metallic implant 10 is provided in a “deliverable configuration” wherein some or all of the expandable metallic implant 10 is folded and pleated together with a balloon 110 in a manner wherein portions of the wall 30 of the expandable portion 15 of the expandable metallic implant 10 are squeezed or pressed together with portions of the wall 137 of a balloon 110 to facilitate passage through guide catheters or guide sheaths 200 or maneuvered into or through arteries, veins, aneurysms, chambers of the heart, biological conduits, other blood-containing and fluid-containing spaces, or biological spaces to a treatment site. As used herein, a biological space can mean a continuous area or expanse in a human patient, including a continuous area or expanse that is free, available, or unoccupied. When describing these devices, the proximal end refers to the end that is closer to the operator, and the distal end refers to the end that is advanced first into the patient. For individual components of devices described herein, the same proximal and distal orientation is maintained.

Examples of the expandable metallic implant 10 of the current disclosure solve several long-standing limitations of other devices intended for obstructing, occluding, embolizing, sealing, or reducing the flow of blood or other biological fluids in arteries, veins, aneurysms, parent vessels of saccular aneurysms, paravalvular leak pathways, biological conduits, or other blood-containing, fluid-containing, or biological spaces in human patients. For example, coil expandable implants 301 and vascular plug expandable implants used for vascular embolization present a porous barrier to the flow of blood, often resulting in a slow or incomplete treatment effect. The present disclosure describes examples of expandable implant devices that place an expanded expandable metallic implant 11 at the target location that presents a solid surface to the flow of blood or biological fluid and is capable of providing immediate and complete occlusion and cessation of flow, including blood flow.

FIGS. 4A and 4B are side and perspective views of an example of an expanded expandable metallic implant 11 device 3 with an expanded expandable metallic implant 11 and an inflated balloon 111, with overall geometric dimensions defined.

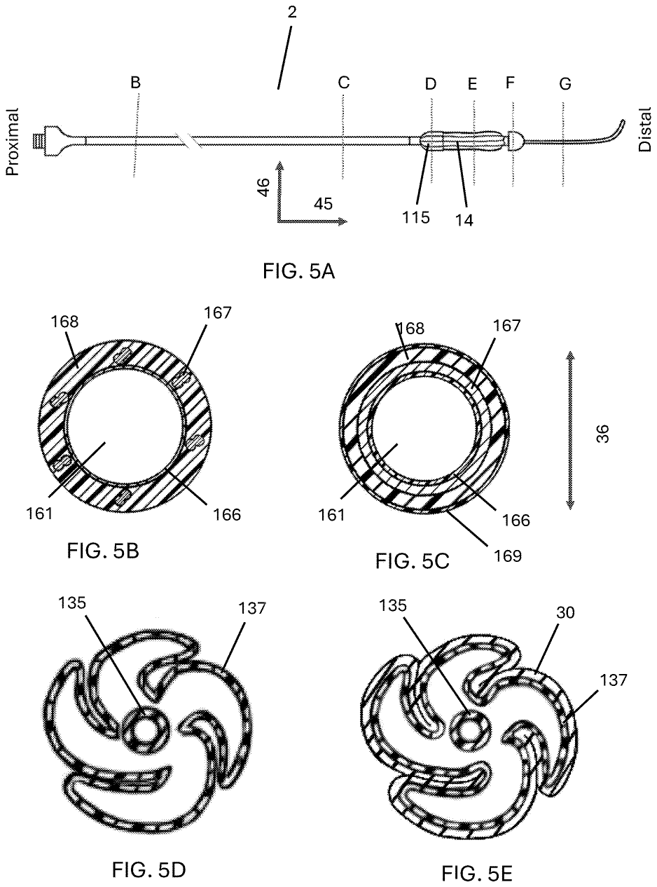

FIG. 5A shows an example of a folded and pleated expandable metallic implant device 2 with a folded and pleated expandable metallic implant 14 and folded and pleated balloon 115, FIG. 5B-G shows cross-sectional views at locations B-G shown in FIG. 5A The individual cross sections are not shown equivalent in scale.

In some examples, the wall 165 of the elongate body 160 is continuous from the proximal end to the distal end. In some examples, the elongate body 160 comprises: an outer layer 168 comprising polymer; an inner layer 166 comprising polymer; and a middle layer 167 comprising metal, wherein the middle layer 167 is disposed between the outer layer 168 and the inner layer 166. In some examples, the inner layer 166, outer layer 168, or both the inner layer 166 and outer layer 168 of the elongate body 160 comprises polymer with successively decreasing durometer from the proximal to the distal end of the elongate body 160.

A folded and pleated expandable metallic implant device 2 is disclosed herein, where the flexibility and deliverability of the device is optimized. As a general approach, in order to optimize the flexibility and deliverability of the distal portion of a folded and pleated expandable metallic implant device 2, the rigid segments are optimized for short length, and the flexible segments are optimized for flexibility. The rigid segments of the distal portion of the folded and pleated expandable metallic implant device 2 may include the folded and pleated core portion 14 of the expandable metallic implant 10, the neck 60 of the expandable metallic implant 10, and some or all additional neck components 66 of the expandable metallic implant 10. The flexible segments of the distal portion of the folded and pleated expandable metallic implant device 2 may include the elongate body 160, the proximal portion of the folded and pleated balloon 115 that is not covered by the folded and pleated expandable metallic implant 13, the neck transitional region 77 between the transition zone 19 and the neck 60 of the expandable metallic implant 10, and some or all additional neck components 66 of the expandable metallic implant 10, including the guidewire segment 69.

Starting from the elongate body 160 and progressing distally, the following optimizations can be made to optimize the flexibility and deliverability of the folded and pleated expandable metallic implant device 2. In some examples, the elongate body 160 is optimized for flexibility to enable bending, especially the distal portion and the junction between the elongate body 160 and the proximal neck 123, proximal neck assembly, or neck bridging segment 135 of the balloon 110, and potentially improving deliverability of the folded and pleated expandable metallic implant device 2. Optimizing the flexibility of the distal portion of the elongate body 160 could include forming the distal portion of the elongate body 160 with a flexible polymer and providing a flexible metal middle layer 167 of the elongate body 160 in this region, including in the form of a nitinol coil or braid, or a laser cut nitinol hypotube.

In some examples, the length of the proximal neck 123 of the balloon 110 is optimized for a short length, thereby reducing the overall length of this rigid segment, while still enabling a secure attachment to the balloon 110 and the elongate body 160, potentially improving deliverability of the folded and pleated expandable metallic implant device 2.

In some examples, a flexible material is chosen to form the wall 137 of the balloon 110 to increase the flexibility of the folded and pleated balloon 115 and the folded and pleated expandable metallic implant device 2. In some examples, a flexible material is chosen to form the wall of the neck bridging segment 135 to increase the flexibility of the folded and pleated balloon 115 and the folded and pleated expandable metallic implant device 2.

The length of the folded and pleated expandable portion 15 of an expandable metallic implant 10 is optimized for a short length, thereby reducing the overall length of this rigid portion of the folded and pleated expandable metallic implant device 2. The length of the proximal zone 17 (if any), seal zone 18, and transition zone 19 can be individually and collectively optimized for the shortest possible length that still provides an effective obstruction or sealing of arteries, veins, aneurysms, parent vessels of saccular aneurysms, paravalvular leak pathways, other blood-containing structures or biological conduits, potentially improving deliverability of the folded and pleated expandable metallic implant device 2.

In some examples, the region between the transition zone 19 and the neck 60 of the expandable metallic implant 10 is optimized for flexibility so this region can flex and bend in response to forces routinely encountered when the folded and pleated expandable metallic implant device 2 is moving along a tortuous path, potentially improving deliverability of the folded and pleated expandable metallic implant device 2. In some examples, of expandable metallic implants 10, the wall 29 of the neck transitional region 77 of the core portion 14 of the expandable metallic implant 10 is comprised of a thin layer of gold that is capable of bending in response to forces routinely encountered when the folded and pleated expandable metallic implant device 2 is moving along a tortuous path, thereby providing a hinge-like region between the transition zone 19 and the neck 60 of the folded and pleated expandable metallic implant 13, potentially improving deliverability of the folded and pleated expandable metallic implant device 2. In some examples, at least a portion of the distal neck 129 of the balloon 110 and the neck 60 of the expandable metallic implant 10 are overlapping, reducing the combined length of these rigid portions of the folded and pleated expandable metallic implant device 2 and aligning the adjacent flexible regions, potentially improving deliverability.

The length of the neck 60 and rigid additional neck components 66 of the expandable metallic implant 10 are optimized for a short length, thereby reducing the overall length of these rigid segments, while still enabling an effective attachment or bonding to additional neck components 66, such as a guidewire segment 69 and a nose cone 68. In some examples, the flexibility of the guidewire segment 69 of expandable metallic implant 69 is optimized, potentially improving deliverability of the folded and pleated expandable metallic implant device 2. The guidewire segment 69 may help in the insertion of the implant into a Tuohy Borst adaptor 406 and guide catheter or guide sheath 200. The guidewire segment 69 may help align the implant and catheter when advancing in catheter. The guidewire segment 69 may provide guidance in artery or vein, allowing an operator to steer the implant into the target vessel or conduit segment.

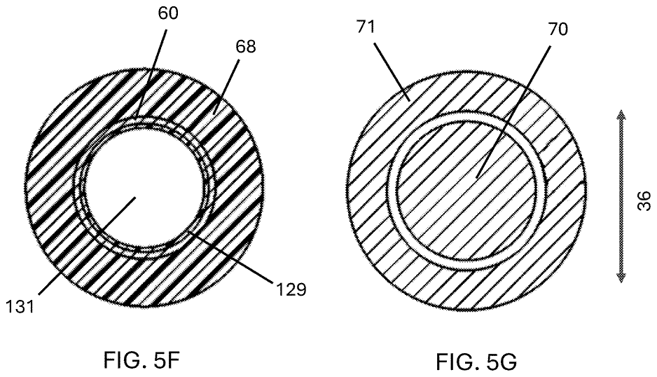

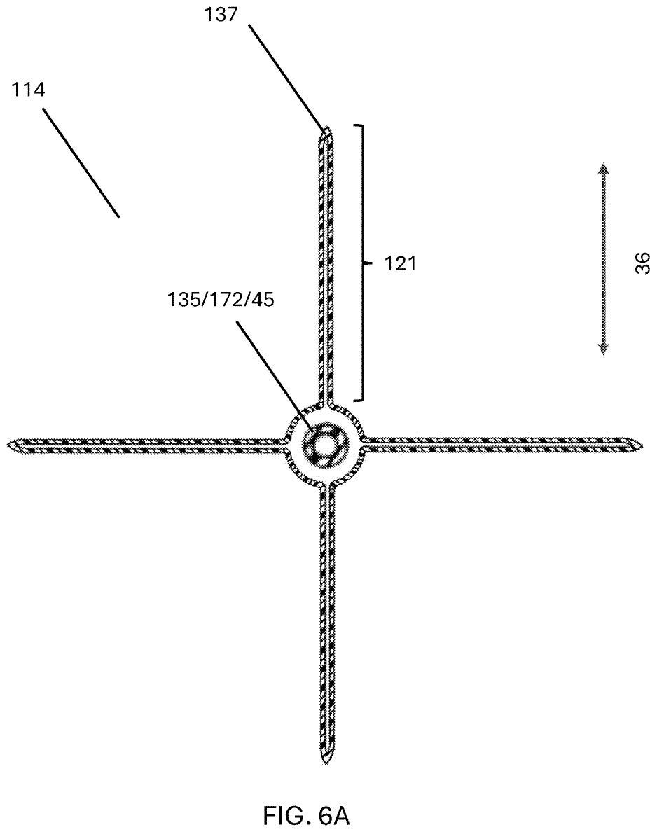

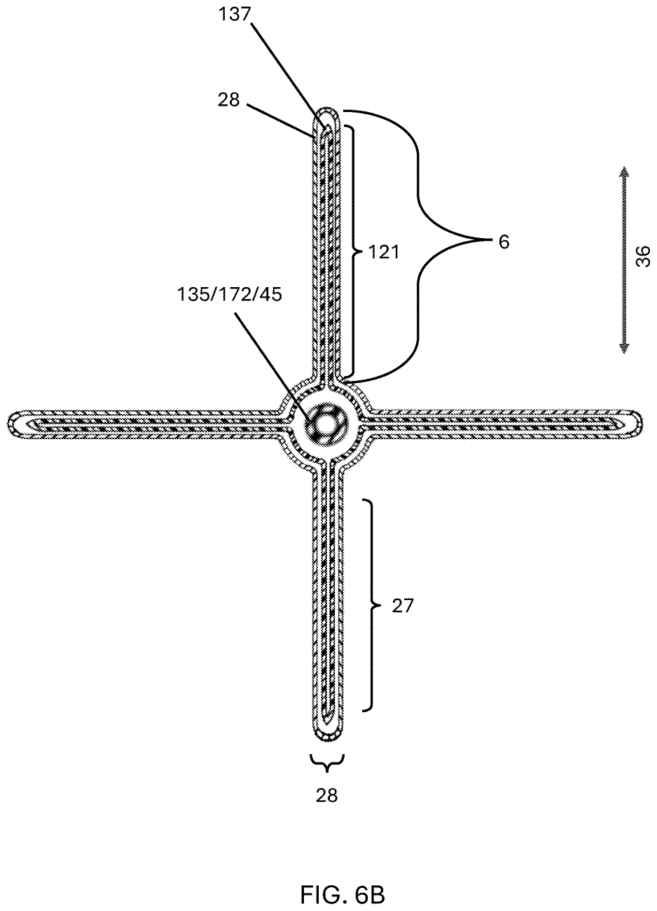

FIG. 6A is a cross-sectional view of a folded balloon 114 with four balloon wings 121. FIG. 6B is a cross-sectional view of a folded balloon 137 and a pleated expandable metallic implant 13, wherein the balloon 110 and expandable metallic implant 10 have been folded together with four wings 6. FIG. 6B also shows a straight portion 29 of a wing 6 of the expandable metallic implant 10 and a curved portion 28 of the wing 6 of the expandable metallic implant 10.

In some examples, at least one of the balloon 110 or the expandable metallic implant 10 may be folded into wings. The wings may be compressed or pleated over around a first axis 45 of the balloon 110. The wings can be formed as ridges separated by troughs. Each trough can extend longitudinally from a ridgeline.

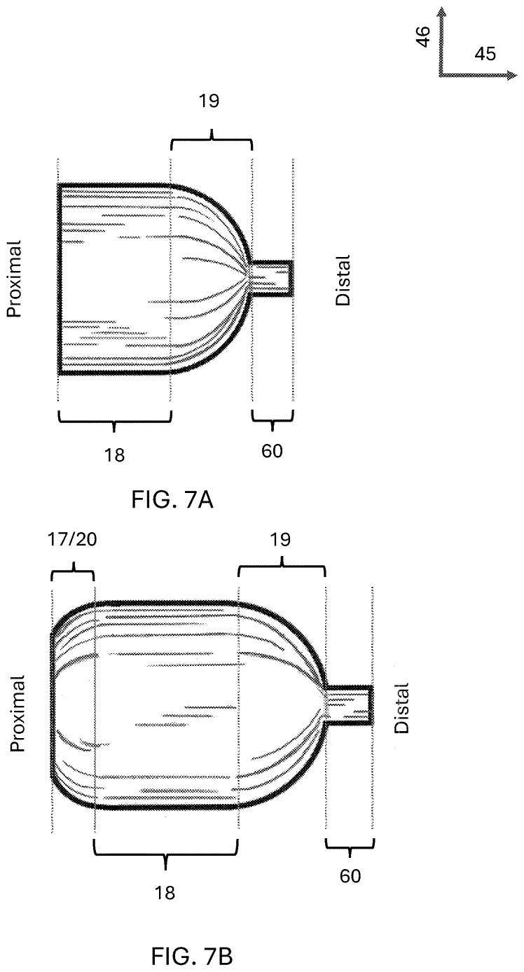

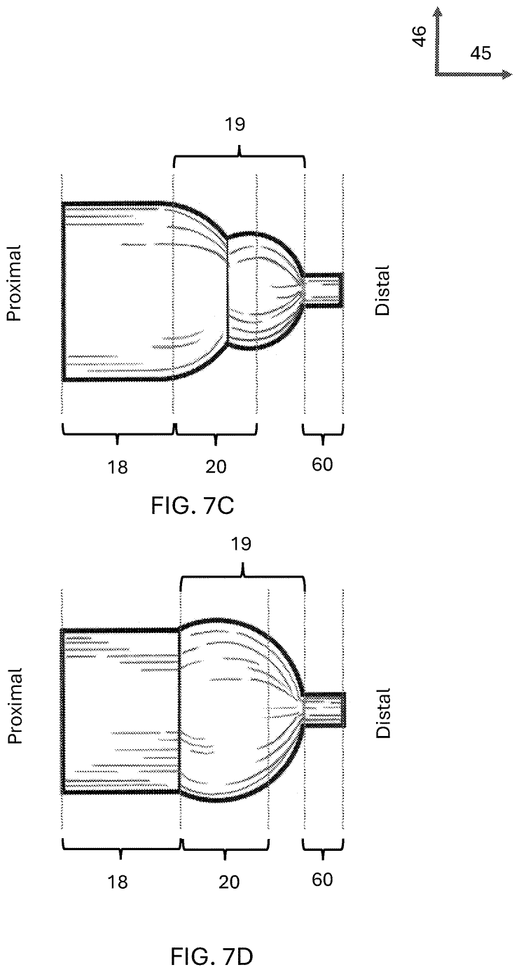

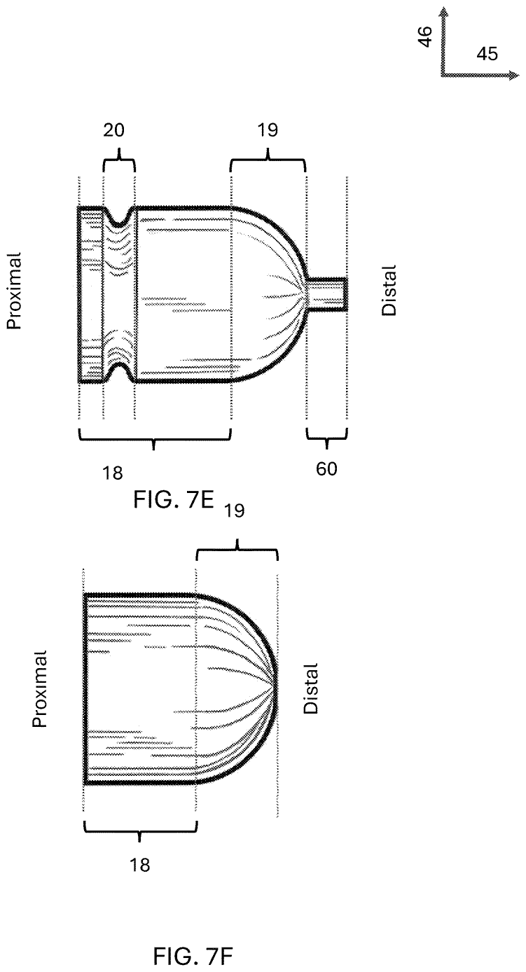

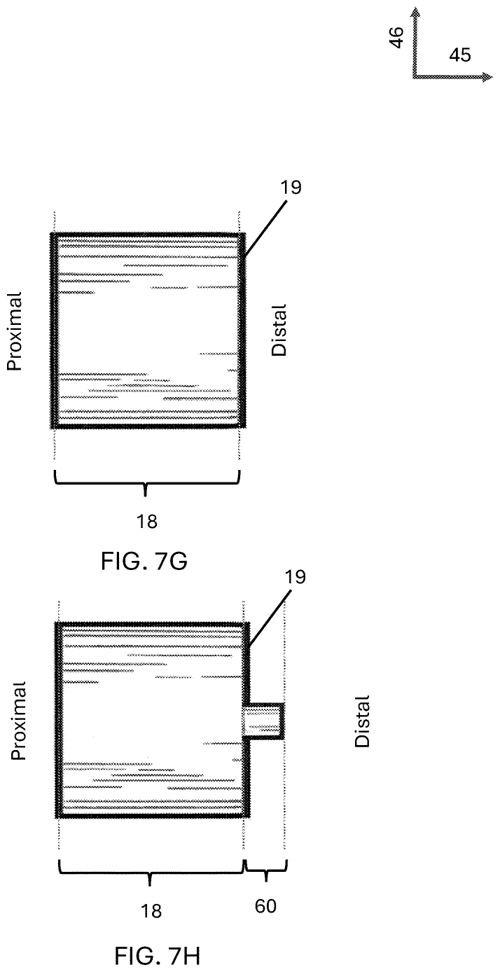

FIG. 7A is a side view of an example of an expanded expandable metallic implant 11 having a seal zone 18, a transition zone 19, and a neck 60. FIG. 7B is side view of an example of an expanded expandable metallic implant 11 having a proximal zone 17, a seal zone 18, a transition zone 19, and a neck 60. The proximal zone 17 is also a balloon retention region 20. FIG. 7C is side view of an example of an expanded expandable metallic implant 11 having a seal zone 18, a transition zone 19, and a neck 60. This example of expanded expandable metallic implant 11 has a balloon retention region 20 between the seal zone 18 and the neck 60 and thus within the transition zone 19. FIG. 7D is side view of an example of an expanded expandable metallic implant 11 having a seal zone 18, a transition zone 19, and a neck 60. This example of expanded expandable metallic implant 11 has a balloon retention region 20 between the seal zone 18 and the neck 60 and thus within the transition zone 19. FIG. 7E is a side view of an example of an expanded expandable metallic implant 11 having a seal zone 18, a transition zone 19, and a neck 60. This example of expanded expandable metallic implant 11 has a balloon retention region 20 within the seal zone 18. FIG. 7F is a side view of an example of an expanded expandable metallic implant 11 having a seal zone 18, a transition zone 19. This example does not have a neck 60. The transition zone 19 is rounded. FIG. 7G is a side view of an example of an expanded expandable metallic implant 11 having a seal zone 18, and a transition zone 19. This example does not have a neck 60 and the transition zone 19 is flat. FIG. 7H is a side view of an example of an expanded expandable metallic implant 11 having a seal zone 18, a transition zone 19, and a neck 60. The transition zone 19 is flat.

As shown in FIG. 7A-H, expanded expandable metallic implants 10 have a seal zone 18 and a transition zone 19. In such examples, the diameter of the transitional region 19 decreases from the seal zone 18 to central axis 44 of the expanded expandable metallic implant 11. Some examples of expanded expandable metallic implants 10 also have a proximal zone 17. In some examples, the wall 29 of the core portion 29 of an expandable metallic implant 10 extends generally continuously from the proximal zone 17 (if any), through the seal zone 18, through the transition zone 19, and through the 60 neck (if any). The wall 30 has an exterior surface 41 such that, when the expandable metallic implant 10 is expanded, the exterior surface 41 faces the vessel wall or other adjacent tissue and the interior surface 42 faces the central hollow region 16. As shown in FIGS. 1B and 1D, In some examples, the expandable metallic implant 10 has a neck 60 with a neck opening 61. In some examples, the lumen 62 of the neck 60 is configured to accept a portion of a guidewire segment 69. In examples, wherein the expanded expandable metallic implant 11 comprises a neck 60 distal to the transitional region 19, the diameter of the transitional region 19 decreases from the seal zone 18 to the neck 60.

In some examples, the expanded expandable metallic implant 11 includes a seal zone 18 and a transition zone 19. In some examples, the seal zone 18 is configured to form a circumferential seal against the vessel wall (or other tissue). In some examples, the transition zone 19 configured to establish a solid barrier to the flow of blood or other biological fluids, provide an acceptable end profile for the folded and pleated expandable metallic implant 13, and to facilitate pleating and folding of the expandable metallic implant 10. In some examples, the expanded expandable metallic implant 11 further comprises a proximal zone 17, with a function separate and distinct from the seal zone 18, including holding the inflated balloon 111 and the expanded expandable metallic implant 11 together or provide additional resistance to migration of the expanded expandable metallic implant 11 after separation from the balloon catheter 100.

The proximal zone 17 (if present), the seal zone 18, the transition zone 19, and the neck 60 (if present) of the expandable metallic implant 10 form the “core” portion of the expandable metallic implant 10. In some examples, the core portion 14 of an expandable metallic implant 10 is made by electroforming, including made of gold by electroforming. The proximal zone 17 (if present), the seal zone 18, and the transition zone 19 of the expandable metallic implant 10 form the portion of the expandable metallic implant 10 which are folded and pleated prior to use and referred to as the “expandable” portion.

In some examples, of an expandable metallic implant device 1, the expanded expandable metallic implant 11 has a balloon retention region 20 which is designed to secure the attachment of the inflated balloon 111 to the expanded expandable metallic implant 11 after inflation of the balloon 110, or to provide resistance to the withdrawal of the inflated balloon 111 from within the central hollow region 16 of the expanded expandable metallic implant 11. In some examples, some diameter of the balloon retention region 20 is smaller than the region adjacent to, and distal to the balloon retention region 20, as shown in FIG. 7B. In some examples, some diameter of the balloon retention region 20 is smaller than the region adjacent and proximal to the balloon retention region 20 and smaller than the region and adjacent and distal to the balloon retention region 20, as shown in FIGS. 7C and 7E. In some examples, some diameter of the balloon retention region 20 is larger than the region adjacent and proximal to the balloon retention region 20, as shown in FIG. 7D. In some examples, the balloon retention region 20 keeps the expanded expandable metallic implant 11 and the inflated balloon 111 together after expansion by “capturing” the portion of the inflated balloon 111 distal to the balloon retention region 20 within the expanded expandable metallic implant 11. In some examples, the smallest diameter of the balloon retention region 20 is 90-95%, 80-89%, 70-79%, 60-69%, 50-99%, 40-49%, 30-39% of the diameter of an adjacent region. In some examples, the largest diameter of the balloon retention region 20 is 105-110%, 111-120%, 121-130%, 131-%, or 141-150% of the diameter of an adjacent region.

In some examples, a balloon retention region 20 is present at the proximal end of the expanded expandable metallic implant 11 in a proximal zone 17, as shown in FIG. 7B. In some examples, a balloon retention region 20 is present in the seal zone 18 of the expanded expandable metallic implant 11, as shown in FIG. 7E. In some examples, a balloon retention region 20 is present in the transition zone 19 of the expanded expandable metallic implant 11, as shown in FIGS. 7C and 7D.

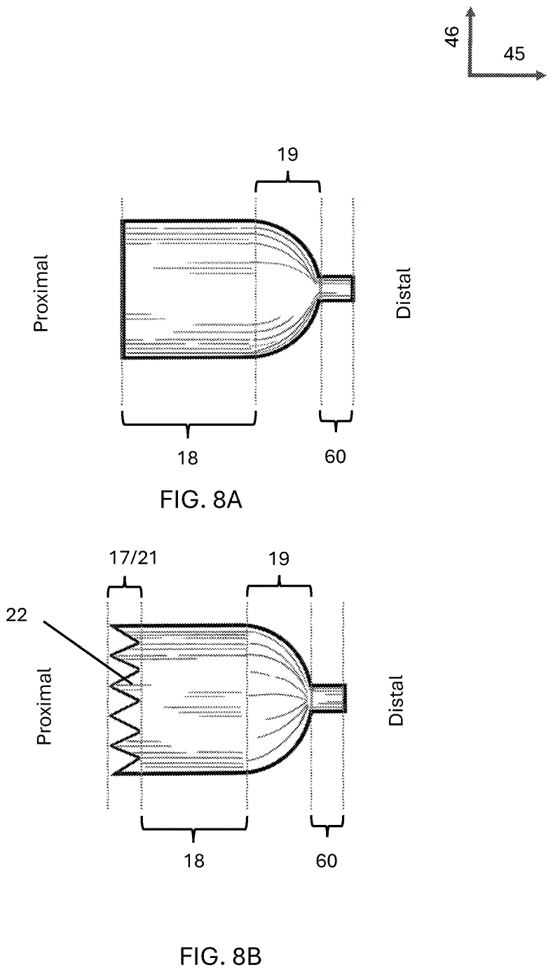

FIG. 8A is a side view of an example of an expanded expandable metallic implant 11 having a seal zone 18, a transition zone 19, and a neck 60. FIG. 8B is a perspective view of an example of an expanded expandable metallic implant 11 having a seal zone 18, a transition zone 19, and a neck 60. The proximal end of this expanded expandable metallic implant 11 has a fixation region 21 with pointed flaps 22.

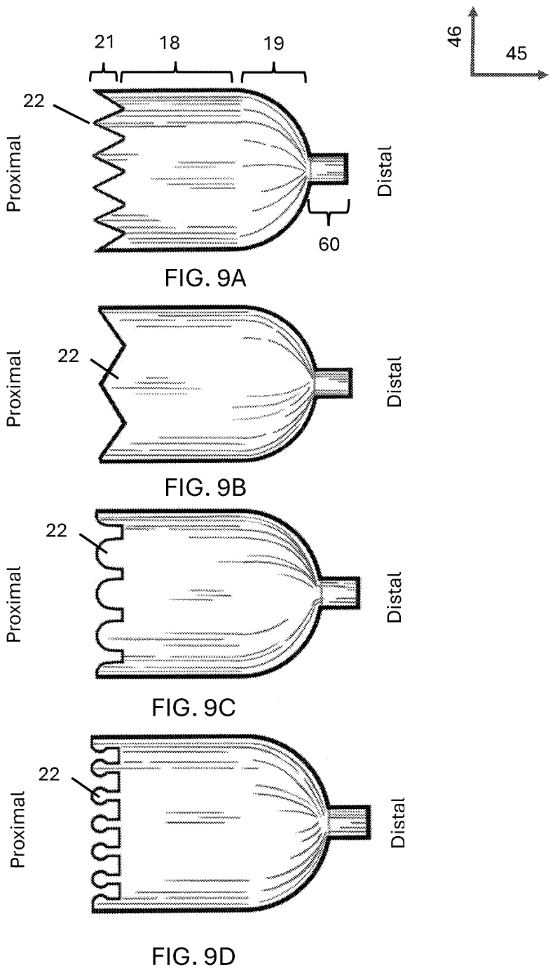

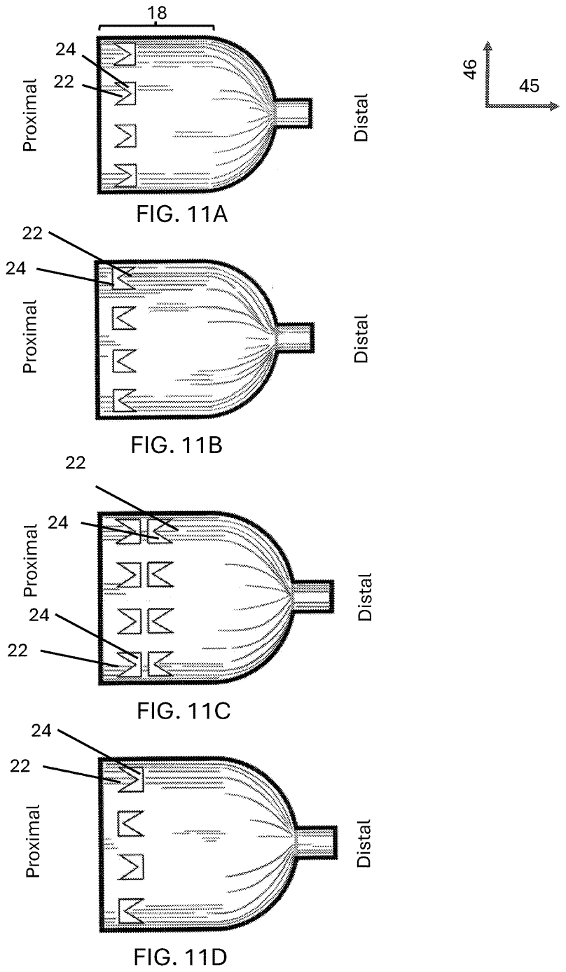

In some examples, of an expandable metallic implant device 1, the expanded expandable metallic implant 11 comprises a fixation region 21 to facilitate attachment or fixation of the expanded expandable metallic implant 11 to the adjacent vessel wall (or other tissue) after expansion and separation from the balloon catheter 100. In some examples, of an expandable metallic implant device 1, the expanded expandable metallic implant 11 comprises a fixation region 21 to increase sliding friction between the expanded expandable metallic implant 11 the adjacent vessel wall (or other tissue) after expansion and separation from the balloon catheter 100. In some examples, of an expandable metallic implant device 1, the expanded expandable metallic implant 11 comprises a fixation region 21 to increase the expanded expandable metallic implant's 11 resistance to migration after expansion and separation from the balloon catheter 100. In some examples, a fixation region 21 is present at the proximal end of the expanded expandable metallic implant 11 in a proximal zone 17, as shown in FIG. 9A-D. In some examples, a fixation region 21 is present in the seal zone 18 of the expanded expandable metallic implant 11, as shown in FIG. 11A-D. In some examples, a fixation region 21 is present in the transition zone 19 of the expanded expandable metallic implant 11 as shown in FIGS. 7C and 7D.

FIG. 9A is a perspective view of an example of an expanded expandable metallic implant 11 having a seal zone 18, a transition zone 19, and a neck 60. The proximal end of this expanded expandable metallic implant 11 has a fixation region 21 with narrow pointed flaps 22. FIG. 9B is a perspective view of an example of an expanded expandable metallic implant 11 having a seal zone 18, a transition zone 19, and a neck 60. The proximal end of this expanded expandable metallic implant 11 has a fixation region 21 with broad pointed flaps 22. FIG. 9C is a perspective view of an example of an expanded expandable metallic implant 11 having a seal zone 18, a transition zone 19, and a neck 60. The proximal end of this expanded expandable metallic implant 11 has a fixation region 21 with rounded flaps 22. FIG. 9D is a perspective view of an example of an expanded expandable metallic implant 11 having a seal zone 18, a transition zone 19, and a neck 60. The proximal end of this expanded expandable metallic implant 11 has a fixation region 21 with stalk-and-bud shaped flaps 22.

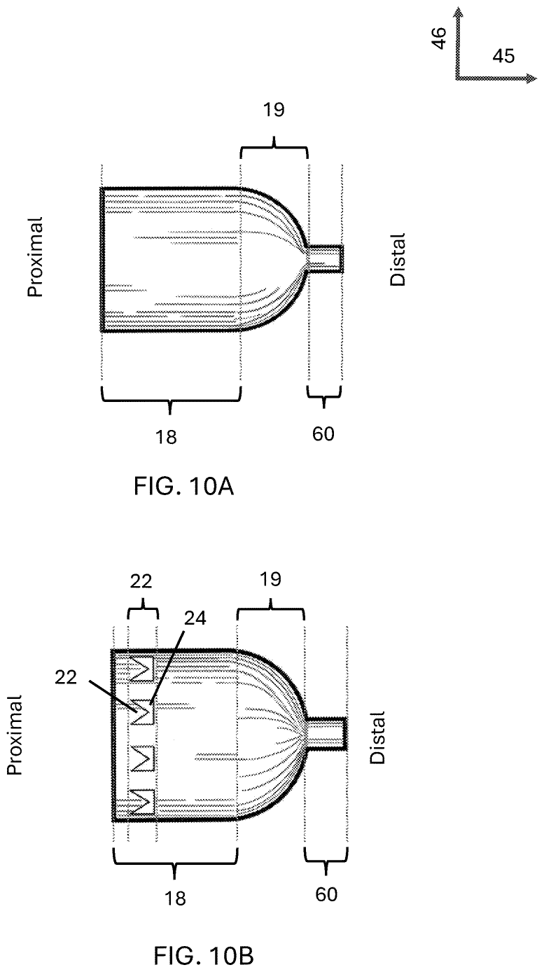

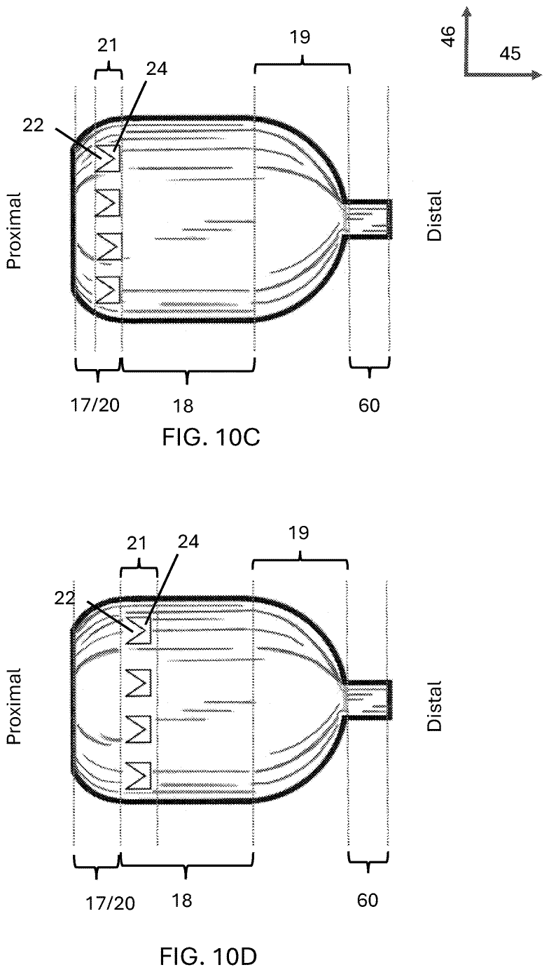

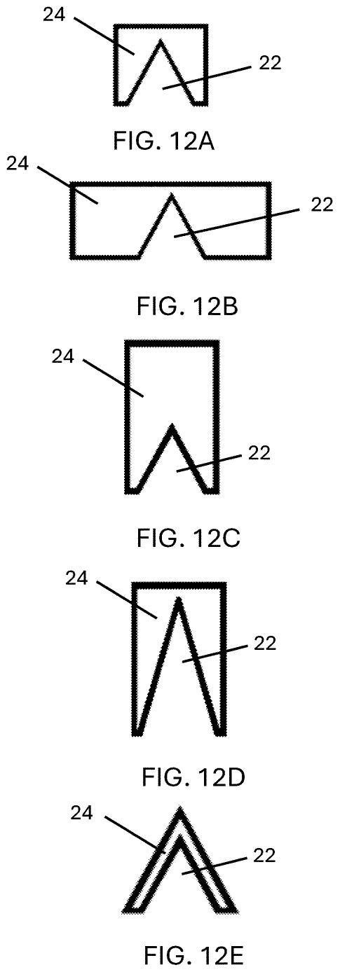

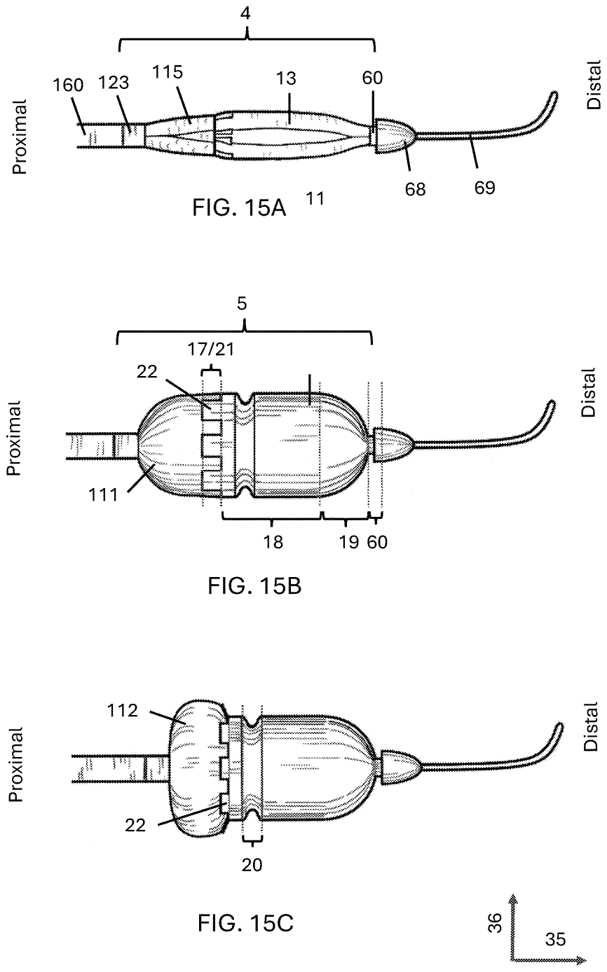

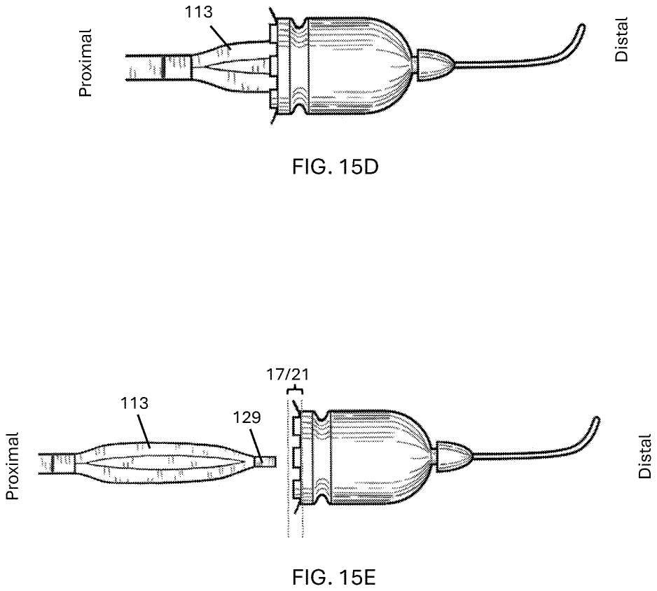

In some examples, of an expandable metallic implant device 1, the fixation region 21 of the expanded expandable metallic implant 11 comprises one or more flaps 22 on the proximal end of the expanded expandable metallic implant 11, as shown in FIG. 9A-D. In some examples, the fixation region 21 comprises one or more internal flaps 22, which are present within a flap window 24, as shown in FIG. 10B-D, 12A-D and FIGS. 11, 12, 13 and 14. In some examples, the proximal zone 17 of the expanded expandable metallic implant 11 is configured with an internal flap 22 within a flap window 24. In some examples, the seal zone 18 of the expanded expandable metallic implant 11 is configured with one or more internal flaps 22 within a flap window 24, as shown in FIG. 10B, C and 12A-D, and in FIGS. 11, 13 and 14. In some examples, the transition zone 19 of the expanded expandable metallic implant 11 is configured with one or more internal flaps 22 within a flap window 24. A variety of shapes and sizes of the flap 22 and flap window 24 of the expanded expandable metallic implant 11 are described herein, including as shown in FIG. 9A-D, FIG. 12 A-E, and FIG. 15B-E. In some examples, the tip of the flap 22 of an expanded expandable metallic implant 11 can be pointed, rounded, squared, or complex in shape, as shown in FIG. 9A-D. In some examples, at least one flap 22 is configured such that the free end is distal to the fixed end, at least one flap 22 is configured such that the free end is proximal to the fixed end, or at least one flap 22 is configured such that the free end is distal to the fixed end and at least one flap 22 is configured such that the free end is proximal to the fixed end (FIG. 11).

FIG. 10A is a perspective view of an embodiment of an expanded expandable metallic implant 11 having a seal zone 18, a transition zone 19, and a neck 60, with its overall geometric dimensions defined. FIG. 10B is a perspective view of an example of an expanded expandable metallic implant 11 having a seal zone 18, a transition zone 19, and a neck 60. This example of an expanded expandable metallic implant 11 can have a fixation region 21 within the seal zone 18. The fixation region 21 can include pointed flaps 22 with a proximal-distal alignment within a square flap window 24.

FIG. 11A is a perspective view of an example of an expanded expandable metallic implant 11 having a seal zone 18, a transition zone 19, and a neck 60. This example of expanded expandable metallic implant 11 has a fixation region 21 with flaps 22 within the seal zone 18. The fixation region 21 comprises one row of pointed flaps 22 with a proximal-distal alignment within a square flap window 24.

FIG. 11B is a perspective view of an example of an expanded expandable metallic implant 11 having a seal zone 18, a transition zone 19, and a neck 60. This example of expanded expandable metallic implant 11 has a fixation region 21 within the seal zone 18. The fixation region 21 comprises one row of pointed flaps 22 with a distal-proximal alignment within a square flap window 24.

FIG. 11C is a perspective view of an example of an expanded expandable metallic implant 11 having a seal zone 18, a transition zone 19, and a neck 60. This example of expanded expandable metallic implant 11 has a fixation region 21 within the seal zone 18. The fixation region 21 comprises two rows of pointed flaps 22, one with a distal-proximal alignment within a square flap window 24 and the other with a proximal-distal alignment within a square flap window 24.

FIG. 11D is a perspective view of an example of an expanded expandable metallic implant 11 having a seal zone 18, a transition zone 19, and a neck 60. This example of expanded expandable metallic implant 11 has a fixation region 21 within the seal zone 18. The fixation region 21 comprises one row of pointed flaps 22, wherein some flaps 22 have a distal-proximal alignment within a square flap window 24 and other flaps 22 have a proximal-distal alignment within a square flap window 24.

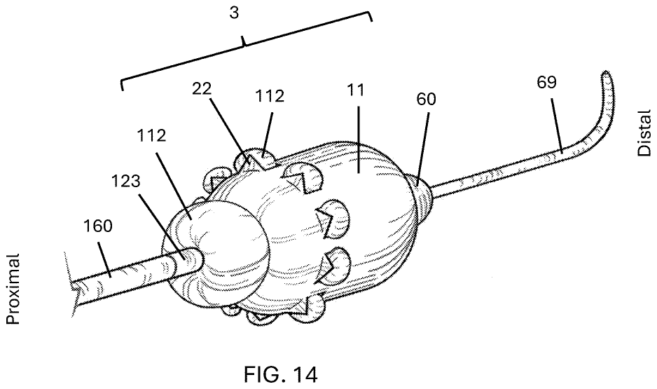

In some examples, overinflating the balloon 110 can cause the over-inflated balloon 112 to protrude through the flap window 24 and cause one or more flaps 22 to protrude radially outward, as shown in FIG. 12C and FIG. 14.

FIG. 12A is a planar view of a pointed flap 22 of an expanded expandable metallic implant 11 with a square flap window 24. FIG. 12B is a planar view of a pointed flap 22 of an expanded expandable metallic implant 11 with a rectangular flap window 24 that is wider than tall. FIG. 12C is a planar view of a pointed flap 22 of an expanded expandable metallic implant 11 with a rectangular flap window 24 that is taller than wide. FIG. 12D is a planar view of a pointed flap 22 of an expanded expandable metallic implant 11 with a rectangular flap window 24 that is taller than wide. FIG. 12E is a planar view of a pointed flap 22 of an expanded expandable metallic implant 11 with a triangular flap window 24.

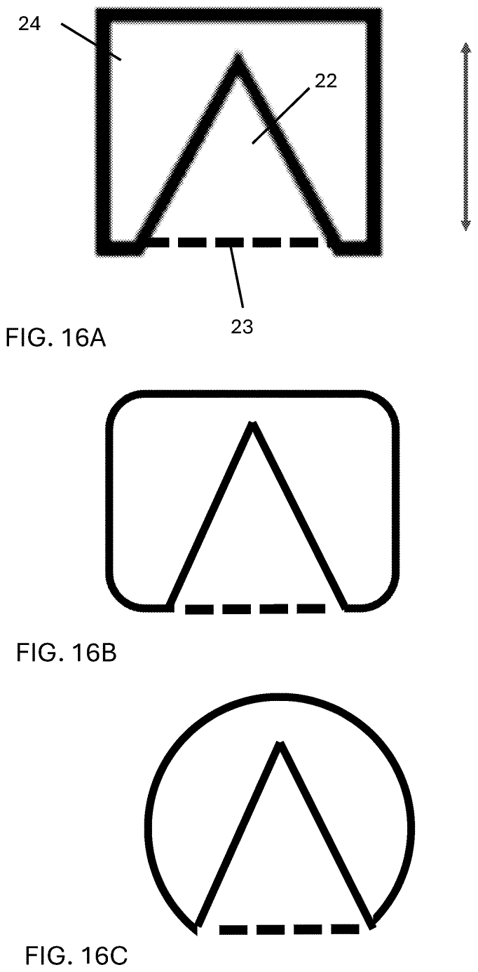

The expandable metallic implant 10 can include any combination of the flaps 22 and/or flap windows 24 shown in FIG. 12A-E. It should be appreciated that the disclosed examples of square, rectangular or triangular windows is not a limitation. The window can have any shape including rectangular, round oval or any shape having a combination of straight and curved elements, as shown in FIG. 16.

A preferred example of the expanded expandable metallic implant 11 is shown in FIGS. 26A, 26B and also in 27A, 27B and 27C. A preferred example has an expandable region 15 comprising a wall 30 that has a seal zone 18, a proximal zone 17 comprising a balloon retention zone 20 that tapers down to a reduced diameter for the retention of the ballon 110, proximal flaps 22, and a transition zone 19 that tapers down to a neck transitional region 77. A preferred example of the expandable metallic implant 11 also comprises a neck 60. The neck 60 may house or be joined to additional components including a valve 79 in the neck lumen 62 and/or in the neck opening 61. In some examples, the expandable metallic implant 11 can include multiple valves. For example, the expandable metallic implant 11 can include 1-5 valves.

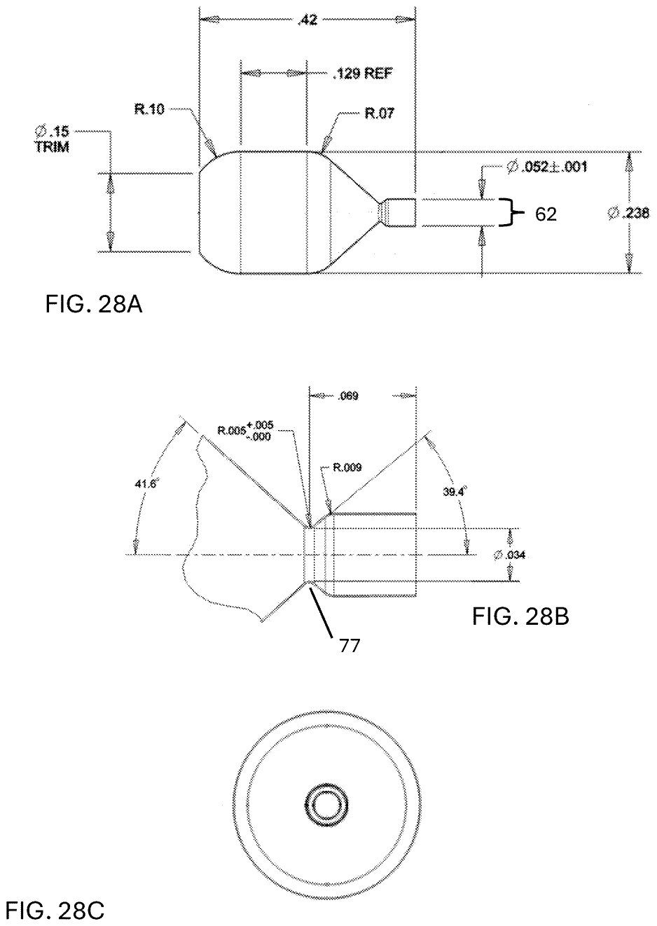

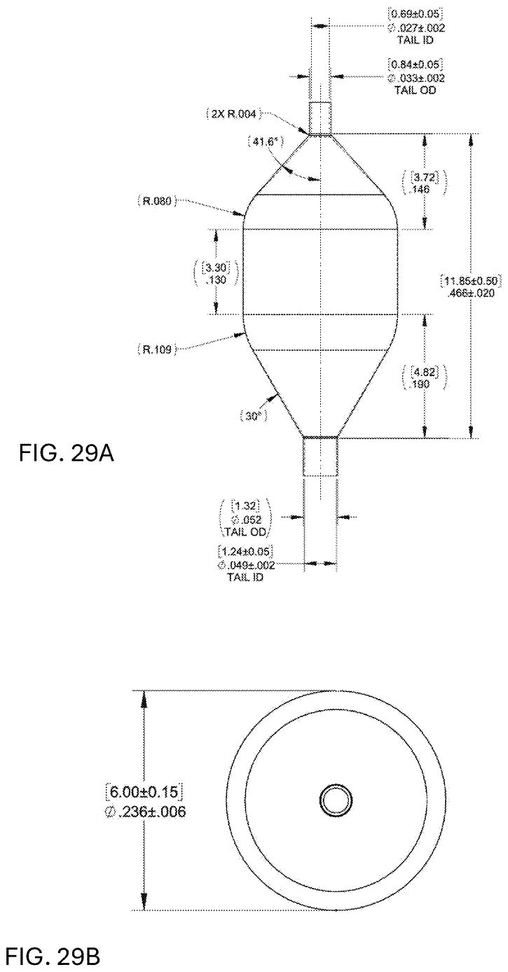

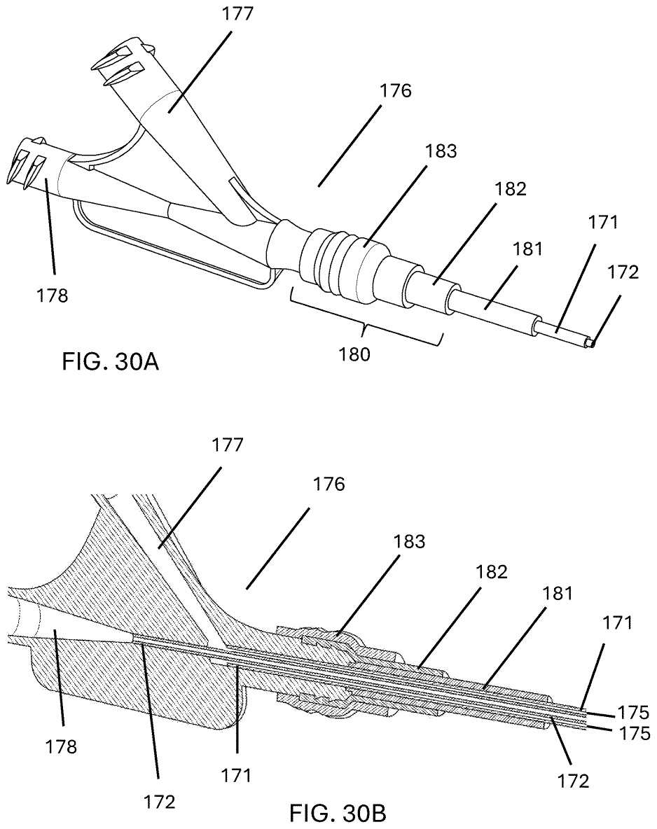

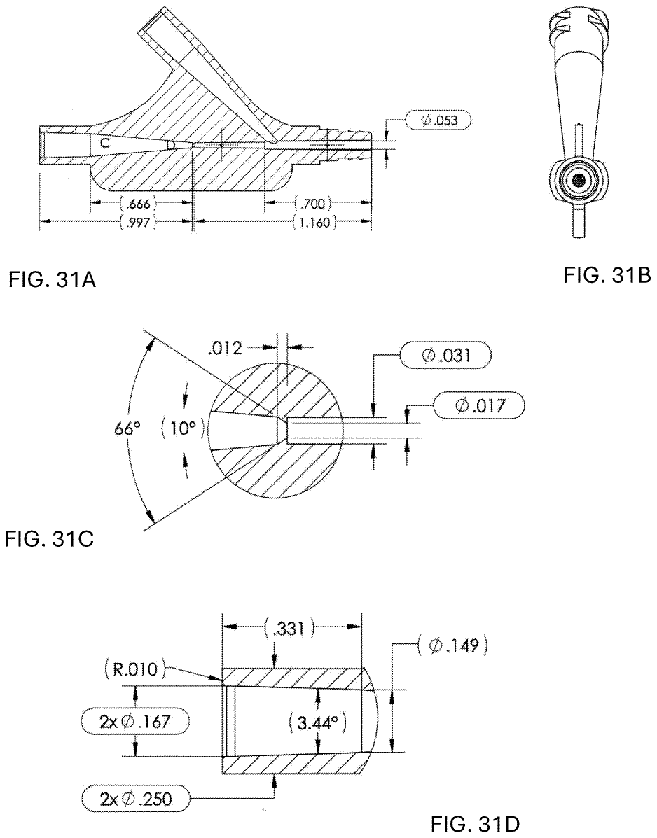

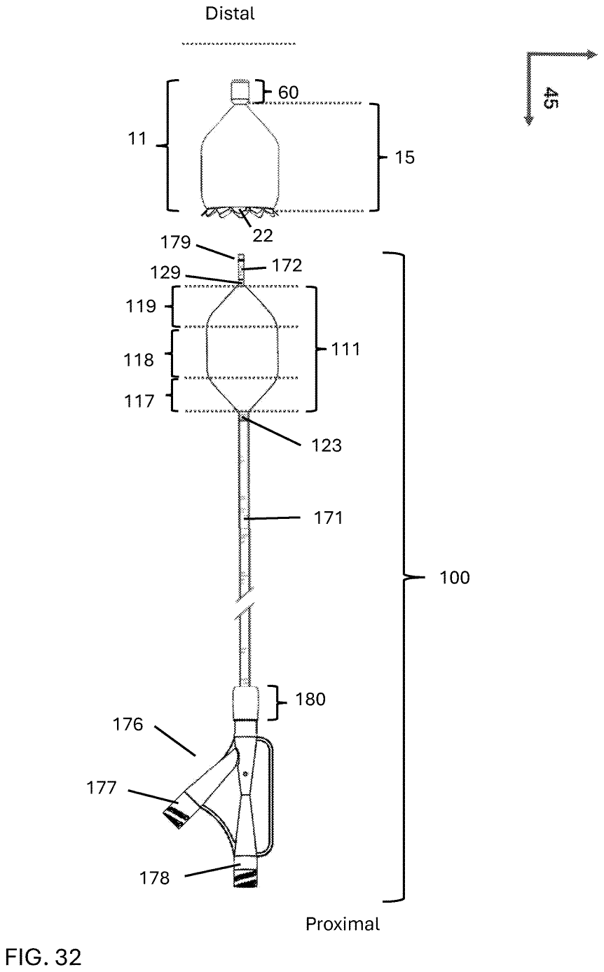

The specific dimensions of a 6 mm version of a preferred example of the expanded expandable metallic implant 11 are shown in FIGS. 28A and 28B. The folded and pleated expandable metallic implant 13 is expanded by inflation of the associated folded and pleated balloon 115. The geometry of the inflated balloon 115 is shaped to match the geometry of the expanded expandable metallic implant 11. The specific dimensions of a 6 mm version of a preferred example of the inflated balloon 111 are shown in FIGS. 29A and 29B.

The design, materials, and manufacturing methods of the various components of expandable metallic implant devices 1 can provide optimal safety and efficacy for patients. In some examples, the metal(s) used to form the wall 29 of the core portion 14 of expandable metallic implants 10 may be: biocompatible; malleable enough to allow for pleating and folding; and strong enough to resist compression after expansion and implantation in patients. In some examples, the material(s) used to form the wall 137 of a balloon 110, must be carefully selected to provide: acceptable biocompatibility; enough strength to enable high pressure inflation and resist puncture; and enough compliance to allow for bulging of the over-inflated balloon 112 into the balloon retention region 20 of the expandable metallic implant 10, at the proximal edge of the expandable metallic implant 10, and into the flap window 24 of the expanded expandable metallic implant 11 so as to lift of the free edge of the one more flaps 22 of the expanded expandable metallic implant 11 from the exterior surface 41 of the expanded expandable metallic implant 11 with balloon overinflation. In another example, the metal(s) used to form the wall 29 of the core portion 14 of expandable metallic implants 10, the thickness of the wall 29 of the core portion 14 of expandable metallic implants 10, the material(s) used to form the wall 137 of the balloon 110, and the thickness of the wall 137 of the balloon 110 can be carefully selected and balanced to provide: a balloon 110 capable of accepting a high enough pressure to expand a folded and pleated expandable metallic implant 13; a balloon 110 compliant enough to allow for bulging of the over-inflated balloon 112 into the balloon retention region 20 of the expandable metallic implant 10, at the proximal edge of the expanded expandable metallic implant 11, and/or into the flap window 24 of the expanded expandable metallic implant 11 so as to lift of the free edge of the one more flaps 22 of the expanded expandable metallic implant 11 from the exterior surface 41 of the expanded expandable metallic implant 11 with balloon overinflation; and provide adequate resistance to compression of the expanded expandable metallic implant 11 after separation from the balloon catheter 100 in a patient. In another example, the length (along the first axis 45) of the seal zone 18 of the expanded expandable metallic implant 11 may be chosen carefully to provide: a long enough length to provide adequate obstruction or reduction of flow in arteries, veins, aneurysms, parent vessels of saccular aneurysms, paravalvular leak pathways, biological conduits, or other blood-containing, fluid-containing, or biological spaces; and a short enough length to provide acceptability deliverability of folded and pleated expandable metallic implant devices 2 to the treatment site in patients. In another example, the shape of the balloon retention region 20 of expandable metallic implant 10 may be chosen carefully to provide: a reduction in the diameter of the expanded expandable metallic implant 11 in the balloon retention region 20 of the expandable metallic implant 10 large enough to provide adequate attachment of the inflated balloon 111 and the expanded expandable metallic implant 11; and a reduction in the diameter of the expanded expandable metallic implant 11 in the balloon retention region 20 of the expanded expandable metallic implant 11 small enough to allow for a low force removal of a deflated or collapsed balloon 113 from an expanded expandable metallic implant 11.

In some examples, the thickness of the wall 31 of a flap 22 of the expandable metallic implant 10 may be made thinner than the adjacent wall 30 of the expandable portion 15 of the expandable metallic implant 10 by adding a mask or coating to all or a portion of the wall of a flap 22 during electroforming. In some examples, the thickness of the wall of a flap 22 of the expandable metallic implant 10 may be made thinner than the adjacent wall 30 of the expandable portion 15 of the expandable metallic implant 10 by removing metal from the wall of the flap 22 after electroforming or electrolysis, including by methods wherein metal is removed with a laser. In some examples, the thickness of the wall of a region of the base of a flap 22 of the expandable metallic implant 10 may be made thinner than the adjacent wall 30 of the expandable portion 15 of the expandable metallic implant 10 by adding a mask or coating to a portion of the wall of a flap 22 during electroforming or electrolysis. In some examples, the thickness of the wall of a region of the base of a flap 22 may be made thinner than the adjacent wall 30 of the expandable portion 15 of the expandable metallic implant 10 by removing metal from a portion of the wall of a flap 22 after electroforming, including by methods wherein metal is removed with a laser. In some examples, the expandable metallic implant 10 is configured with a wall that is open on the proximal end and closed or obstructed on the distal end and wherein the wall continuously from the proximal end to the distal end, except for one or more flap windows 24. In some examples, the wall 29 of the core portion 29 of an expandable metallic implant 10 extends generally continuously from the proximal zone 17 (if any), through the seal zone 18, through the transition zone 19, and through the 60 neck (if any), except for one or more flap windows 24.

In some examples, of an expandable metallic implant device 1 comprising a compliant or semi-compliant balloon 110 and at least one flap 22, the free edge of the flap 22 of the expandable metallic implant 10 can be raised from the adjacent wall 30 of the expandable portion 15 of the expandable metallic implant 10 during overinflation of the compliant or semi-compliant balloon 110. In some examples, of an expandable metallic implant device 1 comprising a compliant or semi-compliant balloon 110 and at least one flap 22, the free edge of the flap 22 of the expandable metallic implant 10 can be raised from the adjacent wall 30 of the expandable portion 15 of the expandable metallic implant 10 when the compliant or semi-compliant balloon 110 is inflated at a pressure of 1, 2, 3, 4, 5, 6, 7, 8, 9, 10, 11, 12, 13, 14, 15, 16, 17, 18, 19, or 20 atmospheres.

FIG. 13A is a side view of the distal end of an example of a folded and pleated expandable metallic implant device 2 with a folded and pleated balloon 115 and a folded and pleated expandable metallic implant 13. FIG. 13B is a side view of the distal end of the example of and expanded expandable metallic implant 11 device 2 shown in FIG. 13A after inflation of the balloon 110 showing an inflated balloon 111 and an expanded expandable metallic implant 11. This example of an expanded expandable metallic implant 11 has a proximal zone 17, a seal zone 18, a transition zone 19, and a neck 60. The proximal zone 17 comprises a balloon retention region 20 and a fixation region 21. The fixation region 21 comprises one row of pointed flaps 22 with a proximal-distal alignment within a rectangular flap window 24. FIG. 13C is a side view of the distal end of the example of an expanded expandable metallic implant 11 device 3 shown in FIG. 13B after over-inflation of the balloon 110 showing an over-inflated balloon 112 and flaps 22 with free ends that are raised from the surface of the expanded expandable metallic implant 11. FIG. 13D is a side view of the distal end of the example of the expandable metallic implant device 1 shown in FIG. 13C after deflation or collapse of the balloon 110 showing a deflated or collapsed balloon 113. FIG. 13E is a side view of the distal end of the example of the expandable metallic implant device 1 shown in FIG. 13D after separation of the deflated or collapsed balloon 113 and the expanded expandable metallic implant 11.

FIG. 14 is a magnified, angled perspective view of the distal end of an example of the expanded expandable metallic implant 11 device 3 shown in FIG. 13C.

In some examples, of an expandable metallic implant device 1, the thickness of the wall of a flap 22 of an expandable metallic implant 10 may be thinner than the adjacent wall 30 of the expandable portion 15 of the expandable metallic implant 10 to facilitate the lifting of the free edge of the flap 22 from the adjacent wall 30 of the expandable portion 15 of the expandable metallic implant 10 during overinflation of a compliant or semi-compliant balloon 110.

FIG. 15A is a side view of the distal end of an example of a folded and pleated expandable metallic implant device 2 with a folded and pleated balloon 115 and a folded and pleated expandable metallic implant 13. FIG. 15B is a side view of the distal end of the example of the expanded expandable metallic implant 11 device 3 shown in FIG. 15A after inflation of the balloon 110 showing an inflated balloon 111 and an expanded expandable metallic implant 11. This example of an expanded expandable metallic implant 11 has a proximal zone 17, a seal zone 18, a transition zone 19, and a neck 60. The proximal zone 17 comprises a fixation region 21 with one row of square flaps 22 on the proximal end of the expanded expandable metallic implant 11 wherein the flaps 22 have a distal-proximal alignment. The seal zone 18 comprises a balloon retention region 20. FIG. 15C is a side view of the distal end of the example of the expanded expandable metallic implant 11 device 3 shown in FIG. 15B after over-inflation of the balloon 110 showing an over-inflated balloon 112 and flaps 22 with free ends that are raised from the surface of the expanded expandable metallic implant 11. FIG. 15D is a side view of the distal end of the example of the expandable metallic implant device 1 shown in FIG. 15C after deflation or collapse of the balloon 110 showing a deflated or collapsed balloon 113. FIG. 15E is a side view of the distal end of the example of the expandable metallic implant device 1 shown in FIG. 15D after separation of the deflated or collapsed balloon 113 and the expanded expandable metallic implant 11.

FIG. 16 are exemplary planar views of a pointed flap 22 of an expanded expandable metallic implant 11 with a linear hinge region 23 at the base of the flap 22 and within a flap window 24 of variable shape.

In some examples, the thickness of the wall of the base of the flap 22 may be thinner than the adjacent wall 30 of the expandable portion 15 of the expandable metallic implant 10 and the wall of the region adjacent the base of the flap 22 to facilitate the lifting of the free edge of the flap 22 from the adjacent wall 30 of the expandable portion 15 of the expandable metallic implant 10 during overinflation of a compliant or semi-compliant balloon 110 by forming a hinge region 23 to facilitate the lifting of the flap 22. In some examples, the region of the wall of a flap 22 of the expandable portion 15 of the expandable metallic implant 10 with the smaller thickness is linear and located at the base of the flap 22, forming a hinge region 23 to facilitate the lifting of the flap 22 by a compliant or semi-compliant balloon 110.

The expandable metallic implant 10 may be dimensioned and configured to fill at least a portion of the lumen an artery, vein, fusiform aneurysm, parent vessel of a saccular aneurysm, paravalvular leak pathway, biological conduit, or other blood-containing, fluid-containing, or biological space wherein the expandable metallic implant 10 remains in place in an expanded state after deflation or collapse of the balloon 110 and removal of the balloon catheter 100. A variety of shapes and sizes of the expanded expandable metallic implant 11 are described herein. In some examples, the expanded expandable metallic implant 11 is configured with a generally cylindrical or tubular seal zone 18. In some examples, the cross-sectional shape of the seal zone 18 is generally round in shape. In some examples, the expanded expandable metallic implant 11 is configured with a generally rounded, hemispherical, or conical transition zone 19. In some examples, the expanded expandable metallic implant 11 is configured with a transition zone 19 with a generally oblate hemispheroid, prolate hemispheroid, or a paraboloid shape. In some examples, the expanded expandable metallic implant 11 is configured with cylindrical or tubular seal zone 18 with a flat distal end, as shown in FIG. 7G and H. In some examples, the expanded expandable metallic implant 11 is configured with a generally cylindrical or tubular seal zone 18 and a generally rounded, hemispherical, conical, oblate hemispheroid, prolate hemispheroid, or a paraboloid shaped transition zone 19, wherein the exterior surface 41 of the seal zone 18 form a seal against the adjacent vessel wall (or other tissue) and the wall 30 of the transition zone 19 provides a solid barrier to the flow of blood or other fluids. Some preferred shapes of the expandable metallic implants 10 for the treatment of arteries, veins, fusiform aneurysms, parent vessels of saccular aneurysms, paravalvular leak pathways, and other biological conduits include shapes with a cylindrical seal zone 18 and a rounded or conical transition zone 19.

Expandable metallic implants 10 may be defined by a first axis 45 and a second axis 46, wherein the first axis 45 extends along the centerline of an expanded expandable metallic implant 11 that begins at the proximal zone 17 or the seal zone 18 and ends at the distal aspect of the transition zone 19, neck 60, or neck assembly 65; and the second axis 46 is transverse to the first axis 45. Expandable metallic implant devices may be defined by a first axis 45 and a second axis 46 to the first axis 45, wherein the first axis 45 extends along a centerline present in the center of the lumen 161 of the elongate body 160 that begins at the proximal end of the hub 162 of the elongate body 160 and ends at the distal end of the neck 60 or neck assembly 65 of the expandable metallic implant 10; and the second axis 46 is transverse to the first axis 45.

The length of the seal zone 18 of expandable metallic implants 10 can affect occlusion performance. Longer seal zones 18 are generally preferred, when possible, due to the reduced leakage of blood or other fluids around an expanded expandable metallic implant 11 in vivo with a long contact surface with adjacent tissues. For the occlusion of arteries and veins, a longer seal zone 18 is preferred, if possible. Therefore, expandable metallic implants 10 for the occlusion of arteries, veins, and other biological conduits are often longer than they are wide. However, increasing the length of the seal zone 18 of an expandable metallic implant 10 increases the length of the rigid folded and pleated expandable metallic implant 13 portion of the corresponding folded and pleated expandable metallic implant device 2, potentially reducing deliverability and representing a trade-off.

In some examples, the seal zone 18 of the expanded expandable metallic implant 11 has a length (along the first axis 45) that is greater than, equal to, or less than the largest diameter (along the first axis 46) of the seal zone 18. In some examples, the transition zone 19 of the expanded expandable metallic implant 11 has a length (along the first axis 45) that is greater than, equal to, or less than the largest diameter (along the first axis 46) of the seal zone 18. In some examples, the expandable portion 15 of the expanded expandable metallic implant 11 has a length (along the first axis 45) that is greater than, equal to, or less than the largest diameter (along the first axis 46) of the seal zone 18. In some examples, the expanded expandable metallic implant 11 is configured to have a maximum diameter of between 2-50 mm when measured parallel to the second axis 46. In some examples, seal zone 18 of the expanded expandable metallic implant 11 is configured to have a maximum diameter of between 2-50 mm when measured parallel to the second axis 46.

In some examples, the expandable portion 15 of the expandable metallic implant 10, when expanded, is configured to have a maximum length of between 3-100 mm when measured parallel to the first axis 45. In some examples, the exterior surface 41 of the expandable metallic implant 10 comprises a lubricous or hydrophilic coating layer. In certain instances, this lubricous or hydrophilic coating layer reduces the frictional forces between the exterior surface 41 of the expandable metallic implant 10 and the adjacent tissue in vivo, thereby reducing the risk of tissue injury or device damage during placement and expansion of the expandable metallic implant 10.

In some examples, of an expandable metallic implant device 1, the wall 30 of the expandable portion 15 of the expandable metallic implant 10 or the wall 29 of the core portion 14 of an expandable metallic implant 10 may comprise a single metal layer with a thickness of 3-500 microns (a “single layered expandable metallic implant”). In some examples, of a single layered expandable metallic implant 32, the wall 30 of the expandable portion 15 of the expandable metallic implant 10 or the wall 29 of the core portion 14 of an expandable metallic implant 10 may comprise a single metal layer with a thickness in the range of 5-30 microns or 3-100 microns. In some examples, the wall 30 of the expandable portion 15 of the expandable metallic implant 10 or the wall 29 of the core portion 14 of an expandable metallic implant 10 comprises gold with a thickness of ≤5 microns, ≤10 microns, ≤15 microns, ≤20 microns, ≤25 microns, ≤30 microns, ≤35 microns, or ≤40 microns. In some examples, the layer is a layer of gold or a gold alloy. In some examples, of an expandable metallic implant device 1, the wall 30 of the expandable portion 15 of the expandable metallic implant 10 or the wall 29 of the core portion 14 of an expandable metallic implant 10 may comprise more than one layer (a “multilayered expandable metallic implant”). The overall thickness of the wall 30 of the expandable portion 15 of a multilayered expandable metallic implant 33 or the wall 29 of the core portion 14 of a multilayered expandable metallic implant 33 may range between 3-2000 microns.

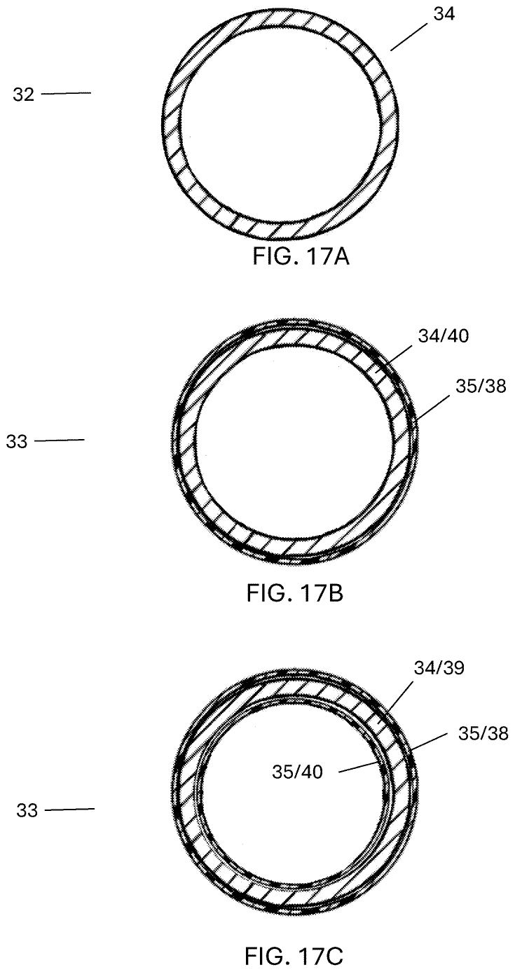

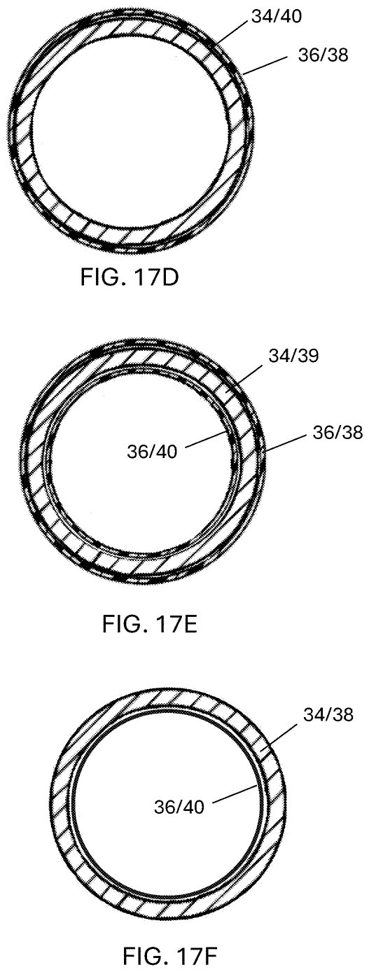

FIG. 17A is an end planar view of an example of an expanded expandable metallic implant 11 having a single structural metal layer 34. FIG. 17B is an end planar view of an example of an expanded expandable metallic implant 11 having a structural metal layer 34 as an inner layer 40 and a functional metal layer 35 as an outer layer 38. FIG. 17C is an end planar view of an example of an expanded expandable metallic implant 11 having a structural metal layer 34 as a middle layer 39, a functional metal layer 35 as an outer layer 38, and a functional metal layer 35 as an inner layer 40. FIG. 17D is an end planar view of an example of an expanded expandable metallic implant 11 having a structural metal layer 34 as an inner layer 40 and a polymer layer 36 as an outer layer 38. FIG. 17E is an end planar view of an example of an expanded expandable metallic implant 11 having a structural metal layer 34 as a middle layer 39, a polymer layer 36 as an outer layer 38, and a polymer layer 36 as in inner layer 40. FIG. 17F is an end planar view of an example of an expanded expandable metallic implant 11 having a structural metal layer 34 as an outer layer 38 and a polymer layer 36 as in inner layer 40.

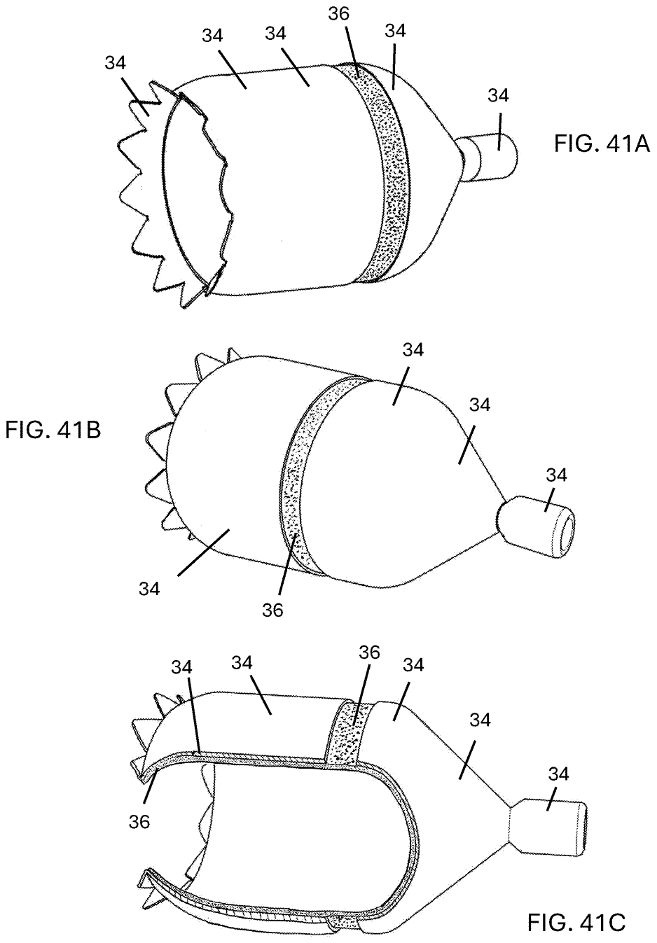

In some examples, the internal polymer layer 36 may act as a connector between segmented metal (including gold) sections of the expanded expandable metallic implant 11 in the event that a longer seal zone 18 is desired, as shown in FIG. 41. In some examples, it may be desirable for the length of the seal zone 18 of the expanded expandable metallic implant to be 0.25, 0.5, 1.0, 1.5, 2.0, 3.0 or many times the diameter of the seal zone 18. In some examples, the segmented metal (including gold) sections of the expanded expandable metallic implant 11 can be fabricated on a continuous inner polymer layer 3. In some examples, the segmented metal (including gold) sections of the expanded expandable metallic implant 11 can be fabricated on a segmented inner polymer layer 36, with the multiple segmented metal (including gold) sections of the expanded expandable metallic implant 11 joined together by the segmented inner polymer layer 36.

In some examples, the wall 30 of the expandable portion 15 of the expandable metallic implant 10 or the wall 29 of the core portion 14 of an expandable metallic implant 10 may comprise a single metal layer or multiple metal layers. In some examples, the wall 30 of the expandable portion 15 of the expandable metallic implant 10 or the wall 29 of the core portion 14 of an expandable metallic implant 10 may be comprised of a single metal, a single metal alloy, two different metals, two different metal alloys, or more than two different metals, or metal alloys. In some examples, the wall 30 of the expandable portion 15 of the expandable metallic implant 10 or the wall 29 of the core portion 14 of an expandable metallic implant 10 may be formed by electroforming. In some examples, the wall 30 of the expandable portion 15 of the expandable metallic implant 10 or the wall 29 of the core portion 14 of an expandable metallic implant 10 may comprise gold, platinum, alloys thereof, and combinations thereof. In some examples, the wall 30 of the expandable portion 15 of the expandable metallic implant 10 or the wall 29 of the core portion 14 of an expandable metallic implant 10 may have a thickness of 3-300 microns, or a thickness of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47, 48, 49, or 50 microns. In some examples, the wall 30 of the expandable portion 15 of the expandable metallic implant 10 or the wall 29 of the core portion 14 of an expandable metallic implant 10 may comprise a metal layer wherein the primary purpose of the metal is load bearing (“a structural metal layer”). In some examples, the structural metal layer 34 may have a thickness of 3-300 microns. In some examples, the structural metal layer 34 may be produced through a process of electroforming. In some examples, the structural metal layer 34 may have a thickness of 3-300 microns. In some examples, the structural metal layer 34 may comprise gold or platinum, alloys thereof, and combinations thereof. In some examples, the structural metal layer 34 may have a thickness of 3-300 microns, or a thickness of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47, 48, 49, or 50 microns. For some of these examples, expandable portion 15 of the expandable metallic implant comprises a structural metal layer 34 comprising gold with a thickness of 3-300 that can be folded and pleated with a balloon 110, advanced to a target location in a human patient with the balloon catheter 100 and expanded at the target location by expansion of the balloon 110. In some examples, a portion of the wall 30 of the expandable portion 15 of the expandable metallic implant 10 or the wall 29 of the core portion 14 of an expandable metallic implant 10 may also comprise a metal layer wherein the primary purpose of the metal is functional and not load bearing (“a functional metal layer”), such as for increasing fluoroscopic visualization, conducting electricity enhancing biocompatibility, and inducing a biological response in the adjacent tissue, including by stimulating the growth of an endothelial or fibrous layer on the surface of the functional metal layer 35, among other purposes. In some examples, the structural metal layer 34 may be produced through a process of electroforming. In some examples, the structural metal layer 34 may have a thickness of 0.0005 to 3 microns. In some examples, the functional metal layer 35 may be present on all, or only a portion, of the wall 30 of the expandable portion 15 of the expandable metallic implant 10 or the wall 29 of the core portion 14 of an expandable metallic implant 10. By way of example and not limitation, the functional metal layer 35 may be present on 100% or less than 100% of the wall 30 of the expandable portion 15 of the expandable metallic implant 10 or the wall 29 of the core portion 14 of an expandable metallic implant 10. In some examples, the functional metal layer 35 comprises gold, platinum, silver, titanium, vanadium, aluminum, nickel, tantalum, zirconium, chromium, magnesium, niobium, scandium, cobalt, palladium, manganese, molybdenum, alloys thereof, and combinations thereof.

In some examples, the wall 30 of the expandable portion 15 of the expandable metallic implant 10 or the wall 29 of the core portion 14 of an expandable metallic implant 10 may comprise a single metal layer and one or more polymer layers 36, or a more than one metal layer and one or more polymer layers 36. In some examples, of multilayered expandable metallic implants 10, the wall 30 of the expandable portion 15 of the expandable metallic implant 10 or the wall 29 of the core portion 14 of an expandable metallic implant 10 may be comprised of one or more of polyurethane, silicone, poly(p-xylylene), Parylene, or any other synthetic or natural polymer known in the art wherein the primary purpose of the polymer is functional and not load bearing (“a polymer layer”), such as for enhancing biocompatibility or inducing a biological response in the adjacent tissue, including by stimulating the growth of an endothelial or fibrous layer on the surface of the multilayered expandable metallic implant 33, among other purposes. In some examples, the polymer may be formed in a layer 33. In some examples, the portion of the wall 30 of the expandable portion 15 of the expandable metallic implant 10 or the wall 29 of the core portion 14 of an expandable metallic implant 10 with a polymer layer 36 may be formed by coating, electroplating, sputter deposition, or vapor deposition. In some examples, the portion of the wall 30 of the expandable portion 15 of the expandable metallic implant 10 or the wall 29 of the core portion 14 of an expandable metallic implant 10 with a polymer layer 36 may have a thickness of 0.0005-2000 microns. In some examples, the wall 30 of the expandable portion 15 of the expandable metallic implant 10 or the wall 29 of the core portion 14 of an expandable metallic implant 10 with a polymer layer 36 may have a thickness of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47, 48, 49, or 50 microns. In some examples, the polymer layer 36 may be present on all, or only a portion, of the wall 30 of the expandable portion 15 of the expandable metallic implant 10 or the wall 29 of the core portion 14 of an expandable metallic implant 10. By way of example and not limitation, the polymer layer 36 may be present on 100% or less than 100% of the wall 30 of the expandable portion 15 of the expandable metallic implant 10 or the wall 29 of the core portion 14 of an expandable metallic implant 10. For some of these examples, one or more of the polymers may comprise an adhesive or glue.

In some examples, one or more zones, regions, or portions of the wall 29 of the core portion 14 of the expandable metallic implant 10 may be thicker or thinner than one or more other zones, regions, or portions of the wall 29 of the core portion 14 of the expandable metallic implant 10. By way of example and not limitation, all or a portion of the wall in the proximal zone 17, seal zone 18, transition zone 19,balloon retention region 20, fixation region 21, expandable portion 15, flap 22, flap hinge region 23, neck 60, or neck transitional region 77 between the transition zone 19 and the neck 60 may be thicker or thinner than another zone, region, or portion of the wall 29 of the core portion 14 of the expandable metallic implant 10. In some examples, the wall 30 of the expandable portion 15 of the expandable metallic implant 10 in the flap hinge region 23 may be thinner than the wall 30 of the expandable portion 15 of the expandable metallic implant 10 in the flap 22. In some examples, the thinner region may be linear. In another example, the wall 29 of the core portion 14 of the expandable metallic implant 10 in the neck transitional region 77 between the transition zone 19 and neck 60 may thinner than the wall 29 of the transition zone 19 or neck 60. In another example, the wall 29 of the expandable portion 15 of the expandable metallic implant 10 in the seal zone 18 may be thicker than the wall 30 of the expandable portion 15 of the expandable metallic implant 10 in the transition zone 19. In another example, the wall 30 of the expandable portion 15 of the expandable metallic implant 10 in the transition zone 19 may be thicker than the wall 30 of the expandable portion 15 of the expandable metallic implant 10 in the seal zone 18.

Metal isn't generally considered compliant in the way some polymers are. Metals tend to be rigid, but they can exhibit some degree of compliance under certain conditions, such as when they are thin or when they undergo plastic deformation. In some examples, after expansion of an expandable metallic implant 10 to its nominal diameter, the expandable portion 15 of an expanded expandable metallic implant 11 of an expanded expandable metallic implant 11 device 3 is non-compliant, including when the pressure inside the inflated balloon 111 or over-inflated balloon 112 is in a range between 1-20 atmospheres. In some examples, after expansion of an expandable metallic implant 10 to its nominal diameter, the expandable portion 15 of an expanded expandable metallic implant 11 of an expanded expandable metallic implant 11 device 3 is semi-compliant. In some examples, after expansion of an expandable metallic implant 10 with a balloon retention region 20 to its nominal diameter, the expandable portion 15 of the expanded expandable metallic implant 11 increases by ≤2%, ≤4%, ≤6%, ≤8%, ≤10% in diameter when fluid is injected into the interior space 120 of the inflated balloon 111 or over-inflated balloon 112 at a pressure of 1, 5, 10, or 20 atmospheres.

The exterior surface 41 of expandable metallic implants 10 may be formed or modified to improve biocompatibility, to promote rapid endothelialization of blood-contacting surfaces, to roughen the surface texture and reduce the risk of migration of the expandable metallic implant 10, or to increase the strength of the bonds that form between the expandable metallic implant 10 and the surrounding tissue to increase implant anchoring after placement in vivo. Surface modifications may include surface roughening or smoothing, changes in surface chemistry, attachment of molecules or biomolecules, or various combinations of these methods.

In some examples, at least a portion of the exterior surface 41 of an expandable metallic implant 10 may comprise a rounded, pebbled, granular, or textured surface, wherein the pebbles or granules have a surface height of 0.0001-10 microns or the distance between the highest and the lowest portions is 0.0001-10 microns. In some examples, the exterior surface 41 of an expandable metallic implant 10 comprises surface structures 43. In certain instances, the rounded, pebbled, or granular surface, and the surface structures 43 can increase surface roughness and increase frictional forces between the exterior surface 41 of an expandable metallic implant 10 and the adjacent tissue, including the internal surface of an artery, vein, aneurysm, parent vessel of a saccular aneurysm, paravalvular leak pathway, biological conduit, or other blood-containing, fluid-containing, or biological space, thereby reducing the risk of movement or migration of the expanded expandable metallic implant 11 after placement in vivo. At least a portion of the wall 30 of the expandable portion 15 of the expandable metallic implant 10 may be formed by electroplating or electroforming, a process that can produce a rounded, pebbled, or granular surface. In some examples, the exterior surface 41 of the expandable metallic implant 10 comprises a lubricous or hydrophilic coating layer. In certain instances, this lubricous or hydrophilic coating layer reduces the frictional forces between the exterior surface 41 of the multilayered expandable metallic implant 33 and the adjacent tissue in vivo, thereby reducing the risk of tissue injury or device damage during placement and expansion of the multilayered expandable metallic implant 33.

In some examples, the expandable metallic implant 10 comprises a neck 60 distal to the transition zone 19. In some examples, the neck 60 extends distal to the distal edge of the transition zone 19 of an expanded expandable metallic implant 11. In some examples, the neck 60 extends away from the expandable metallic implant 10. In some examples, the neck 60 has a lumen 62. In some examples, the neck 60 has an opening 61. The length of neck 60 is between 0.3 mm-5 mm. The neck 60 may define an opening 61 having a diameter between 0.010-0.100 inch, as measured parallel to the first axis 46 of the expandable metallic implant. The thickness of the wall 31 of the neck 60 may be the same, thicker, or thinner than the thickness of the wall 30 of the expandable portion 15 of the expandable metallic implant 10. In some examples, the wall 63 of the neck 60 may have a thickness between 3 microns and 2000 microns. In some examples, at least a portion of the neck 60 comprises a layer of radiopaque metal that is visible under fluoroscopy, including a layer of platinum, iridium, gold, silver, or alloys or combinations thereof.

One or more additional neck components 66 may be joined to the neck 60, forming a neck assembly 65. By way of example and not limitation, additional neck components 66 may have the form of bodies, structures, ring structures, tubular segments or structures 67, telescoping structures, guidewire segments 69, catheter segments 75, or telescoping catheter segments. These additional neck components 66 may comprise metal, polymer, adhesive, and combinations thereof. The length of the neck assembly 65 is between 2 mm and 50 mm. These additional neck components 66 may comprise a radiopaque metal that is visible during fluoroscopy, including platinum, iridium, gold, silver, or alloys or combinations thereof. In some examples, a tubular segment or structure 67 is bonded to at least a portion of the neck 60 or neck assembly 65. In some examples, the neck tubular segment or structure 67 comprises a radiopaque metal that is visible under fluoroscopy, including platinum, iridium, gold, silver, or alloys or combinations thereof. In some examples, neck structures may comprise one piece, while in other examples, these bodies, structures, and segments may comprise two or more pieces that are bonded together, including bonded together with a glue or adhesive.

The additional neck components 66 of expandable metallic implants 10 may comprise guidewire segment 69 joined to the neck 60 and extends distally from the expandable metallic implant 10 to enable easier insertion of the folded and pleated expandable metallic implant 13 into and through the hub 203 of a guide catheter or guide sheath 200, to guide the folded and pleated expandable metallic implant 13 through the lumen 202 of the guide catheter or guide sheath 200, and to guide the advancement of the folded and pleated expandable metallic implant 13 in vivo. In some examples, the guidewire segment 69 is configured as a wire assembly comprising a core wire 70 surrounded by an outer externally wound (or coiled) wire 71. The core wire 70 has a distal end, a middle portion, and a proximal end. The middle portion of the core wire 70 may have a diameter that is greater than the diameter of the distal end, the proximal end, or both the distal and proximal end. In some examples, the core wire 70 is tapered at the distal end, the proximal end, or both the distal and proximal end. The wound wire 71 can be wound in a right-hand manner or a left-hand manner around the core wire 70. The wound wire 71 may be securely attached to the core wire 70 at the distal end, the proximal end, or both the distal end and the proximal end with an end cap 72. In some examples, the core wire 70 comprises stainless steel, nitinol, platinum, or gold, or combinations thereof. In some examples, the guidewire segment 69 comprises a lubricious coating layer or covering. In some examples, the wound wire 71 comprises platinum, gold, or combinations or alloys thereof. In some examples, the guidewire segment 69 comprises a polymer strand or a polymer strand plated or coated with a radiopaque metal such as platinum, iridium, gold, tungsten, or combinations thereof. In some examples, the polymer strand portion of the guidewire segment 69 further comprises one or more radiopaque markers that are visible during fluoroscopy, including radiopaque marker comprised of platinum, iridium, gold, tungsten, or combinations thereof, and including radiopaque markers in the form of a ring or band around a portion of the polymer strand. In some examples, the guidewire segment 69 further comprises a coating layer, covering, or outer layer 73. In some examples, the coating layer, covering, or outer layer 73 is lubricious. In some examples, the coating layer, covering, or outer layer 73 comprises a polytetrafluoroethylene, polyimide, or composites thereof. The length of the guidewire segment 69 is between 5-50 mm, as measured parallel to the first axis 45 of the expandable metallic implant. In some examples, the guidewire segment 69 is joined or bonded to the neck 60 or a portion of the neck assembly 65 of the expandable metallic implant 10 and extends distally from the distal end of the neck 60 or neck assembly 65 of the expandable metallic implant. In some examples, the proximal end of the core wire 70 and wound wire 71 are inserted into the neck 60 or a portion of the neck assembly 65 of the expandable metallic implant 10 and the outer surface of the wound wire 71 is bonded to the inner surface of neck 60 or portion of the neck assembly 65 of the expandable metallic implant 10. In some examples, the proximal end of the core wire 70 is inserted into the neck 60 or a portion of the neck assembly 65 of the expandable metallic implant 10, the outer surface of the core wire 70 is bonded to the inner surface of the neck 60 or other portion of the neck assembly 65 of the expandable metallic implant 10, and the proximal portion of the wound wire 71 is wrapped around the core wire 70. In some examples, the proximal end of the core wire 70 is inserted into the neck 60 or a portion of the neck assembly 65 of the expandable metallic implant 10, the outer surface of the core wire 70 is bonded to the inner surface of the neck 60 or neck assembly 65 of the expandable metallic implant 10, and the proximal portion of the wound wire 71 is wrapped around a portion of the neck 60 or neck assembly 65 of the expandable metallic implant 10. The additional neck components 66 of expandable metallic implants 10 may comprise a segment of catheter 75 joined to the neck 60 that extends distally from the expandable metallic implant 10 to enable easier insertion of the folded and pleated expandable metallic implant 13 into and through the hub 203 of a guide catheter or guide sheath 200, to guide the folded and pleated expandable metallic implant 13 through the lumen 202 of the guide catheter or guide sheath 200, and to guide the advancement of the folded and pleated expandable metallic implant 13 in vivo.

The additional neck components 66 of expandable metallic implants 10 may further comprise a nose cone 68 or other structure joined to the neck 60 that may be configured to provide a smooth transition from a portion of the expandable metallic implant 10 that has a smaller outer diameter to a portion of the expandable metallic implant 10 that has a larger outer diameter (a neck transitional body or “nose cone”). In some examples, this nose cone 68 or other structure joined may be designed to reduce the risk of tissue injury or device damage when advancing or retracting the expandable metallic implant device 1 in vivo. In some examples, the outer diameter of the proximal portion of a nose cone 68 is within 0.01 inch of the outer diameter of the adjacent portion of the folded and pleated expandable metallic implant 13. In some examples, the outer diameter of the distal portion of the nose cone 68 is within 0.01 inch of the outer diameter of the neck 60 or additional neck components 66, such as a guidewire segment 69. In some examples, the outer diameter of the proximal portion of a nose cone 68 is within 0.01 inch of the outer diameter of the adjacent portion of the folded and pleated expandable metallic implant 13 and the outer diameter of the distal portion of the nose cone 68 is within 0.01 inch of the outer diameter of the neck 60 or additional neck components 66, such as a guidewire segment 69. In some examples, a nose cone 68 may be conical in shape. In some examples, a nose cone 68 may comprise a radiopaque metal that is visible during fluoroscopy, including platinum, iridium, gold, silver, or alloys or combinations thereof. In some examples, a nose cone 68 may comprise one or more polymers, including polyether ether ketone, polycarbonate, nylon, polyimide, polyether block amide, polytetrafluoroethylene, silicone, polyurethane, co-polyester polymer, thermoplastic rubber, silicone-polycarbonate copolymer, polyethylene ethyl-vinyl-acetate co-polymer, a biocompatible elastomer, a biocompatible resilient material, or a biocompatible adhesive. In some examples, at least a portion of the neck 60 and the nose cone 68, the neck 60 and the guidewire segment 69, the nose cone 68 and the guidewire segment 69; or the neck 60, the guidewire segment 69, and the neck 60 are bonded together and overlapping along a plane parallel to the first axis 45 of the expandable metallic implant.

In some examples, a nose cone 68 may comprise one piece, while in other examples, a nose cone 68 may comprise two or more pieces that are bonded together, including bonded together with a glue or adhesive. In some examples, a nose cone 68 is bonded to the neck 60. In some examples, a nose cone 68 is bonded to an additional neck component 66. In some examples, a nose cone 68 is bonded to the neck 60 and an additional neck component 66. In some examples, a portion of the inner surface of a nose cone 68 is bonded to a portion of the exterior surface 41 of a neck 60, or a portion of an additional neck component 66.

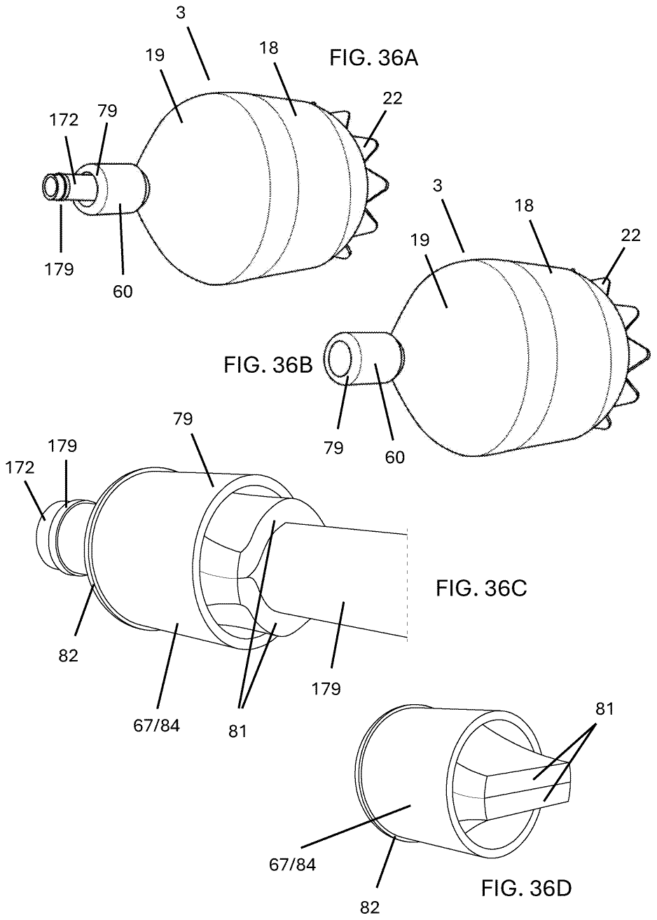

As shown in FIG. 36A to 36D, In some examples, the additional neck components 66 may comprise a valve 79 (including a valve) or similar flow modifying devices that are intended to reduce or block the flow of blood through the neck lumen 62 once the inner shaft 185 of balloon catheter 100 is removed (FIG. 36B). In some examples, the additional neck components 66 may comprise additional layers of material intended to guide catheters or wires through the neck lumen 62, to provide a lubricious coating to facilitate friction free movement of the balloon catheter 100 or guidewires 280. In some examples, the additional neck components 66 may comprise a glue layer to prevent the movement of additional neck components 66 relative to each other or to the neck 60.

When the inner shaft 185 of a balloon catheter 100 is present between the leaflets 81 of a valve 79, the leaflets 81 deflect aside allowing the inner shaft 185 to pass through (FIG. 36C). When the inner shaft 185 is removed, the leaflets move back to their closed position (FIG. 36D) and thus prevent flow through the neck lumen 62 or neck opening 61.

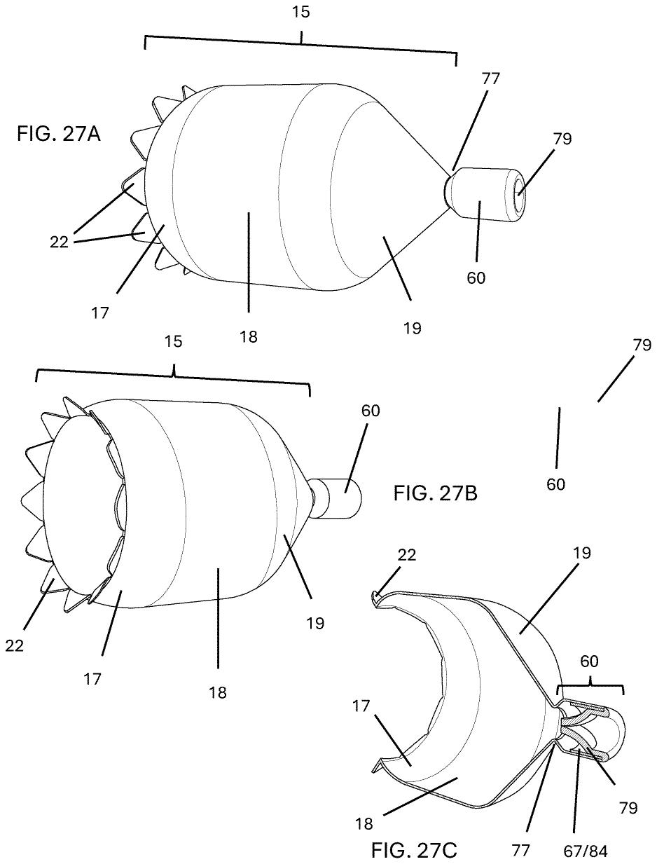

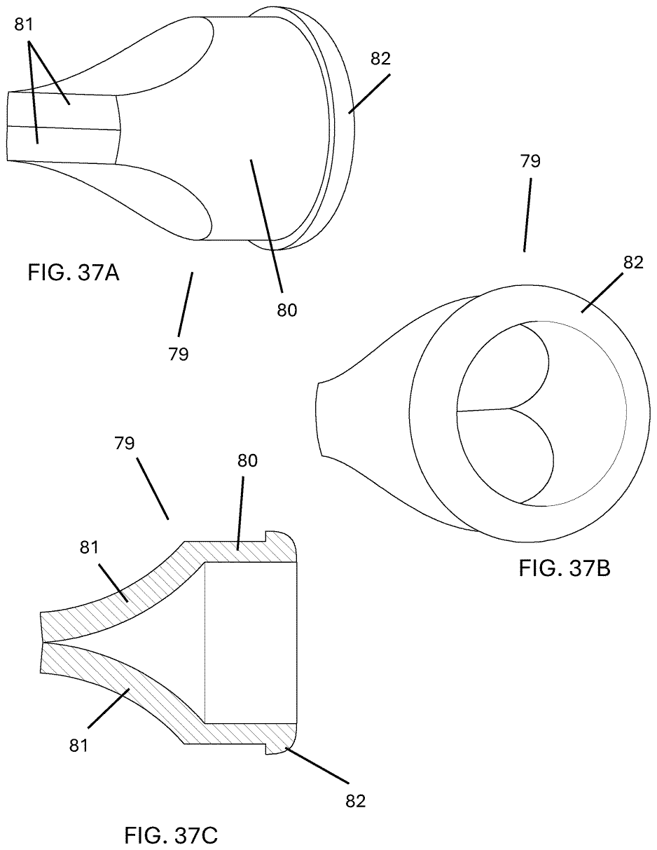

In some examples, where the additional neck components 66 may comprise a hemostatic valve, the valve 79 may comprise a tubular body 80, one or more leaflets 81 and one or more flanges 82 for effecting mounting or sealing between the valve 79 and the neck 60 or other neck components 66 (FIG. 27C). In one example, one flange can have a rounded surface (FIG. 37C) so as to enable the atraumatic sliding of the neck 60 against the body's tissues during the insertion and positioning of the expandable metallic implant device 1.

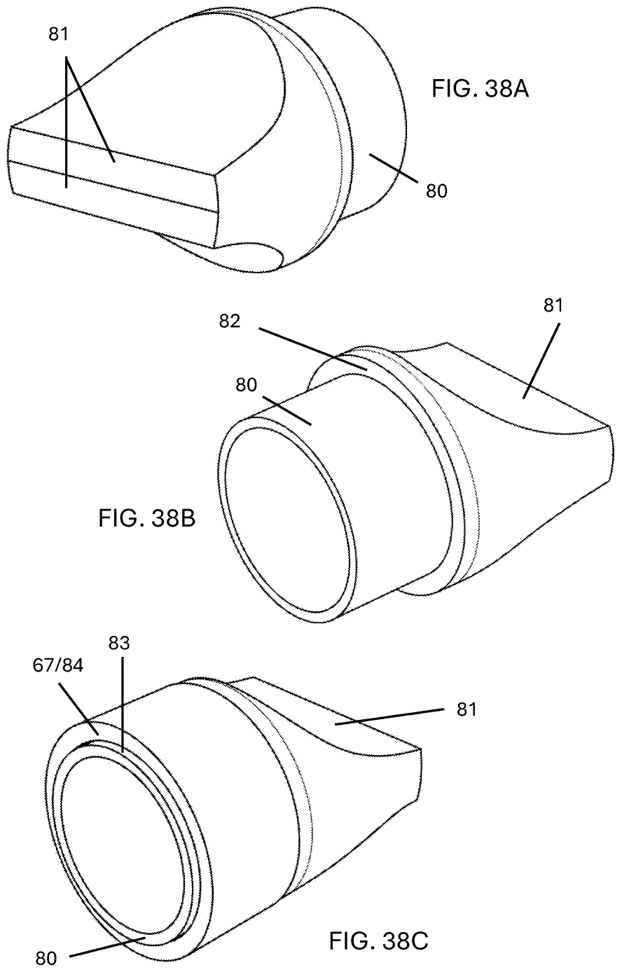

In some examples, the valve 79 can have a reduced diameter tubular body 80 so that a valve metal ring 85 can be fitted over the tubular valve body 80 to provide additional structural support and/or to facilitate mounting within the neck 60 (FIG. 38). The valve metal ring 84 may be attached to the valve tubular body 80 via an adhesive, forming valve assembly glue layer between the valve metal ring 84 and the valve tubular body 80.

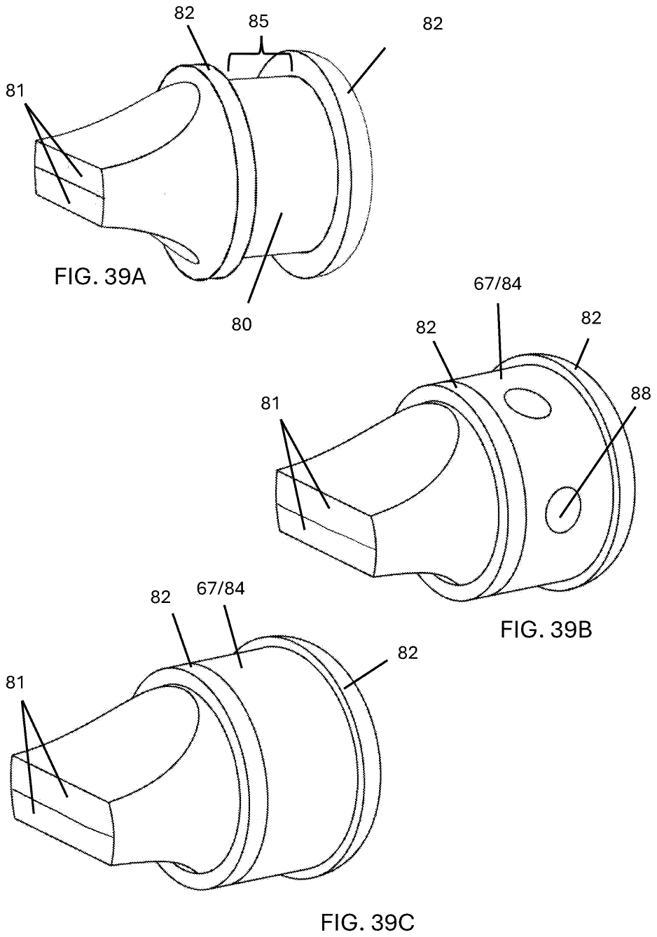

In some examples, the valve 79 can comprise a valve annular grove 85 sized to fit a valve metal ring 84 between two flanges 82 (FIG. 39A). In some examples, the valve 79 can be overmolded with a metal ring 84, having a number of holes or similar features that enable the valve material to integrate with the metal ring (FIG. 39B). The outer surface of the metal ring can then be glued to the inner surface of the neck 60. In some examples, the metal ring 84 can be positioned between two flanges 82 thus capturing the valve 79 (FIG. 39C) and then the outer surface of the metal ring can then be glued to the inner surface of the neck 60.

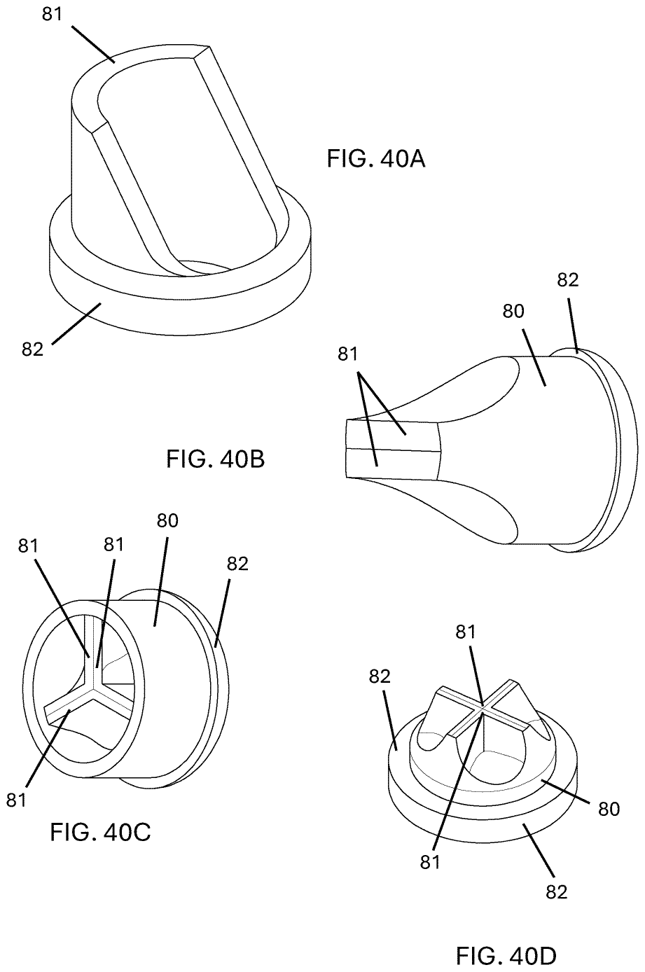

In some examples, the valve 79 can be a monocusp valve (FIG. 40A), a bi-leaflet valve (FIG. 40B), also called a duckbill valve, a tri-leaflet valve (FIG. 40C) similar to a native aortic valve or prosthetic valves, a four-leaflet, cross-slit valve (FIG. 40D) or any such valve capable of partially or fully closing, sealing, or obstructing the neck lumen 62 when balloon catheter 100 is removed, including when an inner shaft 172 of the balloon catheter 100. In some examples, the portion of the inner shaft 172 of the balloon catheter 100 comprising the distal neck 129 of the balloon 110 is positioned within the neck lumen 62 and/or within the valve 79 during insertion and positioning of the expandable metallic implant device 1.

In some examples, the valve 79 can be mounted in the neck lumen 62 directly via the application of a glue layer 83 between the outer surface of the valve and the inner surface of the neck 60. In some examples, the valve 79 can be overmolded with a metal ring 84, having a number of holes 83 or similar features that enable the valve material to integrate with the metal ring 84 (FIG. 39B). The outer surface of the metal ring 84 can then be glued to the inner surface of the neck 60. In some examples, the metal ring 84 can be positioned between two flanges 82 thus capturing the valve 79 and
the outer surface of the metal ring can then be glued to the inner surface of the neck 60 (FIG. 39C). In some examples, the metal ring 84 can be positioned in place by only one flange 82, as in FIG. 38.

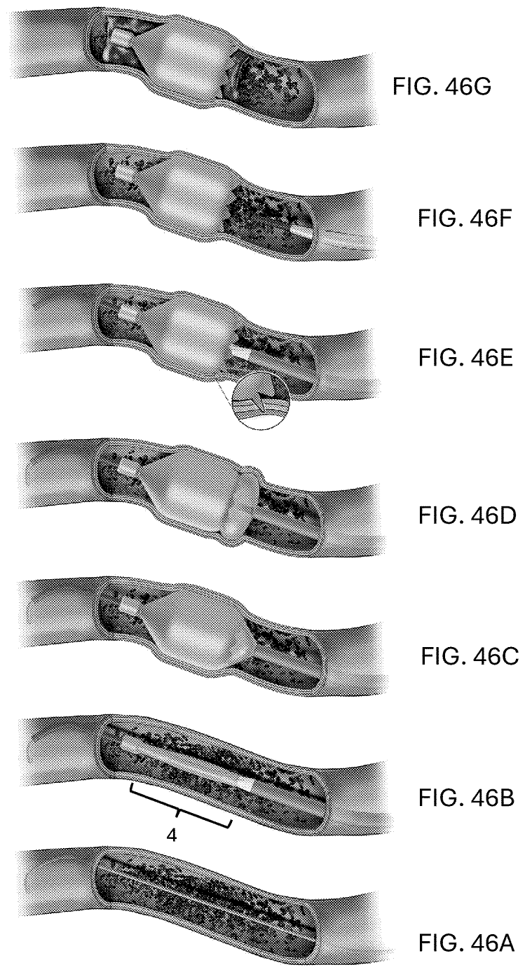

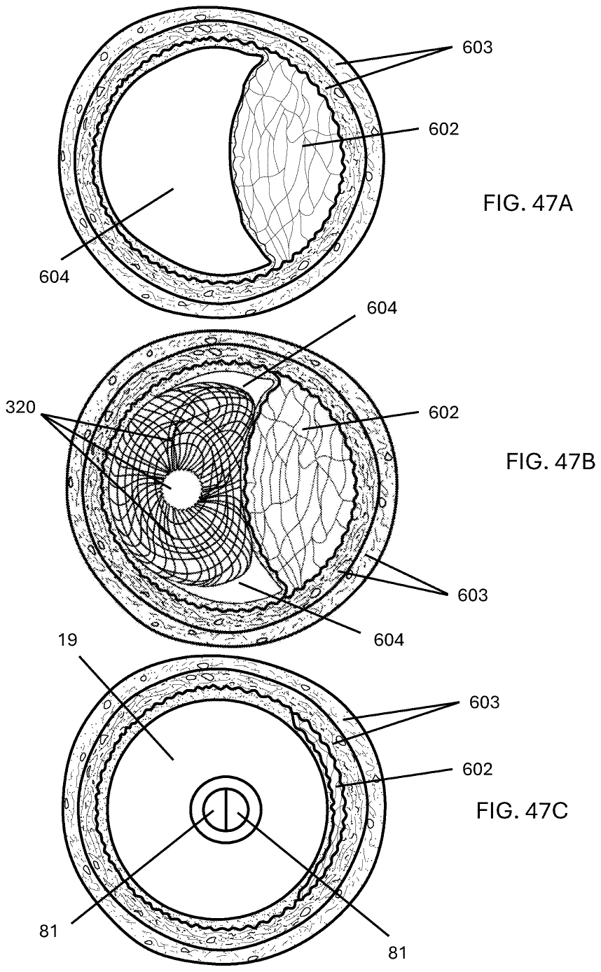

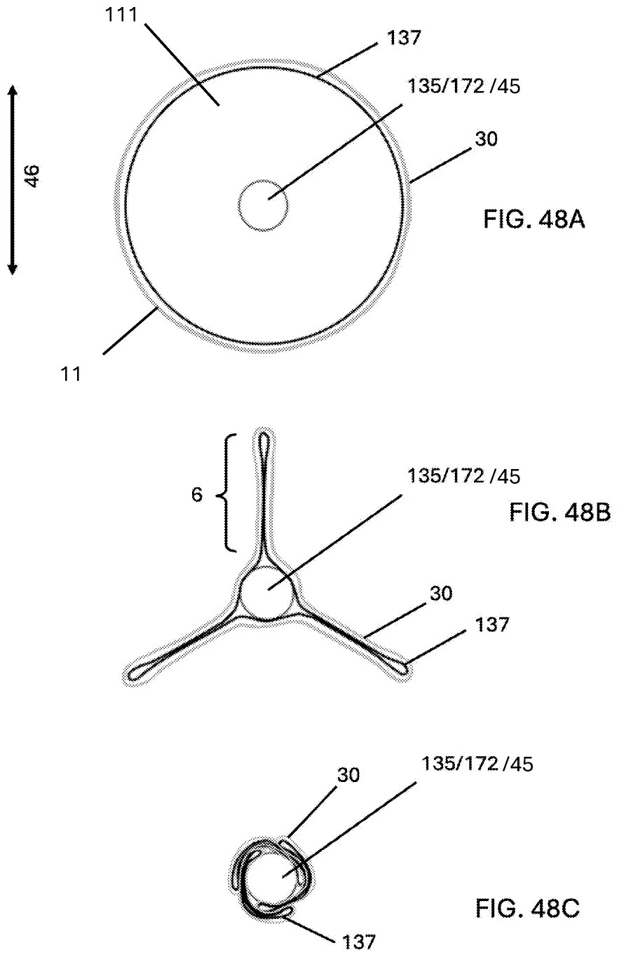

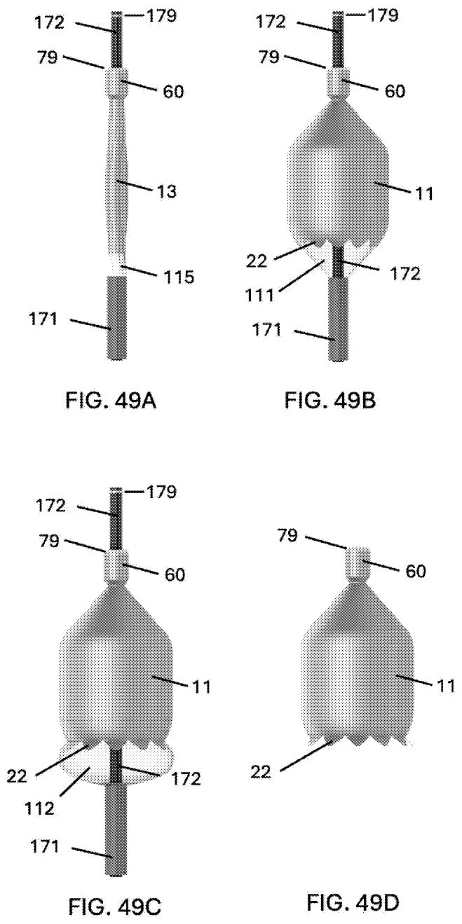

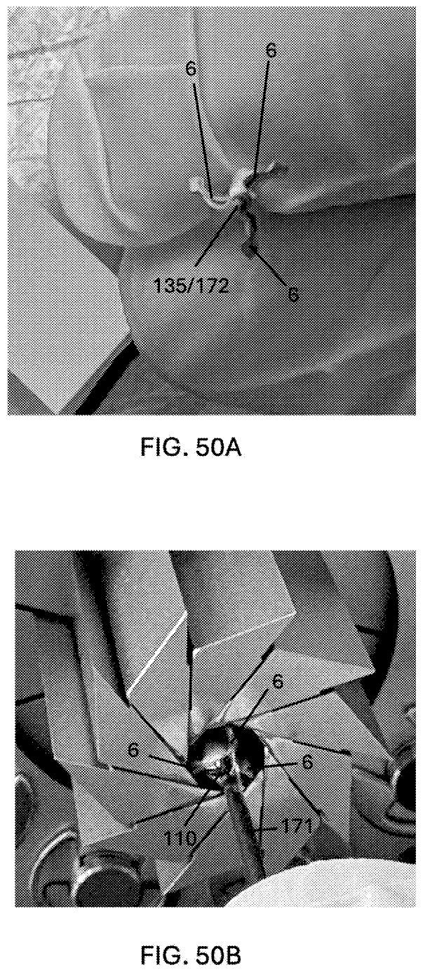

When using expandable implant devices, operators often remove air from the devices before inserting them into patients, a process sometimes known as “de-airing”. De-airing can be accomplished by injecting fluid into the devices to expel air and replace it with fluid. De-airing can also be accomplished by aspirating fluid into the devices to remove air. Incorporating one or more small openings (“flush openings”) in the balloon 110, proximal neck 123 of the balloon 110 or distal neck 129 of the balloon 110 would allow the injection or aspiration of fluid and the removal of air from the interior space 120 of the balloon 110 and the lumen 161 of the elongate body 160. The diameter of the flush opening(s) 138 would need to be large enough to allow for de-airing in a reasonable amount of time and small enough to allow for inflation of the balloon 110 and the expandable metallic implant 10 at the treatment site. In some examples, the flush opening(s) 138 may be located on the elongate body 160. In some examples, during the manufacturing of an expandable metallic implant device 1, the distal end of an inflated balloon 111 of a balloon catheter 100 is inserted into the central hollow region of an expanded expandable metallic implant 11 that is shaped such that the inflated balloon 111 and the expanded expandable metallic implant 11 come together in a close fit, as shown in FIG. 48A. The expanded expandable metallic implant 11 and the inflated balloon 111 can then be folded together, as shown in FIG. 48B. The folded wings 6 can then be pleated, as shown in FIG. 48C to reduce the overall diameter of the expandable metallic implant 10 and the balloon 110 and produce an expandable metallic implant device 1 that can be inserted into the lumen 604 of a blood vessel and advanced to a target vessel segment wherein the operator desires to obstruct or reduce the flow of blood in the a target vessel segment, as shown in FIG. 46B. Once at the target vessel segment, the folded and pleated expandable metallic implant and balloon assembly 4 can be expanded by inflation of the balloon, as shown in FIG. 46B. After deflation or collapse of the overinflated balloon 112 the balloon catheter 100 can be retracted from expanded expandable metallic implant 11 such that the expanded expandable metallic implant 11 is retained in the target vessel segment, as shown in FIG. 46G.

In some examples, the expandable metallic implant device 1 and/or the balloon catheter 100 can be advanced or retracted over a guidewire. The distal end of the balloon catheter and the guidewire can extend through a valve 79 in the neck 60 of the expandable metallic implant 10, as shown in FIGS. 36B and 46B. When the distal end of the balloon catheter and the guidewire are withdrawn, the valve 79 can be configured to close, as shown in FIGS. 36C-D and FIGS. 46F-G.

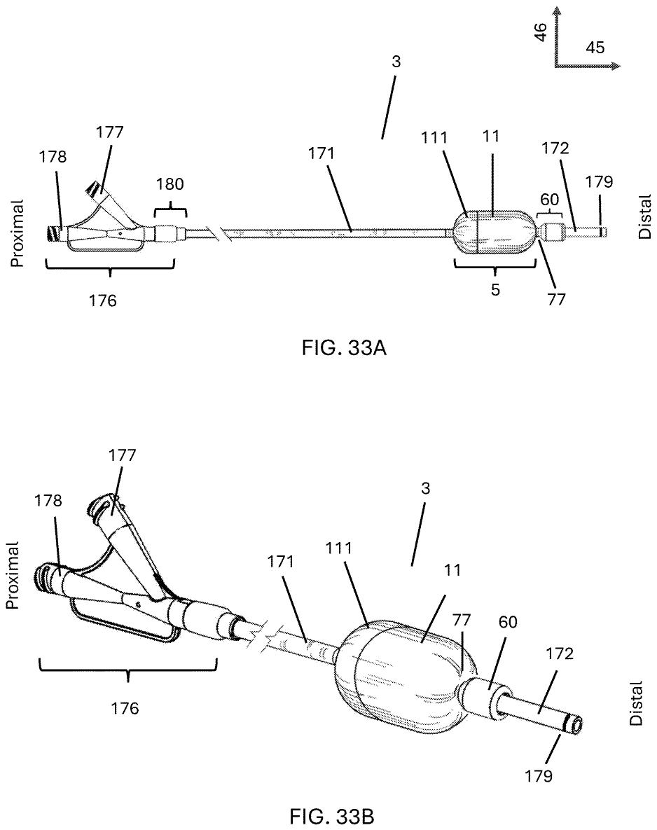

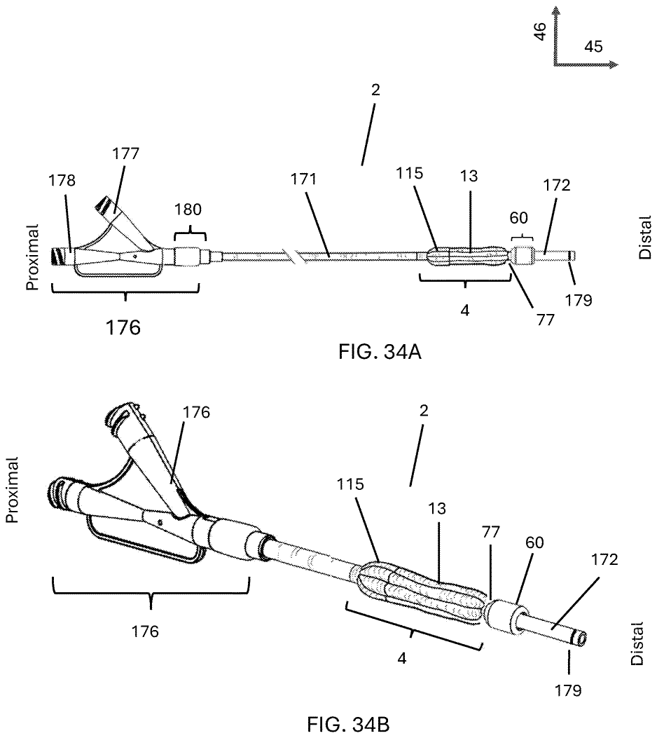

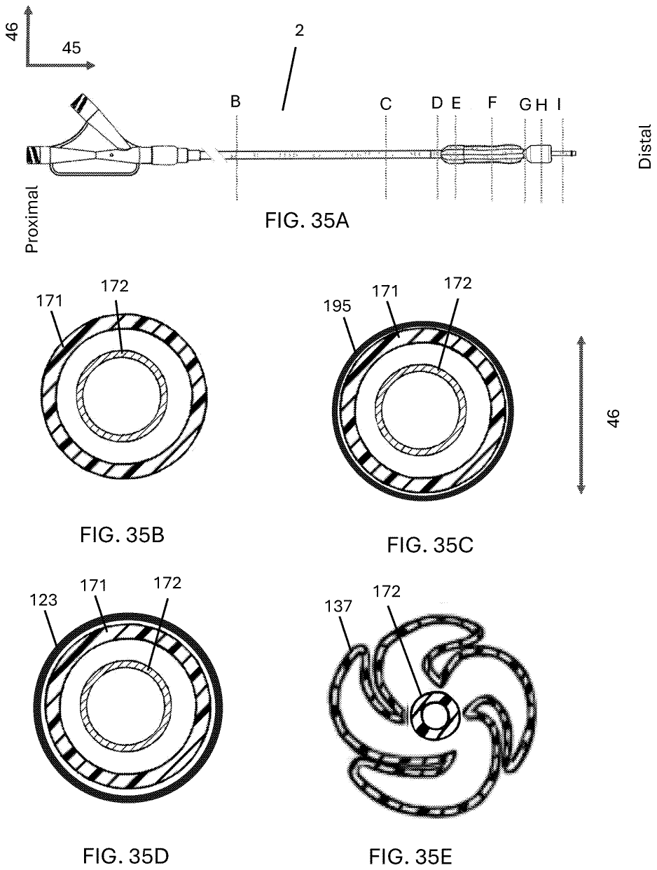

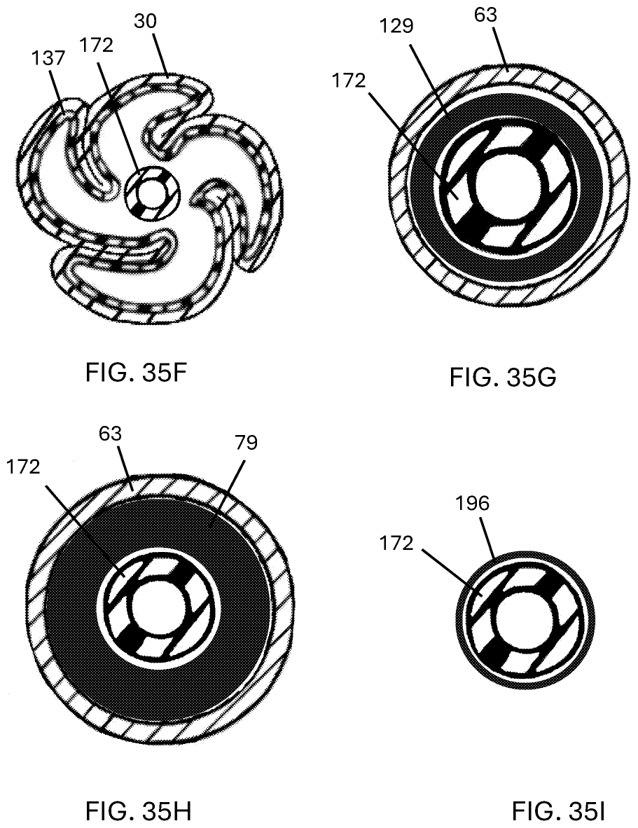

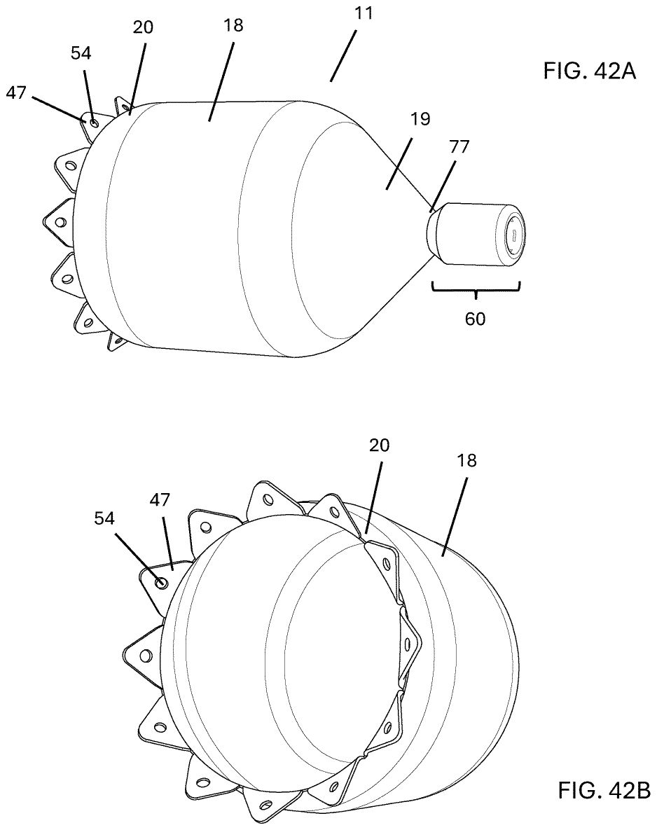

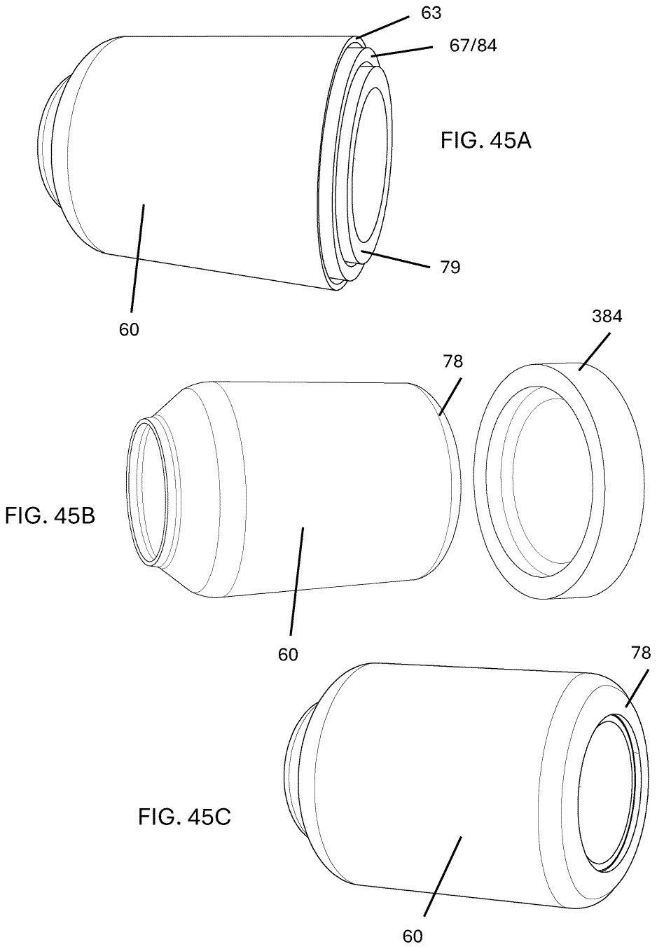

In some examples, the expandable metallic implant 10 can include any and/or all of the features described with respect to FIGS. 1A-17D and 21-24C. The expandable metallic implant can include a neck at or near a distal end of the implant. In some examples, the neck comprises a neck lumen 62 in fluid communication with an opening 61 on the distal end expandable metallic implant 10 and also in fluid communication with an opening in the neck transitional region 77 between the transition zone of the expandable metallic implant 19 and the neck of the expandable metallic implant 60. In some examples, there can be a narrowing in the neck transitional region 77 between the transition zone of the expandable metallic implant 19 and the neck of the expandable metallic implant 60, wherein the lumen of the transition zone of the expandable metallic implant 19 and the lumen of the neck 60 are larger than the lumen of the neck transitional region 77, as shown in FIGS. 41-42 . The larger diameter of the transition zone of the expanded expandable metallic implant 11 obstructs the lumen of an artery or vein. The larger diameter of the seal zone 18 of the expanded expandable metallic implant 11 engages the wall of an artery or vein to increase sliding friction and reduce the risk of migration of the expanded expandable metallic implant 11. The larger diameter of the neck lumen 62 provides a space for a valve to be contained within the expandable metallic implant 10. In some examples, a catheter or catheter shaft can be configured to be positioned through the implant such that a distal portion of the catheter or catheter shaft extends through the opening of the neck. In some examples, the catheter shaft is an inner shaft of the elongate body of an over-the-guidewire balloon catheter, as shown in FIGS. 33-36 and 46. In some examples, the catheter or catheter shaft can be configured to accept a guidewire, such that the guidewire can also be positioned through the implant such that a distal portion of the guidewire extends through the opening of the neck. The opening of the neck can be at least partially obstructed, entirely obstructed, or occluded when the catheter or catheter shaft and guidewire is retracted from the expandable metallic implant 10. For example, the neck of the expandable metallic implant 60 and/or neck lumen of the expandable metallic implant 62 can include a valve 79. The valve 79 can be configured to open when a catheter or catheter shaft and/or guidewire is advanced therethrough. The valve 79 can be configured to close when a catheter or catheter shaft and/or guidewire is retracted from the valve 79. In some examples, the valve 79 can be configured to open when another element is advanced distally therethrough. In some examples, the valve 79 can be configured to close when another element is not positioned within the valve 79, including radially within the valve 79. In some examples, the valve 79 can be surrounded circumferentially by electroformed gold, including an electroformed gold component of then neck 60. In some examples, the valve 79 can be surrounded circumferentially by another element, including a ring or platform. In some examples, the electroformed gold and/or ring or platform can be positioned distal to the valve 79 as shown in FIG. 45A. In some examples the electroformed gold can be crimped around the valve and/or metallic ring or platform, as shown in FIG. 45C.

In some examples, the balloon catheter 100 can include a hub 173 with two ports, one port 177 configured to receive a syringe 402 or inflation device 403 that can be used to inflate the balloon 110 and a second port 178 configured to receive a guidewire 280 and a coil expandable implant 301 or vascular plug expandable implant 321, as shown in FIG. 45. In some examples, when the hub 176 is joined with an outer shaft of an elongate body 171 and an inner shaft of an elongate body 172, the hub 176 comprises two lumens, one for balloon 110 inflation and the other for insertion of a guidewire 280, coil expandable implant 301 or vascular plug expandable implant 321. In some examples, the elongate body 197 of a balloon catheter 100 can include a radiopaque marker band along the body of the catheter to provide visualization of the balloon catheter 100 during fluoroscopy. In one example, the inner shaft 172 comprises a radiopaque marker band near the distal tip of the inner shaft 172. In another example, the inner shaft 172 comprises a radiopaque marker band near the distal tip of the inner shaft 172 and another marker band more proximal to facilitate the detachment of a coil expandable implant 301. For example, the more proximal may be placed at a location on the inner shaft 172 such that the entire body of a coil expandable implant 301 is distal to the distal tip of the inner shaft 172 when a marker band on the coil expandable implant device is lined up with the proximal marker band on the inner shaft 172. Detachment of the coil expandable implant 301 from the coil expandable implant device 300 can allow for the release of the coil expandable implant 301 in the vein or artery to promote obstruction of the artery or vein. In some examples, the expandable metallic implant device 1 is configured such that a coil expandable implant 301 can be placed in an artery or vein both before or after expansion of the expandable metallic implant 10 and both proximal and distal to the expandable metallic implant 10.

In some examples, the balloon 110 described herein can include a cone-shaped proximal region 117 and a cone-shaped distal region 119, with an intermediate portion 118 therebetween. In some examples, the cone-shaped proximal region 117 can be longer than the cone-shaped distal region 119. In some examples, the cone-shaped proximal region 117 is elongated to reduce the overall diameter of the pleated and folded profile of the cone-shaped proximal region 117. In some examples, the slope of the cone-shaped proximal region 117 is reduced to reduce the overall diameter of the pleated and folded profile of the cone-shaped proximal region 117. In some examples, the proximal zone 17 proximal of the expandable metallic implant 10 is configured to match the cone-shaped proximal region 117 of the balloon 110 and is similarly elongated or with a similarly reduced slope. In some examples, the cone-shaped distal region 119 of the balloon 110 can be shorter than the cone-shaped proximal region 117. In some examples, the cone-shaped distal region 119 is shortened to reduce the length of the corresponding transition zone 19 of the expandable metallic implant 10. In some examples, the slope of the cone-shaped distal region 117 of the balloon 110 and the slope of the transition zone 19 of the expandable metallic implant 10 is reduced to reduce the overall length of the rigid folded and pleated expandable metallic implant 13 and improve the flexibility of the folded and pleated expandable metallic implant device 2 in vivo. In some examples, the balloon 110 can have an asymmetrical profile along the length of the balloon, with a higher volume proximal to the center of the central portion.

In some examples, a distal portion of the neck 10 of the expandable metallic implant 10 can be crimped or folded over a valve 79 or valve assembly 87 that is positioned within the lumen of the neck 10 of the expandable metallic implant 10, as shown in FIG. 45C. For example, the electroformed gold wall can be bent at the distal end such that the bent gold 78 at least partially covers the distal end of the valve 79, the valve assembly 87, or a structure surrounding the valve 79. This can allow for retention of the valve 79 with respect to the expandable metallic implant.

In some examples, the expandable metallic implant 10 can be comprised of a gold layer deposited on a flexible polymer layer such that the gold layer is radially outside the flexible polymer layer. The gold layer can include gaps such that the modulus of elasticity of portions of the implant is increased and these gaps can provide a flexible “hinge” region when the expandable metallic implant 10 is folded and pleated with a balloon 110 to improve device flexibility in vivo during device advancement and retraction. For example, the gaps in the gold layer can be between the distal portion of the seal zone 18 and the proximal portion of the transition zone 19, as shown in FIGS. 41A-C or in other regions.

In some examples, the expandable metallic implant 10 can be comprised of a gold layer deposited on a flexible, compliant polymer layer such that the gold layer is radially outside the polymer layer. The gold layer can include gaps that create at least one strut in the gold layer wherein the gaps between the struts can expose the flexible, compliant polymer layer. In some examples, the at least one strut is at least partially closed due to the proximity of the struts and expansion of the balloon can cause elongation of the at least one strut and allow for an increase in the diameter of the expanded expandable metallic implant 11.

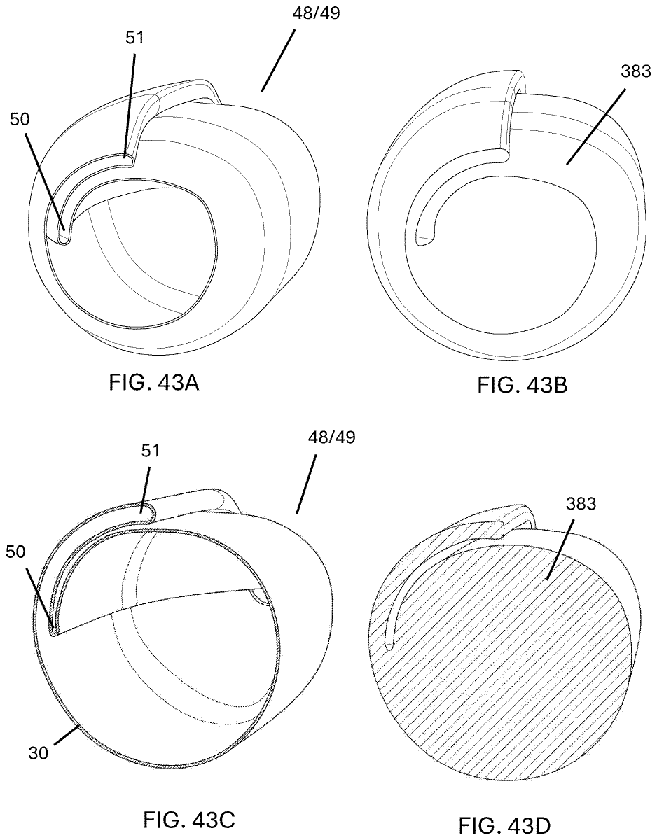

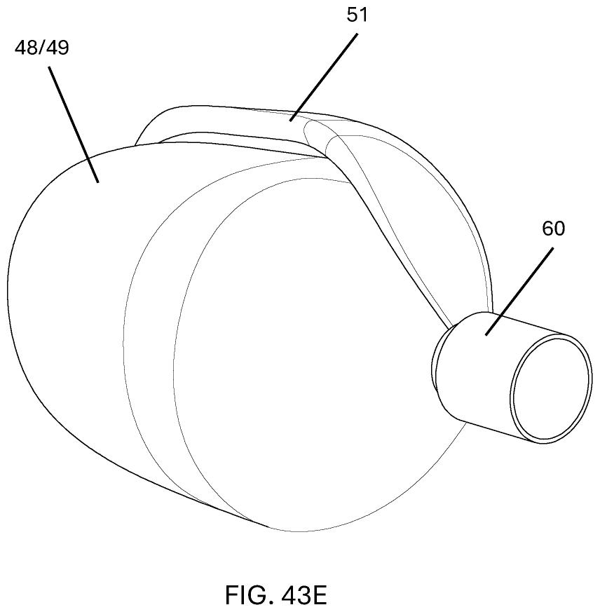

In some examples, the expandable metallic implant 10 can include secondary folds and pleats along the first axis, as shown in FIGS. 43A-E. The secondary folds and pleats can be on one side of the expandable metallic implant, extending along an axis from the proximal end to the distal end of the expandable portion of the expandable metallic implant 15, or can be on more than one side. In some examples, the secondary folds and pleats can extend from the proximal end to the neck of the implant. In some examples, the expandable metallic implant can include 1-5 secondary folds and pleats. A secondary folded and pleated variable size expandable metallic implant 49 can be folded and pleated in a conventional manner, as shown in FIGS. 48A-C. In some examples, a secondary folded and pleated variable size expandable metallic implant 49 comprises a substantially round cross-section, including by compression of the pleats. A secondary folded and pleated variable size expandable metallic implant 49 that is subsequently be folded and pleated in a conventional manner (forming primary folds and pleats), as shown in FIGS. 48A-C can be expanded such that the secondary folds and pleats remains folded and pleated, such that the implant expands to a first expanded diameter. The expandable metallic implant can be expanded further such that at least one of the secondary folds and pleats is unfolded and unpleated, such that the implant expands to a second expanded diameter that is larger than the first expanded diameter. The expandable metallic implant with the second expanded diameter can have a substantially round cross-section, as the secondary pleats and folds can open to become part of a continuous and round wall of the expandable metallic implant after unfolding and unpleating of the primary and secondary folds and pleats. The expandable metallic implant can be expanded to the first expanded diameter in order to secure in the implant within a vessel having a diameter equal to or less than the first expanded diameter. In some examples, the expandable metallic implant can be expanded to the second expanded diameter in order to secure in the implant within a vessel having a diameter larger than the first expanded diameter and equal to or less than the second expanded diameter. In this way, the secondary folds and pleats can allow for the user to change the expanded diameter of the expandable metallic implant based on the vessel diameter.

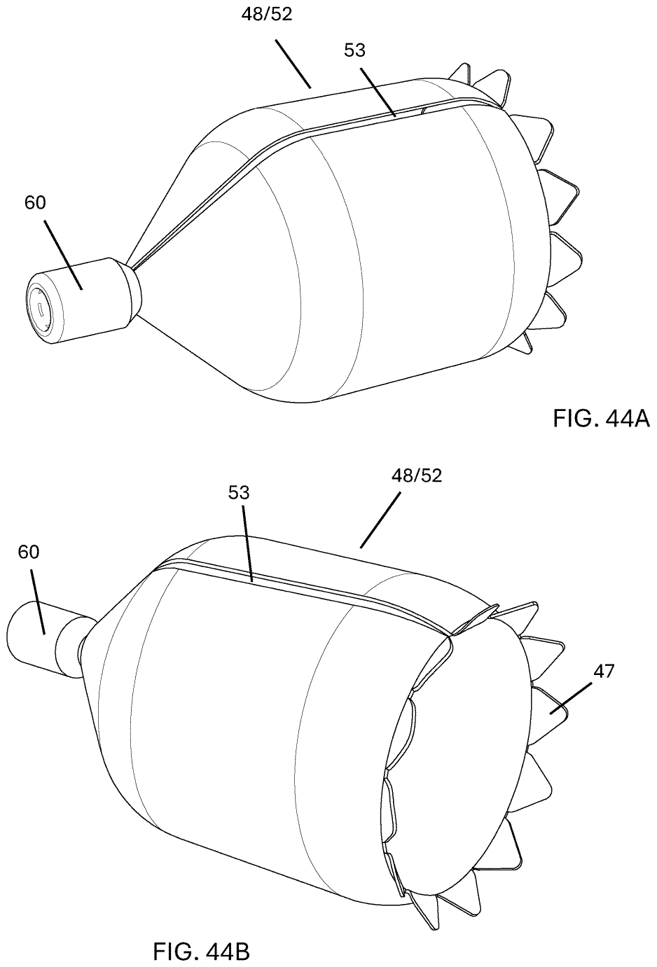

In some examples, the expandable metallic implant 10 can include an outer metal layer and an inner metal layer nested therewithin but not joined or bonded. The expandable metallic implant 10 can have an unexpanded configuration in which the outer metal layer and the inner metal layer are pleated and folded together with a balloon 110. In some examples, both the outer metal layer and the inner metal layer can include a longitudinal linear discontinuity or slot 53, as shown in FIG. 44A-B. In some examples the slots 53 of the outer metal layer and the inner metal layer are offset, including examples wherein they are offset by 45, 90, or 180 degrees. In some examples, the outer metal layer can include a discontinuity configured to expose the inner metal layer in an expanded configuration. The expandable metallic implant can be expanded such that the portions of the implant on either side of discontinuity remain in proximity, such that the implant expands to a first expanded diameter. The expandable metallic implant can be expanded further such that the portion of the implant on one side of discontinuity moves relative to the portion of the implant on the other side of the discontinuity, such that the implant expands to a second expanded diameter that is larger than the first expanded diameter. The overlapping structure with the one or more discontinuities can allow for the user to change the expanded diameter of the expandable metallic implant based on the vessel diameter while maintaining a solid surface to obstruct blood flow.

In some examples, the expandable metallic implant can include one or more flaps 47, for example on the proximal end of the expandable metallic implant. Each flap 47 may comprise a perforation, hole or opening extending from an outer surface of the flap 47 to an inner surface of the flap 47. In some examples, each flap 47 can include 1-5 perforations, holes or openings 54 therethrough. The perforations, holes or openings can provide additional fixation of the expanded expandable metallic implant 11 to the vessel wall 603 as tissue ingrowth can occur through the perforations, holes or openings while the flaps 47 are at least partially in contact with the vessel wall 603. The tissue ingrowth through the perforations, holes or openings 54 of the flaps 47 can provide increased adhesion of the expanded expandable metallic implant 11 and reduce the risk of migration of the expanded expandable metallic implant 11.

In some examples, the expandable metallic implant 10 can be formed by depositing metal on a mandrel. For example, gold can be deposited on a solid polymer mandrel with an outer surface having the desired size and shape. Once the expandable metallic implant 10 is formed, the polymer can be removed by melting the polymer and/or performing chemical dissolution of the polymer. In some examples, the expandable metallic implant 10 can be folded and pleated after the mandrel is removed, as shown in FIGS. 48A-C.

In some examples, the expandable metallic implant 10 can be formed by depositing gold on a flexible or flexible and compliant polymer balloon. For example, gold can be deposited on a flexible or flexible and compliant polymer balloon with an outer surface having the desired size and shape. Once the expandable metallic implant 10 is formed, the excess balloon material can be trimmed such that an inner polymer layer remains within the gold layer. In some examples, the expandable metallic implant can be folded and pleated after the excess balloon material is removed.

Resistance to migration after placement is a key performance criterion for expandable bodies such as expanded expandable metallic implants 11, coil expandable implants 301, and vascular plug expandable implants 321. Expanded expandable metallic implants 11 are designed to resist migration after placement. The operator intentionally chooses an expandable metallic implant device 1 with an expanded expandable metallic implant 11 with a seal zone 18 diameter that is larger than the diameter of the lumen of the target vessel segment. Given that most blood vessel walls are compliant, after inflation of the balloon 110 of the balloon catheter 100 and expansion of the expandable metallic implant 10, the expanded expandable metallic implant 11 will cause a focal enlargement in the target blood vessel segment to accommodate the larger expanded expandable metallic implant 11 and reduce the risk of sliding or migration of the expanded expandable metallic implant 11, as shown in FIGS. 46C-G. The engagement of angulated flaps 22 with the blood vessel wall 603 also reduces the risk of sliding or migration of the expanded expandable metallic implant 11, as shown in FIGS. 46E-G. The gold electroforming process used to manufacture gold expandable metallic implants 10 creates a granular or microtextured surface. After placement of an expanded expandable metallic implant 11 in a target vessel segment, the external surface of the seal zone 18 of the expanded expandable metallic implant 11 comes into contact with the luminal surface of the vessel wall 603 wherein the granular or microtextured surface creates friction to sliding and migration of the expanded expandable metallic implant 11, including through Van der Waals forces.

In some examples, the expandable metallic implant 10 is comprised of one or more flaps 22 and the balloon 110 is compliant or semi-compliant For these examples, the compliant or semi-compliant balloon 110 can be further inflated, as shown in FIG. 46C, causing the uncovered proximal region of the balloon 117 to further expand or bulge, resulting in the angulation of the flaps 22 as shown in FIG. 46D-E and 49C. After deflation or collapse of the inflated balloon 111, the angulated flaps 22 can make contact or engage with the wall of the vessel 603, as shown in FIG. 46E. After deflation or collapse of the inflated balloon 111 the deflated or collapsed balloon 113 and the balloon catheter 100 can be retracted from expanded expandable metallic implant 11 with the angulated flaps 22 such that the expanded expandable metallic implant 11 with the angulated flaps 22 is retained in the target vessel segment.

In some examples, the expandable metallic implant 10 can include flaps 22 at the proximal end of the implant, as shown in FIGS. 9A-D. In some examples, the expandable metallic implant 10 can include flaps 22 in the proximal zone 17 of the implant, as shown in FIG. 10C. In some examples, the expandable metallic implant 10 can include flaps 22 in the seal zone 18 of the implant, as shown in FIGS. 10D and 11A-D. The flaps 22 can be configured to be deployed such that they project radially outward with respect to the expandable metallic implant 10. The balloon 110 bulging radially outward from the expanded expandable metallic implant 11 at the proximal end can cause the flaps 22 to tilt to a deployed position. In the deployed position, the flaps 22 can the wall of the vessel at the target site. In some examples, the flaps can be shaped to engage the vessel wall 603. In some examples, the flaps 22 can be shaped to press into or pierce into the vessel wall 63. In some examples, the flaps 22 can be shaped to promote tissue ingrowth to further secure the implant to the vessel wall, as shown in FIG. 42B. In some examples, the flaps 22 can be integrally formed with the expandable metallic implant 10. In some examples, the flaps 22 can be fixed to a wall of the expandable metallic implant 10.

In some examples, the expandable metallic implant 10 can have a proximal zone 17 with a smaller diameter in the expanded state than the seal zone 18 in the expanded state. The seal zone 18 can be configured to make contact with the wall of the blood vessel 603, while the proximal zone 18 can be configured to engage the balloon 110 such that the inflated balloon 111 and the expanded expandable metallic implant 111 remain secured together while the balloon is inflated, as shown in FIG. 46C-D and such that the deflated or collapsed balloon 113 can be withdrawn or retracted from within the central hollow region 16 of the expanded expandable metallic implant 11, as shown in FIG. 46F.

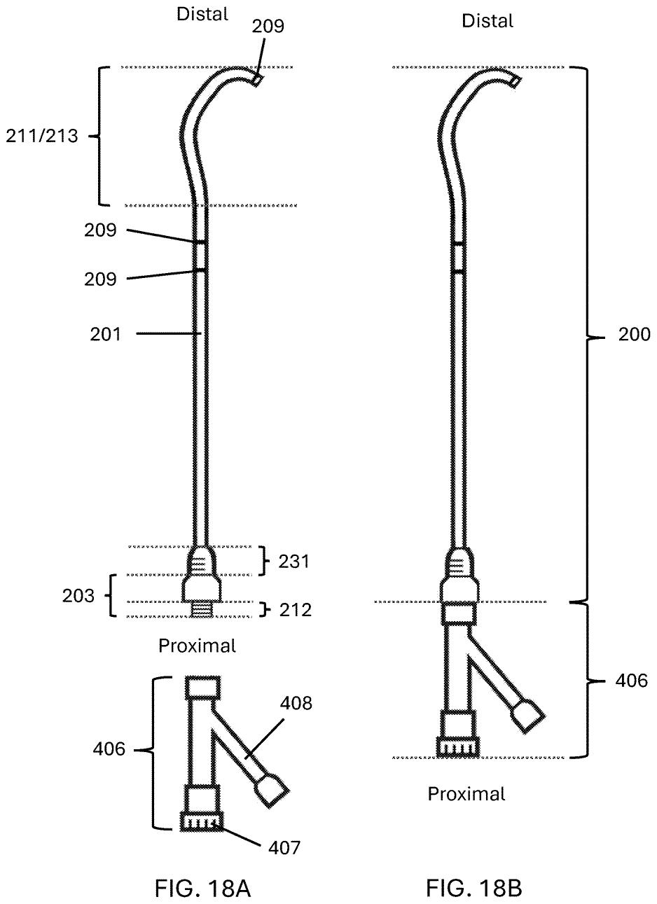

FIG. 18A is a planar view of a guide catheter or guide sheath 200 and a Tuohy Borst adaptor 406, wherein the guide catheter or guide sheath 200 and a Tuohy Borst adaptor 406 are separated. FIG. 18B is a planar view of a guide catheter or guide sheath 200 and a Tuohy Borst adaptor 406, wherein the guide catheter or guide sheath 200 and a Tuohy Borst adaptor 406 are joined, wherein the Tuohy Borst adaptor 406 is configured for flushing the lumen 202 of the guide catheter or guide sheath 200.

In some examples, guide catheter and guide sheath devices 200 may include an elongate body 201, a hub 203 joined or bonded to the proximal end the elongate body 201, and a lumen 202 extending from the proximal to the distal end, wherein the guide catheter and guide sheath are configured for use in selecting, delivering, and detaching various examples of expandable metallic implants 10 of expandable metallic implant devices 1, and methods of use thereof. The guide catheter or guide sheaths 200 have a proximal end, a distal end that is open, and a lumen 202 configured to accept an expandable metallic implant 10 configured for delivery (including a folded and pleated expandable metallic implant 13) and the associated elongate body 160. In some examples, the guide catheter or guide sheath 200 is also configured for detaching various examples of the expanded expandable implants 11 from the elongate body 160 after deflation or collapse of the balloon 110. In some examples, the lumen 202 of the guide catheter or guide sheath 200 is configured for the injection of fluids, including water, saline, radiographic contrast, solutions comprising therapeutic agents or drugs, and mixtures therein.

In some examples, the length of the elongate body 201 is between 20-200 cm. In some examples, the total length of the guide catheter or guide sheath 200 is between 20.5-200.5 cm. In some examples, the outer diameter of the elongate body 201 is between 0.067-0.140 inch. In some examples, the internal or luminal diameter of the elongate body 201 is between 0.055-0.126 inch.

In some examples, the wall 211 of the elongate body 201 is continuous from the proximal end to the distal end. In some examples, the elongate body 201 comprises: 1) an outer layer 207 comprising polymer; 2) an inner layer 205 comprising polymer; and a middle layer 206 comprising metal; wherein the middle layer 206 is disposed between the outer layer 207 and an inner layer 205. In some examples, the inner layer 205, outer layer 207, or both the inner layer 205 and outer layer 207 of the elongate body 201 comprises polymer with successively decreasing durometer from the proximal to the distal end of the elongate body 201.

In some examples, the distal segment of the outer layer 207 of the wall 211 of the elongate body 201 comprises an aliphatic polyether polyurethane or a polyether block amide. In some examples, the aliphatic polyether polyurethane is Tecoflex. In some examples, a distal segment of the outer layer 207 of the of the wall 211 of the elongate body 201 comprises a material with a Shore durometer hardness of 20-60 D. In some examples, the middle segment of the outer layer 207 of the wall 211 of the elongate body 201 comprises a polyether block amide or a nylon. In some examples, the polyether block amide is Pebax. In some examples, the range of the durometer of the polyether block amide is between Pebax 7233 and Pebax 2522 distally. In some examples, the proximal segment of the wall 211 of the outer layer 207 of the elongate body 201 comprises a nylon. In some examples, the nylon is Grilamid. In some examples, a proximal segment of the outer layer 207 of the wall 211 of the elongate body 201 comprises a material with a Shore durometer hardness of 40-90 D. In some examples, the outer layer 207 of at least a portion of the elongate body 201 comprises polyimide.

In some examples, the inner layer 205 of the wall 211 of the elongate body 201 comprises a lubricious polymer. In some examples, the lubricious polymer comprises polytetrafluoroethylene, polyimide, or a composite or mixture of polytetrafluoroethylene (PTFE) and polyimide.

In some examples, the metal of the middle layer 206 of the elongate body 201 is configured as wire. In some examples, the wire is configured in a spiral, coil, braid, woven, or straight pattern, or combinations thereof. In some examples, at least some of the metal in the middle layer 206 of the elongate body 201 is configured as wire with a cross-sectional shape that is round, oval, square, or rectangular. In some examples, the wire comprises nitinol or stainless steel. In some examples, the wire is round and has a diameter between 0.0005-0.0030 inch. In some examples, the wire is configured in a coil with a pitch of between 0.0010-0.0060 inch. In some examples, the wire is flat and has a thickness of between 0.0005-0.0060 inch and a width of between 0.001-0.030 inch. In some examples, wherein the wire is configured in a braid, the braid has a picks per inch of length (PPI) of between 50-300. In some examples, the metal of the middle layer 206 of the elongate body 201 is configured as a laser cut hypotube. In some examples, the laser cut hypotube comprises nitinol. In some examples, the metal or metal wire is absent from the distal segment of the elongate body 201.

In some examples, the wall 210 of the elongate body 201 of the guide catheter or guide sheath 200 further comprises a tie layer. In some examples, the wall 210 of the elongate body 201 comprises one or more liquid crystal polymer fibers. In some examples, the liquid crystal polymer fibers are oriented parallel to the first axis 45 of the elongate body 201. In some examples, the one or more liquid crystal polymer fibers are coiled around the elongate body 201. In some examples, one or more polymers of the wall 210 of the elongate body 201 comprises barium sulfate. In some examples, the polymer comprises barium sulfate at a concentration of 10-30%.

In some examples, the elongate body 201 comprises one or more marker bands 209 that are radiopaque or conspicuous during fluoroscopy. In some examples, the elongate body 201 comprises a single marker band 209 that is conspicuous during fluoroscopy configured for identifying the tip region of the guide catheter or guide sheath 200 during fluoroscopy or a single marker band 209 that is radiopaque or conspicuous during fluoroscopy at a location ≤10 mm proximal to the distal end of the elongate body 201. In some examples, the elongate body 201 comprises 2-5 marker bands 209 that are radiopaque or conspicuous during fluoroscopy and configured for making measurements of the diameter of blood-containing structures during angiography, or 2-5 marker bands 209 that are radiopaque or conspicuous during fluoroscopy at a location >10 mm proximal to the distal end of the elongate body 201. In some examples, the elongate body 201 comprises a single marker band 209 that is radiopaque or conspicuous during fluoroscopy configured for identifying the tip region of the guide catheter or guide sheath 200 during fluoroscopy, and two or three additional marker bands 209 that are radiopaque or conspicuous during fluoroscopy and configured for making measurements of the diameter of blood-containing structures during angiography. In some examples, the elongate body 201 comprises a single marker band 209 that is radiopaque or conspicuous during fluoroscopy at a location ≤10 mm proximal to the distal end of the elongate body 201 and two or three additional marker bands 209 that are radiopaque or conspicuous during fluoroscopy at a location >10 mm proximal to the distal end of the elongate body 201. In some examples, the 2-5 marker bands 209 are located on a straight portion of the elongate body 201.

In some examples, the proximal portion of the elongate body 201 adjacent to the hub 203 comprises a segment of polymer that provides strain relief 204 to the junction between the hub 203 and the elongate body 201. In some examples, the outer diameter of the distal end of the elongate body 201 has a radius.

In some examples, the elongate body 201 comprises a lubricious or hydrophilic coating layer. In some examples, the lubricious or hydrophilic coating layer is present on the inner surface, the outer surface, or both the inner and outer surface of the elongate body 201. In some examples, the lubricious or hydrophilic coating layer is present on the outer surface of the distal portion of the elongate body 201, on the outer surface of the middle and distal portion of the elongate body 201, or on the entire outer surface of the elongate body 201.

In some examples, the outer diameter of the distal end of the elongate body 201 has a radius. In some examples, the elongate body 201 is straight or is straight when in an unconstrained configuration. In some examples, a portion of the elongate body 201 has a portion with a pre-formed shape 211 or has a portion with a pre-formed shape 211 when in an unconstrained configuration, including a distal portion 213. In some examples, the pre-formed shape portion 211 comprises an Angled, Multi-Purpose, Berenstein, Vertebral, Hockey Stick, Simmons 1, Simmons 2, Simmons 3, Cobra 1, Cobra 2, Headhunter, JB1, JB2, and Renal Double Curve shape, or any other shape needed to facilitate advancement.

In some examples, the guide catheter or guide sheath 200 is provided with an obturator or dilator 412 to provide a smooth transition from a guidewire 280 to the tip of the guide catheter or guide sheath 200 during insertion. In some examples, the obturator or dilator 412 comprises: a lumen 202 configured to accept a guidewire 280; an outer diameter that is between 0.001-0.006 inch smaller than the inner (luminal diameter) of the guide catheter or guide sheath 200; a longer elongate body than the guide catheter or guide sheath 200 so that it can pass through the lumen 202 of the guide catheter or guide sheath 200 and exit the tip of the guide catheter or guide sheath 200; and a hub 415 that can reversibly couple with the hub 203 of the guide catheter or guide sheath 200. In some examples, the obturator or dilator 412 comprises a lubricious or hydrophilic coating layer. In some examples, the lubricious or hydrophilic coating layer is present on the inner surface, the outer surface, or both the inner and outer surface of the obturator or dilator 412. In some examples, the lubricious or hydrophilic coating layer is present on the distal portion of the outer surface of the obturator or dilator 412, on the middle and distal portion of the outer surface of the obturator or dilator 412, or on the entire outer surface of the obturator or dilator 412. In some examples, the lumen 202 of the obturator or dilator 412 is configured to accept a 0.010-, 0.014-, 0.018-, 0.025-, 0.035-, or 0.038-inch guidewire 280.

In some examples, the system can include a guide catheter or guide sheath 200 and an expandable metallic implant device 1 is used to obstruct, occlude, embolize, or reduce flow in arteries, veins, aneurysms, parent vessels of saccular aneurysms, paravalvular leak pathways, biological conduits, or other blood-containing, fluid-containing, or biological spaces, the inner (luminal) diameter of a guide catheter or guide sheath 200 is larger than the outer diameter of elongate body 160. If the expandable metallic implant device 1 is inserted directly into the guide catheter or guide sheath 200, blood will enter the lumen 202 of the guide catheter or guide sheath 200 and leak around the elongate body 160 and out of the patient at the hub 203 of the guide catheter or guide sheath 200. Therefore, operators will sometimes join a Tuohy Borst adaptor 406 with a rotating valve 407 or a rotating valve without a side arm to the hub 203 of the guide catheter or guide sheath 200 and then insert the expandable metallic implant device 1 through the Tuohy Borst adaptor 406 into the lumen 202 of the guide catheter or guide sheath 200 and use the valve on the Tuohy Borst adaptor 406 to prevent the leading of blood. The present disclosure includes systems comprising a guide catheter or guide sheath 200, an expandable metallic implant device 1, and a Tuohy Borst adaptor 406. Opening and closing of a Tuohy Borst adaptor 406 can introduce air into the Tuohy Borst adaptor 406 and the lumen 202 of the guide catheter or guide sheath 200. A Tuohy Borst adaptor 406 comprises a side arm 408 that can be used to aspirate air from the Tuohy Borst adaptor 406 and to flush the Tuohy Borst adaptor 406. A rotating valve comprises a valve but not a side arm for flushing.

### Intermediate Catheters and Methods of Use

Further implementations of this disclosure relate to systems further comprising an intermediate catheter 220 comprising an elongate body 221 with a lumen 222 and a hub 223 joined or bonded to the proximal end the elongate body 221, wherein the elongate body 221 of the intermediate catheter 220 is configured to pass through the lumen 202 of the guide catheter or guide sheath 200. In some examples, the addition of the intermediate catheter 220 to the system is intended to reduce friction when advancing the guide catheter or guide sheath 200 in vivo. In some examples, the proximal portion 228 of the elongate body 221 of the intermediate catheter 220 has a larger inner and outer diameter than the middle portion 229 and distal portion 230 of the elongate body 221 of the intermediate catheter 220.

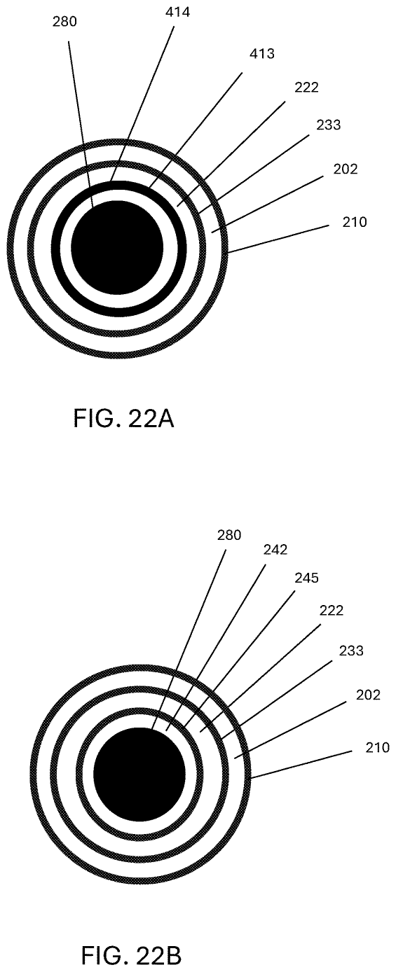

FIG. 22A is a cross-sectional view of an example of an assembly of a guide catheter or guide sheath 200, intermediate catheter 220, and obturator 412. FIG. 22B is a cross-sectional view of an example of an assembly of a guide catheter or guide sheath 200, intermediate catheter 220, selective catheter 240, and guidewire 280. In some examples, the length of the elongate body 221 is between 20.5-200.5 cm. In some examples, the total length of the intermediate catheter 220 is between 21-201 cm. In some examples, the outer diameter of the proximal portion 228 of the elongate body 221 is between 0.076-0.122 inch. In some examples, the outer diameter of the distal portion 230 of the elongate body 221 is between 0.076-0.087 inch. In some examples, the internal or luminal diameter of the proximal portion 228 of elongate body 221 is between 0.065-0.105 inch. In some examples, the internal or luminal diameter of the distal portion 230 of elongate body 221 is between 0.065-0.073 inch.

In some examples, the wall 233 of the elongate body 221 is continuous from the proximal end to the distal end. In some examples, the elongate body 221 comprises: 1) an outer layer 226 comprising polymer; 2) an inner layer 224 comprising polymer; and a middle layer 228 comprising metal; wherein the middle layer 228 is disposed between the outer layer 226 and an inner layer 224. In some examples, the inner layer 224, outer layer 226, or both the inner layer 224 and outer layer 226 of the elongate body 221 comprises polymer with successively decreasing durometer from the proximal to the distal end of the elongate body 221.

In some examples, the distal segment of the outer layer 226 of the wall 233 of the elongate body 221 comprises an aliphatic polyether polyurethane or a polyether block amide. In some examples, the aliphatic polyether polyurethane is Tecoflex. In some examples, a distal segment of the outer layer 226 of the wall 233 of the elongate body 221 comprises a material with a Shore durometer hardness of 20-60 D. In some examples, the distal segment of the outer layer 226 of the wall 233 end of the elongate body 221 comprises a material with a Shore durometer hardness of 40-90 D. In some examples, the middle segment of the outer layer 226 of the wall 233 of the elongate body 221 comprises a polyether block amide or a nylon. In some examples, the polyether block amide is Pebax. In some examples, the range of the durometer of the polyether block amide is between Pebax 7233 and Pebax 2522 distally. In some examples, the proximal segment of the outer layer 226 of the wall 233 of the elongate body 221 comprises a nylon. In some examples, the nylon is Grilamid. In some examples, a proximal segment of the outer layer 226 of the wall 233 of the elongate body 221 comprises a material with a Shore durometer hardness of 40-90 D. In some examples, the outer layer 226 of at least a portion of the elongate body 221 comprises polyimide.

In some examples, the metal of the middle layer 228 of the elongate body 221 is configured as wire. In some examples, the wire is configured in a spiral, coil, braid, woven, or straight pattern, or combinations thereof. In some examples, the wire comprises nitinol or stainless steel. In some examples, the metal of the middle layer 228 of the elongate body 221 is configured as a laser cut hypotube. In some examples, the laser cut hypotube comprises nitinol. In some examples, the metal or metal wire is absent from the distal segment of the elongate body 221.

In some examples, the inner layer 224 of the wall 233 of the elongate body 221 comprises a lubricious polymer. In some examples, the lubricious polymer comprises polytetrafluoroethylene, polyimide, or a composite or mixture of polytetrafluoroethylene (PTFE) and polyimide.

In some examples, the wall 233 of the elongate body 221 of the selective catheter 240 further comprises a tie layer. In some examples, the wall 210 of the elongate body 221 comprises one or more liquid crystal polymer fibers. In some examples, the liquid crystal polymer fibers are oriented parallel to the first axis 45 of the elongate body 221. In some examples, the one or more liquid crystal polymer fibers are coiled around the elongate body 221. In some examples, one or more polymers of the wall 245 of the elongate body 221 comprises barium sulfate. In some examples, the polymer comprises barium sulfate at a concentration of 10-30%.

In some examples, the elongate body 221 comprises one or more marker bands 231 that are radiopaque or conspicuous during fluoroscopy. In some examples, the elongate body 221 comprises a single marker band 231 that is conspicuous during fluoroscopy configured for identifying the tip region of the guide catheter or guide sheath 200 during fluoroscopy or a single marker band 231 that is radiopaque or conspicuous during fluoroscopy at a location ≤10 mm proximal to the distal end of the elongate body 221.

In some examples, the elongate body 221 comprises a lubricious or hydrophilic coating layer. In some examples, the lubricious or hydrophilic coating layer is present on the inner surface, the outer surface, or both the inner and outer surface of the elongate body 221. In some examples, the lubricious or hydrophilic coating layer is present on the distal portion 230 of the outer surface of the elongate body 221, on the distal portion 230 and middle portion 229 of the outer surface of the elongate body 221, or on the entire outer surface of the elongate body 221. The proximal portion 228 of the elongate body 221 adjacent to the hub 223 may comprise a segment of polymer that provides strain relief 232 to the junction between the hub 223 and the elongate body 221.

In some examples, the outer diameter of the distal end of the elongate body 221 has a radius. In some examples, the elongate body 221 is straight or is straight when in an unconstrained configuration. In some examples, the elongate body 221 has a portion with a pre-formed shape 235 or has a portion with a pre-formed shape 235 when in an unconstrained configuration, including a distal portion 230. In some examples, the portion with a pre-formed shape 235 comprises an Angled, Multi-Purpose, Berenstein, Vertebral, Hockey Stick, Simmons 1, Simmons 2, Simmons 3, Cobra 1, Cobra 2, Headhunter, JB1, JB2, or Renal Double Curve shape, or any other shape needed to facilitate advancement.

In some examples, the intermediate catheter 220 is provided with an obturator or dilator 412 to provide a smooth transition from a guidewire 280 to the tip of the intermediate catheter 220 during insertion. In some examples, the obturator or dilator 412 comprises: a wall 413 with a lumen 414 configured to accept a guidewire 280; an outer diameter that is between 0.001-0.008 inch smaller than the inner (luminal diameter) of the distal portion 230 of the intermediate catheter 220; a longer elongate body than the intermediate catheter 220 so that it can pass through the lumen 222 of the intermediate catheter 220 and exit the tip of the intermediate catheter 220; and a hub 415 that can reversibly couple with the hub 223 of the intermediate catheter 220. In some examples, the obturator or dilator 412 comprises a lubricious or hydrophilic coating layer. In some examples, the lubricious or hydrophilic coating layer is present on the inner surface, the outer surface, or both the inner and outer surface of the obturator or dilator 412. In some examples, the lubricious or hydrophilic coating layer is present on the outer surface of the distal portion of the obturator or dilator 412, on the outer surface of the middle and distal portion of the obturator or dilator 412, or on the entire outer surface of the obturator or dilator 412. In some examples, the lumen 414 of the obturator or dilator 412 is configured to accept a 0.010-, 0.014-, 0.018-, 0.025-, 0.035-, or 0.038-inch guidewire 280.

In some examples, wherein a system comprising a guide catheter or guide sheath 200, an intermediate catheter 220, and a guidewire 280, the inner (luminal) diameter of the distal portion 230 of the intermediate catheter 220 is larger than the outer diameter of the guidewire 280. If the guidewire 280 is inserted directly into the intermediate catheter 220, blood will enter the lumen 222 of the intermediate catheter 220 and leak around the guidewire 280 and out of the patient at the hub 223 of the intermediate catheter 220. Therefore, operators may choose to join a Tuohy Borst adaptor 406 with a rotating valve 407 or a rotating valve without a side arm to the hub 223 of the intermediate catheter 220 to prevent the leading of blood and to facilitate flushing of the lumen 222 of the intermediate catheter 220. The present disclosure includes systems comprising a guide catheter or guide sheath 200, a first Tuohy Borst adaptor 406 joined to the hub 203 of the guide catheter or guide sheath 200 to reduce bleeding and to facilitate flushing of the lumen 202 of the guide catheter or guide sheath 200, an intermediate catheter 220, a second Tuohy Borst adaptor 409 joined to the hub 223 of the intermediate catheter 220 to reduce bleeding and to facilitate flushing of the lumen 222 of the intermediate catheter 220, and a guidewire 280.

### Selective Catheters and Methods of Use

Further implementations of this disclosure relate to selective catheters 240 comprising an elongate body 241 with a single lumen 242, a hub 243 joined or bonded to the proximal end the elongate body 241, and a single lumen 242 extending from the proximal to the distal end, wherein a portion of the elongate body 241 has a pre-formed shape 244 or has a portion with a pre-formed shape 244 when in an unconstrained configuration, including a distal portion 251, and wherein the selective catheters 240 are configured for facilitating the advancement of a guide catheter or guide sheath 200, the advancement of a guide catheter or guide sheath 200 and an intermediate catheter 220 that are joined together, or the advancement of a guidewire 280 in a human patient, and methods of use thereof. The selective catheters 240 have a proximal end, a distal end that is open, and a lumen 242 configured to accept a guidewire 280 and for the injection of fluids, including water, saline, radiographic contrast, solutions comprising therapeutic agents or drugs, and mixtures therein. In some examples, the pre-formed shape portion 244 comprises an Angled, Multi-Purpose, Berenstein, Vertebral, Hockey Stick, Simmons 1, Simmons 2, Simmons 3, Cobra 1, Cobra 2, Headhunter, JB1, JB2, and Renal Double Curve shape, or any other shape needed to facilitate advancement.

In some examples, the length of the elongate body 241 is between 21-201 cm. In some examples, the total length of the selective catheter 240 is between 21.5-201.5 cm. In some examples, the outer diameter of the proximal portion 249 of the elongate body 241 is between 0.051-0.069 inch. In some examples, the outer diameter of the distal portion 251 of the elongate body 241 is between 0.050-0.058 inch. In some examples, the internal or luminal diameter of the proximal portion 249 of elongate body 241 is between 0.37-0.057 inch. In some examples, the internal or luminal diameter of the distal portion 251 of elongate body 241 is between 0.038-0.046 inch.

In some examples, the wall 245 of the elongate body 241 is continuous from the proximal end to the distal end. In some examples, the elongate body 241 comprises: 1) an outer layer 254 comprising polymer; 2) an inner layer 252 comprising polymer; and a middle layer 253 comprising metal; wherein the middle layer 253 is disposed between the outer layer 254 and an inner layer 252. In some examples, the inner layer 252, outer layer 254, or both the inner layer 252 and outer layer 254 of the elongate body 241 comprises polymer with successively decreasing durometer from the proximal to the distal end of the elongate body 241.

In some examples, the distal segment of the outer layer 254 of the wall 245 of the elongate body 241 comprises an aliphatic polyether polyurethane or a polyether block amide. In some examples, the aliphatic polyether polyurethane is Tecoflex. In some examples, a distal segment of the outer layer 254 of the wall 245 of the elongate body 241 comprises a material with a Shore durometer hardness of 20-60 D. In some examples, the middle segment of the outer layer 254 of the wall 245 of the elongate body 241 comprises a polyether block amide or a nylon. In some examples, the polyether block amide is Pebax. In some examples, the range of the durometer of the polyether block amide is between Pebax 7233 and Pebax 2522 distally. In some examples, the proximal segment of the outer layer 254 of the wall 245 of the elongate body 241 comprises a nylon. In some examples, the nylon is Grilamid. In some examples, a proximal segment of the outer layer 254 of the wall 245 of the elongate body 241 comprises a material with a Shore durometer hardness of 40-90 D. In some examples, the outer layer 254 of at least a portion of the wall 245 of the elongate body 241 comprises polyimide.

In some examples, the metal of the middle layer 253 of the elongate body 241 is configured as wire. In some examples, the wire is configured in a spiral, coil, braid, woven, or straight pattern, or combinations thereof. In some examples, at least some of the metal in the middle layer 253 of the elongate body 241 is configured as wire with a cross-sectional shape that is round, oval, square, or rectangular. In some examples, the wire comprises nitinol or stainless steel. In some examples, the wire is round and has a diameter of between 0.0005-0.0030 inch. In some examples, the wire is configured in a coil with a pitch of between 0.0010-0.0060 inch. In some examples, the wire is flat and has a thickness of between 0.0005-0.0060 inch and a width of between 0.001-0.030 inch. In some examples, wherein the wire is configured in a braid, the braid has a picks per inch of length (PPI) of between 50-300. In some examples, the metal of the middle layer 253 of the elongate body 241 is configured as a laser cut hypotube. In some examples, the laser cut hypotube comprises nitinol. In some examples, the metal or metal wire is absent from the distal segment of the elongate body 241.

In some examples, the inner layer 252 of the wall 245 of the elongate body 241 comprises a lubricious polymer. In some examples, the lubricious polymer comprises polytetrafluoroethylene, polyimide, or a composite or mixture of polytetrafluoroethylene (PTFE) and polyimide.

In some examples, the wall 245 of the elongate body 241 of the selective catheter 240 further comprises a tie layer. In some examples, the wall 210 of the elongate body 241 comprises one or more liquid crystal polymer fibers. In some examples, the liquid crystal polymer fibers are oriented parallel to the first axis 45 of the elongate body 241. In some examples, the one or more liquid crystal polymer fibers are coiled around the elongate body 241. In some examples, one or more polymers of the wall 245 of the elongate body 241 comprises barium sulfate. In some examples, the polymer comprises barium sulfate at a concentration of 10-30%.

In some examples, the elongate body 241 comprises one or more marker bands 247 that are radiopaque or conspicuous during fluoroscopy. In some examples, the elongate body 241 comprises a marker band 247 that is radiopaque or conspicuous during fluoroscopy configured for identifying the tip region of the selective catheter 240 during fluoroscopy or a marker band 247 that is radiopaque or conspicuous during fluoroscopy at a location ≤10 mm proximal to the distal end of the elongate body 241.

In some examples, the proximal portion of the elongate body 241 adjacent to the hub 243 comprises a segment of polymer that provides strain relief 248 to the junction between the hub 243 and the elongate body 241.

In some examples, the elongate body 241 comprises a lubricious or hydrophilic coating layer. In some examples, the lubricious or hydrophilic coating layer is present on the inner surface, the outer surface, or both the inner and outer surface of the elongate body 241. In some examples, the lubricious or hydrophilic coating layer is present on the outer surface of the distal portion 251 of the elongate body 241, on the outer surface of the middle 250 and distal 251 portion of the elongate body 241, or on the entire outer surface of the elongate body 241.

In some examples, the outer diameter of the distal end of the elongate body 241 has a radius. In some examples, the tip portion of the elongate body 241 is tapered.

### Coiling or Delivery Catheter Medical Devices and Methods of Use

Further implementations of this disclosure relate to coiling or delivery catheters 260 comprising an elongate body 261, a hub 263 joined or bonded to the proximal end the elongate body 261, and a single lumen 262 extending from the proximal to the distal end, wherein the coiling or delivery catheters 260 are configured for facilitating the placement of coil expandable implant 301 devices and vascular plug expandable metallic implant devices, and methods of use thereof. The coiling or delivery catheters 260 have a proximal end, a distal end that is open, and a lumen 262 configured to accept a guidewire 280, deliver coil expandable implant devices 300 and vascular plug expandable metallic implant devices, and for the injection of fluids, including water, saline, radiographic contrast, solutions comprising therapeutic agents or drugs, and mixtures therein. In some examples, the coiling or delivery catheters 260 can include 1-5 lumens. In some examples, the single lumen 262 can be a guidewire lumen and a delivery lumen. In some examples, the lumen 262 can include lumens therewithin. For example, the coiling or delivery catheters 260 can include lumens that are concentric, tangential, or otherwise overlapping.

Coil expandable implants can be delivered to the treatment site through a thin, flexible specialty catheter developed for the implantation of coil expandable implants 301 (a “coiling or delivery catheter”), or through another type of catheter such as a selective catheter 240. A coiling or delivery catheter 260 is advanced into the body and once the desired position is reached, one more coil expandable implants 301 are pushed through the coiling or delivery catheter 260 and placed at the treatment site.

In some examples, the length of the elongate body 261 is between 21-201 cm. In some examples, the total length of the coiling or delivery catheter 260 is between 21.5-201.5 cm. In some examples, the outer diameter of the elongate body 261 is between 0.033-0.041 inch. In some examples, the internal or luminal diameter of the elongate body 261 is between 0.021-0.029 inch.

In some examples, the wall 265 of the elongate body 261 is continuous from the proximal end to the distal end. In some examples, the elongate body 261 comprises: 1) an outer layer 269 comprising polymer; 2) an inner layer 267 comprising polymer; and a middle layer 268 comprising metal; wherein the middle layer 268 is disposed between the outer layer 269 and an inner layer 267. In some examples, the inner layer 267, outer layer 269, or both the inner layer 267 and outer layer 269 of the elongate body 261 comprises polymer with successively decreasing durometer from the proximal to the distal end of the elongate body 261.

In some examples, the distal segment of the outer layer 269 of the wall 265 of the elongate body 261 comprises an aliphatic polyether polyurethane or a polyether block amide. In some examples, the aliphatic polyether polyurethane is Tecoflex. In some examples, a distal segment of the outer layer 269 of the wall 265 of the elongate body 261 comprises a material with a Shore durometer hardness of 20-60 D. In some examples, the middle segment of the outer layer 269 of the wall 265 of the elongate body 261 comprises a polyether block amide or a nylon. In some examples, the polyether block amide is Pebax. In some examples, the range of the durometer of the polyether block amide is between Pebax 7233 and Pebax 2522 distally. In some examples, the proximal segment of the outer layer 269 of the wall 265 of the elongate body 261 comprises a nylon. In some examples, the nylon is Grilamid. In some examples, a proximal segment of the outer layer 269 of the wall 265 of the elongate body 261 comprises a material with a Shore durometer hardness of 40-90 D. In some examples, the outer layer 269 of at least a portion of the wall 265 of the elongate body 261 comprises polyimide.

In some examples, the metal of the middle layer 268 of the elongate body 261 is configured as wire. In some examples, the wire is configured in a spiral, coil, braid, woven, or straight pattern, or combinations thereof. In some examples, at least some of the metal in the middle layer 268 of the elongate body 261 is configured as wire with a cross-sectional shape that is round, oval, square, or rectangular. In some examples, the wire comprises nitinol or stainless steel. In some examples, the wire is round and has a diameter of between 0.0005-0.0030 inch. In some examples, the wire is configured in a coil with a pitch of between 0.0010-0.0060 inch. In some examples, the wire is flat and has a thickness of between 0.0005-0.0060 inch and a width of between 0.001-0.030 inch. In some examples, wherein the wire is configured in a braid, the braid has a picks per inch of length (PPI) of between 50-300. In some examples, the metal of the middle layer 268 of the elongate body 261 is configured as a laser cut hypotube. In some examples, the laser cut hypotube comprises nitinol. In some examples, the metal or metal wire is absent from the distal segment of the elongate body 261.

In some examples, the inner layer 267 of the wall 265 of the elongate body 261 comprises a lubricious polymer. In some examples, the lubricious polymer comprises polytetrafluoroethylene, polyimide, or a composite or mixture of polytetrafluoroethylene (PTFE) and polyimide. In some examples, a layer of the proximal end of the elongate body 261 comprises a material with a Shore durometer hardness of 40-90 D. In some examples, a layer of the distal end of the elongate body 261 comprises a material with a Shore durometer hardness of 20-60 D.

In some examples, the wall 265 of the elongate body 261 of the coiling or delivery catheter 260 further comprises a tie layer. In some examples, the wall 210 of the elongate body 261 comprises one or more liquid crystal polymer fibers. In some examples, the liquid crystal polymer fibers are oriented parallel to the first axis 45 of the elongate body 261. In some examples, the one or more liquid crystal polymer fibers are coiled around the elongate body 261. In some examples, one or more polymers of the wall 265 of the elongate body 261 comprises barium sulfate. In some examples, the polymer comprises barium sulfate at a concentration of 10-30%.

In some examples, the elongate body 261 comprises one or more marker bands 264 that are radiopaque or conspicuous during fluoroscopy. In some examples, the elongate body 261 comprises a marker band 264 that is radiopaque or conspicuous during fluoroscopy configured for identifying the tip region of the coiling or delivery catheter 260 during fluoroscopy or a marker band 264 that is radiopaque or conspicuous during fluoroscopy at a location ≤10 mm proximal to the distal end of the elongate body 261. In some examples, the elongate body 261 comprises two marker bands 264 that are radiopaque or conspicuous during fluoroscopy and configured for assisting the detachment of coil expandable implants 301 or two marker bands 264 that are radiopaque or conspicuous during fluoroscopy and are located on a straight portion of the elongate body 261 at a location >10 mm proximal to the distal end of the elongate body 261. In some examples, the elongate body 261 comprises a single marker band 264 that is radiopaque or conspicuous during fluoroscopy and configured for identifying the tip region of the coiling or delivery catheter 260 during fluoroscopy and two additional marker bands 264 that are radiopaque or conspicuous during fluoroscopy and configured for assisting the detachment of coil expandable implants 301. In some examples, the elongate body 261 comprises a single marker band 264 that is radiopaque or conspicuous during fluoroscopy at a location ≤10 mm proximal to the distal end of the elongate body 261 and two additional marker bands 264 that are radiopaque or conspicuous during fluoroscopy and are located on a straight portion of the elongate body 261 at a location >10 mm proximal to the distal end of the elongate body 261.

In some examples, the proximal portion of the elongate body 261 adjacent to the hub 263 comprises a segment of polymer that provides strain relief 248 to the junction between the hub 263 and the elongate body 261. In some examples, the outer diameter of the distal end of the elongate body 261 has a radius. In some examples, the tip portion of the elongate body 261 is tapered. In some examples, the outer diameter of the distal end of the elongate body 261 has a radius.

In some examples, the elongate body 261 comprises a lubricious or hydrophilic coating layer. In some examples, the lubricious or hydrophilic coating layer is present on the inner surface, the outer surface, or both the inner and outer surface of the elongate body 261. In some examples, the lubricious or hydrophilic coating layer is present on the outer surface of the distal portion of the elongate body 261, on the outer surface of the middle and distal portion of the elongate body 261, or on the entire outer surface of the elongate body 261.

In some examples, the outer diameter of the distal end of the elongate body 261 has a radius. In some examples, the distal portion of the elongate body 261 of the coiling or delivery catheter 260 is straight or is straight when in an unconstrained configuration. In some examples, a portion of the elongate body 261 of the coiling or delivery catheter 260 has a pre-formed shape or has a portion with a pre-formed shape when in an unconstrained configuration, including a distal portion.

### Coil Expandable Implants and Vascular Plug Expandable Implants

Further implementations of this disclosure include the use of coil expandable implants 301. Coil expandable implants (not shown) can be complex, flexible, elongated metallic structures with a pre-formed shape configured for permanent implantation in a patient. In some examples, coil expandable implants 301 are configured for use in obstructing or reducing the flow of blood or other biological fluids in a human patient after being placed in the lumen or space where the blood or other biological fluids are flowing. In some examples, coil expandable implant devices 300 comprise a coil expandable implant 301 and an elongate body 307 reversibly joined to the coil expandable implant 301 and configured to advance or retract the coil expandable implant 301 in a human patient.

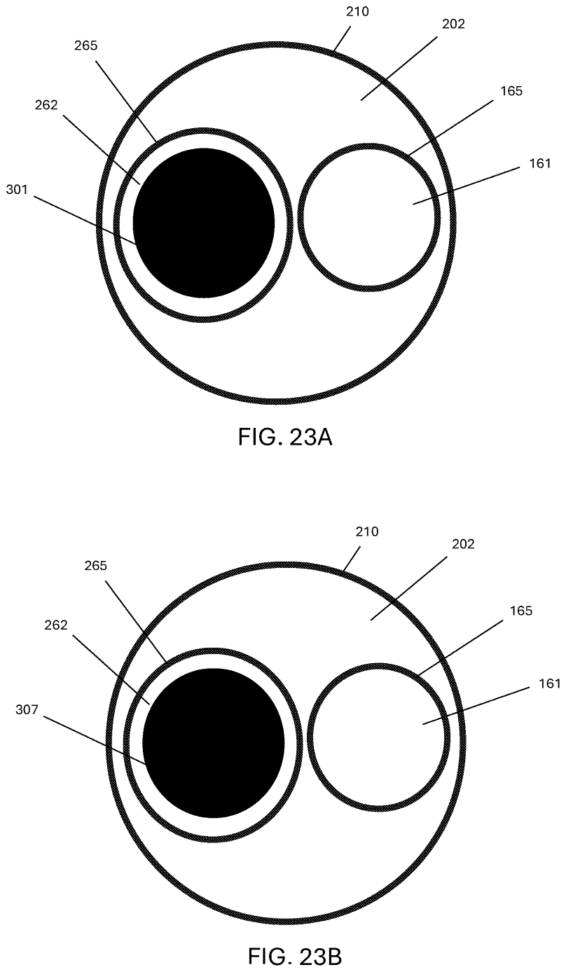

FIG. 23A is a cross-sectional view of an example of an assembly of a guide catheter or guide sheath 200, expandable metallic implant device 1, coiling or delivery catheter 260, and coil expandable implant device 300, shown with the coil expandable implant 301 of the coil expandable implant device 300 passing through the lumen 262 of the coiling or delivery catheter 260. FIG. 23B is a cross-sectional view of an example of an assembly of a guide catheter or guide sheath 200, expandable metallic implant device 1, coiling or delivery catheter 260, and coil expandable implant device 300, shown with the elongate body 307 of the coil expandable implant device 300 passing through the lumen 262 of the coiling or delivery catheter 260.

In some examples, coil expandable implants 301 are configured as a wire assembly comprising a core wire 302 surrounded by an outer coiled or externally wound wire 303. The core wire 302 has a distal end, a middle portion, and a proximal end. The middle portion of the core wire 302 may have a diameter that is greater than the diameter of the distal end, the proximal end, or both the distal and proximal end. In some examples, the core wire 302 is tapered at the distal end, the proximal end, or both the distal and proximal end. The outer wound or coiled wire can be wound in a right-hand manner or a left-hand manner around the core wire 302. The outer wound or coiled wire may be securely attached to the core wire 302 at the distal end, the proximal end, or both the distal end and the proximal end with a cap. In some examples, the coil expandable implant 301 comprises a lubricious coating layer or covering 305.

In some examples, the core wire 302 comprises stainless steel or nitinol, or combinations thereof. In some examples, outer wound or coiled wire may comprise platinum or platinum alloys, such as platinum-tungsten or platinum-iridium, or gold. Platinum is a preferred material due to its radiopacity (ability to be visualized under X-ray), conspicuity under fluoroscopy, flexibility, and biocompatibility.

The design of coil expandable implants 301 can vary depending on factors such as the size and shape of the region being treated. Coil expandable implants may have a coating layer 305 or adherent threads or fibers or other material to promote thrombosis (clotting). The coating layer 305 or adherent threads or fibers or other material encourages the formation of a stable blood clot or thrombus around the coil expandable implants 301, effectively sealing off the treated area. Coil expandable implants are designed to be detached from the elongate body 307 when they are properly positioned in vivo. This detachment can be achieved through mechanical, electrolytic, or thermal detachment, or other various types of detachment mechanisms. Coil expandable implants are radiopaque, meaning they are visible under X-ray fluoroscopy. This allows the operator to monitor the placement of the coil expandable implants 301 in real-time during the procedure. Coil expandable implants are designed to be biocompatible, meaning they are well-tolerated by the body and does not cause adverse reactions, excessive local or systemic host responses or tissue damage.

The primary structure of a coil expandable implant 301 is the “stock” wire, which is fabricated in linear form with a diameter (D1) of any range. Most stock wires used for coil expandable implant 301 manufacturing are between 0.00122 to 0.003 inch. The stock wire diameter, D1, is the central factor in determining coil “stiffness.” The stock wire is wound around a mandrel, also of varying diameter, to produce the secondary structure of the coil expandable implant 301. The diameter (D2) of the secondary structure, in conjunction with the number of turns per unit of length around the mandrel, represents two additional factors that impact product stiffness. The secondary diameter, D2, dictates the historic coil expandable implant 301 grouping, in which coil expandable implants 301 deemed “10” coils are typically wound to approximately 0.010 inch and coils deemed “18” coils are typically wound to approximately 0.015 inch. The secondary structure can be shaped into any number of tertiary configurations (including helical, complex, and spherical), which also are developed with a specific diameter (D3) and length (L), parameters that serve as important measurements shown on package labeling and serve as important factors in the selection of expandable implants during interventional procedures. For instance, coils are typically packaged as “3 mm×4 cm,” where the millimeter measurement is that of the tertiary diameter, D3, and the centimeter measurement is that of L.

In some examples, coil expandable implants 301 are joined to an elongate body 307 by a bond or joint that can be separated after placement of the coil expandable implant 301 into an artery, vein, aneurysm, parent vessel of a saccular aneurysm, paravalvular leak pathway, biological conduit, or other blood-containing, fluid-containing, or biological space, or the interior space 120 of the balloon 110 so that the elongate body 307 can be removed from the patient while the coil expandable implant 301 remains in the patient. In some examples, the coil expandable implants 301 are configured to stimulate thrombus formation and fibrosis in vivo, including by the addition of coatings, coverings, threads or fibers, including those comprised of polymers like polyesters, nylon, and polyvinyl alcohols or natural materials such as wool.

Coil expandable implants may be made from wire, polymer, and other flexible materials, and combinations therein. Coil expandable implants may be formed from self-expanding materials or generally formed in a manner that renders the coil expandable implant 301 self-expanding. Coil expandable implants may be formed from materials that are not self-expanding or generally formed in a manner wherein the coil expandable implant 301 is not self-expanding. Examples of coil expandable implants 301 include coils, metallic coils, metallic coils, polymer coils, coils comprising metal and polymer, coiled wires, coiled metallic wires, coiled metallic wires, coiled wires comprising metal and polymer, strands, polymer strands, metallic strands, metallic strands, strands comprising polymer and metal, vascular coils, assemblies of wires, assemblies of metallic wires, assemblies of metallic wires, assemblies of polymer strands, assemblies of wires or strands comprising metal and polymer, assemblies of coiled wires, assemblies of coiled metallic wires, assemblies of coiled metallic wires, assemblies of coiled polymer strands, assemblies of coiled structures comprising metal and wire, assemblies of strands, assemblies of polymer strands, assemblies of metallic strands, assemblies of metallic strands, and assemblies of strands comprising polymer and metal, and combinations thereof.

In some examples, of coil expandable implants 301, the coils, metallic coils, metallic coils, polymer coils, coils comprising metal and polymer, coiled wires, coiled metallic wires, coiled metallic wires, coiled wires comprising metal and polymer, polymer strands, metallic strands, metallic strands, strands comprising polymer and metal, vascular coils, assemblies of wires, assemblies of metallic wires, assemblies of metallic wires, assemblies of polymer strands, assemblies of wires or strands comprising metal and polymer, assemblies of coiled wires, assemblies of coiled metallic wires, assemblies of coiled metallic wires, assemblies of coiled polymer strands, assemblies of coiled structures comprising metal and wire, assemblies of polymer strands, assemblies of metallic strands, assemblies of metallic strands, and assemblies of strands comprising polymer and metal are self-expanding. In some examples, the coils, metallic coils, metallic coils, coils comprising metal and polymer, coiled wires, coiled metallic wires, coiled metallic wires, coiled wires comprising metal and polymer, metallic strands, metallic strands, strands comprising polymer and metal, vascular coils, assemblies of wires, assemblies of metallic wires, assemblies of metallic wires, assemblies of wires or strands comprising metal and polymer, assemblies of coiled wires, assemblies of coiled metallic wires, assemblies of coiled metallic wires, assemblies of coiled structures comprising metal and wire, assemblies of metallic strands, assemblies of metallic strands, and assemblies of strands comprising polymer and metal comprise nitinol.

In some examples, the coil expandable implant 301 is a vascular coil. In some examples, the coil expandable implant 301 comprises platinum, iridium, nickel, tungsten, or combinations thereof.

In some examples, coil expandable implant 301 may comprise a primary wire having a diameter of between 0.00122-0.003 inch. In some examples, coil expandable implants 301 may comprise a primary wire having a diameter of between 0.005-0.050 inch. This primary wire may be wound against itself to provide an overall or secondary diameter of the coil expandable implant 301 that is between 0.010-0.040 inch in diameter. Furthermore, this secondary shape of the coil expandable implants 301 may be formed into tertiary shapes having a diameter of approximately 2 to 100 mm. The secondary diameter of coil expandable implants 301 are generally similar to the standard diameters for guidewires 280 (0.010, 0.014, 0.018, 0.025, 0.035, and 0.038 inch) given that the same catheters are often used to place guidewires 280 and coil expandable implants 301.

Coil expandable implants come in various shapes and sizes. In some examples, at least a portion of a coil expandable implant 301 has a looped, coiled, helical, spherical, or complex tertiary structure. In some examples, at least a portion of a coil expandable implant 301 is configured to form a coiled, helical, or complex tertiary shape when relaxed. In some examples, the coil expandable implant 301 has a tertiary diameter of the looped, coiled, or formed portion with a tertiary diameter between 2-100 mm.

In various examples, the coil expandable implants 301 have a tertiary structure without pre-formed loops or shapes when relaxed (“unformed coil expandable implants”). In some examples, unformed coil expandable implants 301 are configured to form a straight or unformed tertiary shape when relaxed. In various examples, these coil expandable implants 301 are elongated and generally straight, and have a length between 10 cm-500 cm. The unformed coil expandable implants 301 may be deployed within an artery, vein, aneurysm, parent vessel of a saccular aneurysm, paravalvular leak pathway, biological conduit, or other blood-containing, fluid-containing, or biological space, or the central hollow region 16 of an expanded expandable metallic implant 11. The unformed coil expandable implants 301 may be used with various expandable metallic implants 10 of any size and shape. In one aspect, unformed coil expandable implants 301 are formed into a secondary shape when placed within the central hollow region 16 of an expanded expandable metallic implant 11. The expanded expandable metallic implant 11 gives the unformed coil expandable implants 301 the ideal secondary shape by constraining them inside the expanded expandable metallic implant 11. Unformed coil expandable implants 310 are desirable as they reduce friction during passage through coiling or delivery catheters 260. The unformed coil expandable implants 310 also permits the use of fewer unformed coil expandable implants 310 and reduces overall treatment times.

In some examples, unformed coil expandable implants 310 may be manufactured to include one or more proximal or distal loops. The loops may be deformed under tension for delivery through catheters and then return to their pre-formed loop shape after they exit the catheter. The loops on the unformed coil expandable implants 310 may reduce the risk of tissue injury during placement that could occur when using unformed coil expandable implants 310 lacking loops. The loops on the unformed coil expandable implants 310 may present a flatter surface to the adjacent tissue as the unformed coil expandable implant 301 is pushed forward, reducing the risk of tissue injury. In some examples, the distal portion of a coil expandable implant 301 comprises 1, 2, 3 or 4 loops of tertiary structure and the remainder of the coil expandable implant 301 comprises a tertiary structure without pre-formed loops or shapes when relaxed. In some examples, the distal portion of a coil expandable implant 301 comprises 1, 2, 3 or 4 or more than 4 loops of tertiary structure and the remainder of the coil expandable implant 301 comprises a tertiary structure configured to form a straight or unformed tertiary shape when relaxed. The tertiary diameter of the looped, coiled, formed, or tertiary portion of the coil expandable implant 301 is between 2-100 mm.

In some methods, the elongate body 307 of a coil expandable implant device 300 is a wire, laser cut nitinol tube, or catheter.

In some methods, the operator can gently push all or a portion of a coil expandable implant 301 out of a coiling or delivery catheter 260 and use fluoroscopy or angiography to evaluate the placement of the coil expandable implant 301 and the degree of obstruction or reduction in flow, before detaching the coil expandable implant 301 from the elongate body 307. If the placement of the coil expandable implant 301 or the degree of obstruction or reduction in flow is not appropriate then the coil expandable implant 301 can be repositioned and detached, the coil expandable implant 301 can be removed, the expandable metallic implant 10 can be deflated and repositioned (if possible), the expandable metallic implant 10 can be removed and replaced (if possible), an additional expandable metallic implant 10 can be placed, or additional coil expandable implants 301 can be placed.

In some examples, at least a portion of a coil expandable implant 301 is configured to contact the interior surface 42 of an expanded expandable metallic implant 11 of the first device. In some examples, the largest overall diameter or tertiary diameter of the coil expandable implant 301 is in a range from 5% smaller than the largest diameter of the expanded expandable metallic implant 11 to 20% larger than the largest diameter of the expanded expandable metallic implant 11. In some examples, the largest overall or tertiary diameter of the expanded expandable metallic implant 11 is 1, 2, 3, 4, 5, 6, 7, 8, 9, or 10 mm larger than the largest diameter of the expanded expandable metallic implant 11.

In some examples, vascular plug expandable implants are joined to a longitudinal body by a bond or joint that can be separated after placement of the vascular plug expandable metallic implant into an artery, vein, aneurysm, parent vessel of a saccular aneurysm, paravalvular leak pathway, biological conduit, or other blood-containing, fluid-containing, or biological space, so that the longitudinal body can be removed from the patient while the vascular plug expandable metallic implant remains in the patient. In some examples, the vascular plug expandable metallic implant is configured to stimulate thrombus formation and fibrosis in vivo, including by the addition coatings, coverings, threads, or fibers, including those comprised of polymers like polyesters, nylon, and polyvinyl alcohols or natural materials such as wool.

Vascular plug expandable implants may be made from wire, polymer, and other flexible materials, and combinations therein. Vascular plug expandable implants are generally formed from self-expanding materials or generally formed in a manner that renders the expandable metallic implant 10 self-expanding. Examples of vascular plug expandable implants include self-expanding wires, nitinol wires, assemblies of wires, assemblies of metallic wires, assemblies of metallic wires, assemblies of polymer strands, assemblies of wires or strands comprising metal and polymer, assemblies of coiled wires, assemblies of coiled metallic wires, assemblies of coiled metallic wires, assemblies of coiled polymer strands, assemblies of coiled structures comprising metal and wire, assemblies of strands, assemblies of polymer strands, assemblies of metallic strands, assemblies of metallic strands, and assemblies of strands comprising polymer and metal, assemblies of braided wires, assemblies of braided metallic wires, assemblies of braided metallic wires, assemblies of braided wires comprising metal and polymer, assemblies of braided strands, assemblies of braided polymer strands, assemblies of braided strands comprising polymer and metal, assemblies of woven wires, assemblies of woven metallic wires, assemblies of woven metallic wires, assemblies of woven wires comprising metal and polymer, assemblies of woven strands, assemblies of woven polymer strands, assemblies of woven strands comprising polymer and metal, and combinations thereof.

In some methods, the elongate body of a vascular plug expandable metallic implant device is a wire, laser cut nitinol tube, or catheter.

In some examples, the vascular plug expandable metallic implant, or a similar appearing implant, is configured to contact the interior surface 42 of the expanded expandable metallic implant 11. In some examples, the largest overall diameter of the vascular plug expandable metallic implant is in a range from 5% smaller than the largest diameter of the expanded expandable metallic implant 11 to 20% larger than the largest diameter of the expanded expandable metallic implant 11. In some examples, the largest overall diameter of the vascular plug expandable metallic implant is 1, 2, 3, 4, 5, 6, 7, 8, 9, or 10 mm larger than the largest diameter of the expanded expandable metallic implant 11.

### Guidewires and Methods of Use

A guidewire 280 can be a form of elongate body that is configured for use in guiding and advancing a guide catheter or guide sheath 200, intermediate catheter, selective catheter 240, or coiling or delivery catheter 260 in a human patient. Guidewires 280 generally have a secondary diameter between 0.010-0.038 inch, including guidewires 280 with diameters of 0.010, 0.011, 0.012, 0.013, 0.014, 0.015, 0.016, 0.017, 0.018, 0.033, 0.034, 0.035, 0.036, 0.037, or 0.038 inch. Guidewires 280 generally have a length between 45-500 cm. The lumen 202 of a guide catheter or guide sheath 200, intermediate catheter 220, selective catheter 240, or coiling or delivery catheter 260 can be configured to accept guidewires 280 of various diameters and lengths.

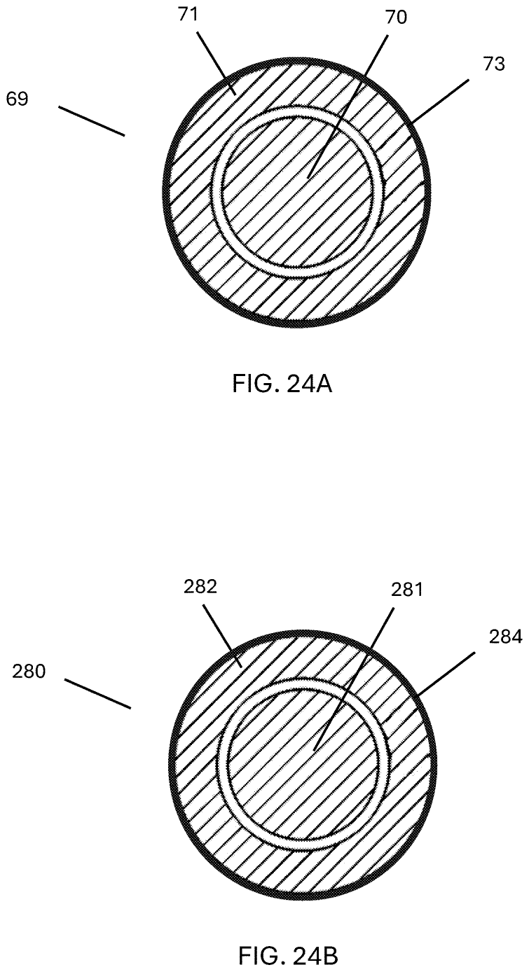

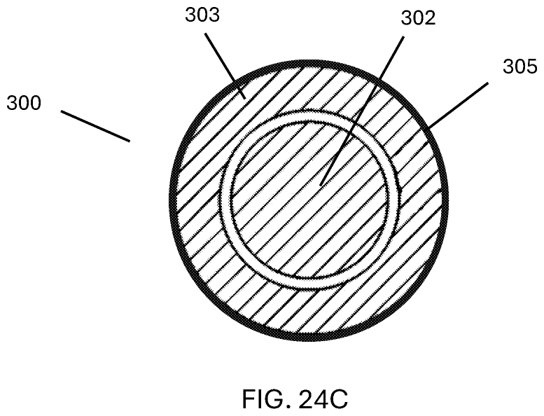

FIG. 24A is a cross-sectional view of an example of a guidewire segment 69 of an expandable metallic implant 10. FIG. 24B is a cross-sectional view of an example of a guidewire 280. FIG. 24C is a cross-sectional view of an example of a coil expandable implant device 300.

In some examples, guidewires 280 are configured as a wire assembly comprising a core wire 281 surrounded by an outer coiled or externally wound wire 303. The core wire 281 has a distal end, a middle portion, and a proximal end. The middle portion of the core wire 281 may have a diameter that is greater than the diameter of the distal end, the proximal end, or both the distal and proximal end. In some examples, the core wire 281 is tapered at the distal end, the proximal end, or both the distal and proximal end. The outer wound or coiled wire 282 can be wound in a right-hand manner or a left-hand manner around the core wire 281. The outer wound or coiled wire 281 may be securely attached to the core wire 281 at the distal end, the proximal end, or both the distal end and the proximal end with a cap 283.

In some examples, the core wire 281 comprises stainless steel, nitinol, or combinations thereof. In some examples, the outer coiled or externally wound wire 282 comprises platinum, gold, or combinations thereof. In some examples, the guidewire 280 comprises a lubricious coating layer or covering 284. In some examples, a portion of the guidewire 280 has a pre-formed shape 285 or has a portion with pre-formed shape when in an unconstrained configuration, including a distal portion, to assist in the selection of vessel branches.

### Systems Comprising Guide Sheaths or Guide Catheters, Intermediate Catheters, Selective Catheters, and Guidewires, and Methods of Use

Further implementations of this disclosure relate to systems comprising a guide catheter or guide sheath 200 and a guidewire 280, wherein the guide catheter or guide sheath 200 comprises an elongate body 201 and a hub 203, wherein the diameter and length of the lumen 202 of the guide catheter or guide sheath 200 is configured so that the guidewire 280 can be passed through the lumen 202 of the guide catheter or guide sheath 200 and positioned at the target location in vivo. In some examples, of a system comprising a guide catheter or guide sheath 200 and a guidewire 280, the system may comprise one or more of the following: a system wherein the outer diameter of the guidewire 280 is at least 0.002 inch smaller than the diameter of the lumen 202 of the guide catheter or guide sheath 200, including a system wherein a portion of the guidewire 280 passes through the lumen 202 of the guide catheter or guide sheath 200; a system wherein the lumen 202 of the guide catheter or guide sheath 200 is configured to allow for the passage of the guidewire 280 through the lumen 202 of the guide catheter or guide sheath 200; a system wherein length of the guidewire 280 is at least 5 mm longer than the length of the guide catheter or guide sheath 200; a system wherein the guidewire 280 can be moved forward or backward while the guide catheter or guide sheath 200 remains fixed in position; a system wherein the guide catheter or guide sheath 200 can be moved forward or backward while the guidewire 280 remains fixed in position; a system further comprising a Tuohy Borst adaptor 406 operably and reversibly coupled to the hub 203 of the guide catheter or guide sheath 200, wherein the Tuohy Borst adaptor 406 comprises a side arm 408 configured for the injection of fluids, including water, saline, radiographic contrast, solutions comprising therapeutic agents or drugs, and mixtures therein.

The present disclosure also relates to systems comprising a guide catheter or guide sheath 200 device and a guidewire 280, further comprising an intermediate catheter 220 comprising an elongate body 221 and a hub 223, wherein the elongate body 221 of the intermediate catheter 220 is configured to pass through the lumen 202 of the guide catheter or guide sheath 200. In some examples, the proximal portion 228 and middle portion 229 of the elongate body 221 of the intermediate catheter 220 has a larger inner and outer diameter than the distal portion 230 of the elongate body 221 of the intermediate catheter 220. In some examples, the addition of the intermediate catheter 220 to the system is intended to reduce friction when advancing the guide catheter or guide sheath 200 in vivo. In some examples, of a system comprising a guide catheter or guide sheath 200 and a guidewire 280, the system may comprise one or more of the following: a system wherein the outer diameter of the proximal portion 228 of the intermediate catheter 220 is at least 0.002 inch smaller than the diameter of the lumen 202 of the guide catheter or guide sheath 200, including a system wherein a portion of the elongate body 221 of the intermediate catheter 220 passes through the lumen 202 of the guide catheter or guide sheath; a system wherein the diameter of the lumen 202 of the distal portion 230 of the intermediate catheter 220 is at least 0.002 inch larger than the outer diameter of the guidewire 280, including a system wherein a portion of the guidewire 280 passes through the lumen 222 of the intermediate catheter 220; a system wherein the elongate body 221 of the intermediate catheter 220 is at least 5 mm longer than the length of the guide catheter or guide sheath 200; a system wherein the guidewire 280 is at least 5 mm longer than the length of the intermediate catheter 220; a system wherein the lumen 222 of the intermediate catheter 220 is configured to allow for the passage of the guidewire 280 through the lumen 222 of the intermediate catheter 220; a system wherein the hub 223 of the intermediate catheter 220 and the hub 203 of the guide catheter or guide sheath 200 can be reversibly joined or engaged, including wherein the reversible engagement or joining of the hub 223 of the intermediate catheter 220 and the hub 203 of the guide catheter or guide sheath 200 prevents or reduces the leaking of blood from the guide catheter or guide sheath 200 during use, and wherein, once joined, the hub 223 of the intermediate catheter 220 and hub 203 of the guide catheter or guide sheath 200 can be separated and the intermediate catheter 220 can be removed from the guide catheter or guide sheath 200, and wherein the joining or engagement and the separation of the hub 223 of the intermediate catheter 220 and hub 203 of the guide catheter or guide sheath 200 can occur by rotating a lock; a system further comprising a second Tuohy Borst adaptor 409 operably and reversibly coupled to the hub 223 of the intermediate catheter 220, the second Tuohy Borst adaptor 409 comprising a side arm 408 configured for the injection of fluids, including water, saline, radiographic contrast, solutions comprising therapeutic agents or drugs, and mixtures therein, wherein the guidewire 280 can be inserted through the second Tuohy Borst adaptor 409 and into the lumen 222 of the intermediate catheter 220; a system wherein the guidewire 280 can be moved forward or backward while the guide catheter or guide sheath 200 and intermediate catheter 220 remain fixed in position; a system wherein the intermediate catheter 220 can be moved forward or backward while the guide catheter or guide sheath 200 and the guidewire 280 remain fixed in position; a system wherein the guide catheter or guide sheath 200 can be moved forward or backward while the intermediate catheter 220 and the guidewire 280 remain fixed in position; and a system wherein the guide catheter or guide sheath 200 and intermediate catheter 220 can be moved forward or backward while the guidewire 280 remains fixed in position.

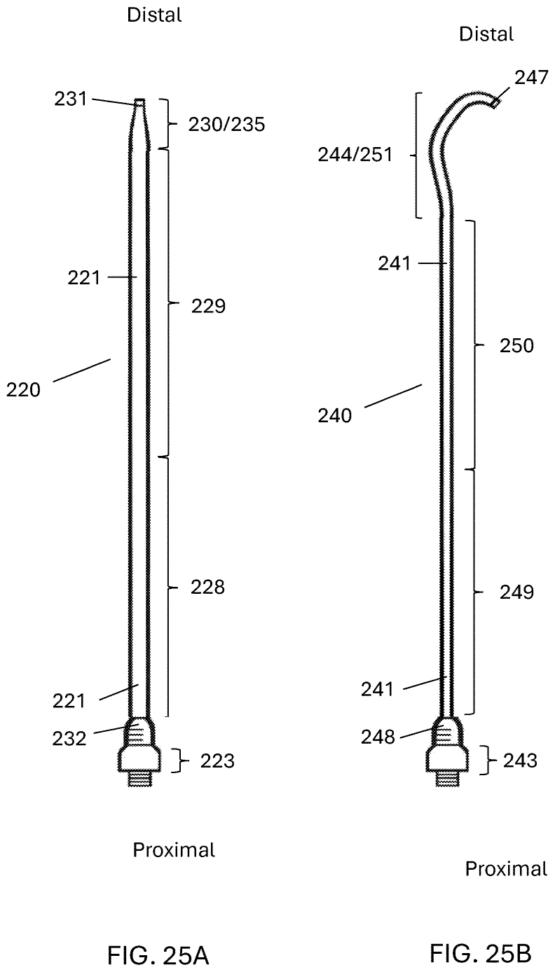

FIG. 25A is a planar view of an example of an intermediate catheter 220. FIG. 25B is a planar view of an example of a selective catheter 240.

Further implementations of this disclosure relate to systems comprising a guide catheter or guide sheath 200 device, a guidewire 280, and an intermediate catheter 220, further comprising a selective catheter 240 comprising an elongate body 241 and a hub 243, wherein the elongate body 241 of the selective catheter 240 is configured to pass through the lumen 202 of the guide catheter or guide sheath 200 and the intermediate catheter 220, if present. In some examples, the addition of the selective catheter 240 to the system is intended to assist in advancing the guide catheter or guide sheath 200 to the treatment site. In some examples, the distal portion 251 of the elongate body 241 of the selective catheter 240 has a pre-formed shape 246 to direct a guidewire 280 or assist in the selection of vessel branches. In some examples, of a system comprising a guide catheter or guide sheath 200 device, a guidewire 280, an intermediate catheter 220 and a selective catheter 240, the system may comprise one or more of the following: a system wherein the diameter of the lumen 242 of the proximal portion 249 of the elongate body 241 of the selective catheter 240 is between 0.037-0.053 inch and the lumen 242 of the distal portion 251 of the elongate body 241 of the selective catheter 240 is between 0.037-0.057 inch; a system wherein the largest outer diameter of the selective catheter 240 is at least 0.002 inch smaller than the smallest diameter of the lumen 222 of the intermediate catheter 220, including systems wherein a portion of the elongate body 241 of a selective catheter 240 passes through the lumen 222 of the intermediate catheter 220; a system wherein the outer diameter of the guidewire 280 is at least 0.002 inch smaller than the diameter of the lumen 242 of the selective catheter 240, including a system wherein a portion of the guidewire 280 passes through the lumen 242 of the selective catheter 240; a system wherein the distal portion 251 of the elongate body 241 of the selective catheter 240 has a pre-formed shape 244 or has a pre-formed shape 244 when in an unconstrained configuration; a system wherein the elongate body 241 of a selective catheter 240 is at least 5 mm longer than the length of the intermediate catheter 220; a system wherein the guidewire 280 is at least 5 mm longer than the length of the selective catheter 240; a system wherein the lumen 222 of the intermediate catheter 220 is configured to allow for the passage of the selective catheter 240 through the lumen 222 of the intermediate catheter 220; a system wherein the lumen 222 of the selective catheter 240 is configured to allow for the passage of the guidewire 280 through the lumen 222 of the selective catheter 240; a system wherein the selective catheter 240 can be moved forward or backward while the guide catheter or guide sheath 200, intermediate catheter 220, and guidewire 280 remain fixed in position; a system wherein the guide catheter or guide sheath 200 and intermediate catheter 220 can be moved forward or backward while the selective catheter 240 and guidewire 280 remain fixed in position; a system wherein the intermediate catheter 220 can be moved forward or backward while the guide catheter or guide sheath 200, selective catheter 240, and the guidewire 280 remain fixed in position; a system wherein the selective catheter 240 can be moved forward or backward while the guide catheter or guide sheath 200, intermediate catheter 220, and the guidewire 280 remain fixed in position; and a system wherein the guidewire 280 can be moved forward or backward while the guide catheter or guide sheath 200, intermediate catheter 220, and selective catheter 240 remain fixed in position.

The present disclosure relates to systems comprising a guidewire 280 that is configured to pass through the lumen 202 of the guide catheter or guide sheath 200, the intermediate catheter 220 (if present), or the selective catheter 240 (if present). In some examples, the addition of the guidewire 280 to the system is intended to assist in advancing the guide catheter or guide sheath 200, the intermediate catheter 220 (if present), or the selective catheter 240 (if present) to the treatment site.

### Systems Comprising Expandable Implant Medical Devices, Guide Catheters or Guide Sheaths, and Coil Expandable Implants, and Vascular Plug Expandable Implants, and Methods of Use

Further implementations of this disclosure relate to devices that comprise a coil expandable implant 301 or vascular plug expandable implant. Coil expandable implants 300 and vascular plug expandable implants are flexible structures that can be pushed or carried to a desired location in a patient in an unexpanded, constrained, collapsed, compressed, straightened, elongated, or folded and pleated form and implanted in the patient, wherein at least portions of the expandable metallic implant can expand in size or shape during or after placement. Some types of these other expandable implant devices can be pushed through coiling catheters or delivery catheters 260 using an elongate body 307 and deposited at the target location, including coil expandable implants 301 and vascular plug expandable implants. Some types of these vascular plug expandable implants are advanced over a guidewire 280 to the target location.

The present disclosure describes devices, systems, and methods to obstruct, occlude, embolize, or reduce flow in arteries, veins, aneurysms, parent vessels of saccular aneurysms, paravalvular leak pathways, biological conduits, or other blood-containing, fluid-containing, or biological spaces, wherein an expandable metallic implant 10 is placed in the artery, vein, aneurysm, parent vessel of a saccular aneurysm, paravalvular leak pathway, biological conduit, or other blood-containing, fluid-containing, or biological space, and maintained there in an expanded configuration. In some examples, the present disclosure relates to devices, systems, and methods for delivering and positioning various examples of coil expandable implants 301 and vascular plug expandable implants before, during, and after expansion of an expandable metallic implant 10 and wherein the expandable metallic implants 10, coil expandable implants 301, and vascular plug expandable implants are dimensioned and configured to fill or seal at least a portion of the artery, vein, aneurysm, parent vessel of a saccular aneurysm, paravalvular leak pathway, biological conduit, or other blood-containing, fluid-containing, or biological space, and wherein the expandable metallic implants 10, coil expandable implants 301, and vascular plug expandable implants remain in place in an expanded or elongated state. In some examples, one or more coil expandable implants 301 and vascular plug expandable implants are placed adjacent to an expandable metallic implant 10 to increase the rate or completeness of the obstruction, occlusion, embolization, or flow reduction. In some examples, one or more coil expandable implants 301 and vascular plug expandable implants are placed adjacent to an expandable metallic implant 10 to reduce the risk of migration of the expanded expandable metallic implant 11.

In some examples, an expandable metallic implant 10 in a unexpanded or folded and pleated form is advanced through the lumen 202 of a guide catheter or guide sheath 200 and positioned in an artery, vein, aneurysm, parent vessel of a saccular aneurysm, paravalvular leak pathway, biological conduit, or other blood-containing, fluid-containing, or biological space, and partially or fully expanded but, prior to separation of the expandable metallic implant 10 and the elongate body 160, a coiling or delivery catheter 260 is advanced through the lumen 202 of the guide catheter or guide sheath 200 alongside the elongate body 160 and one or more coil expandable implants 301 or vascular plug expandable implants are advanced through the lumen 262 of the coiling or delivery catheter 260 and placed proximal to, distal to, or adjacent to the expandable metallic implant 10.

Optionally, the present disclosure also describes the placement of one or more coil expandable implants 301 inside the central hollow region 16 of the expanded expandable metallic implant 11 to help maintain the size and shape of the expanded expandable metallic implant 11 and provide reinforcement and resist collapse, compression, and compaction of the expanded expandable metallic implant 11. Optionally, the present disclosure also describes the placement of one or more coil expandable implants 301 or vascular plug expandable implants distal to, proximal to, or adjacent to an expanded expandable metallic implant 11, either before or after expansion, to help reduce blood flow, support the position of the expanded expandable metallic implant 11, and reduce the risk of migration of the expanded expandable metallic implant 11. In some examples, the one or more coil expandable implants 301 or vascular plug expandable implants can be placed through the lumen 262 of a coiling or delivery catheter 260 and implanted in the patient proximal to, distal to, or adjacent to the expandable metallic implant 10, either before or after expansion, or before or after separation of the deflated or collapsed balloon 110 from the expanded expandable metallic implant 11.

In some examples, the expandable metallic implant 10 is expanded by the injection of fluid through the lumen 161 of the elongate body 160 and into the interior space 120 of the balloon 110. Expandable metallic implant devices with larger diameter expandable metallic implants 10 and balloons 110 require injection of larger amounts of fluid to achieve full expansion of the expandable metallic implants 10 and balloons 110. The rate of fluid injection into the interior space 120 of the balloon 110 is dependent on several factors, including the pressure of the injection, the resistance to the flow of fluid in the lumen 161 of the elongate body 160, the resistance to the flow of fluid through the proximal neck 123 of the balloon 110, the resistance to expansion of the folded and pleated balloon 115, and the resistance to expansion of the folded and pleated expandable metallic implant 13. The resistance to the flow of fluid in the lumen 161 of the elongate body 160 is reduced when the lumen 161 diameter increases. In certain examples, expandable metallic implant devices 1 with a larger diameter expanded expandable metallic implant 11 also have an elongate body 160 with a larger lumen 161 diameter in order to keep expansion times low or relatively similar between the various device sizes. In certain examples, systems comprising an expandable metallic implant device 1, a guide catheter or guide sheath 200, and a coiling or delivery catheter 260 are configured so that the devices can work together to obstruct, occlude, embolize, or reduce flow in arteries, veins, aneurysms, parent vessels of a saccular aneurysms, paravalvular leak pathways, biological conduits, or other blood-containing, fluid-containing, or biological spaces. In such system examples, the lumen 202 of the guide catheter or guide sheath 200 can be large enough to allow the folded and pleated expandable metallic implant 13 and the elongate body 160 of the folded and pleated expandable metallic implant device 2 to pass through the lumen 202 of the guide catheter or guide sheath 200, and to the target location in the patient. In some examples, the inner diameter of the guide catheter or guide sheath 200 (the diameter of the lumen 202) and the outer diameter of the elongate body 160 of the folded and pleated expandable metallic implant device 2 are configured such that a coiling or delivery catheter 260 for placing coil expandable implants 301 or vascular plug expandable implants can be advanced through the lumen 202 of the guide catheter or guide sheath 200 while the elongate body 160 is also present in the lumen 202 of the guide catheter or guide sheath 200, so that coil expandable implants 301 or vascular plug expandable implants can be placed distal to, adjacent to, or proximal to the expandable metallic implant 10, including prior to or after expansion of the expandable metallic implant 10. In some examples, the coiling or delivery catheter 260 is configured to accept both guidewires 280 and coil expandable implants 301 or vascular plug expandable implants, or similar types of implants. In some examples, the system is designed so that the coiling or delivery catheter can be moved forward or backward while the expandable metallic implant device 1 and the guide catheter or guide sheath 200 remain fixed in position. In some examples, the system is designed so that the expandable metallic implant device 1 can be moved forward or backward while the coiling or delivery catheter 260 and the guide catheter or guide sheath 200 remain fixed in position. In some examples, the system is designed so that the guide catheter or guide sheath 200 can be moved forward or backward while the expandable metallic implant device 1 and coiling or delivery catheter 260 remain fixed in position. In some examples, the system is designed so that coil expandable implants 301 or vascular plug expandable implants can be moved forward or backward while the coiling or delivery catheter 260, expandable metallic implant device 1, and guide catheter or guide sheath 200 and remain fixed in position. In some examples, coil expandable implants 301 or vascular plug expandable implants can be placed at the target location while the expandable metallic implant 10 is folded and pleated, partially expanded, or fully expanded. These types of systems wherein expandable metallic implants 10, coil expandable implants 301, or vascular plug expandable implants are used together allows operators to obstruct, occlude, embolize, or reduce flow in-arteries, veins, aneurysms, parent vessels of a saccular aneurysms, paravalvular leak pathways, biological conduits, or other blood-containing, fluid-containing, or biological spaces in less time, to a greater degree, and to a greater extent than can be achieved by using the expandable metallic implant 10 alone. These types of systems wherein expandable metallic implants 10, coil expandable implants 301, or vascular plug expandable implants are used together allows operators to reduce the risk of migration of both the expanded expandable metallic implant 11 and the coil expandable implants 301 or vascular plug expandable implants. In some examples, a system of one or more expandable metallic implants 10 and one or more coil expandable implants 301 or vascular plug expandable implants is assembled in vivo to obstruct, occlude, embolize, or reduce flow in arteries, veins, aneurysms, parent vessels of saccular aneurysms, paravalvular leak pathways, biological conduits, or other blood-containing, fluid-containing, or biological spaces. In some examples, a system of one or more expandable metallic implants 10 and one or more coil expandable implants 301 or vascular plug expandable implants is assembled in vivo to reduce the risk of migration of both the expanded expandable metallic implant 11 and the coil expandable implants 301 or vascular plug expandable implants.

Coil expandable implants can be configured for use with the expandable metallic implant devices 1, including by incorporating a design wherein the coil expandable implants 301 are straight or mostly straight, enabling the adjunctive use of one or a few long, straight, or mostly straight, coil expandable implants 301 for use in various clinical situations and thereby potentially reducing procedure time and cost.

### Systems Comprising Expandable Metallic Implant Medical Devices and Guide Sheath or Guide Catheter Medical Devices, Coiling Catheter or Delivery Catheter Medical Devices, and Coil Expandable Implant Medical Devices, or Vascular Plug Expandable Metallic Implant Medical Devices, and Methods of Use

Further implementations of this disclosure relate to systems comprising an expandable metallic implant device 1 and a guide catheter or guide sheath 200 device, wherein the guide catheter or guide sheath 200 comprises an elongate body 201 and a hub 203, wherein the diameter and length of the lumen 202 of the guide catheter or guide sheath 200 is configured so that the folded and pleated expandable metallic implant 13 and elongate body 160 of a folded and pleated expandable metallic implant device 2 can be passed through the lumen 202 of the guide catheter or guide sheath 200 and positioned at the target location in vivo. In some examples, of a system comprising an expandable metallic implant device 1 and a guide catheter or guide sheath 200, the system may comprise one or more of the following: a system wherein the difference between the diameter of the lumen 202 of the guide catheter or guide sheath 200 and the outer diameter of the elongate body 160 is >0.020 inch, including a system wherein the elongate body 160 passes through the lumen 202 of the guide catheter or guide sheath 200; a system wherein the elongate body 160 passes through the lumen 202 of the guide catheter or guide sheath 200; a system wherein the elongate body 160 is at least 5 mm longer than the overall length of the guide catheter or guide sheath 200; a system wherein the lumen 202 of the guide catheter or guide sheath 200 is configured to allow for the passage of the folded and pleated portion of the folded and pleated expandable metallic implant device 2 through the guide catheter or guide sheath 200 and into the body of the patient distal to the guide catheter or guide sheath 200; a system wherein the guide catheter or guide sheath 200 can be moved forward or backward while the expandable metallic implant device 1 remains fixed in position; a system wherein the folded and pleated expandable metallic implant device 2 can be moved forward or backward while the guide catheter or guide sheath 200 remains fixed in position; a system further comprising a Tuohy Borst adaptor 406 operably and reversibly coupled to the hub 203 of the guide catheter or guide sheath 200, the Tuohy Borst adaptor 406 comprising a side arm 408 configured for the injection of fluids, including water, saline, radiographic contrast, solutions comprising therapeutic agents or drugs, and mixtures therein.

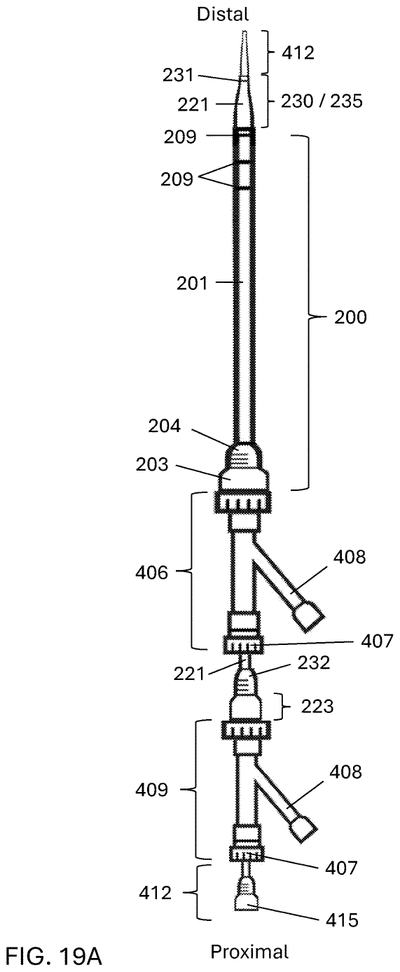

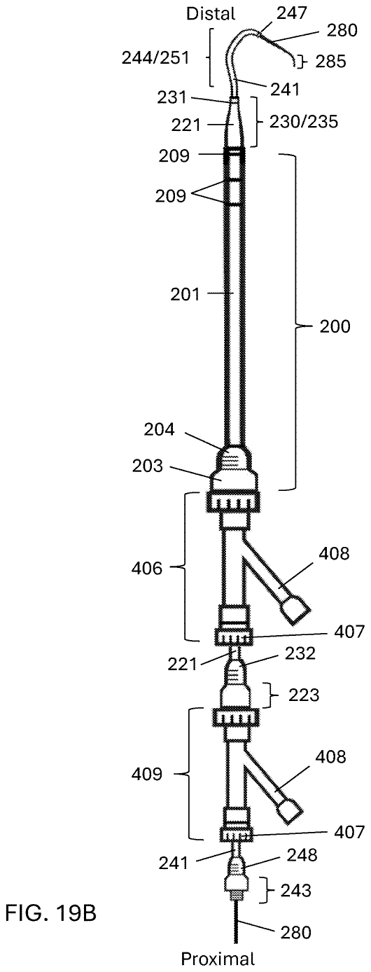

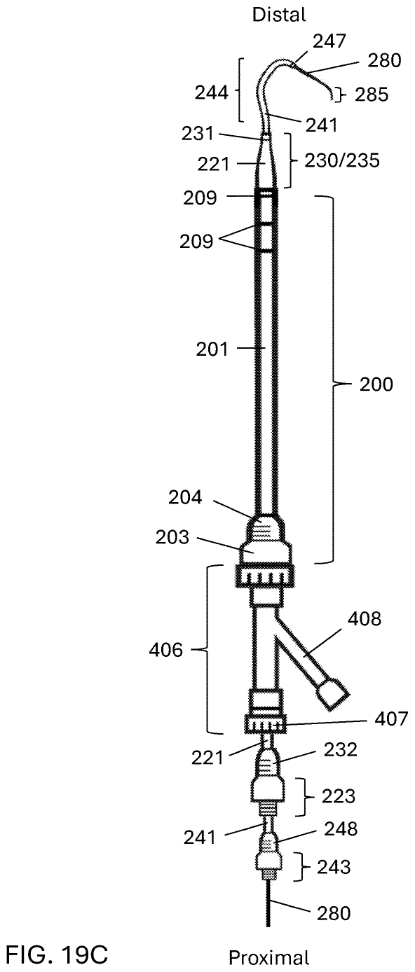

FIG. 19A is a planar view of an assembly of a guide catheter or guide sheath 200, a first Tuohy Borst adaptor 406 joined to the hub 203 of the guide catheter or guide sheath 200 and configured to reduce bleeding and to facilitate flushing of the lumen 202 of the guide catheter or guide sheath 200, an intermediate catheter 220, a second Tuohy Borst adaptor 409 joined to the hub 223 of the intermediate catheter 220 and configured to reduce bleeding and to facilitate flushing of the lumen 222 of the intermediate catheter 220, and an obturator 412. FIG. 19B is a planar view of an assembly of a guide catheter or guide sheath 200, a first Tuohy Borst adaptor 406 joined to the hub 203 of the guide catheter or guide sheath 200 and configured to reduce bleeding and to facilitate flushing of the lumen 202 of the guide catheter or guide sheath 200, an intermediate catheter 220, a second Tuohy Borst adaptor 409 joined to the hub 223 of the intermediate catheter 220 and configured to reduce bleeding and to facilitate flushing of the lumen 222 of the intermediate catheter 220, a selective catheter 240, and guidewire 280. FIG. 19C is a planar view of an assembly of a guide catheter or guide sheath 200, a Tuohy Borst adaptor 406 joined to the hub 203 of the guide catheter or guide sheath 200 and configured to reduce bleeding and to facilitate flushing of the lumen 202 of the guide catheter or guide sheath 200, an intermediate catheter 220, selective catheter 240, and guidewire 280.

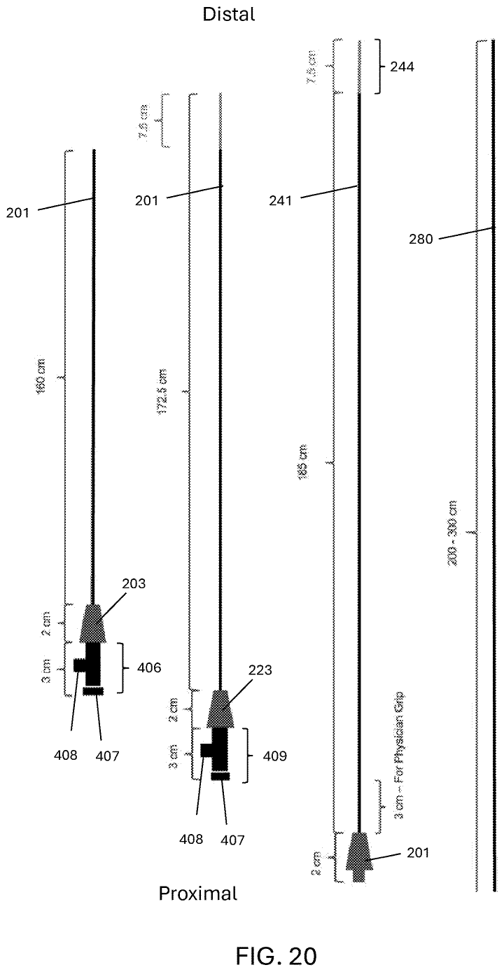

FIG. 20 is a planar view of an example of a system comprising a guide sheath 200, first Tuohy Borst adaptor 406, intermediate catheter 220, second Tuohy Borst adaptor 409, selective catheter 240, and a guidewire 280.

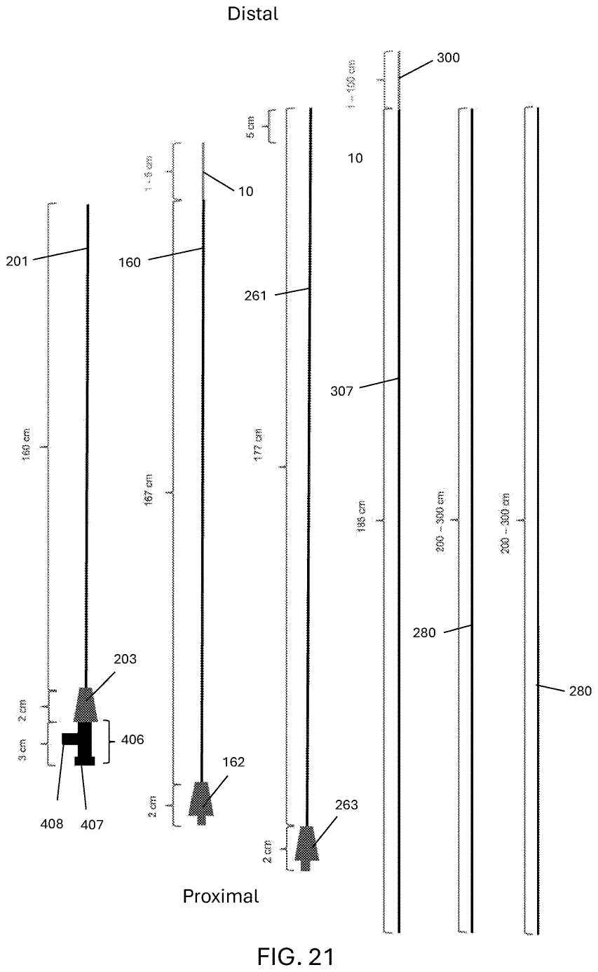

FIG. 21 is a planar view of an example of a system comprising a guide sheath 200, first Tuohy Borst adaptor 406, expandable metallic implant device 1, coiling or delivery catheter 260, and a coil expandable implant device 300.

In some examples, the distal end of the guide catheter or guide sheath 200 is configured for use in assisting the separation of the expanded expandable metallic implant 11 and the balloon catheter 100. The present disclosure also relates to the use of systems comprising an expandable metallic implant device 1 and a guide catheter or guide sheath 200, including using the distal end of the guide catheter or guide sheath 200 to separate or detach an expanded expandable metallic implant 11 from a balloon catheter 100. In some examples, of the system comprising an expandable metallic implant device 1 and a guide catheter or guide sheath 200, a Tuohy Borst adaptor 406 with a side arm 408 for injection of fluids is joined to the hub 203 of the guide catheter or guide sheath 200 to enable the injection of radiographic contrast material through the lumen 202 of the guide catheter or guide sheath 200 and into patients using the Tuohy Borst adaptor 406, including: after positioning the expandable metallic implant 10 in a patient but before the expansion of the expandable metallic implant to evaluate the position of the expandable metallic implant; after expansion of the expandable metallic implant 10 to evaluate the shape and position of the expandable metallic implant 10 and the obstruction or reduction in the rate of flow at the target location; and after removal of the balloon catheter 100 from the patient to evaluate the shape and position of the expanded expandable metallic implant 11 and the obstruction or reduction in the rate of flow at the target location.

The present disclosure can also relate to systems comprising an expandable metallic implant device 1 and a guide catheter or guide sheath 200 device, further comprising a coiling or delivery catheter 260, and one or more coil expandable implant devices 300 or vascular plug expandable metallic implant devices. In some examples, of a system comprising an expandable metallic implant device 1, a guide catheter or guide sheath 200, a coiling or delivery catheter 260, and one or more coil expandable implant devices 300 or vascular plug expandable metallic implant devices, the system may comprise one or more of the following: a system wherein the difference between the diameter of the lumen 202 of the guide catheter or guide sheath 200 and the sum of the outer diameter of the elongate body 160 of the balloon catheter 100 and the elongate body 261 of the coiling or delivery catheter 260 is >0.020 inch, including a system wherein the elongate body 160 of the balloon catheter 100 and the elongate body 261 of the coiling or delivery catheter 260 pass through the lumen 202 of the guide catheter or guide sheath 200; a system wherein the lumen 202 of the guide catheter or guide sheath 200 is configured to allow for the simultaneous passage of the elongate body 160 of the balloon catheter 100 and the elongate body 261 of the coiling or delivery catheter 260 through the lumen 202 of the guide catheter or guide sheath 200; a system wherein, when the elongate body 160 of the balloon catheter 100 passes through the lumen 202 of the guide catheter or guide sheath 200 and the proximal end of the balloon 110 is distal to the distal end of the guide catheter or guide sheath 200, the elongate body 261 of the a coiling or delivery catheter 260 can be passed through the lumen 202 of the guide catheter or guide sheath 200; a system wherein the elongate body 261 of the coiling or delivery catheter 260 is at least 5 mm longer than the overall length of the expandable metallic implant device 1, including examples where the expandable metallic implant is folded and pleated or expanded; a system wherein the coiling or delivery catheter 260 can be moved forward or backward while the guide catheter or guide sheath 200 and the folded and pleated expandable metallic implant device 2 remain fixed in position; a system wherein the folded and pleated expandable metallic implant device 2 can be moved forward or backward while the guide catheter or guide sheath 200 and the coiling or delivery catheter 260 remain fixed in position; a system wherein the guide catheter or guide sheath 200 can be moved forward or backward while the folded and pleated expandable metallic implant device 2 and the coiling or delivery catheter 260 remain fixed in position; or a system wherein the coil expandable implant device 300 or the vascular plug device can be moved forward or backward while the guide catheter or guide sheath 200, the folded and pleated expandable metallic implant device 2, and the coiling or delivery catheter 260 remain fixed in position.

### Manufacturing of Expandable Metallic Implants

Various configurations of expandable metallic implants 10 are described, along with the associated methods of manufacturing and use. In one example, the expandable metallic implant 10 portion of an expandable metallic implant device 1 is configured in a folded and pleated form and configured for permanent implantation in the lumen of an artery or vein in a human patient. Alternatively, the expandable metallic implant 10 of an expandable metallic implant device 1 is folded and pleated (or wrapped or compressed) together with a balloon 110, in a manner wherein portions of the wall 30 of the expandable portion 15 of the expandable metallic implant 10 and the balloon 110 are squeezed, pressed, or wrapped together into a condensed space to facilitate passage through guide catheters or guide sheaths 200 or maneuvering into or through arteries, veins, aneurysms, chambers of the heart, biological conduits, biological spaces, and other blood-containing and fluid-containing spaces to a treatment or target site.

Metal layers of expandable metallic implants 10 and multilayered expandable metallic implants 10 may be formed by various methods. In some examples, a structural metal layer 34 or functional metal layer 35 can be formed by electroplating. In some examples, an expandable metallic implant 10 may be manufactured by a method wherein a metal such as gold is deposited by electroforming over a sacrificial conductive mandrel made from an electrically conductive material such as aluminum. The sacrificial conductive mandrel may then be removed from the interior of the expandable metallic implant 10 structure by processes such as drilling and acid etching. By creating conductive mandrels of various sizes and shapes and then applying a layer of metal by electroforming or otherwise using electroforming techniques, expandable metallic implants 10 can be made that match the size and shape of various arteries, veins, aneurysms, parent vessels of saccular aneurysms, paravalvular leak pathways, biological conduits, or other blood-containing, fluid-containing, or biological spaces. The expandable metallic implant 10 may further undergo an annealing process to improve the flexibility and ductility of the wall 29 of the core portion 14 of the expandable metallic implant 10, including before and after pleating and folding. 10. In some examples, a functional metal layer 35 can be formed by sputter deposition, wherein material is eroded from a target and is then deposited onto a substrate, such as a sacrificial conductive mandrel, an expandable metallic implant 10, the core portion 14 of an expandable metallic implant 10, or the expandable portion 15 of the expandable metallic implant 10, forming a thin layer on the substrate. In some examples, a functional metal layer 35 is formed by vapor deposition, wherein vapors from one or metals or metal alloys are condensed upon a substrate, such as a sacrificial conductive mandrel, an expandable metallic implant 10, the core portion 14 of an expandable metallic implant 10, or the expandable portion 15 of an expandable metallic implant 10.

In some examples, various metallic regions of an expandable metallic implant 10 or the core portion 14 or expandable portion 15 of an expandable metallic implant 10 may have different compositions or thickness. In some examples, the different composition or thickness is produced by masking portions or regions of the expandable metallic implant 10 or the core portion 14 or expandable portion 15 of an expandable metallic implant 10 prior to adding metal. In some examples, the additional metal may be applied to all or portions or regions of the expandable metallic implant 10 or the core portion 14 or expandable portion 15 of the expandable metallic implant 10 by electroplating or electroforming, vapor deposition, or sputter deposition. In some examples, regions of the exterior surface 41 or interior surface 42 of an expandable metallic implant 10 or the core portion 14 or expandable portion 15 of an expandable metallic implant 10 may be masked or covered prior to adding additional metal layers or adding additional material to an existing metal layer, including when additional metal layers or additional metal is added by electroplating or electroforming, vapor deposition, and sputter deposition. In some of these examples, the additional metal layer or additional metal may be applied to the portions of an expandable metallic implant 10 or the core portion 14 or expandable portion 15 of an expandable metallic implant 10 that have not been masked or covered prior to electroplating or electroforming, vapor deposition, or sputter deposition.

The thickness of a metal layer in any portion of an expandable metallic implant 10, or the core portion 14 or expandable portion 15 of an expandable metallic implant 10, can be modified by varying process time or conditions, or by adding additional processes or conditions. In some examples, a thicker structural metal layer 34 can be formed by increasing the time of electroforming. In some examples, a thicker functional metal layer 35 can be formed by increasing the time of electroplating, vapor deposition or sputter deposition.

In some examples, structural metal layers 34 or functional metal layers 35 of expandable implants may be formed in various patterns. For some examples, one or more masks or coverings may be applied to the exterior surface of a sacrificial conductive mandrel, an expandable metallic implant 10, or the core portion 14 or the expandable portion 15 of the expandable metallic implant 10. In some examples, after one or more masks or coverings are applied, a functional metal layer 35 is added to the unmasked or uncovered regions by sputter deposition by electroplating, vapor deposition, or sputter deposition and the one or more masks or coverings are removed. In some examples, various portions of an expandable metallic implant 10, or the core portion 14 or the expandable portion 15 of the expandable metallic implant 10 may have one or more structural metal layers 34. In some examples, various portions of an expandable metallic implant 10, or the core portion 14 or the expandable portion 15 of the expandable metallic implant 10 may have one or more structural metal layers 34 and one or more functional metal layers 35. Structural metal layers 34 are generally applied as continuous layers. Functional metal layers 35 may be applied as continuous layers or in discontinuous layers in a wide range of patterns.

Polymer layers 36 of multilayered expandable metallic implants 10 may be formed by various methods. In some examples, a polymer layer 36 can be formed by electroplating, vapor deposition, sputter deposition, or by applying a coating. In some examples, a polymer layer 36 may be applied to modify the mechanical, electrical, biocompatibility, or surface texture characteristics of an expandable metallic implant 10. The polymer coating may comprise Parylene, polyurethane, PTFE, silicone, or other biocompatible polymers. In some examples, the additional coating may include an exterior coating of a polyurethane-containing solution. The coating may be applied by dipping, spinning, spraying, or other deposition processes specialized for the specific polymer. The coating may be applied to the entire exterior of the expandable metallic implant 10 or to only selected regions by masking some regions. In various examples, all or a portion of the exterior surface 41 or interior 42 surface of expandable metallic implants 10 may be coated with a variety of materials having properties ranging from hydrophobic to hydrophilic, polyurethanes, metals, or combinations thereof.

In some examples, a polymer layer 36 can be formed by sputter deposition, wherein material is eroded from a target and is then deposited onto a substrate, such as an expandable metallic implant 10, the core portion 14 of an expandable metallic implant 10, or the expandable portion 15 of the expandable metallic implant 10, forming a thin layer on the substrate. In some examples, a polymer layer 36 is formed by vapor deposition, wherein vapors from one or polymers are condensed upon a substrate such as an expandable metallic implant 10, the core portion 14 of an expandable metallic implant 10, or the expandable portion 15 of an expandable metallic implant 10.

In some examples, various polymer regions or layers 36 of a multilayered expandable metallic implant 33, or the core portion 14 or expandable portion 15 of a multilayered expandable metallic implant 33 may have different compositions or thickness. In some examples, the different composition or thickness is produced by masking portions or regions of an expandable metallic implant 10, or the core portion 14 or expandable portion 15 of an expandable metallic implant 10, prior to adding polymer. In some examples, polymer may be applied to all or portions or regions of the expandable metallic implant 10, or the core portion 14 or expandable portion 15 of the expandable metallic implant 10, by electroplating, vapor deposition, sputter deposition, or by applying a coating. In some examples, regions of the exterior 41 surface or interior surface 42 of an expandable metallic implant 10, or the core portion 14 or expandable portion 15 of an expandable metallic implant 10, may be masked or covered prior to adding polymer, including when polymer is added by electroplating, vapor deposition, sputter deposition, or applying a coating. In some of these examples, the polymer may be applied to the portions of an expandable metallic implant 10, or the core portion 14 or expandable portion 15 of an expandable metallic implant 10, that have not been masked or covered prior to electroplating, vapor deposition, sputter deposition, or coating.

The thickness of a polymer layer 36 in any portion of an expandable metallic implant 10, or the core portion 14 or expandable portion 15 of an expandable metallic implant 10, can be modified by varying process time or conditions, or by adding additional processes or conditions. In some examples, a thicker structural metal layer 34 can be formed by increasing the time of electroforming. In some examples, a thicker functional metal layer 35 can be formed by increasing the time of electroplating, vapor deposition or sputter deposition. In some examples, a thicker polymer layer 36 can be formed by increasing the time of electroplating, vapor deposition or sputter deposition. In some examples, a thicker polymer layer 36 can be formed by applying more polymer material to the exterior 41 surface or interior surface 42 of the expandable metallic implant 10, or the core portion 14 or expandable portion 15 of an expandable metallic implant 10.

In some examples, a polymer layer 36 of a multilayered expandable metallic implant 33 may be formed in various patterns. For some examples, one or more masks or coverings may be applied to the exterior 41 surface or interior surface 42 of an expandable metallic implant 10, or the core portion 14 or the expandable portion 15 of the expandable metallic implant 10, and a polymer layer 36 is added to the unmasked or uncovered regions by electroplating, vapor deposition, sputter deposition, or by applying a coating, and the one or more masks or coverings are removed. In some examples, various portions of an expandable metallic implant 10, or the core portion 14 or the expandable portion 15 of the expandable metallic implant 10 may have one or more structural metal layers 34. In some examples, various portions of an expandable metallic implant 10, or the core portion 14 or the expandable portion 15 of the expandable metallic implant 10 may have one or more structural metal layers 34, one or more structural metal layers 34 and one or more functional metal layers 35, one or more structural metal layers 34 and one or more polymer layers 36, or one or more structural metal layers 34 and one or more functional metal layers 35 and one or more polymer layers 36. Polymer layers 36 may be applied as continuous layers or in discontinuous layers in a wide range of patterns.

In some examples, the neck transitional region 77 between the transition zone 19 and the neck 60 of an expandable metallic implant 10, if properly configured, can serve as a flexible “hinge” region that can flex or bend when the folded and pleated expandable metallic implant 13 is advanced inside the body, or in response to forces encountered when a folded and pleated expandable metallic implant device 2 with such a folded and pleated expandable metallic implant 13 is moving along a tortuous path. The wall of the neck transitional region 77 may comprise metal or metal and polymer. In some examples, the wall of the neck transitional region 77 has a thickness in the range of 3-20 microns. In some examples, the wall of the neck transitional region 77 comprises gold or a gold layer. In some examples, the wall of the neck transitional region 77 comprises a layer of gold with a thickness of 3, 4, 5, 6, 7, 8, 9, 10, 11, 12, 13, 14, 15, 16, 17, 18, 19, or 20 microns. In some examples, the wall of the neck transitional region 77 comprises a layer of gold with a thickness of <5 microns, <10 microns, <15 microns, or <20 microns. In some examples, the wall of the neck transitional region 77 comprises a layer of gold with a thickness less than the thickness of the layer of gold in the neck 60 of the expandable metallic implant, a thickness less than the thickness of the layer of gold in the transition zone 19 of the expandable metallic implant, or a thickness less than the thickness of the layer of gold in the neck 60 of the expandable metallic implant and the thickness of the layer of gold in the transition zone 19 of the expandable metallic implant.

In some examples, wherein the wall 29 of the core portion 14 of an expandable metallic implant 10 has one layer, that layer is a metal layer. The one layer of the wall 29 of the core portion 14 of an expandable metallic implant 10 may be formed by any suitable method, including by electroforming. For some single layer examples of an expandable metallic implant 10, the single layer comprises gold, platinum, alloys thereof, and combinations thereof. For some single layer examples of an expandable metallic implant 10, the single layer is a gold layer.

In some examples, the wall 29 of the core portion 14 of a multilayered expandable metallic implant 33 has two layers. The inner layer 40 and the outer layer 38 of the wall 29 of the core portion 14 of a multilayered expandable metallic implant 33 may be formed by any suitable method, including by electroforming or electroplating, sputter deposition, or vapor deposition. The inner layer 40 and the outer layer 38 of the wall 29 of the core portion 14 of a multilayered expandable metallic implant 33 may be formed by any suitable method, including by electroforming or electroplating, sputter deposition, or vapor deposition. For some two-layer examples of a multilayered expandable metallic implant 33, one or both of the inner layer 40 and outer layer 38 of the wall 29 of the multilayered expandable metallic implant 33 comprises metal, including wherein the metal is gold, platinum, silver, titanium, vanadium, aluminum, nickel, tantalum, zirconium, chromium, magnesium, niobium, scandium, cobalt, palladium, manganese, molybdenum, alloys thereof, and combinations thereof. For some two-layer examples of a multilayered expandable metallic implant 33, one or both of the inner layer 40 and outer layer 38 of the wall 29 of the multilayered expandable metallic implant 33 comprises polymer, including wherein the polymer is polyethylene terephthalate, nylon, polyether block amide or other aliphatic or semi-aromatic polyamides, polyurethane, polyvinyl chloride, polyethylene, silicone elastomer, polytetrafluoroethylene (PTFE), and combinations thereof.

In some examples, the wall 29 of the core portion 14 of a multilayered expandable metallic implant 33 has three layers. The inner layer 40 and outer layer 38 of the wall 29 of the core portion 14 of a multilayered expandable metallic implant 33 may be formed by any suitable method, including by electroforming or electroplating, sputter deposition, or vapor deposition. For some three-layer examples of a multilayered expandable metallic implant 33, one or more of the inner layer 40 and outer layer 38 of the wall 29 of the multilayered expandable metallic implant 33 comprises metal, including wherein the metal is gold, platinum, silver, titanium, vanadium, aluminum, nickel, tantalum, zirconium, chromium, magnesium, niobium, scandium, cobalt, palladium, manganese, molybdenum, alloys thereof, and combinations thereof. For some three-layer examples of a multilayered expandable metallic implant 33, one or more of the inner layer 40 and outer layer 38 of the wall 29 of the multilayered expandable metallic implant 33 comprises polymer, including wherein the polymer is polyethylene terephthalate, nylon, polyether block amide or other aliphatic or semi-aromatic polyamides, polyurethane, polyvinyl chloride, polyethylene, silicone elastomer, polytetrafluoroethylene (PTFE), and combinations thereof.

In some examples, the structural metal layer 34 of a multilayered expandable metallic implant 33 can be segmented (FIG. 41) and the segments connected together by an underlying polymer layer 36. This has the advantage of increasing the flexibility of an implant during insertion and positioning of a folded and pleated expandable metallic implant device 2 in a patient, especially when the folded and pleated expandable metallic implant 13 is long. In the example shown in FIG. 41A-C, the seal zone 18 comprises a first segment of structural metal layer 34 and the transition zone 19 and neck 60 comprise a second segment of structural metal layer 34 joined to an underlying and flexible polymer layer 36.

In some examples, the flaps 22 of an expanded expandable metallic implant 11 have holes to enable tissue ingrowth through the wall of the flaps 22 after placement of the expanded expandable metallic implant 11 in a target vessel segment (FIGS. 42A and B) to affix the expanded expandable metallic implant 11 to the wall 603 of the target vessel segment and reduce the risk of migration or displacement of the expanded expandable metallic implant 11.

In some examples, the wall 29 of the core portion 14 of a multilayered expandable metallic implant 33 comprises a glue or adhesive layer 37.

In some examples, the expandable metallic implant 10 can be manufactured to have one or more secondary folds and pleats oriented along the first axis of the expandable metallic implant 10 prior to undergoing the primary folding and pleating process shown in FIG. 48A-C. An expanded expandable metallic implant 11 with secondary folds and pleats is shown in FIGS. 43A, 43C, and 43E. This folded and pleated variable size expandable metallic implant 49 provides the advantage of enabling the diameter of the folded and pleated variable size expandable metallic implant 49 to be expanded to the nominal diameter by initial inflation of the balloon 110 of the balloon catheter and then to be expanded to a larger diameter by overinflation of the balloon 110 of the balloon catheter 100 or by removing the balloon catheter 100 and using a balloon catheter with a larger balloon to further expand the folded and pleated variable size expandable metallic implant 49. In practice, an operator would expand the folded and pleated variable size expandable metallic implant 49 to its nominal size with the internal fold 50 and external fold 51 configured as shown in FIGS. 43A and 43D. If it is found that there is insufficient anchoring against the vessel wall or insufficient vessel obstruction or occlusion, the folded and pleated variable size expandable metallic implant 49 can be expanded further by overinflation of the balloon 110 of the balloon catheter 100 or by removing the balloon catheter 100 and using a balloon catheter with a larger balloon to further expand the folded and pleated variable size expandable metallic implant 49. During the secondary expansion, the inner 50 and outer fold 51 are straightened out so that the folded and pleated variable size expandable metallic implant 49 assumes the conventional cylindrical, non-pleated configuration of the previous examples, such as that shown in FIG. 26. In some examples, a folded and pleated variable size expandable metallic implant 49 is fabricated by applying a structural metal layer 34 (including a gold structural metal layer 34) to a mandrel 383 with a shape similar to the mandrel show in in FIGS. 43B and 43D, removing the mandrel 383, and then executing a primary folding and pleating process on the folded and pleated variable size expandable metallic implant 49 using a process similar to the process shown in FIG. 48A-C for primary folding and pleating. In some examples, a folded and pleated variable size expandable metallic implant 49 is fabricated by applying a structural metal layer 34 (including a gold structural metal layer 34) to a mandrel 383 with a shape similar to the mandrel show in in FIGS. 43B and 43D, removing the mandrel 383, forming the folded and pleated variable size expandable metallic implant 49 into a generally circular shape by compressing the folds and pleats, and then executing a primary folding and pleating process on the folded and pleated variable size expandable metallic implant 49 using process similar to the process shown in FIG. 48A-C for primary folding and pleating. A cross-sectional image of an example of a folded and pleated variable size expandable metallic implant 49 is shown in FIG. 43C. A cross-sectional image of an example of a mandrel 383 used to form a folded and pleated variable size expandable metallic implant 49 is shown in FIG. 43D.

In some examples, the expandable metallic implant 10 can be manufactured to have a longitudinal cut or slit 53 oriented along the first axis of the expandable metallic implant 10 prior to undergoing the primary folding and pleating process shown in FIG. 48A-C. An expanded expandable metallic implant 11 with a longitudinal cut or slit is shown in FIGS. 44A and 44B. This slotted variable size expandable metallic implant 52 provides the advantage of enabling the diameter of the slotted variable size expandable metallic implant 52 to be expanded to the nominal diameter by initial inflation of the balloon 110 of the balloon catheter and then to be expanded to a larger diameter by overinflation of the balloon 110 of the balloon catheter 100 or by removing the balloon catheter 100 and using a balloon catheter with a larger balloon to further expand the slotted variable size expandable metallic implant 52. In practice, an operator would expand the slotted variable size expandable metallic implant 52 to its nominal size. If it is found that there is insufficient anchoring against the vessel wall or insufficient vessel obstruction or occlusion, the slotted variable size expandable metallic implant 52 can be expanded further by overinflation of the balloon 110 of the balloon catheter 100 or by removing the balloon catheter 100 and using a balloon catheter with a larger balloon to further expand the slotted variable size expandable metallic implant 52. During the secondary expansion, the slot of variable size expandable metallic implant 53 widens. In some examples, a structure comprising the expandable portion of an expandable metallic implant is slotted and then nested together with a slotted variable size expandable metallic implant 52 with the slots of the two components offset so that the wall of one component covers the slot of the other component and then the two nested components are folded and pleated together with the balloon 110 of a balloon catheter to produce an expandable metallic implant device 1 In some examples, the two components can be placed within each other with the slits opposite each other so as the sealing surface remains contiguous around its circumference. In some examples, a slotted variable size expandable metallic implant 52 is fabricated by cutting a slot (including using a laser to cut the slot) into an expanded expandable metallic implant 11, and then executing a primary folding and pleating process on the slotted variable size expandable metallic implant 52 using a process similar to the process shown in FIG. 48A-C for primary folding and pleating.

In some examples, the distal end of the neck 60 of the expandable metallic implant 10 is substantially flush to the valve tubular body 80 and/or to the valve metallic ring 84 (FIG. 45A). In some examples where the distal end of the neck 60 of the expandable metallic implant 10 is substantially flush to the valve 79 and/or to the valve metal ring 84 (FIG. 45A). the distal end can be smoothed or formed by adding glue, including by adding glue layers 83 between the valve, the ring, and the neck surfaces.

In some examples, the distal end of the neck 60 of the expandable metallic implant 10 extends distally beyond the valve tubular body 80 and/or the valve metallic ring 84 (FIGS. 45B and 43C). In some examples where the distal end of the neck 60 of the expandable metallic implant 10 extends distally beyond the valve tubular body 80 and/or the valve metallic ring 84, the distal end can be smoothed or formed by folding the distal end of the neck 60 of the expandable metallic implant 10 over the ends of the valve 79 and/or the valve metal ring 84, as shown in FIG. 45C. In some examples, this can be achieved by pushing the distal end of the neck 60 of the expandable metallic implant 10 against a concave surface 86 so that the metal will bend over the ends of the valve 79 and/or the valve metal ring 84 and assume the smooth convex surface created by the concave fixture 86.

### Manufacturing of Balloons

In some examples, a balloon 110 can be formed by blow molding, compression molding, extrusion molding, or injection molding, or any molding means known in the art. The thickness of a polymer layer of the wall 137 of a balloon 110 can be modified by changing the method of making the layer, including by changing the time of a particular formative process time or conditions, or by adding additional formative process time or conditions. For example, when making a single layer balloon 110, the thickness of the wall 137 of the balloon 110 can be changed by changing the thickness of the extrusion used to make the balloon 110.

In some examples, a balloon 110 may be fabricated with a proximal neck 123 with a proximal neck opening 124, a distal neck 129 with a distal neck opening 130, or a proximal neck 123 with a proximal neck opening 124 and a distal neck 129 with a distal neck opening 130. In some examples, the distal neck opening 130 can be closed, obstructed, or sealed. In some examples, molds may be used for fabrication of various balloons 110 wherein, the shape and dimensions of the molds are selected based upon the size and shape of various arteries, veins, aneurysms, parent vessels of saccular aneurysms, paravalvular leak pathways, biological conduits, or other blood-containing, fluid-containing, or biological spaces that are routinely treated in patients.

### Pleating and Folding of Balloons and Expandable Metallic Implant Devices

To facilitate advancement and retraction of an expandable metallic implant device 1 in vivo, the expandable portion 15 of the expandable metallic implant 10 can be folded, pleated, wrapped, and/or compressed into various condensed forms, shapes, and dimensions to facilitate passage through guide catheters or guide sheaths 200 or maneuvered into or through arteries, veins, aneurysms, chambers of the heart, biological conduits, biological spaces, and other blood-containing and fluid-containing spaces to a treatment site. In some examples, wall 30 of the expandable portion 15 of an expandable metallic implant 10 can be folded, pleated, wrapped, and/or compressed down to a diameter small enough to pass through guide catheters or guide sheaths 200 or maneuvered into or through guide catheters or guide sheaths 200 or maneuvered into or through arteries, veins, aneurysms, chambers of the heart, biological conduits, biological spaces, and other blood-containing and fluid-containing spaces. In some examples, the expandable metallic implant 10 is configured to expand from a folded and pleated configuration (also known as a “deliverable configuration”) to an expanded configuration at the target location during inflation of the balloon 110 of the balloon catheter 100.

In some examples, an inflated balloon 111 is mated with an expanded expandable metallic implant 11 of a similar size and shape, wherein the inflated balloon 111 is positioned within the central hollow region 16 of the expanded expandable metallic implant 11 and then the expandable metallic implant 11 and the inflated balloon 111 are subject to a first “folding” operation wherein folds are made in the expandable metallic implant 11 and then the expandable metallic implant 11, producing a distinctive star shape. The radially extending elements are called wings 6 and comprise two layers of the wall 137 of the balloon 100 and two layers of the wall 30 of the expandable portion 15 of the expandable metallic implant 15. The wings 6 of the folded expandable metallic implant device 7 are then subject to a second “pleating” operation wherein the wings 6 are folded over, wrapped and/or compressed around the central axis 44. For some examples wherein the inflated balloon 111 is longer than the expanded expandable metallic implant 11, in the proximal portion 117 of the balloon 110, the wings 25 comprise two layers of the wall 137 of the balloon 100 but do not comprise layers of the wall 30 of the expandable portion 15 of the expandable metallic implant 15.

In some examples, the expandable metallic implant 10 and the balloon 110 are formed into a plurality of wings 6, wherein each wing 6 of the plurality of wings comprises a ridge line extending proximal to distal and radially away from the first axis 45 and each wing 6 is separated from any immediately adjacent wing 6 by an interposed trough extending proximal to distal. In some examples, the expandable metallic implant 10 and the balloon 110 are formed into a plurality of wings 6 wherein the folded configuration comprises an alternating ridge-trough arrangement. In some examples, the expandable metallic implant 10 is in the folded and pleated configuration 13, wherein the wall 30 of the expandable portion 15 of the expandable metallic implant 10 assumes a folded and pleated configuration comprising a plurality of wings that folded over in a clockwise direction relative to the first axis 45 or a counterclockwise direction relative to the first axis 45 to form a folded and pleated region of the expandable metallic implant 10 and wherein, when the expandable metallic implant 10 is expanded, the plurality of wings are not folded. In some examples, each wing 6 of the plurality of wings 6 of the expandable metallic implant 10 comprises a ridge line extending proximal-distal along a plane parallel to the first axis 45 and radially along a plane parallel to the second axis 46. In some examples, each wing 6 comprises a ridge line extending proximal to distal along a plane parallel to the first axis 45 and each wing 6 of the plurality of wings 6 of expandable metallic implant 10 is separated from any immediately adjacent wing 6 by an interposed trough extending proximal to distal along a plane parallel to the first axis 45. In some examples, the folded configuration of a pleated expandable metallic implant 13 comprises an alternating ridge-trough arrangement. In some examples, each wing 6 of the plurality of wings 6 of a folded expandable metallic implant 13 is folded over an immediately adjacent wing 6 in a clockwise direction or a counterclockwise direction. In some examples, an individual wing 6 is formed by compressing a portion of the wall 30 of the expandable portion 15 of the expandable metallic implant 10 between two or more plates. These wings 6 are then folded around the central axis 44 of the expandable metallic implant 10 in one direction which causes the expandable metallic implant 10 to be formed into a generally cylindrical shape. In some examples, the expandable metallic implant 10 is configured to assume the expanded configuration by substantially eliminating the folded and pleated configuration or by substantially eliminating a plurality of the wings 6 that are present in the folded and pleated configuration.

In some examples, of an expandable metallic implant device 1, the folded and pleated expandable metallic implant 13 can be expanded by injection of water, saline, radiographic contrast, or combinations thereof, into the interior space 120 of the balloon 110 under pressure. In some examples, of an expandable metallic implant device 1, the folded and pleated expandable metallic implant 13 can be expanded by injection of water, saline, radiographic contrast, or combinations thereof, into the interior space 120 of the balloon 110 at a pressure of 1, 2, 3, 4, 5, 6, 7, 8, 9, 10, 11, 12, 13, 14, 15, 16, 17, 18, 19, or 20 atmospheres. In some examples, an expandable metallic implant catheter 1 is configured to enable the injection of water, saline, radiographic contrast, or combinations thereof from the hub 162 of the elongate body 160, through the lumen 161 of the elongate body 160, and into the interior space 120 of the balloon 110. In some examples, the interior space 120 of the balloon 110 can be pressurized during the passage of a fluid from the hub 162 of the elongate body 160, through the lumen 161 of the elongate body 160, and into the interior space 120 of the balloon 110. In some examples, an expandable metallic implant catheter 1 is configured to enable the injection of fluid from the hub 162 of the elongate body 160, through the lumen 161 of the elongate body 160, and into the interior space 120 of the balloon 110 at a rate higher than the leaking of fluid from the interior space 120 of the balloon 110 into the space adjacent to the balloon 110.

In some examples, of an expandable metallic implant device 1, expansion of the folded and pleated expandable metallic implant 13 results in a foreshortening of the expandable portion 15 of the folded and pleated expandable metallic implant 13 along a plane parallel to the first axis 45. In some examples, of an expandable metallic implant device 1, expansion of the folded and pleated expandable metallic implant 13 does not result in a foreshortening of the expandable portion 15 of the expandable metallic implant 10 along a plane parallel to the first axis 45. In some examples, of an expandable metallic implant device 1, the expandable portion 15 of the folded and pleated expandable metallic implant 13 is 0%, <1%, <2%, <3%, <5%, or <10% longer than the expanded expandable metallic implant 11 along a plane parallel to the first axis 45.

Various equipment and methods can be used to fold, pleat, wrap, or compress expandable metallic implants 10 and balloons 110 into various forms, shapes, and dimensions to enable the passage of expandable metallic implants 10 through the lumen 202 of guide catheters or guide sheaths 200, arteries, veins, aneurysms, chambers of the heart, biological conduits, biological spaces, and other blood-containing and fluid-containing spaces and expansion at a treatment site. In some examples, portions of the wall 30 of the expandable portion 15 of the expandable metallic implant 10 and portions of the wall 137 of the balloon 110 are squeezed or pressed together to form wings 6 prior to pleating. The number and length of wings 6 can be optimized for the various expandable metallic implant 10 diameters, lengths, shapes, and thicknesses of the wall 30 of the expandable portion 15 of the expandable metallic implant 10. By way of example and not limitation, an expanded expandable metallic implant 11 with a seal zone 18 with a 6 mm diameter and a balloon 110 with an intermediate region 118 with a 6 mm diameter can be folded and pleated together to an outer diameter small enough to pass through a 5 Fr or a 6 Fr guide catheter or guide sheath 200.

In some examples, an expanded expandable metallic implant 11 and an inflated balloon 111 can be folded and pleated together into a folded and pleated expandable metallic implant and balloon assembly 4 with a cylindrical shape. In some examples, the folded and pleated expandable metallic implant and balloon assembly 4 is compressed to a smaller diameter in a secondary operation. Polymers used to form medical grade balloons, such as PET, Pebax, and nylon are highly resistant to tearing during pleat and fold operations. Metals used to form medical grade balloons, such as gold, can tear during pleat and fold operations. In some examples, of the expandable metallic implant device 1, the folded and pleated expandable metallic implant and balloon assembly 4 can be folded, pleated, wrapped, and/or compressed to a small diameter while still maintaining the ability of the inflate and expand, as the tough, flexible polymer material can resist tearing or leaking.

In some examples, an expanded expandable metallic implant 11 and an inflated balloon 111 are folded and pleated together and then the folded and pleated balloon 115 is joined to an elongate body 160 of a balloon catheter 100. In some examples, a balloon 110 is joined to an elongate body 160 of a balloon catheter 100 and then the balloon 110 is inflated or expanded and an expanded expandable metallic implant 11 and the inflated balloon 111 are folded and pleated together.

In some examples, an expanded expandable metallic implant 11 and an inflated balloon 111 may be folded and pleated around a central folding and pleating mandrel acting as central fixation point. In some examples, a folding and pleating mandrel may pass through: the lumen 125 of the proximal neck 123 of the balloon 110; the interior space 120 of the balloon 110; the lumen 131 of the distal neck 129 of the balloon 110; the lumen 125 of the proximal neck 123 and the interior space 120 of the balloon 110; the lumen 131 of the distal neck 129 and the interior space 120 of the balloon 110; or the lumen 125 of the proximal neck 123, the interior space 120, and the lumen 131 of the distal neck 129 of the balloon 110. In other examples, the expandable metallic implant 10 is folded and pleated without a folding and pleating mandrel.

The pleated expandable metallic implant 13 and balloon 110 may have a plurality of wings 6. In some examples, the pleated expandable metallic implant 13 and balloon 110 may have three wings 6, four wings 6, five wings 6, six wings 6, seven wings 6, eight wings 6, nine wings 6, 10 wings 6, 11 wings 6, or 12 wings 6. One of skill in the art will appreciate that the pleated expandable metallic implant 13 and balloon 110 may have as many wings as necessary to pleat, wrap, or compress the folded expandable metallic implant 13 and balloon 110 into a size that allows the folded and pleated expandable metallic implant and balloon assembly 4 to be advanced through a guide catheter or guide sheath 200, arteries, veins, aneurysms, chambers of the heart, biological conduits, biological spaces, and other blood-containing and fluid-containing spaces.

The present disclosure also describes methods of manufacturing expandable implant devices. In some examples, elongation of the expanded expandable metallic implant 11 and inflated balloon 111 is allowed during pleating, folding, or compression to reduce the outer diameter of the folded and pleated expandable metallic implant and balloon assembly 4 or reduce the profile of the folded and pleated expandable metallic implant and balloon assembly 4. In some examples, elongation of the expanded expandable metallic implant 11 and inflated balloon 111 is limited during pleating and folding to reduce the overall length of the rigid portions of the folded and pleated expandable metallic implant and balloon assembly 4 to improve deliverability. In some examples, elongation of the expanded expandable metallic implant 11 and inflated balloon 111 during pleating and folding is limited by adding elements to the folding and pleating mandrel before or after loading the expanded expandable metallic implant 11 and the inflated balloon 111 onto the folding and pleating mandrel.

### Kits

The present disclosure also relates to kits. In some examples, a kit comprising an expandable metallic implant device 1 and a guide catheter or guide sheath 200 is provided.

In some examples, a kit comprising an expandable metallic implant device 1 and a guide catheter or guide sheath 200 is provided, wherein: the folded and pleated expandable metallic implant and balloon assembly 4 can advance through the lumen 202 of the guide catheter or guide sheath 200; and the elongate body 160 of the balloon catheter 100 is longer than the elongate body 201 of the guide catheter or guide sheath 200.

In some examples, a kit comprising an expandable metallic implant device 1, a guide catheter or guide sheath 200, and a coiling or delivery catheter 260 is provided. In some examples, a kit comprising an expandable metallic implant device 1, a guide catheter or guide sheath 200, and a coiling or delivery catheter 260 is provided wherein: the folded and pleated expandable metallic implant and balloon assembly 4 can advance through the lumen 202 of the guide catheter or guide sheath 200; the elongate body 160 of the balloon catheter 100 is longer than the elongate body 201 of the guide catheter or guide sheath 200; the elongate body 261 of the coiling or delivery catheter 260 is longer than the elongate body 160 of the balloon catheter 100; and the elongate body 160 of the balloon catheter 100 and the elongate body 261 of the coiling or delivery catheter 260 can simultaneously advance through the lumen 202 of the guide catheter or guide sheath 200.

In some examples, a kit comprising a guide catheter or guide sheath 200 and an intermediate catheter 220 is provided. In some examples, a kit comprising a guide catheter or guide sheath 200 and a selective catheter 240 is provided. In some examples, a kit comprising a guide catheter or guide sheath 200, an intermediate catheter 220, and a selective catheter 240 is provided.

### Methods of Using an Expandable Metallic Implant Device to Obstruct or Reduce the Rate of Flow

Methods for obstructing or reducing the rate of flow in arteries, veins, aneurysms, parent vessels of saccular aneurysms, paravalvular leak pathways, biological conduits, or other blood-containing, fluid-containing, or biological spaces of human patients with expandable implant devices are described. In one example, a guide catheter or guide sheath 200 is inserted into an artery of a human patient and advanced over a guidewire 280 until the distal end of the guide catheter or guide sheath 200 is located in a segment of artery indicated for an obstruction or reduction in the rate of blood flow. In some examples, the guide catheter or guide sheath 200 is advanced without a guidewire 280. In some examples, a guide sheath 200 is advanced over a selective catheter 240 that is advanced over a guidewire 280. In some examples, a guide sheath 200 is advanced over an intermediate catheter 220, which is advanced over a selective catheter 240 that is advanced over a guidewire 280. In some examples, the target segment is a vein, aneurysm, parent vessel of a saccular aneurysm, paravalvular leak pathway, biological conduit, or other blood-containing, fluid-containing, or biological space. In some examples, contrast material is injected through the guide catheter or guide sheath 200 and into the segment of artery indicated for obstruction or reduction in the rate of blood flow during angiography, including injecting through the side arm 408 of a Tuohy Borst adaptor 206 and a measurement is made of the diameter and length of the target segment using one or more angiography images. In some examples, the guide catheter or guide sheath 200 has one or more marker bands 209 and the marker bands 209 are used to facilitate the making of the measurements. A folded and pleated expandable metallic implant device 2 with an appropriately sized expandable metallic implant 10 is selected. In some examples, the expandable metallic implant 10 of the expandable metallic implant device 1 has an expanded diameter that is 10% 50% larger than the diameter of the lumen of the segment of artery indicated for obstruction or reduction in the rate of blood flow. Any intermediate catheter 220, selective catheter 240, or guidewire 280 remaining in the lumen 202 of the guide catheter or guide sheath 200 is removed and the distal portion of the folded and pleated expandable metallic implant device 2 is inserted through the hub 203 of the guide catheter or guide sheath 200 and into the lumen 202 of the guide catheter or guide sheath 200. In some examples, a Tuohy Borst adaptor 406 is joined to the hub 203 of the guide catheter or guide sheath 200 and the folded and pleated expandable metallic implant device 2 is first advanced through the Tuohy Borst adaptor 406 before advancement into the lumen 202 of the guide catheter or guide sheath 200. In some examples, the expandable metallic implant comprises a guidewire segment 69 joined or bonded to the neck 60 or neck assembly 65 of the folded and pleated expandable metallic implant 13 and the guidewire segment 69 is used to guide the folded and pleated expandable metallic implant 13 into the Tuohy Borst adaptor 406 or guide catheter or guide sheath 200. The folded and pleated expandable metallic implant device 2 is advanced through the lumen 202 of the guide catheter or guide sheath 200 until the folded and pleated expandable metallic implant 13 exits the distal end of the guide catheter or guide sheath 200 and is positioned in the segment of artery indicated for obstruction or reduction in the rate of blood flow. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 to evaluate the size and position of the folded and pleated expandable metallic implant 13, and the size and amount of blood flow in the segment of artery indicated for obstruction or reduction in the rate of blood flow. After positioning of the folded and pleated expandable metallic implant 13 in the segment of artery indicated for obstruction or reduction in the rate of blood flow, a fluid is injected: into the hub 162; through the lumen 161 of the elongate body 160; and into the interior space 120 of the balloon 110, causing the folded and pleated balloon 115 and the folded and pleated expandable metallic implant 13 to expand. In some examples, the injected fluid is water, saline, contrast material, and mixtures thereof. In some examples, the balloon 110 is compliant or semi-compliant and the expandable metallic implant 10 comprises one or more flaps 22, and the balloon 110 is inflated at a pressure that raises at least one flap 24 from the surface of the expanded expandable metallic implant 11 during balloon 110 inflation. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after expansion of the expandable metallic implant 10 to evaluate the size and position of the expanded expandable metallic implant 11, and the amount of blood flow in the segment of artery indicated for obstruction or reduction in the rate of blood flow. After expansion of the expandable metallic implant 10, fluid is removed from the interior space 120 of the inflated balloon 111, causing the balloon 110 to deflate or collapse while the expandable metallic implant remains in an expanded configuration. After deflation or collapse of the balloon 110, the deflated or collapsed balloon 113 is removed from the central hollow region 16 of the expanded expandable metallic implant 11 by retracting elongate body 160, and the balloon catheter 100 and the guide catheter or guide sheath 200 are removed from the patient. In some examples, after removing fluid from the interior space 120 of the balloon 110, the distal tip of the guide catheter or guide sheath 200 is advanced over the balloon 110 prior to removing the balloon 110 from the central hollow region 16 of the expanded expandable metallic implant 11 and the balloon 110 is removed from the central hollow region 16 of the expanded expandable metallic implant 11 by retracting the longitudinal body 201 of the guide catheter or guide sheath 200. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after removal of the balloon catheter 100 to evaluate the size and position of the expanded expandable metallic implant 11 and the amount of blood flow in the segment of artery indicated for obstruction or reduction in the rate of blood flow.

### Methods of Using an Expandable Metallic Implant Device and One or More Coil Expandable implants or Vascular Plug Expandable implants to Obstruct or Reduce the Rate of Flow

Further implementations of this disclosure can include coil expandable implants 301 or vascular plug expandable implants.

Methods for obstructing or reducing the rate of flow in arteries, veins, aneurysms, parent vessels of saccular aneurysms, paravalvular leak pathways, biological conduits, or other blood-containing, fluid-containing, or biological spaces of human patients with expandable implant devices and coil expandable implant devices 300 or vascular plug expandable metallic implant devices 320 are described. In one example, a guide catheter or guide sheath 200 is inserted into an artery of a human patient and advanced over a guidewire 280 until the distal end of the guide catheter or guide sheath 200 is located in a segment of artery indicated for an obstruction or reduction in the rate of blood flow. In some examples, the guide catheter or guide sheath 200 is advanced without a guidewire 280. In some examples, a guide sheath 200 is advanced over a selective catheter 240 that is advanced over a guidewire 280. In some examples, a guide sheath 200 is advanced over an intermediate catheter 220, which is advanced over a selective catheter 240 that is advanced over a guidewire 280. In some examples, the target segment is a vein, aneurysm, parent vessel of a saccular aneurysm, paravalvular leak pathway, biological conduit, or other blood-containing, fluid-containing, or biological space. In some examples, contrast material is injected through the guide catheter or guide sheath 200 and into the segment of artery indicated for obstruction or reduction in the rate of blood flow during angiography, including injecting through the side arm 408 of a Tuohy Borst adaptor 206 and a measurement is made of the diameter and length of the target segment using one or more angiography images. In some examples, the guide catheter or guide sheath 200 has one or more marker bands 209 and the marker bands 209 are used to facilitate the making of the measurements. A folded and pleated expandable metallic implant device 2 with an appropriately sized expandable metallic implant 10 is selected. In some examples, the expandable metallic implant 10 of the expandable metallic implant device 1 has an expanded diameter that is 10% 50% larger than the diameter of the lumen of the segment of artery indicated for obstruction or reduction in the rate of blood flow. Any intermediate catheter 220, selective catheter 240, or guidewire 280 remaining in the lumen 202 of the guide catheter or guide sheath 200 is removed and the distal portion of the folded and pleated expandable metallic implant device 2 is inserted through the hub 203 of the guide catheter or guide sheath 200 and into the lumen 202 of the guide catheter or guide sheath 200. In some examples, a Tuohy Borst adaptor 406 is joined to the hub 203 of the guide catheter or guide sheath 200 and the folded and pleated expandable metallic implant device 2 is first advanced through the Tuohy Borst adaptor 406 before advancement into the lumen 202 of the guide catheter or guide sheath 200. In some examples, the expandable metallic implant comprises a guidewire segment 69 joined or bonded to the neck 60 or neck assembly 65 of the folded and pleated expandable metallic implant 13 and the guidewire segment 69 is used to guide the folded and pleated expandable metallic implant 13 into the Tuohy Borst adaptor 406 or guide catheter or guide sheath 200. The folded and pleated expandable metallic implant device 2 is advanced through the lumen 202 of the guide catheter or guide sheath 200 until the folded and pleated expandable metallic implant 13 exits the distal end of the guide catheter or guide sheath 200 and is positioned in the segment of artery indicated for obstruction or reduction in the rate of blood flow. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 to evaluate the size and position of the folded and pleated expandable metallic implant 13, and the size and amount of blood flow in the segment of artery indicated for obstruction or reduction in the rate of blood flow. After positioning of the folded and pleated expandable metallic implant device 2, a guidewire 280 is placed into the lumen 262 of a coiling or delivery catheter 260 and the coiling or delivery catheter 260 and guidewire 280 are inserted into the hub 203 of the guide catheter or guide sheath 200 and advanced alongside the elongate body 160 of the balloon catheter 100 until the distal end of the coiling or delivery catheter 260 exits the distal end of the guide catheter or guide sheath 200 is positioned in artery indicated for obstruction or reduction in the rate of blood flow at a position distal to the folded and pleated expandable metallic implant 12 of the folded and pleated expandable metallic implant device 2. After positioning the coiling or delivery catheter 260, a fluid is injected: into the hub 162 of the folded and pleated expandable metallic implant device 2; through the lumen 161 of the elongate body 160; and into the interior space 120 of the balloon 110, causing the folded and pleated balloon 115 and the folded and pleated expandable metallic implant 13 to partially expand. In some examples, the injected fluid is water, saline, contrast material, and mixtures thereof. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after expansion of the expandable metallic implant 10 to evaluate the size and position of the expanded expandable metallic implant 11, and the amount of blood flow in the segment of artery indicated for obstruction or reduction in the rate of blood flow. After partial expansion of the expandable metallic implant 10, the guidewire 280 is removed from the lumen 262 of the coiling or delivery catheter 260 and one or more coil expandable implants 301 is pushed through the lumen 262 of the coiling or delivery catheter 260 and placed in the segment of artery or vein indicated for obstruction or reduction in the rate of blood flow at a position distal to the partially expanded expandable metallic implant 11. In some examples, one or more coil expandable implants 301 is placed adjacent to, or proximal to, the partially expanded expandable metallic implant 11. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after, one or more coil expandable implants 301 to evaluate the size and position of the expanded expandable metallic implant 11 and the one or more coil expandable implants 301, and the amount of blood flow in the segment of artery indicated for obstruction or reduction in the rate of blood flow. After placement of the one or more coil expandable implants 301, additional fluid is injected: into the hub 162; through the lumen 161 of the elongate body 160; and into the interior space 120 of the balloon 110, causing the folded and pleated balloon 115 and the folded and pleated expandable metallic implant 13 to fully expand. In some examples, the balloon 110 is compliant or semi-compliant and the expandable metallic implant 10 comprises one or more flaps 22, and the balloon 110 is inflated at a pressure that raises at least one flap 22 from the surface of the expanded expandable metallic implant 11 during balloon 110 inflation. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after expansion of the expandable metallic implant 10 to evaluate the size and position of the expanded expandable metallic implant 11 and the one or more coil expandable implants 301, and the amount of blood flow in the segment of artery indicated for obstruction or reduction in the rate of blood flow. After full expansion of the expandable metallic implant 10, fluid is removed from the interior space 120 of the inflated balloon 111, causing the balloon 110 to deflate or collapse while the expandable metallic implant remains in an expanded configuration. After deflation or collapse of the balloon 110, the deflated or collapsed balloon 113 is removed from the central hollow region 16 of the expanded expandable metallic implant 11 by retracting elongate body 160, and the balloon catheter 100 and the guide catheter or guide sheath 200 are removed from the patient. In some examples, after removing fluid from the interior space 120 of the balloon 110, the distal tip of the guide catheter or guide sheath 200 is advanced over the balloon 110 prior to removing the balloon 110 from the central hollow region 16 of the expanded expandable metallic implant 11, including wherein the balloon 110 is removed from the central hollow region 16 of the expanded expandable metallic implant 11 by retracting the longitudinal body 201 of the guide catheter or guide sheath 200. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after removal of the balloon catheter 100 to evaluate the size and position of the folded and pleated expandable metallic implant 13 and the one or more coil expandable implants 301, and the amount of blood flow in the segment of artery indicated for obstruction or reduction in the rate of blood flow.

### Methods of Using an Over-the-guidewire Expandable Metallic Implant Device to Obstruct or Reduce the Rate of Flow

Methods for obstructing or reducing the rate of flow in arteries, veins, aneurysms, parent vessels of saccular aneurysms, paravalvular leak pathways, biological conduits, or other blood-containing, fluid-containing, or biological spaces of human patients with over-the-guidewire expandable implant devices are described. In one example, a guide catheter or guide sheath 200 is inserted into an artery of a human patient and advanced over a guidewire 280 until the distal end of the guide catheter or guide sheath 200 is located in a segment of artery proximal to the segment of artery indicated for an obstruction or reduction in the rate of blood flow. In some examples, a guide sheath 200 is advanced over a selective catheter 240 that is advanced over a guidewire 280. In some examples, a guide sheath 200 is advanced over an intermediate catheter 220, which is advanced over a selective catheter 240 that is advanced over a guidewire 280. In some examples, the target segment is a vein, aneurysm, parent vessel of a saccular aneurysm, paravalvular leak pathway, biological conduit, or other blood-containing, fluid-containing, or biological space. In some examples, contrast material is injected through the guide catheter or guide sheath 200 and into the segment of artery indicated for obstruction or reduction in the rate of blood flow during angiography, including injecting through the side arm 408 of a Tuohy Borst adaptor 206 and a measurement is made of the diameter and length of the target segment using one or more angiography images. In some examples, the guide catheter or guide sheath 200 has one or more marker bands 209 and the marker bands 209 are used to facilitate the making of the measurements. A folded and pleated expandable over-the-guidewire metallic implant device 2 with an appropriately sized expandable metallic implant 10 is selected. In some examples, the expandable metallic implant 10 of the expandable metallic implant device 1 has an expanded diameter that is 10%-50% larger than the diameter of the lumen of the segment of artery indicated for obstruction or reduction in the rate of blood flow. Any intermediate catheter 220 or selective catheter 240 remaining in the lumen 202 of the guide catheter or guide sheath 200 is removed and the distal portion of the folded and pleated over-the-guidewire expandable metallic implant device 2 is inserted over a guidewire 280 and through the hub 203 of the guide catheter or guide sheath 200 and into the lumen 202 of the guide catheter or guide sheath 200. In some examples, a Tuohy Borst adaptor 406 is joined to the hub 203 of the guide catheter or guide sheath 200 and the folded and pleated over-the-guidewire expandable metallic implant device 2 is first advanced through the Tuohy Borst adaptor 406 before advancement into the lumen 202 of the guide catheter or guide sheath 200. The folded and pleated over-the-guidewire expandable metallic implant device 2 is advanced over the guidewire 280 through the lumen 202 of the guide catheter or guide sheath 200 until the folded and pleated over-the-guidewire expandable metallic implant 13 exits the distal end of the guide catheter or guide sheath 200 and is positioned in the segment of artery indicated for obstruction or reduction in the rate of blood flow. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 to evaluate the size and position of the folded and pleated over-the-guidewire expandable metallic implant 13, and the size and amount of blood flow in the segment of artery indicated for obstruction or reduction in the rate of blood flow. After positioning of the folded and pleated expandable metallic implant 13 in the segment of artery indicated for obstruction or reduction in the rate of blood flow, a fluid is injected: into the inflation port 177 of the hub of the folded and pleated over-the-guidewire expandable metallic implant 13; through the inflation lumen 161 of the of the folded and pleated over-the-guidewire expandable metallic implant 13; and into the interior space 120 of the balloon 110, causing the folded and pleated balloon 115 and the folded and pleated expandable metallic implant 13 to expand. In some examples, the injected fluid is water, saline, contrast material, and mixtures thereof. In some examples, the balloon 110 is compliant or semi-compliant and the expandable metallic implant 10 comprises one or more flaps 22, and the balloon 110 is inflated at a pressure that raises at least one flap 24 from the surface of the expanded expandable metallic implant 11 during balloon 110 inflation. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after expansion of the expandable metallic implant 10 to evaluate the size and position of the expanded expandable metallic implant 11, and the amount of blood flow in the segment of artery indicated for obstruction or reduction in the rate of blood flow. After expansion of the folded and pleated expandable metallic implant 13, fluid is removed from the interior space 120 of the inflated balloon 111, causing the balloon 110 to deflate or collapse while the expanded expandable metallic implant 111 remains in an expanded configuration. After deflation or collapse of the balloon 110, the deflated or collapsed balloon 113 is removed from the central hollow region 16 of the expanded expandable metallic implant 11; and the distal end of the inner shaft of the elongate body of the over-the-guidewire balloon catheter is removed from the neck lumen of the expandable metallic implant 62 resulting in closure or partial closure of the valve 79 in the neck lumen 62 of the expanded expandable metallic implant 11; and then the guide catheter or guide sheath 200 are removed from the patient. In some examples, after removing fluid from the interior space 120 of the balloon 110, the distal tip of the guide catheter or guide sheath 200 is advanced over the balloon 110 prior to removing the balloon 110 from the central hollow region 16 of the expanded expandable metallic implant 11 and the balloon 110 is removed from the central hollow region 16 of the expanded expandable metallic implant 11 by retracting the longitudinal body 201 of the guide catheter or guide sheath 200. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after removal of the balloon catheter 100 and guidewire 280 to evaluate the size and position of the expanded expandable metallic implant 11 and the amount of blood flow in the segment of artery indicated for obstruction or reduction in the rate of blood flow.

### Methods of Using an Over-the-Guidewire Expandable Metallic Implant Device and One or More Coil Expandable Implants or Vascular Plug Expandable Implants to Obstruct or Reduce the Rate of Flow-Distal Coil Expandable Implant or Vascular Plug Expandable Implant Placement

Methods for obstructing or reducing the rate of flow in arteries, veins, aneurysms, parent vessels of saccular aneurysms, paravalvular leak pathways, biological conduits, or other blood-containing, fluid-containing, or biological spaces of human patients with over-the-guidewire expandable implant devices and coil expandable implant devices 300 or vascular plug expandable metallic implant devices 320 are described are described. In one example, a guide catheter or guide sheath 200 is inserted into an artery of a human patient and advanced over a guidewire 280 until the distal end of the guide catheter or guide sheath 200 is located in a segment of artery proximal to the segment of artery indicated for an obstruction or reduction in the rate of blood flow. In some examples, a guide sheath 200 is advanced over a selective catheter 240 that is advanced over a guidewire 280. In some examples, a guide sheath 200 is advanced over an intermediate catheter 220, which is advanced over a selective catheter 240 that is advanced over a guidewire 280. In some examples, the target segment is a vein, aneurysm, parent vessel of a saccular aneurysm, paravalvular leak pathway, biological conduit, or other blood-containing, fluid-containing, or biological space. In some examples, contrast material is injected through the guide catheter or guide sheath 200 and into the segment of artery indicated for obstruction or reduction in the rate of blood flow during angiography, including injecting through the side arm 408 of a Tuohy Borst adaptor 206 and a measurement is made of the diameter and length of the target segment using one or more angiography images. In some examples, the guide catheter or guide sheath 200 has one or more marker bands 209 and the marker bands 209 are used to facilitate the making of the measurements. A folded and pleated expandable over-the-guidewire metallic implant device 2 with an appropriately sized expandable metallic implant 10 is selected. In some examples, the expandable metallic implant 10 of the expandable metallic implant device 1 has an expanded diameter that is 10%-50% larger than the diameter of the lumen of the segment of artery indicated for obstruction or reduction in the rate of blood flow. Any intermediate catheter 220 or selective catheter 240 remaining in the lumen 202 of the guide catheter or guide sheath 200 is removed and the distal portion of the folded and pleated over-the-guidewire expandable metallic implant device 2 is inserted over a guidewire 280 and through the hub 203 of the guide catheter or guide sheath 200 and into the lumen 202 of the guide catheter or guide sheath 200. In some examples, a Tuohy Borst adaptor 406 is joined to the hub 203 of the guide catheter or guide sheath 200 and the folded and pleated over-the-guidewire expandable metallic implant device 2 is first advanced through the Tuohy Borst adaptor 406 before advancement into the lumen 202 of the guide catheter or guide sheath 200. The folded and pleated over-the-guidewire expandable metallic implant device 2 is advanced over the guidewire 280 through the lumen 202 of the guide catheter or guide sheath 200 until the folded and pleated over-the-guidewire expandable metallic implant 13 exits the distal end of the guide catheter or guide sheath 200 and is positioned in the segment of artery indicated for obstruction or reduction in the rate of blood flow. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 to evaluate the size and position of the folded and pleated over-the-guidewire expandable metallic implant 13, and the size and amount of blood flow in the segment of artery indicated for obstruction or reduction in the rate of blood flow. After positioning of the folded and pleated expandable metallic implant 13 in the segment of artery indicated for obstruction or reduction in the rate of blood flow, a fluid is injected: into the inflation port 177 of the hub of the folded and pleated over-the-guidewire expandable metallic implant 13; through the inflation lumen 161 of the of the folded and pleated over-the-guidewire expandable metallic implant 13; and into the interior space 120 of the balloon 110, causing the folded and pleated balloon 115 and the folded and pleated expandable metallic implant 13 to expand. In some examples, the injected fluid is water, saline, contrast material, and mixtures thereof. In some examples, the balloon 110 is compliant or semi-compliant and the expandable metallic implant 10 comprises one or more flaps 22, and the balloon 110 is inflated at a pressure that raises at least one flap 24 from the surface of the expanded expandable metallic implant 11 during balloon 110 inflation. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after expansion of the expandable metallic implant 10 to evaluate the size and position of the expanded expandable metallic implant 11, and the amount of blood flow in the segment of artery indicated for obstruction or reduction in the rate of blood flow.

After expansion of the folded and pleated metallic implant 13, the guidewire 280 is removed from the lumen 174 of the inner shaft 172 of the over-the-guidewire balloon catheter 100 and one or more coil expandable implants 301 is pushed through the hub 176 of the over-the-guidewire balloon catheter 100 and through the lumen 174 of the inner shaft 172 of the over-the-guidewire balloon catheter and placed in the segment of artery or vein distal to the expanded expandable metallic implant 11. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after placement of one or more coil expandable implants 301 to evaluate the size and position of the expanded expandable metallic implant 11 and the one or more coil expandable implants 301, and the amount of blood flow in the segment of artery indicated for obstruction or reduction in the rate of blood flow. After placement of the one or more coil expandable implants 301, fluid is removed from the interior space 120 of the inflated balloon 111, causing the balloon 110 to deflate or collapse while the expanded expandable metallic implant 111 remains in an expanded configuration. After deflation or collapse of the balloon 110, the deflated or collapsed balloon 113 is removed from the central hollow region 16 of the expanded expandable metallic implant 11; and the distal end of the inner shaft of the elongate body of the over-the-guidewire balloon catheter is removed from the neck lumen of the expandable metallic implant 62 resulting in closure or partial closure of the valve 79 in the neck lumen 62 of the expanded expandable metallic implant 11; and then the guide catheter or guide sheath 200 are removed from the patient. In some examples, after removing fluid from the interior space 120 of the balloon 110, the distal tip of the guide catheter or guide sheath 200 is advanced over the balloon 110 prior to removing the balloon 110 from the central hollow region 16 of the expanded expandable metallic implant 11 and the balloon 110 is removed from the central hollow region 16 of the expanded expandable metallic implant 11 by retracting the longitudinal body 201 of the guide catheter or guide sheath 200. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after removal of the balloon catheter 100 and guidewire 280 to evaluate the size and position of the expanded expandable metallic implant 11, the one or more coil expandable implants 301, and the amount of blood flow in the segment of artery indicated for obstruction or reduction in the rate of blood flow.

### Methods of Using an Over-the-Guidewire Expandable Metallic Implant Device and One or More Coil Expandable Implants or Vascular Plug Expandable Implants to Obstruct or Reduce the Rate of Flow—Proximal Coil Expandable Implant or Vascular Plug Expandable Implant Placement

Methods for obstructing or reducing the rate of flow in arteries, veins, aneurysms, parent vessels of saccular aneurysms, paravalvular leak pathways, biological conduits, or other blood-containing, fluid-containing, or biological spaces of human patients with over-the-guidewire expandable implant devices and coil expandable implant devices 300 or vascular plug expandable metallic implant devices 320 are described are described. In one example, a guide catheter or guide sheath 200 is inserted into an artery of a human patient and advanced over a guidewire 280 until the distal end of the guide catheter or guide sheath 200 is located in a segment of artery proximal to the segment of artery indicated for an obstruction or reduction in the rate of blood flow. In some examples, a guide sheath 200 is advanced over a selective catheter 240 that is advanced over a guidewire 280. In some examples, a guide sheath 200 is advanced over an intermediate catheter 220, which is advanced over a selective catheter 240 that is advanced over a guidewire 280. In some examples, the target segment is a vein, aneurysm, parent vessel of a saccular aneurysm, paravalvular leak pathway, biological conduit, or other blood-containing, fluid-containing, or biological space. In some examples, contrast material is injected through the guide catheter or guide sheath 200 and into the segment of artery indicated for obstruction or reduction in the rate of blood flow during angiography, including injecting through the side arm 408 of a Tuohy Borst adaptor 206 and a measurement is made of the diameter and length of the target segment using one or more angiography images. In some examples, the guide catheter or guide sheath 200 has one or more marker bands 209 and the marker bands 209 are used to facilitate the making of the measurements. A folded and pleated expandable over-the-guidewire metallic implant device 2 with an appropriately sized expandable metallic implant 10 is selected. In some examples, the expandable metallic implant 10 of the expandable metallic implant device 1 has an expanded diameter that is 10%-50% larger than the diameter of the lumen of the segment of artery indicated for obstruction or reduction in the rate of blood flow. Any intermediate catheter 220 or selective catheter 240 remaining in the lumen 202 of the guide catheter or guide sheath 200 is removed and the distal portion of the folded and pleated over-the-guidewire expandable metallic implant device 2 is inserted over a guidewire 280 and through the hub 203 of the guide catheter or guide sheath 200 and into the lumen 202 of the guide catheter or guide sheath 200. In some examples, a Tuohy Borst adaptor 406 is joined to the hub 203 of the guide catheter or guide sheath 200 and the folded and pleated over-the-guidewire expandable metallic implant device 2 is first advanced through the Tuohy Borst adaptor 406 before advancement into the lumen 202 of the guide catheter or guide sheath 200.

The folded and pleated over-the-guidewire expandable metallic implant device 2 is advanced over the guidewire 280 through the lumen 202 of the guide catheter or guide sheath 200 until the folded and pleated over-the-guidewire expandable metallic implant 13 exits the distal end of the guide catheter or guide sheath 200 and is positioned in the segment of artery indicated for obstruction or reduction in the rate of blood flow. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 to evaluate the size and position of the folded and pleated over-the-guidewire expandable metallic implant 13, and the size and amount of blood flow in the segment of artery indicated for obstruction or reduction in the rate of blood flow. After positioning of the folded and pleated expandable metallic implant 13 in the segment of artery indicated for obstruction or reduction in the rate of blood flow, a fluid is injected: into the inflation port 177 of the hub of the folded and pleated over-the-guidewire expandable metallic implant 13; through the inflation lumen 161 of the of the folded and pleated over-the-guidewire expandable metallic implant 13; and into the interior space 120 of the balloon 110, causing the folded and pleated balloon 115 and the folded and pleated expandable metallic implant 13 to expand. In some examples, the injected fluid is water, saline, contrast material, and mixtures thereof. In some examples, the balloon 110 is compliant or semi-compliant and the expandable metallic implant 10 comprises one or more flaps 22, and the balloon 110 is inflated at a pressure that raises at least one flap 24 from the surface of the expanded expandable metallic implant 11 during balloon 110 inflation. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after expansion of the expandable metallic implant 10 to evaluate the size and position of the expanded expandable metallic implant 11, and the amount of blood flow in the segment of artery indicated for obstruction or reduction in the rate of blood flow.

After expansion of the folded and pleated expandable metallic implant 13, fluid is removed from the interior space 120 of the inflated balloon 111, causing the balloon 110 to deflate or collapse while the expanded expandable metallic implant 111 remains in an expanded configuration. After deflation or collapse of the balloon 110, the deflated or collapsed balloon 113 is removed from the central hollow region 16 of the expanded expandable metallic implant 11; and the distal end of the inner shaft of the elongate body of the over-the-guidewire balloon catheter is removed from the neck lumen of the expandable metallic implant 62 resulting in closure or partial closure of the valve 79 in the neck lumen 62 of the expanded expandable metallic implant 11. The balloon catheter is retracted proximal to the expanded expandable metallic implant, the guidewire 280 is removed from the lumen 174 of the inner shaft 172 of the over-the-guidewire balloon catheter 100 and one or more coil expandable implants 301 is pushed through the hub 176 of the over-the-guidewire balloon catheter 100 and through the lumen 174 of the inner shaft 172 of the over-the-guidewire balloon catheter and placed in the segment of artery or vein proximal to the expanded expandable metallic implant 11. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after placement of one or more coil expandable implants 301 to evaluate the size and position of the expanded expandable metallic implant 11 and the one or more coil expandable implants 301, and the amount of blood flow in the segment of artery indicated for obstruction or reduction in the rate of blood flow. After placement of the one or more coil expandable implants 301 the balloon catheter 100 and the guide catheter or guide sheath 200 are removed from the patient. In some examples, wherein a Tuohy Borst adaptor 406 is joined to the hub 203 of the guide catheter or guide sheath 200, radiographic contrast material is injected into the segment of artery indicated for obstruction or reduction in the rate of blood flow using the side arm 408 of the Tuohy Borst adaptor 406 after removal of the balloon catheter 100 and guidewire 280 to evaluate the size and position of the expanded expandable metallic implant 11, the one or more coil expandable implants 301, and the amount of blood flow in the segment of artery indicated for obstruction or reduction in the rate of blood flow.

### EXAMPLES OF USE

Methods of treatment of arteries, veins, aneurysms, parent vessels of saccular aneurysms, paravalvular leak pathways, biological conduits, or other blood-containing, fluid-containing, or biological spaces using expandable implant devices with or without adjunctively using one or more coil expandable implants 301 or vascular plug expandable implants are also described. Goals of treatment include obstructing, occluding, embolizing, sealing, or reducing the flow of blood or other biological fluids in these structures.

### Example 1: Treatment of a Gastroduodenal Artery Using an Expandable Metallic Implant Medical Device

A 5 Fr guide catheter or guide sheath 200 is provided with an overall length of 80 cm. The length of the elongate body 201 of the 5 Fr guide catheter or guide sheath 200 is 79 cm. The distal end of the 5 Fr guide catheter or guide sheath 200 is formed with a Simmons 1 shape. The distal portion of the outer layer 207 of the elongate body 201 of the 5 Fr guide catheter or guide sheath 200 comprises polyether polyurethane, the middle portion of the outer layer 207 comprises polyether block amide, and the proximal portion of the outer layer 207 comprises nylon. The middle layer 206 of the 5 Fr guide catheter or guide sheath 200 comprises a nitinol coiled wire. The inner layer 205 of the 5 Fr guide catheter or guide sheath 200 comprises polytetrafluoroethylene. The outer surface of the distal 30 cm of the 5 Fr guide catheter or guide sheath 200 comprises a lubricious coating layer. A Tuohy Borst adaptor 406 with a side arm 408 is provided and attached to the hub 203 of the 5 Fr guide catheter or guide sheath 200.

A 5 Fr selective catheter 240 is provided with an overall length of 100 cm. The length of the elongate body 241 of the selective catheter 240 is 99 cm. The distal end of the 5 Fr selective catheter 240 is formed with an angled shape.

A 0.035-inch guidewire 280 is provided with a lubricious coating layer 284. The overall length of the 0.035-inch guidewire 280 is 300 cm. The distal tip of the 0.035-inch guidewire 280 is formed with an angled shape.

An expandable metallic implant device 1 is provided. The overall length of the expandable metallic implant device 1 is 150 cm. The diameter of the seal zone 18 of the expanded expandable metallic implant 11 is 4 mm. The core portion 14 of the expandable metallic implant 10 comprises a single gold layer with a thickness of 20 microns. The expandable metallic implant 10 comprises a neck 60, a conical nose cone 68, and a guidewire segment 69. The diameter of the intermediate region 118 of the inflated balloon 111 is 4 mm. The wall 137 of the balloon 110 comprises a single layer of PET with a thickness of 10 microns. The balloon 110 comprises a proximal neck 123, a distal neck 129, and a neck bridging segment 135. There is a cylindrical radiopaque marker in the lumen 131 of the distal neck 129 of the balloon 110. There is a radiopaque marker band 163 on the elongate body 160 near the distal end. The length of the elongate body 160 is 145 cm. A hub 162 configured for coupling to a syringe or inflation device is bonded to the proximal end of the elongate body 160. The distal portion of the outer layer 168 of the elongate body 160 comprises polyether polyurethane, the middle portion of the outer layer 168 of the elongate body 160 comprises polyether block amide, and the proximal portion of the outer layer 168 of the elongate body 160 comprises nylon. The middle layer 167 of the elongate body 160 comprises a nitinol coiled wire. The inner layer 166 of the elongate body 160 comprises polytetrafluoroethylene. The outer surface of the distal 30 cm of the elongate body 160 comprises a lubricious coating layer 169. The expandable metallic implant 10 and the balloon 110 of the expandable metallic implant device 1 are folded and pleated together.

The tip of a needle is advanced from the skin into the lumen right femoral artery in a retrograde direction in a patient with liver metastasis who is scheduled for radioembolization. The 0.035-inch guidewire 280 is inserted through the needle into the artery in a retrograde fashion. When the tip of the needle is in the lumen of the femoral artery, the 0.035-inch guidewire 280 is inserted into the artery in a retrograde fashion. The needle is removed, and a 5 Fr introducer sheath is inserted into the right femoral artery in a retrograde direction over the 0.035-inch guidewire 280. 5 Fr introducer sheath is flushed with saline containing heparin. The 5 Fr guide catheter or guide sheath 200 is advanced over the 0.035-inch guidewire 280 through the 5 Fr introducer sheath and into the femoral artery. The Tuohy Borst adaptor 406 is joined to the hub 203 of the 5 Fr guide catheter or guide sheath 200 and the lumen of the Tuohy Borst adaptor 406 and the 5 Fr guide catheter or guide sheath 200 are flushed with heparinized saline.

The 5 Fr guide catheter or guide sheath 200 is advanced into the ascending aorta over the 0.035-inch guidewire 280 and the Simmons 1 shape of the distal end of the 5 Fr guide catheter or guide sheath 200 is formed over the aortic valve. The 0.035-inch guidewire 280 and 5 Fr guide catheter or guide sheath 200 are retracted until the tip of the 5 Fr guide catheter or guide sheath 200 is near the origin of the celiac artery. The 0.035-inch guidewire 280 is removed. A syringe containing a mixture of iodinated radiographic contrast and heparinized saline solution is joined to the side arm 408 of the Tuohy Borst adaptor 406 and the tip of the 5 Fr guide catheter or guide sheath 200 is advanced into the celiac artery using small injections of contrast/saline solution mixture for guidance. A mixture of heparinized saline and iodinated radiographic contrast is injected into the patient during angiography using the side arm 408 of the Tuohy Borst adaptor 406. The diameter of the target gastroduodenal artery segment is measured at 3 mm, using the distance between the marker bands 209 on the 5 Fr guide catheter or guide sheath 200 as a distance reference marker. The 0.035-inch guidewire 280 is inserted into the lumen 242 of the 5 Fr selective catheter 240 and the 0.035-inch guidewire 280 and the 5 Fr selective catheter 240 and 0.035-inch guidewire 280 are advanced through the Tuohy Borst adaptor 406, through the lumen 202 of the 5 Fr guide catheter or guide sheath 200, and into the patient until the tip of the 5 Fr selective catheter 240 is located in the target gastroduodenal artery segment. The 5 Fr guide catheter or guide sheath 200 is advanced over the 5 Fr selective catheter 240 until the tip of the 5 Fr guide catheter or guide sheath 200 is located in the target gastroduodenal artery segment.

The operator removes residual air from the expandable metallic implant device 1 by placing the folded and pleated expandable metallic implant 13 in a bowl of heparinized saline solution and aspirating air from the interior space 120 of the balloon 110 and the lumen 161 of the elongate body 160 using a syringe. The heparinized saline solution is drawn through the flush opening(s) 138 and into the interior space 120 of the balloon 110 and the air in the interior space 120 of the balloon 110 and the lumen 161 of the elongate body 160 is aspirated into the syringe.

The operator inserts the folded and pleated expandable metallic implant 13 through the Tuohy Borst adaptor 406, through the lumen 202 of the elongate body 201 of the 5 Fr guide catheter or guide sheath 200, and into the patient until the folded and pleated expandable metallic implant and balloon assembly 4 it is positioned in the target gastroduodenal artery segment. The tip of the 5 Fr guide catheter or guide sheath 200 is retracted until the entire folded and pleated expandable metallic implant and balloon assembly 4 is uncovered. A mixture of heparinized saline and iodinated radiographic contrast is injected into the patient during angiography using the side arm 408 of the Tuohy Borst adaptor 406 to evaluate the position of the folded and pleated expandable metallic implant 13. An inflation device is used to inject a heparinized saline solution into the hub 162 and through the lumen 161 of the elongate body 160, and into the interior space 120 of the balloon 110 at a pressure of 2 atmospheres, resulting in full expansion of the expandable metallic implant 10. A mixture of heparinized saline and iodinated radiographic contrast is injected into the patient during angiography using the side arm 408 of the Tuohy Borst adaptor 406 to evaluate the size and position of the expanded expandable metallic implant 11 and to assess the amount of flow remaining in the target gastroduodenal artery segment. The operator then draws a vacuum with the inflation device and removes fluid from the interior space 120 of the balloon 110 resulting in balloon deflation. After the balloon 110 is deflated, the operator retracts the elongate body 160 and removes the balloon catheter 100 from the patient. A mixture of heparinized saline and iodinated radiographic contrast is injected into the patient during angiography using the side arm 408 of the Tuohy Borst adaptor 406 to evaluate the size and position of the expanded expandable metallic implant 11 and to assess the amount of flow remaining in the target gastroduodenal artery segment. The operator then removes the 5 Fr guide catheter or guide sheath 200 and the 5 Fr introducer sheath from the patient and seals the hole in the right femoral artery with a closure device, having successfully occluded the target gastroduodenal artery segment. Over the next few months, the exterior 41 surface and interior surface 42 of the expanded expandable metallic implant 11 induces the formation of a capsule of fibrous tissue that seals the target artery segment and holds the expanded expandable metallic implant 11 in place.

### Example 2: Treatment of a Gastroduodenal Artery Using an Expandable Metallic Implant Medical Device

A 5 Fr guide catheter or guide sheath 200 is provided with an overall length of 80 cm. The length of the elongate body 201 of the 5 Fr guide catheter or guide sheath 200 is 79 cm. The distal end of the 5 Fr guide catheter or guide sheath 200 is formed with a Simmons 1shape. The distal portion of the outer layer 207 of the elongate body 201 of the 5 Fr guide catheter or guide sheath 200 comprises polyether polyurethane, the middle portion of the outer layer 207 comprises polyether block amide, and the proximal portion of the outer layer 207 comprises nylon. The middle layer 206 of the 5 Fr guide catheter or guide sheath 200 comprises a nitinol coiled wire. The inner layer 205 of the 5 Fr guide catheter or guide sheath 200 comprises polytetrafluoroethylene. The outer surface of the distal 30 cm of the 5 Fr guide catheter or guide sheath 200 comprises a lubricious coating layer. A Tuohy Borst adaptor 406 with a side arm 408 is provided and attached to the hub 203 of the 5 Fr guide catheter or guide sheath 200.

A 4 Fr selective catheter 240 is provided with an overall length of 100 cm. The length of the elongate body 241 of the selective catheter 240 is 99 cm. The distal end of the 4 Fr selective catheter 240 is formed with an angled shape.

A 0.035-inch guidewire 280 is provided with a lubricious coating layer 284. The overall length of the 0.035-inch guidewire 280 is 300 cm. The distal tip of the 0.035-inch guidewire 280 is formed with an angled shape.

An expandable metallic implant device 1 is provided. The overall length of the expandable metallic implant device 1 is 150 cm. The diameter of the seal zone 18 of the expanded expandable metallic implant 11 is 4 mm. The core portion 14 of the expandable metallic implant 10 comprises a single gold layer with a thickness of 20 microns. The expandable metallic implant 10 comprises a neck 60, a conical nose cone 68, and a guidewire segment 69. The expanded expandable metallic implant 11 comprises a balloon retention region 20 in the proximal zone 17 wherein the diameter of the balloon retention region 20 is smaller than the region adjacent and distal to the balloon retention region 20. The expanded expandable metallic implant 11 also comprises four flaps 22 in the proximal portion of the seal zone 18 with pointed free ends, and wherein the free end of the flaps 22 are distal to the fixed end of the flaps 22. The diameter of the intermediate region 118 of the inflated balloon 111 is 4 mm. The wall 137 of the balloon 110 comprises a single layer of PET with a thickness of 10 microns and is semi-compliant. The balloon 110 comprises a proximal neck 123, a distal neck 129, and a neck bridging segment 135. There is a cylindrical radiopaque marker in the lumen 131 of the distal neck 129 of the balloon 110. There is a radiopaque marker band 163 on the elongate body 160 near the distal end. The length of the elongate body 160 is 145 cm. A hub 162 configured for coupling to a syringe or inflation device is bonded to the proximal end of the elongate body 160. The distal portion of the outer layer 168 of the elongate body 160 comprises polyether polyurethane, the middle portion of the outer layer 168 of the elongate body 160 comprises polyether block amide, and the proximal portion of the outer layer 168 of the elongate body 160 comprises nylon. The middle layer 167 of the elongate body 160 comprises a nitinol coiled wire. The inner layer 166 of the elongate body 160 comprises polytetrafluoroethylene. The outer surface of the distal 30 cm of the elongate body 160 comprises a lubricious coating layer 169. The expandable metallic implant 10 and the balloon 110 are folded and pleated together.

The tip of a needle is advanced from the skin into the lumen right femoral artery in a retrograde direction in a patient with liver metastasis who is scheduled for radioembolization. The 0.035-inch guidewire 280 is inserted through the needle into the artery in a retrograde fashion. The needle is removed, and a 5 Fr introducer sheath is inserted into the right femoral artery in a retrograde direction over the 0.035-inch guidewire 280. The 5 Fr introducer sheath is flushed with saline containing heparin. The 5 Fr guide catheter or guide sheath 200 is advanced over the 0.035-inch guidewire 280 through the 5 Fr introducer sheath and into the femoral artery. The Tuohy Borst adaptor 406 is joined to the hub 203 of the 5 Fr guide catheter or guide sheath 200 and the lumen of the Tuohy Borst adaptor 406 and the 5 Fr guide catheter or guide sheath 200 are flushed with heparinized saline.

The 5 Fr guide catheter or guide sheath 200 is advanced into the ascending aorta over the 0.035-inch guidewire 280 and the Simmons 1 shape of the distal end of the 5 Fr guide catheter or guide sheath 200 is formed over the aortic valve. The 0.035-inch guidewire 280 and 5 Fr guide catheter or guide sheath 200 are pulled back until the tip of the 5 Fr guide catheter or guide sheath 200 is near the origin of the celiac artery. The 0.035-inch guidewire 280 is removed. A syringe containing a mixture of iodinated radiographic contrast and heparinized saline solution is joined to the hub of the side arm 408 of the Tuohy Borst adaptor 406 and the tip of the 5 Fr guide catheter or guide sheath 200 is advanced into the celiac artery using small injections of contrast/saline solution mixture for guidance.

A mixture of heparinized saline and iodinated radiographic contrast is injected into the patient during angiography using the side arm 408 of the Tuohy Borst adaptor 406 and the diameter of the target gastroduodenal artery segment is measured at 3 mm using the distance between the marker bands 209 on the 5 Fr guide catheter or guide sheath 200 as a distance reference marker. The 0.035-inch guidewire 280 is inserted into the lumen 242 of the 4 Fr selective catheter 240 and the 0.035-inch guidewire 280 and the 4 Fr selective catheter 240 are advanced along with the 0.035-inch guidewire 280 through the Tuohy Borst adaptor 406 and into the lumen 202 of the 5 Fr guide catheter or guide sheath 200. The 4 Fr selective catheter 240 is advanced over the 0.035-inch guidewire 280 until the tip of the 4 Fr selective catheter 240 is located in the target gastroduodenal artery segment. The 5 Fr guide catheter or guide sheath 200 is advanced over the 0.035-inch guidewire 280 and the 4 Fr selective catheter 240 until the tip of the 5 Fr guide catheter or guide sheath 200 is located in the target gastroduodenal artery segment.

The operator removes residual air from the expandable metallic implant device 1 by placing the folded and pleated expandable metallic implant 13 in a bowl of heparinized saline solution and aspirating air from the interior space 120 of the balloon 110 and the lumen 161 of the elongate body 160 using a syringe. The heparinized saline solution is drawn through the flush opening(s) 138 and into the interior space 120 of the balloon 110 and the air in the interior space 120 of the balloon 110 and the lumen 161 of the elongate body 160 is aspirated into the syringe.

The operator inserts the folded and pleated expandable metallic implant 13 through the Tuohy Borst adaptor 406, through the lumen 161 of the elongate body 160, and into the patient until the folded and pleated expandable metallic implant 13 is positioned in the target gastroduodenal artery segment, using the guidewire segment 69 to lead the way. The tip of the 5 Fr guide catheter or guide sheath 200 is then retracted until the entire expandable metallic implant 10 is uncovered. A mixture of heparinized saline and iodinated radiographic contrast is injected into the patient during angiography using the side arm 408 of the Tuohy Borst adaptor 406 to evaluate the position of the folded and pleated expandable metallic implant 13. An inflation device is used to inject a heparinized saline solution into the hub 162, through the lumen 161 of the elongate body 160, and into the interior space 120 of the balloon 110 at a pressure of 2 atmospheres, resulting in expansion of the expandable metallic implant 10. A mixture of heparinized saline and iodinated radiographic contrast is injected into the patient during angiography using the side arm 408 of the Tuohy Borst adaptor 406 to evaluate the size and position of the expanded expandable metallic implant 11 and to assess the amount of flow remaining in the target gastroduodenal artery segment. The inflation device is then used to inject a heparinized saline solution into the hub 162, through the lumen 161 of the elongate body 160, and into the interior space 120 of the balloon 110 at a pressure of 10 atmospheres, resulting in the balloon over-inflation and bulging of the over-inflated balloon 112 into the flap windows 24 of the expanded expandable metallic implant 11 and the raising of the free end of the flaps 22 from the exterior surface 41 of the seal zone 18 of the expanded expandable metallic implant 11. A mixture of heparinized saline and iodinated radiographic contrast is injected into the patient during angiography using the side arm 408 of the Tuohy Borst adaptor 406 to evaluate the size and position of the expanded expandable metallic implant 11, the position and orientation of the flaps 22, and to assess the amount of flow remaining in the target gastroduodenal artery segment.

The operator then draws a vacuum with the inflation device and removes fluid from the interior space 120 of the balloon 110 resulting in balloon deflation. After the balloon 110 is deflated, the operator retracts the elongate body 160 and removes the balloon catheter 100 from the patient. A mixture of heparinized saline and iodinated radiographic contrast is injected into the patient during angiography using the side arm 408 of the Tuohy Borst adaptor 406 to evaluate the size and position of the expanded expandable metallic implant 11 and to assess the amount of flow remaining in the target gastroduodenal artery segment. The operator then removes the 5 Fr guide catheter or guide sheath 200 and the 4 Fr introducer sheath from the patient and seals the hole in the right femoral artery with a closure device, having successfully occluded the target gastroduodenal artery segment. Over the next few months, the exterior 41 surface and interior surface 42 of the expanded expandable metallic implant 11 induces the formation of a capsule of fibrous tissue that seals the target artery segment and holds the expanded expandable metallic implant 11 in place.

### Example 3: Treatment of a Renal Artery Leading to an Arteriovenous Fistula Using an Expandable Metallic Implant Medical Device and Coil Expandable Implants

A 7 Fr guide sheath 200 is provided with an overall length of 80 cm. The length of the elongate body 201 of the 7 Fr guide sheath 200 is 79 cm. The diameter of the lumen 202 of the 7 Fr guide sheath 200 is 0.081 inch. The distal end of the 7 Fr guide sheath 200 is formed with a straight shape. The distal portion of the outer layer 207 of the elongate body 201 of the 7 Fr guide sheath 200 comprises polyether polyurethane, the middle portion of the outer layer 207 comprises polyether block amide, and the proximal portion of the outer layer 207 comprises nylon. The middle layer 206 of the 7 Fr guide sheath 200 comprises a nitinol coiled wire. The inner layer 205 of the 7 Fr guide sheath 200 comprises polytetrafluoroethylene. The outer surface of the distal 30 cm of the 7 Fr guide sheath 200 comprises a lubricious coating layer.

A 6 Fr intermediate catheter 220 is provided with an overall length of 90 cm. The length of the elongate body 221 of the 6 Fr intermediate catheter 220 is 89 cm. The distal end of the 6 Fr intermediate catheter 220 is formed with a straight shape. The distal portion of the outer layer 226 of the elongate body 221 of the 6 Fr intermediate catheter 220 comprises polyether polyurethane, the middle portion of the outer layer 226 comprises polyether block amide, and the proximal portion of the outer layer 226 comprises nylon. The middle layer 225 of the 6 Fr intermediate catheter 220 comprises a nitinol coiled wire. The inner layer 224 of the 6 Fr intermediate catheter 220 comprises polytetrafluoroethylene. The outer surface of the distal 30 cm of the 7 Fr guide sheath 200 comprises a lubricious coating layer. An obturator 412 with a tapered tip is provided with the 6 Fr intermediate catheter 220 to facilitate vessel insertion over a 0.035-inch guidewire 280.

A Tuohy Borst adaptor 406 with a side arm 408 with a three-way stopcock with Luer hubs is provided and attached to the hub 203 of the 7 Fr guide sheath 200.

A 5 Fr selective catheter 240 is provided with an overall length of 100 cm. The length of the elongate body 241 of the selective catheter 240 is 99 cm. The distal end of the 5 Fr selective catheter 240 is formed with a Cobra shape.

A 0.035-inch guidewire 280 is provided with a lubricious coating layer 284. The overall length of the 0.035-inch guidewire 280 is 300 cm. The distal tip of the 0.035-inch guidewire 280 is formed with an angled shape.

An expandable metallic implant device 1 is provided. The overall length of the expandable metallic implant device 1 is 150 cm. The diameter of the seal zone 18 of the expanded expandable metallic implant 11 is 16 mm. The core portion 14 of the expandable metallic implant 10 comprises a single gold layer with a thickness of 30 microns. The expandable metallic implant 10 comprises a neck 60, a conical nose cone 68, and a guidewire segment 69. The diameter of the intermediate region 118 of the inflated balloon 111 is 16 mm. The wall 137 of the balloon 110 comprises a single layer of PET with a thickness of 20 microns. The balloon 110 comprises a proximal neck 123, a distal neck 129, and a neck bridging segment 135. There is a cylindrical radiopaque marker in the lumen 131 of the distal neck 129 of the balloon 110. There is a radiopaque marker band 163 on the elongate body 160 near the distal end. The length of the elongate body 160 is 145 cm. The outer diameter of the elongate body 160 is 0.042 inch. A hub 162 configured for coupling to a syringe or inflation device is bonded to the proximal end of the elongate body 160. The distal portion of the outer layer 168 of the elongate body 160 comprises polyether polyurethane, the middle portion of the outer layer 168 of the elongate body 160 comprises polyether block amide, and the proximal portion of the outer layer 168 of the elongate body 160 comprises nylon. The middle layer 167 of the elongate body 160 comprises a nitinol coiled wire. The inner layer 166 of the elongate body 160 comprises polytetrafluoroethylene. The outer surface of the distal 30 cm of the elongate body 160 comprises a lubricious coating layer 169. The expandable metallic implant 10 and the balloon 110 are folded and pleated together.

A 2.6 Fr coiling or delivery catheter 260 is provided. The outer diameter of the elongate body 261 of the coiling or delivery catheter 260 is 0.034 inch. The distal end of the 2.6 Fr coiling or delivery catheter 260 is formed with an angled shape. The overall length of the 2.6 Fr coiling or delivery catheter 260 is 160 cm. The length of the elongate body 261 of the coiling or delivery catheter 260 is 159 cm.

A 0.018-inch guidewire 280 is provided. The overall length of the 0.018-inch guidewire 280 is 185 cm. The distal tip of the 0.0.018-inch guidewire 280 is formed with an angled shape.

Four coil expandable implants 301 are provided, each comprised of platinum and measuring 16 mm in tertiary diameter and 60 cm in length, with a secondary diameter of 0.020 inch. The coil expandable implants 301 are joined to a 200 cm long elongate body 307 that is configured as a wire and detached through mechanical means.

A first Tuohy Borst adaptor 406 is joined to hub 203 of the 7 Fr guide sheath 200 and the Tuohy Borst adaptor 406 and the 7 Fr guide sheath 200 are flushed. A second Tuohy Borst adaptor 409 is joined to hub 223 of the 6 Fr intermediate catheter 220 and the second Tuohy Borst adaptor 409 and the 6 Fr intermediate catheter 220 are flushed. The obturator 412 for the 6 Fr intermediate catheter 220 is inserted into the lumen of the 6 Fr intermediate catheter 220 and the assembly of the 6 Fr intermediate catheter 220, second Tuohy Borst adaptor 409, and obturator 412 are inserted through the first Tuohy Borst adaptor 406 joined to the 7 Fr guide sheath 200 and into the lumen 202 of the guide sheath 200.

The tip of a needle is advanced from the skin into the lumen of the right femoral artery in a retrograde direction in a patient who was scheduled for embolization of an arteriovenous fistula in the renal fossa involving a renal artery and vein in a patient who previously had a right partial nephrectomy. The 0.035-inch guidewire 280 is inserted through the needle into the artery in a retrograde fashion. The needle is removed, and an assembly of the 7 Fr guide catheter or guide sheath 200, first Tuohy Borst adaptor 406, 6 Fr intermediate catheter 220, second Tuohy Borst adaptor 409, and the obturator 412 is inserted into the right femoral artery in a retrograde direction over the 0.035-inch guidewire 280 with the obturator 412 leading. The obturator 412 is removed and the first Tuohy Borst adaptor 406, 7 Fr guide sheath 200, second Tuohy Borst adaptor 409, and 6 Fr intermediate catheter 220 are flushed with saline containing heparin.

The 5 Fr selective catheter 240 is advanced over the 0.035-inch guidewire 280 through the second Tuohy Borst adaptor 409 through the lumen 202 of the 6 Fr intermediate catheter 220 and into the patient. The assembly of the 7 Fr guide catheter or guide sheath 200, first Tuohy Borst adaptor 406, 6 Fr intermediate catheter 220, second Tuohy Borst adaptor 409, and 5 Fr selective catheter 240 is advanced over the 0.035-inch guidewire 280 into the ascending aorta, with the 0.035-inch guidewire 280 leading. The Simmons 1 shaped distal end of the 5 Fr selective catheter 240 is formed over the aortic valve. The assembly of the 0.035-inch guidewire 280, 5 Fr selective catheter 240, first Tuohy Borst adaptor 406, 6 Fr intermediate catheter 220, second Tuohy Borst adaptor 409, and 7 Fr guide sheath 200 is pulled back until the tip of the 5 Fr selective catheter 240 is near the origin of the right renal artery. The 0.035-inch guidewire 280 is removed. A syringe containing a mixture of iodinated radiographic contrast and heparinized saline solution is joined to the hub 243 of the 5 Fr selective catheter 240 and the tip of the 5 Fr selective catheter 240 is advanced into the right renal artery using small injections of contrast/saline solution mixture for guidance. A mixture of heparinized saline and iodinated radiographic contrast is injected into the patient during angiography using the side arm 408 of the Tuohy Borst adaptor 406, and the diameter of the target right renal artery segment is measured at 12 mm, using the distance between the marker bands 209 on the 7 Fr guide sheath 200 as a distance reference marker. The 0.035-inch guidewire 280 is inserted into the lumen 242 of the 5 Fr selective catheter 240 and the 0.035-inch guidewire 280 and the 5 Fr selective catheter 240 is advanced along with the 0.035-inch guidewire 280 until the tip of the 5 Fr selective catheter 240 is located in the target renal artery segment. The 6 Fr intermediate catheter 220 and the 7 Fr guide sheath 200 are then advanced over the 5 Fr selective catheter 240 until the tip of the 7 Fr guide sheath 200 is located in the target renal artery segment. The 0.035-inch guidewire 280, 5 Fr selective catheter 240, second Tuohy Borst adaptor 409, and 6 Fr intermediate catheter 220 are removed from the patient, and the 7 Fr guide sheath 200 is flushed with saline containing heparin using the first Tuohy Borst adaptor 406.

The operator removes residual air from the expandable metallic implant device 1 by placing the folded and pleated expandable metallic implant 13 in a bowl of heparinized saline solution and aspirating air from the interior space 120 of the balloon 110 and the lumen 161 of the elongate body 160 using a syringe. The heparinized saline solution is drawn through the flush opening(s) 138 and into the interior space 120 of the balloon 110 and the air in the interior space 120 of the balloon 110 and the lumen 161 of the elongate body 160 is aspirated into the syringe.

The operator inserts the folded and pleated expandable metallic implant 13 of expandable metallic implant device 1 into the first Tuohy Borst adaptor 406, through the lumen 202 of the guide sheath 200, and into the patient until the folded and pleated expandable metallic implant 13 is positioned in the target renal artery segment. The tip of the 7 Fr guide sheath 200 is retracted until the entire folded and pleated expandable metallic implant and balloon assembly 4 is exposed. A mixture of heparinized saline and iodinated radiographic contrast is injected into the patient during angiography using the side arm 408 of the first Tuohy Borst adaptor 406 to evaluate the position of the folded and pleated expandable metallic implant 13.

The 0.018-inch guidewire 280 is inserted into the 2.6 Fr coiling or delivery catheter 260 and the 2.6 Fr coiling or delivery catheter 260 is inserted into the Tuohy Borst adaptor 406, through the lumen 202 of the guide sheath 200, and into the patient, with the 0.018-inch guidewire 280 leading the way. The 2.6 Fr coiling or delivery catheter 260 is advanced alongside the folded and pleated expandable metallic implant 13 until the tip of the 2.6 Fr coiling or delivery catheter 260 is in the target renal artery segment distal to the folded and pleated expandable metallic implant 13. The 0.018-inch guidewire 280 is removed. A mixture of heparinized saline and iodinated radiographic contrast is injected into the patient during angiography using the side arm 408 of the Tuohy Borst adaptor 406 to evaluate the position of the folded and pleated expandable metallic implant 13 and the 2.6 Fr coiling or delivery catheter 260.

An inflation device is used to inject a heparinized saline solution into the hub 162 and through the lumen 161 of the elongate body 160, and into the interior space 120 of the balloon 110 at a pressure of 2 atmospheres until the expandable metallic implant 10 is partially expanded. A mixture of heparinized saline and iodinated radiographic contrast is injected into the patient during angiography using the side arm 408 of the Tuohy Borst adaptor 406 to evaluate the size and position of the expanded expandable metallic implant 11, the position of the 2.6 Fr coiling or delivery catheter 260, and the amount of flow remaining in the target renal artery segment.

One of the coil expandable implants 301 is inserted into the hub 263 of the 2.6 Fr coiling or delivery catheter 260, advanced through the lumen 262 of the 2.6 Fr coiling or delivery catheter 260 and placed in the target renal artery segment distal to the partially expanded expandable metallic implant 11 using the elongate body 307 of the coil expandable implant 301. The coil expandable implant 301 is detached from the elongate body 307 and the elongate body 307 is removed. The other three coil expandable implants 301 are placed using a similar method. A mixture of heparinized saline and iodinated radiographic contrast is injected into the patient during angiography using the side arm 408 of the Tuohy Borst adaptor 406 to evaluate the size and position of the expanded expandable metallic implant 11 and the coil expandable implants 301, the position of the 2.6 Fr coiling or delivery catheter 260, and the amount of flow remaining in the target renal artery segment. The 2.6 Fr coiling or delivery catheter 260 is removed from the patient.

The inflation device is used to inject additional heparinized saline solution into the hub 162, through the lumen 161 of the elongate body 160, and into the interior space 120 of the balloon 110 at a pressure of 2 atmospheres until the expandable metallic implant 10 is completed expanded. A mixture of heparinized saline and iodinated radiographic contrast is injected into the patient during angiography using the side arm 408 of the Tuohy Borst adaptor 406 to evaluate the size and position of the expanded expandable metallic implant 11 and the coil expandable implants 301, and the amount of flow remaining in the target renal artery segment.

The operator then draws a vacuum with the inflation device and removes fluid from the interior space 120 of the balloon 110, resulting in balloon deflation. After the balloon 110 is deflated, the operator retracts the elongate body 160 and removes the balloon catheter 100 from the patient. A mixture of heparinized saline and iodinated radiographic contrast is injected into the patient during angiography using the side arm 408 of the Tuohy Borst adaptor 406 to evaluate the size and position of the expanded expandable metallic implant 11 and the coil expandable implants 301, and the amount of flow remaining in the target renal artery segment.

The operator then removes the 7 Fr guide sheath 200 from the patient and seals the hole in the right femoral artery with a closure device, having successfully occluded the target right renal artery segment. Over the next few months, the exterior 41 surface and interior surface 42 of the expanded expandable metallic implant 11 induces the formation of a capsule of fibrous tissue that seals the target artery segment and holds the expanded expandable metallic implant 11 in place.

### Miscellaneous

One skilled in the art would understand that the methods described in Examples 1-2 can be used to treat other arteries, veins, aneurysms, parent vessels of saccular aneurysms, paravalvular leak pathways, biological conduits, or other blood-containing, fluid-containing, or biological spaces.

It will be appreciated that the devices, systems, and methods of the present disclosure are capable of being incorporated in the form of a variety of examples, only a few of which have been illustrated and described above. The disclosures herein may be embodied in other specific forms without departing from its spirit or essential characteristics. The described examples are to be considered in all respects only as illustrative and not restrictive and the scope of the present examples is, therefore indicated by the appended claims rather than by the foregoing description. All changes that come within the meaning and range of equivalency of the claims are to be embraced within their scope.

### EXAMPLES

Additional systems and methods are disclosed in the examples below, which should not be viewed as limiting in any way.

Example 1. An expandable metallic implant device for use in obstructing or reducing the flow of blood or other biological fluids in a human patient, the device comprising: (a) a hollow expandable metallic implant for permanent implantation in the human body configured to expand from a folded and pleated configuration to an expanded configuration, the expandable metallic implant comprising a wall that is open on the proximal end and closed or obstructed on the distal end; and (b) a balloon catheter comprising: (i) an elongate body comprising: a hub joined or bonded to the proximal end of the elongate body and configured to couple with a syringe or other fluid source; a lumen to allow for the passage of fluid; and (ii) a balloon joined to the distal region of the elongate body, wherein the balloon comprises a proximal region, a distal region generally opposite the proximal region, and an intermediate region between the proximal and distal region, and a first axis extending proximal to distal between the proximal region and the distal region; (c) wherein: (i) the lumen of the elongate body extends from the hub to the distal end of the elongate body and is configured to enable the passage of fluid from the hub, through the lumen of the elongate body, and into the interior space of the balloon to enable inflation and deflation of the balloon; (ii) the overall length of the inflated balloon is equal to or greater than the overall length of the expanded expandable metallic implant; (iii) the expandable metallic implant and balloon are folded and pleated together; (iv) the passage of fluid through the lumen of the elongate body and into the interior space of the balloon under pressure can result in inflation of the balloon and expansion of the folded and pleated expandable metallic implant; (v) after deflation of the balloon, the balloon catheter can be removed from the patient while the expanded expandable metallic implant remains in place in the patient.

Example 2. The expandable metallic implant device of example 1, wherein the wall of the expandable metallic implant has a thickness ranging between 5 microns and 30 microns, between 3 and 100 microns, or between 3 and 500 microns.

Example 3. The expandable metallic implant device of example 1, wherein the wall of the expandable metallic implant comprises at least one layer, with the at least one layer comprising gold or a gold alloy.

Example 4. The expandable metallic implant device of example 3, wherein the gold layer of the expandable metallic implant has a thickness ranging between 3 and 500 microns.

Example 5. The expandable metallic implant device of example 3, wherein the wall of the expandable metallic implant comprises gold with a thickness of <5 microns, <10microns, <15 microns, <20 microns, <25 microns, <30 microns, <35 microns, or <40 microns.

Example 6. The expandable metallic implant device of any one of examples 1 5, wherein, after expansion to the nominal diameter, the expanded expandable metallic implant is non-compliant.

Example 7. The expandable metallic implant device of any one of examples 1 5, wherein, after expansion to the nominal diameter, the expanded expandable metallic implant is noncompliant when the pressure inside the balloon is in a range between 1 and 20 atmospheres.

Example 8. The expandable metallic implant device of any one of examples 1 5, wherein, after expansion to the nominal diameter, the diameter of the expanded expandable metallic implant increases: (a) <5 or <10% when the balloon is inflated to a pressure of 5 atmospheres; (b) <5 or <10% when the balloon is inflated to a pressure of 10 atmospheres; (c) <5 or <10% when the balloon is inflated to a pressure of 15 atmospheres; or (d) <5 or <10% when the balloon is inflated to a pressure of 20 atmospheres.

Example 9. The expandable metallic implant device of example 1, wherein the wall of the balloon has a thickness ranging between 5 and 30 microns or between 3 and 100 microns.

Example 10. The expandable metallic implant device of any one of examples 1 and 40-44, wherein the wall of the balloon comprises a polyethylene terephthalate, a nylon, or a material comprising block copolymers made of rigid polyamide blocks and soft polyether blocks, including Pebax.

Example 11. The expandable metallic implant device of example 10, wherein the wall of the balloon comprises a polyethylene terephthalate with a thickness of <10 microns, <15 microns, or <20 microns.

Example 12. The expandable metallic implant device of any one of examples 1 and 9-11, wherein, after expansion to the nominal diameter, the balloon is compliant or semi-compliant.

Example 13. The expandable metallic implant device of any one of examples 1 and 9-11, wherein, after expansion to the nominal diameter, the balloon is semi-compliant or compliant when the pressure inside the balloon is in a range between 1 and 20 atmospheres.

Example 14. The expandable metallic implant device of any one of examples 1 and 9-11, wherein, after expansion to the nominal diameter, the diameter of the inflated balloon increases: (a) 5-10%, 11-15%, 16-20%, 21-25%, 26-30%, 31-35%, 36-40%, 41-45%, or 46-50% when inflated to a pressure of 5 atmospheres; (b) 5-10%, 11-15%, 16-20%, 21-25%, 26-30%, 31-35%, 36-40%, 41-45%, or 46-50% when inflated to a pressure of 10 atmospheres; (c) 5-10%, 11-15%, 16-20%, 21-25%, 26-30%, 31-35%, 36-40%, 41-45%, or 46-50% when inflated to a pressure of 15 atmospheres; or (d) 5-10%, 11-15%, 16-20%, 21-25%, 26-30%, 31-35%, 36-40%, 41-45%, or 46-50% when inflated to a pressure of 20 atmospheres.

Example 15. The expandable metallic implant device of any one of examples 1-14, wherein the expanded expandable metallic implant comprises a balloon retention region.

Example 16. The expandable metallic implant device of example 15, wherein the diameter of the balloon retention region is: (a) smaller than the region adjacent and distal to the balloon retention region; (b) smaller than the region adjacent and proximal to the balloon retention region and smaller than the region and adjacent and distal to the balloon retention region; or (c) larger than the region adjacent and proximal to the balloon retention region.

Example 17. The expandable metallic implant device of any one of examples 15 and 16, wherein the smallest diameter of the balloon retention region is 90-95%, 80-89%, 70-79%, 60-69%, 50-99%, 40-49%, 30-39% of the diameter of an adjacent region.

Example 18. The expandable metallic implant device of any one of examples 15and 16, wherein the largest diameter of the balloon retention region is 105-110%, 111-120%, 121-130%, 131-%, or 141-150% of the diameter of an adjacent region.

Example 19. The expandable metallic implant device of any one of examples 15 and 16, wherein the balloon retention region is at the proximal end of the expanded expandable metallic implant.

Example 20. The expandable metallic implant device of any one of examples 15 and 16, wherein the balloon retention region is in the proximal zone of the expanded expandable metallic implant.

Example 21. The expandable metallic implant device of any one of examples 15 and 16, wherein the balloon retention region is in the seal zone of the expanded expandable metallic implant.

Example 22. The expandable metallic implant device of any one of examples 15 and 16, wherein the balloon retention region is in the transition zone of the expanded expandable metallic implant.

Example 23. The expandable metallic implant device of any one of examples 1 22, wherein the expanded expandable metallic implant comprises at least one flap, and wherein the free end of at least one flap can be raised from the surface of the expandable metallic implant by balloon inflation.

Example 24. The expandable metallic implant device of example 23, wherein the free end of at least one flap are pointed, rounded, squared, or complex in shape.

Example 25. The expandable metallic implant device of example 23, wherein the fixed end of at least one flap comprises a hinge region.

Example 26. The expandable metallic implant device of example 25, wherein the hinge region comprises a linear region with a wall thickness that is less than the wall thickness of the adjacent wall.

Example 27. The expandable metallic implant device of example 23, wherein the at least one flap is configured such that the free end is distal to the fixed end.

Example 28. The expandable metallic implant device of example 23, wherein at least one flap is configured such that the free end is proximal to the fixed end.

Example 29. The expandable metallic implant device of example 23, wherein at least one flap is configured such that the free end is distal to the fixed end and at least one flap is configured such that the free end is proximal to the fixed end.

Example 30. The expandable metallic implant device of example 23, wherein at least one flap is located at the proximal end of the expanded expandable metallic implant.

Example 31. The expandable metallic implant device of example 23, wherein at least one flap is located in proximal zone of the expanded expandable metallic implant.

Example 32. The expandable metallic implant device of example 23, wherein at least one flap is located in seal zone of the expanded expandable metallic implant.

Example 33. The expandable metallic implant device of example 1, wherein the expandable metallic implant further comprises a neck extending distally from the transition zone of the expandable metallic implant.

Example 34. The expandable metallic implant device of example 33, wherein the internal diameter of the neck of the expandable metallic implant is between 0.010-0.100 inches, as measured parallel to the second axis of the expandable metallic implant.

Example 35. The expandable metallic implant device of example 33, wherein the length of the neck of the expandable metallic implant is between 0.3-10 mm, as measured parallel to the first axis of the expandable metallic implant.

Example 36. The expandable metallic implant device of example 33, wherein the neck of the expandable metallic implant further comprises a neck assembly.

Example 37. The expandable metallic implant device of example 36, wherein a guidewire segment is joined or bonded to the neck or another portion of the neck assembly of the expandable metallic implant and extends distally from the distal end of the neck or neck assembly of the expandable metallic implant.

Example 38. The expandable metallic implant device of example 37, wherein the length of the guidewire segment is between 5-50 mm, as measured parallel to the first axis of the expandable metallic implant.

Example 39. The expandable metallic implant device of example 37, wherein the guidewire segment that is joined or bonded to the neck or neck assembly of the expandable metallic implant comprises a core wire and a wound wire.

Example 40. The expandable metallic implant device of example 39, wherein the proximal end of the core wire and wound wire are inserted into the neck or another portion of the neck assembly of the expandable metallic implant and the outer surface of the wound wire is bonded to the inner surface of the neck or other portion of the neck assembly of the expandable metallic implant.

Example 41. The expandable metallic implant device of example 39, wherein the proximal end of the core wire is inserted into the neck or another portion of the neck assembly of the expandable metallic implant, the outer surface of the core wire is bonded to the inner surface of the neck or other portion of the neck assembly of the expandable metallic implant, and the proximal portion of the wound wire is wrapped around the core wire.

Example 42. The expandable metallic implant device of example 39, wherein the proximal end of the core wire is inserted into the neck or another portion of the neck assembly of the expandable metallic implant, the outer surface of the core wire is bonded to the inner surface of the neck or another portion of the neck assembly of the expandable metallic implant, and the proximal portion of the wound wire is wrapped around a portion of the neck or other portion of the neck assembly of the expandable metallic implant.

Example 43. The expandable metallic implant device of example 36, comprising a nose cone joined or bonded to the neck or neck assembly of the expandable metallic implant.

Example 44. The expandable metallic implant device of example 43, wherein: (a) the outer diameter of the proximal portion of the nose cone of the expandable metallic implant is within 0.02 inches of the outer diameter of the folded and pleated expandable metallic implant; and (b) the outer diameter of the distal portion of the nose cone of the expandable metallic implant is within 0.02 inches of the outer diameter of a guidewire segment joined or bonded to the neck or neck assembly of the expandable metallic implant.

Example 45. The expandable metallic implant device of example 43, wherein at least a portion of the nose cone, the neck, and the guidewire segment are overlapping.

Example 46. The expandable metallic implant device of example 43, wherein the nose cone of the expandable metallic implant is comprised of polymer, stainless steel, gold, or platinum.

Example 47. The expandable metallic implant device of example 46, wherein the polymer is a polyether ether ketone (PEEK), polycarbonate, polyamide, polyimide, polyether block amide, polytetrafluoroethylene, silicone, polyurethane, co-polyester polymer, thermoplastic polymer, silicone-polycarbonate copolymer, polyethylene ethyl-vinyl-acetate (PEVA) co-polymer, or a biocompatible elastomer.

Example 48. The expandable metallic implant device of example 33, wherein the junctional region between the neck portion of the expandable metallic implant and the folded and pleated portion of expandable metallic implant can flex or bend in response to forces encountered when the expandable metallic implant device is moving along a tortuous path.

Example 49. The expandable metallic implant device of example 48, wherein the junctional region of the expandable metallic implant comprises a layer of gold.

Example 50. The expandable metallic implant device of example 48, wherein the junctional region of the expandable metallic implant: (a) comprises a layer of gold with a thickness of 3, 4, 5, 6, 7, 8, 9, 10, 11, 12, 13, 14, 15, 16, 17, 18, 19, or 20 microns; (b) comprises a layer of gold with a thickness of <5 microns, <10 microns, <15 microns, or <20 microns; (c) comprises a layer of gold with a thickness less than the thickness of the layer of gold in the neck of the expandable metallic implant; (d) comprises a layer of gold with a thickness less than the thickness of the layer of gold in the transition zone of the expandable metallic implant; or (e) comprises a layer of gold with a thickness less than the thickness of the layer of gold in the neck of the expandable metallic implant and the thickness of the layer of gold in the transition zone of the expandable metallic implant.

Example 51. The expandable metallic implant device of example 1, wherein the wall of the expandable metallic implant extends continuously from the proximal end to the distal end, with the exception of any flap windows.

Example 52. The expandable metallic implant device of example 1, wherein the seal zone of the expanded expandable metallic implant has a largest diameter is between 2 mm 50 mm when measured parallel to the second axis of the expandable metallic implant.

Example 53. The expandable metallic implant device of example 1, wherein the overall length of the expanded expandable metallic implant is between 3 mm-80 mm when measured parallel to the first axis of the expandable metallic implant, excluding any neck or neck assembly, including the guidewire segment.

Example 54. The expandable metallic implant device of example 1, wherein, when the expandable metallic implant is in the expanded configuration, the expandable metallic implant assumes a shape wherein the seal region is cylindrical.

Example 55. The expandable metallic implant device of example 1, wherein, when the expandable metallic implant is in the expanded configuration, the expandable metallic implant assumes a shape wherein the transition region is generally rounded, hemispherical, conical, or flat when the neck and neck assembly are excluded, including the guidewire segment.

Example 56. The expandable metallic implant device of example 1, wherein, when the expandable metallic implant is in the expanded configuration, the expandable metallic implant assumes a shape wherein the seal region is cylindrical and the transition region is generally rounded, hemispherical, conical, or flat when the neck and neck assembly are excluded, including the guidewire segment.

Example 57. The expandable metallic implant device of example 1, wherein the expandable metallic implant and the balloon are formed into a plurality of wings.

Example 58. The expandable metallic implant device of example 57, wherein each wing of the plurality of wings comprises a ridge line extending proximal to distal and radially away from the first axis.

Example 59. The expandable metallic implant device of example 57, wherein each wing comprises a ridge line extending proximal to distal, and each wing is separated from any immediately adjacent wing by an interposed trough extending proximal to distal.

Example 60. The expandable metallic implant device of example 57, wherein the winged configuration comprises an alternating ridge-trough arrangement.

Example 61. The expandable metallic implant device of example 57, wherein the plurality of wings are pleated in a clockwise or counterclockwise direction relative to the first axis.

Example 62. The expandable metallic implant device of example 57, wherein, the balloon and the expandable metallic implant are pressed together or coupled when they are folded together or folded and pleated together.

Example 63. The expandable metallic implant device of example 57, wherein the wings in the proximal portion of the balloon are comprised of two layers of balloon wall.

Example 64. The expandable metallic implant device of example 57, wherein the wings in the distal portion of the balloon are comprised of two layers of balloon wall and two layers of expandable metallic implant wall.

Example 65. The expandable metallic implant device of example 57, wherein the wings in the proximal portion of the balloon are comprised of two layers of balloon wall and the wings in the distal portion of the balloon are comprised of two layers of balloon wall and two layers of expandable metallic implant wall.

Example 66. The expandable metallic implant device of example 1, wherein the folded and pleated expandable metallic implant is configured to assume the expanded configuration by substantially eliminating the folded and pleated configuration or a plurality of wings of the expandable metallic implant that are present in the folded and pleated configuration.

Example 67. The expandable metallic implant device of example 1, wherein the folded and pleated expandable metallic implant can be expanded by injection of water, saline, radiographic contrast, or combinations thereof, from the hub of the elongate body, through the lumen of the elongate body, and into the interior space of the balloon under pressure.

Example 68. The expandable metallic implant device of example 67, wherein the expandable metallic implant is configured to be expanded from the folded and pleated configuration to the expanded configuration by the injection of fluid into the interior space of the balloon at a pressure of less than 10 atmospheres, less than 5 atmospheres, less than 4 atmospheres, less than 3 atmospheres, less than 2 atmospheres, or less than 1 atmosphere.

Example 69. The expandable metallic implant device of example 1, wherein the expandable metallic implant and the balloon are separated, or can be separated, when they are expanded.

Example 70. The expandable metallic implant device of example 1, wherein the expansion of the expandable metallic implant results in a foreshortening of the expandable metallic implant along a plane parallel to the first axis of the expandable metallic implant.

Example 71. The expandable metallic implant device of example 1, wherein the expansion of the expandable metallic implant does not result in a foreshortening of the expandable metallic implant along a plane parallel to the first axis of the expandable metallic implant.

Example 72. The expandable metallic implant device of example 1, wherein the expansion of the folded and pleated expandable metallic implant results in a foreshortening of the expandable metallic implant along a plane parallel to the first axis of the expandable metallic implant that is 0%, <1%, <2%, <3%, <5%, or <10%, or ≥10% of the length of the folded and pleated expandable metallic implant, excluding any neck or neck assembly, including any guidewire segment.

Example 73. The expandable metallic implant device of example 1, wherein the outer diameter of the elongate body of the balloon catheter is between 0.010-0.070 inches.

Example 74. The expandable metallic implant device of example 1, wherein the length of the elongate body of the balloon catheter is between 26-201 cm.

Example 75. The expandable metallic implant device of example 1, wherein the elongate body of the balloon catheter comprises: (a) an outer layer comprising polymer; (b) an inner layer comprising polymer; and (c) a middle layer comprising metal located between the outer layer and inner layer.

Example 76. The expandable metallic implant device of example 75, wherein the inner or outer layer of the elongate body of the balloon catheter comprises polymer with a successively decreasing durometer from the proximal to the distal end of the elongate body.

Example 77. The expandable metallic implant device of example 75, wherein the distal portion of the outer layer of the elongate body of the balloon catheter comprises an aliphatic polyether polyurethane or a polyether block amide.

Example 78. The expandable metallic implant device of example 75, wherein the middle portion of the outer layer of the elongate body of the balloon catheter comprises a polyether block amide or a nylon.

Example 79. The expandable metallic implant device of example 75, wherein the proximal portion of the outer layer of the elongate body of the balloon catheter comprises a nylon.

Example 80. The expandable metallic implant device of example 75, wherein the wall of the elongate body of the balloon catheter comprises one or more liquid crystal polymer fibers.

Example 81. The expandable metallic implant device of example 80, wherein the one or more liquid crystal polymer fibers are oriented parallel to the first axis of the elongate body of the balloon catheter.

Example 82. The expandable metallic implant device of example 75, wherein the wall of the elongate body of the balloon catheter further comprises a tie layer.

Example 83. The expandable metallic implant device of example 75, wherein at least a portion of a polymer layer of the elongate body of the balloon catheter comprises barium sulfate.

Example 84. The expandable metallic implant device of example 83, wherein the barium sulfate is present at a concentration of 10-30%.

Example 85. The expandable metallic implant device of example 75, wherein at least some of the metal in the middle layer of the elongate body of the balloon catheter comprises nitinol or stainless steel.

Example 86. The expandable metallic implant device of example 75, wherein at least some of the metal in the middle layer of the elongate body of the balloon catheter is configured as wire with a cross-sectional shape that is round, oval, square, or rectangular.

Example 87. The expandable metallic implant device of example 75, wherein at least some of the metal in the middle layer of the elongate body of the balloon catheter is configured as wire in a braid or coil pattern.

Example 88. The expandable metallic implant device of example 75, wherein at least some of the metal in the middle layer of the elongate body of the balloon catheter is configured as a laser cut nitinol tube.

Example 89. The expandable metallic implant device of example 75, wherein the inner layer of the elongate body of the balloon catheter is lubricious.

Example 90. The expandable metallic implant device of example 75, wherein the inner layer of the elongate body of the balloon catheter comprises PTFE.

Example 91. The expandable metallic implant device of example 75, wherein the balloon catheter comprises: (a) a single marker band that is conspicuous during fluoroscopy configured for identifying the tip region of the balloon catheter during fluoroscopy; (b) two marker bands that are conspicuous during fluoroscopy configured for identifying the proximal and distal regions of the balloon during fluoroscopy; or (c) a single marker band that is conspicuous during fluoroscopy configured for identifying the tip region of the balloon catheter during fluoroscopy and two marker bands that are conspicuous during fluoroscopy configured for identifying the proximal and distal regions of the balloon during fluoroscopy.

Example 92. The expandable metallic implant device of example 1, wherein the outer surface of the elongate body of the balloon catheter comprises a lubricious coating.

Example 93. The expandable metallic implant device of example 1, wherein the distal portion of the elongate body of the balloon catheter, when in an unconstrained configuration, is straight.

Example 94. The expandable metallic implant device of example 1, wherein the distal portion of the elongate body of the balloon catheter, when in an unconstrained configuration, has a pre-formed shape.

Example 95. The expandable metallic implant device of example 94, wherein the distal portion of the elongate body of the balloon catheter is angled.

Example 96. The expandable metallic implant device of example 95, wherein the angle is 10-70 degrees.

Example 97. The expandable metallic implant device of example 1, that is configured for use to obstruct or reduce the rate of blood flow in an artery vein, or other blood-containing structure.

Example 98. A guide catheter or guide sheath for use in facilitating the delivery of devices in a human patient, the guide catheter or guide sheath comprising: (a) an elongate body; and (b) a hub joined or bonded to the proximal end the elongate body.

Example 99. The guide catheter or guide sheath of example 98, wherein the elongate body of the guide catheter or guide sheath comprises: (a) an outer layer comprising polymer; (b) an inner layer comprising polymer; and (c) a middle layer comprising metal disposed between the outer layer and an inner layer.

Example 100. The guide catheter or guide sheath of example 99, wherein the outer layer of the elongate body comprises polymer with successively decreasing durometer from the proximal to the distal end of the elongate body.

Example 101. The guide catheter or guide sheath of example 99, wherein the distal portion of the outer layer of the elongate body comprises an aliphatic polyether polyurethane or a polyether block amide.

Example 102. The guide catheter or guide sheath of example 99, wherein the middle portion of the outer layer of the elongate body comprises a polyether block amide or a nylon.

Example 103. The guide catheter or guide sheath of example 99, wherein the proximal portion of the outer layer of the elongate body comprises a nylon.

Example 104. The guide catheter or guide sheath of example 99, wherein the wall of the elongate body of the guide catheter or guide sheath comprises one or more liquid crystal polymer fibers.

Example 105. The guide catheter or guide sheath of example 104, wherein the one or more liquid crystal polymer fibers are oriented parallel to the first axis of the elongate body.

Example 106. The guide catheter or guide sheath of example 99, wherein the wall of the elongate body of the guide catheter or guide sheath further comprises a tie layer.

Example 107. The guide catheter or guide sheath of example 99, wherein at least a portion of the outer layer of the elongate body of the guide catheter or guide sheath comprises barium sulfate.

Example 108. The guide catheter or guide sheath of example 107, wherein the barium sulfate is present at a concentration of 10-30%.

Example 109. The guide catheter or guide sheath of example 99, wherein at least some of the metal in the middle layer of the elongate body comprises nitinol or stainless steel.

Example 110. The guide catheter or guide sheath of example 99, wherein at least some of the metal in the middle layer of the elongate body is configured as wire with a cross-sectional shape that is round, oval, square, or rectangular.

Example 111. The guide catheter or guide sheath of example 99, wherein at least some of the metal in the middle layer of the elongate body is configured as wire in a braid or coil pattern.

Example 112. The guide catheter or guide sheath of example 99, wherein at least some of the metal in the middle layer of the elongate body is configured as a laser cut nitinol tube.

Example 113. The guide catheter or guide sheath of example 99, wherein the inner layer is lubricious.

Example 114. The guide catheter or guide sheath of example 99, wherein the inner layer comprises PTFE.

Example 115. The guide catheter or guide sheath of example 98, wherein the elongate body of the guide catheter or guide sheath comprises: (a) a single marker band that is conspicuous during fluoroscopy configured for identifying the tip region of the guide catheter or guide sheath during fluoroscopy; (b) a single marker band that is conspicuous during fluoroscopy at a location <10 mm proximal to the distal end of the elongate body; (c) 2-5 marker bands that are conspicuous during fluoroscopy and configured for making measurements of the diameter of blood-containing structures during angiography; (d) 2-5 marker bands that are conspicuous during fluoroscopy at a location >10 mm proximal to the distal end of the elongate body; (e) a single marker band that is conspicuous during fluoroscopy configured for identifying the tip region of the guide catheter or guide sheath during fluoroscopy and two or three additional marker bands that are conspicuous during fluoroscopy and configured for making measurements of the diameter of blood-containing structures during angiography; or (f) a single marker band that is conspicuous during fluoroscopy at a location >10 mm proximal to the distal end of the elongate body and two or three additional marker bands at a location >10 mm proximal to the distal end of the elongate body.

Example 116. The guide catheter or guide sheath of example 115, wherein the 2-5 marker bands are located on portion of the elongate body that is straight when in an unconstrained configuration.

Example 117. The guide catheter or guide sheath of example 98, wherein the hub of the guide catheter or guide sheath comprises a rotational hemostatic valve on the distal end and a side arm for administration of fluid into the lumen of the intermediate catheter.

Example 118. The guide catheter or guide sheath of example 98, wherein the outer diameter of the distal end of the elongate body of the guide catheter or guide sheath has a radius.

Example 119. The guide catheter or guide sheath of example 98, wherein the outer surface of the elongate body of the guide catheter or guide sheath comprises a lubricious coating.

Example 120. The guide catheter or guide sheath of example 98, wherein the distal portion of the elongate body of the guide catheter or guide sheath, when in an unconstrained configuration, is straight.

Example 121. The guide catheter or guide sheath of example 98, wherein the distal portion of the elongate body of the guide catheter or guide sheath, when in an unconstrained configuration, has a pre-formed shape.

Example 122. The guide catheter or guide sheath of example 98, wherein the inner diameter of the elongate body is between 0.050-0.110 inches or between 0.050-0.145 inches.

Example 123. The guide catheter or guide sheath of example 98, wherein the length of the guide catheter or guide sheath is between 25-200 cm.

Example 124. The guide catheter or guide sheath of example 98 that is configured for delivering devices capable of obstructing or reducing the flow of blood or other biological fluids.

Example 125. An intermediate catheter for use in facilitating the advancement of a guide catheter or guide sheath in a human patient, the intermediate catheter comprising: (a) an elongate body with a single lumen; and (b) a hub joined or bonded to the proximal end the elongate body, (c) wherein the elongate body has a smaller inner and outer diameter at the distal end when compared with the proximal end.

Example 126. The intermediate catheter of example 125, wherein the elongate body of the intermediate catheter comprises: (a) an outer layer comprising polymer; (b) an inner layer comprising polymer; and (c) a middle layer comprising metal disposed between the outer layer and an inner layer.

Example 127. The intermediate catheter of example 126, wherein the outer layer of the elongate body comprises polymer with decreasing durometer from the proximal to the distal end of the elongate body.

Example 128. The intermediate catheter of example 126, wherein the distal portion of the outer layer of the elongate body comprises an aliphatic polyether polyurethane or a polyether block amide.

Example 129. The intermediate catheter of example 126, wherein the middle portion of the outer layer of the elongate body comprises a polyether block amide or a nylon.

Example 130. The intermediate catheter of example 126, wherein the proximal portion of the outer layer of the elongate body comprises a nylon.

Example 131. The intermediate catheter of example 126, wherein the wall of the elongate body of the guide catheter or guide sheath comprises one or more liquid crystal polymer fibers.

Example 132. The intermediate catheter of example 131, wherein the one or more liquid crystal polymer fibers are oriented parallel to the first axis of the elongate body.

Example 133. The intermediate catheter of example 126, wherein the wall of the elongate body of the guide catheter or guide sheath further comprises a tie layer.

Example 134. The intermediate catheter of example 126, wherein at least a portion of the outer layer of the elongate body of the guide catheter or guide sheath comprises barium sulfate.

Example 135. The intermediate catheter of example 134, wherein the barium sulfate is present at a concentration of 10-30%.

Example 136. The intermediate catheter of example 126, wherein at least some of the metal in the middle layer of the elongate body comprises nitinol or stainless steel.

Example 137. The intermediate catheter of example 126, wherein at least some of the metal in the middle layer of the elongate body is configured as wire with a cross-sectional shape that is round, oval, square, or rectangular.

Example 138. The intermediate catheter of example 126, wherein at least some of the metal in the middle layer of the elongate body is configured as wire in a braid or coil pattern.

Example 139. The intermediate catheter of example 126, wherein at least some of the metal in the middle layer of the elongate body is configured as a laser cut nitinol tube.

Example 140. The intermediate catheter of example 126, wherein the inner layer is lubricious.

Example 141. The intermediate catheter of example 126, wherein the inner layer comprises PTFE.

Example 142. The intermediate catheter of example 125, wherein the elongate body of the intermediate catheter comprises: (a) a single marker band that is conspicuous during fluoroscopy configured for identifying the tip region of the intermediate catheter during fluoroscopy; or (b) a single marker band that is conspicuous during fluoroscopy at a location <10 mm proximal to the distal end of the elongate body.

Example 143. The intermediate catheter of example 125, wherein the distal portion of the elongate body of the intermediate catheter is tapered.

Example 144. The intermediate catheter of example 143, wherein the length of the tapered distal portion of the elongate body of the intermediate catheter with the smaller inner and outer diameter is between 1-40 cm.

Example 145. The intermediate catheter of example 125, wherein the inner diameter of the distal portion of the elongate body is between 0.066-0.076 inches.

Example 146. The intermediate catheter of example 145, wherein the length of the distal portion of the elongate body of the intermediate catheter with the inner diameter of 0.066-0.076 inches is between 1-40 cm.

Example 147. The intermediate catheter of example 125, wherein the distal portion of the elongate body of the intermediate catheter, when in an unconstrained configuration, has a pre-formed shape.

Example 148. The intermediate catheter of example 125, wherein the distal portion of the elongate body of the intermediate catheter, when in an unconstrained configuration, is straight.

Example 149. The intermediate catheter of example 125, wherein the outer diameter of the distal end of the elongate body of the intermediate catheter has a radius.

Example 150. The intermediate catheter of example 125, wherein the outer surface of the elongate body of the intermediate catheter comprises a lubricious coating.

Example 151. The intermediate catheter of example 125, wherein the hub of the intermediate catheter comprises a rotational hemostatic valve on the distal end and a side arm for administration of fluid into the lumen of the intermediate catheter.

Example 152. The intermediate catheter of example 125, wherein the length of the intermediate catheter is between 30-240 cm.

Example 153. A selective catheter for use in facilitating the advancement of a guide catheter or guide sheath, the advancement of a guide catheter or guide sheath and an intermediate catheter that are joined together, or the advancement of a guidewire in a human patient, the selective catheter comprising: (a) an elongate body with a single lumen, wherein the distal portion of the elongate body, when in an unconstrained configuration, has a pre-formed shape; and (b) a hub joined or bonded to the proximal end the elongate body.

Example 154. The selective catheter of example 153, wherein the elongate body of the intermediate catheter comprises: (a) an outer layer comprising polymer; (b) an inner layer comprising polymer; and (c) a middle layer comprising metal disposed between the outer layer and an inner layer.

Example 155. The selective catheter of example 154, wherein the outer layer of the elongate body comprises polymer with successively decreasing durometer from the proximal to the distal end of the elongate body.

Example 156. The selective catheter of example 154, wherein the distal portion of the outer layer of the elongate body comprises an aliphatic polyether polyurethane or a polyether block amide.

Example 157. The selective catheter of example 154, wherein the middle portion of the outer layer of the elongate body comprises a polyether block amide or a nylon.

Example 158. The selective catheter of example 154, wherein the proximal portion of the outer layer of the elongate body comprises a nylon.

Example 159. The selective catheter of example 154, wherein the wall of the elongate body of the selective catheter comprises one or more liquid crystal polymer fibers.

Example 160. The selective catheter of example 154, wherein the wall of the elongate body of the selective catheter further comprises a tie layer.

Example 161. The selective catheter of example 154, wherein at least a portion of the outer layer of the elongate body of the selective catheter comprises barium sulfate.

Example 162. The selective catheter of example 161, wherein the barium sulfate is present at a concentration of 10-30%.

Example 163. The selective catheter of example 154, wherein at least some of the metal in the middle layer of the elongate body comprises nitinol or stainless steel.

Example 164. The selective catheter of example 154, wherein at least some of the metal in the middle layer of the elongate body is configured as wire with a cross-sectional shape that is round, oval, square, or rectangular.

Example 165. The selective catheter of example 154, wherein at least some of the metal in the middle layer of the elongate body is configured as wire in a braid or coil pattern.

Example 166. The selective catheter of example 154, wherein at least some of the metal in the middle layer of the elongate body is configured as a laser cut nitinol tube.

Example 167. The selective catheter of example 154, wherein the inner layer is lubricious.

Example 168. The selective catheter of example 154, wherein the inner layer comprises PTFE.

Example 169. The selective catheter of example 153, wherein the elongate body of the selective catheter comprises: (a) a single marker band that is conspicuous during fluoroscopy configured for identifying the tip region of the intermediate catheter during fluoroscopy; or (b) a single marker band that is conspicuous during fluoroscopy at a location <10 mm proximal to the distal end of the elongate body.

Example 170. The selective catheter of example 154, wherein the distal portion of the elongate body is tapered.

Example 171. The selective catheter of example 154, wherein the outer diameter of the distal end of the elongate body has a radius.

Example 172. The selective catheter of example 154, wherein the outer surface of the elongate body comprises a lubricious coating.

Example 173. The selective catheter of example 154, wherein the inner diameter of the elongate body is between 0.040-0.054 inches.

Example 174. The selective catheter of example 154, wherein the length of the selective catheter is between 35-245 cm.

Example 175. A coiling catheter for use in facilitating the placement of coil expandable implant devices in a human patient, the coiling catheter comprising: (a) an elongate body with a single lumen; and (b) a hub joined or bonded to the proximal end the elongate body.

Example 176. The coiling catheter of example 175, wherein the elongate body of the intermediate catheter comprises: (a) an outer layer comprising polymer; (b) an inner layer comprising polymer; and (c) a middle layer comprising metal disposed between the outer layer and an inner layer.

Example 177. The coiling catheter of example 176, wherein the outer layer of the elongate body comprises polymer with successively decreasing durometer from the proximal to the distal end of the elongate body.

Example 178. The coiling catheter of example 176, wherein the distal portion of the outer layer of the elongate body comprises an aliphatic polyether polyurethane or a polyether block amide.

Example 179. The coiling catheter of example 176, wherein the middle portion of the outer layer of the elongate body comprises a polyether block amide or a nylon.

Example 180. The coiling catheter of example 176, wherein the proximal portion of the outer layer of the elongate body comprises a nylon.

Example 181. The coiling catheter of example 176, wherein the wall of the elongate body of the coiling catheter comprises one or more liquid crystal polymer fibers.

Example 182. The coiling catheter of example 181, wherein the one or more liquid crystal polymer fibers are oriented parallel to the first axis of the elongate body.

Example 183. The coiling catheter of example 176, wherein the wall of the elongate body of the coiling catheter further comprises a tie layer.

Example 184. The coiling catheter of example 176, wherein at least a portion of the outer layer of the elongate body of the coiling catheter comprises barium sulfate.

Example 185. The coiling catheter of example 184, wherein the barium sulfate is present at a concentration of 10-30%.

Example 186. The coiling catheter of example 176, wherein at least some of the metal in the middle layer of the elongate body comprises nitinol or stainless steel.

Example 187. The coiling catheter of example 176, wherein at least some of the metal in the middle layer of the elongate body is configured as wire with a cross-sectional shape that is round, oval, square, or rectangular.

Example 188. The coiling catheter of example 176, wherein at least some of the metal in the middle layer of the elongate body is configured as wire in a braid or coil pattern.

Example 189. The coiling catheter of example 176, wherein at least some of the metal in the middle layer of the elongate body is configured as a laser cut nitinol tube.

Example 190. The coiling catheter of example 176, wherein the inner layer is lubricious.

Example 191. The coiling catheter of example 176, wherein the inner layer comprises PTFE.

Example 192. The coiling catheter of example 175, wherein the elongate body of the coiling catheter comprises: (a) a single marker band that is conspicuous during fluoroscopy configured for identifying the tip region of the coiling catheter during fluoroscopy; (b) a single marker band that is conspicuous during fluoroscopy at a location <10 mm proximal to the distal end of the elongate body; (c) two marker bands that are conspicuous during fluoroscopy and configured for assisting the detachment of coil expandable implants; (d) two marker bands that are located on a straight portion of the elongate body at a location >10 mm proximal to the distal end of the elongate body; (e) a single marker band that is conspicuous during fluoroscopy configured for identifying the tip region of the coiling catheter during fluoroscopy and two additional marker bands that are conspicuous during fluoroscopy and configured for assisting the detachment of coil expandable implants; or (f) a single marker band that is conspicuous during fluoroscopy at a location <10 mm proximal to the distal end of the elongate body and two additional marker bands that are located on a straight portion of the elongate body at a location >10 mm proximal to the distal end of the elongate body.

Example 193. The coiling catheter of example 175, wherein the distal portion of the elongate body of the coiling catheter, when in an unconstrained configuration, has a pre-formed shape.

Example 194. The coiling catheter of example 175, wherein the distal portion of the elongate body of the coiling catheter, when in an unconstrained configuration, is straight.

Example 195. The coiling catheter of example 175, wherein the outer diameter of the distal end of the elongate body has a radius.

Example 196. The coiling catheter of example 175, wherein the outer surface of the elongate body comprises a lubricious coating.

Example 197. The coiling catheter of example 175, wherein the inner diameter of the elongate body is between 0.017-0.054 inches.

Example 198. The coiling catheter of example 175, wherein the length of the coiling catheter is between 40-250 cm.

Example 199. A coil expandable implant device for use in obstructing or reducing the flow of blood or other biological fluids in a human patient, the device comprising: (a) a complex, flexible, elongated metallic structure with a pre-formed shape configured for permanent implantation in a patient; and (b) an elongate body reversibly joined to the complex, elongated metallic structure and configured to advance or retract the complex, elongated metallic structure in a human patient.

Example 200. The coil expandable implant device of example 199, comprising an inner core wire surrounded by an outer coiled or externally wound wire.

Example 201. The coil expandable implant device of example 200, wherein the middle portion of the inner core wire has a diameter that is greater than the diameter of the distal end, the proximal end, or both the distal and proximal end portion of the inner core wire.

Example 202. The coil expandable implant device of example 201, wherein the inner core wire comprises stainless steel or nitinol, or combinations thereof.

Example 203. The coil expandable implant device of example 201, wherein outer coiled or externally wound wire comprises platinum, gold, or combinations thereof.

Example 204. The coil expandable implant device of example 199, comprising a coating or adherent threads or fibers or other material to promote thrombosis.

Example 205. The coil expandable implant device of example 199, wherein the secondary diameter of the complex, elongated metallic structure is between 0.010-0.038 inches.

Example 206. The coil expandable implant device of example 199, wherein the tertiary diameter of the complex, elongated metallic structure is 2-100 millimeters.

Example 207. The coil expandable implant device of example 199, wherein the elongate body is a wire, laser cut nitinol tube, or catheter.

Example 208. The coil expandable implant device of example 199, wherein the complex, elongated metallic structure is detached from the elongate body by mechanical, electrolytic, or thermal means.

Example 209. A guidewire for use in advancing a guide catheter or guide sheath, intermediate catheter, selective catheter, or coiling catheter in a human patient, the guidewire comprising: (a) an inner core wire; and (b) an outer coiled or externally wound wire.

Example 210. The guidewire of example 209, wherein the middle portion of the inner core wire has a diameter that is greater than the diameter of the distal end, the proximal end, or both the distal and proximal end of the inner core wire.

Example 211. The guidewire of example 209, wherein the inner core wire comprises stainless steel, nitinol, or combinations thereof.

Example 212. The guidewire of example 209, wherein the outer coiled or externally wound wire comprises platinum, gold, stainless steel, nitinol, or combinations thereof.

Example 213. The guidewire of example 209, comprising a lubricious coating or covering.

Example 214. The guidewire of example 209, wherein the secondary diameter of the guidewire is between 0.010-0.038 inches.

Example 215. The guidewire of example 209, wherein the secondary diameter of the guidewire is 0.010, 0.011, 0.012, 0.013, 0.014, 0.015, 0.016, 0.017, 0.018, 0.033, 0.034, 0.035, 0.036, 0.037, or 0.038 inches.

Example 216. The guidewire of example 209, wherein the guidewire has a length of 45-500 centimeters.

Example 217. A system comprising devices for use in obstructing or reducing the flow of blood or other biological fluids in a human patient, the system comprising a folded and pleated expandable metallic implant device of any one of examples 1-97 and a guide catheter or guide sheath of any one of examples 98-124.

Example 218. The system of example 217, wherein a portion of the elongate body of the expandable metallic implant device passes through the lumen of the guide catheter or guide sheath.

Example 219. The system of any one of examples 217 and 218, wherein the blood-containing structure is an artery, vein, or para-valvular leak pathway.

Example 220. The system of any one of examples 217 and 218, wherein the difference between the internal diameter of the guide catheter or guide sheath and the outer diameter of the elongate body of the expandable metallic implant device is >0.040 inches.

Example 221. The system of any one of examples 217 and 218, wherein the elongate body of the expandable metallic implant device is at least 1 cm longer than the length of the elongate body of the guide catheter or guide sheath.

Example 222. The system of any one of examples 217 and 218, wherein the lumen of the guide catheter or guide sheath is configured to allow for the passage of the folded and pleated portion of the expandable metallic implant device through the guide catheter or guide sheath and into the body of the patient distal to the guide catheter or guide sheath.

Example 223. The system of any one of examples 217 and 218, further comprising a Tuohy Borst adaptor operably and reversibly coupled to the hub of the guide catheter or guide sheath.

Example 224. The system of example 223, wherein the Tuohy Borst adaptor comprises a side arm configured for the injection of fluids, including water, saline, radiographic contrast, solutions comprising therapeutic agents or drugs, and mixtures therein.

Example 225. The system of any one of examples 217-224, wherein the guide catheter or guide sheath can be moved forward or backward while the folded and pleated expandable metallic implant device remains fixed in position.

Example 226. The system of any one of examples 217-224, wherein the folded and pleated expandable metallic implant device can be moved forward or backward while the guide catheter or guide sheath remains fixed in position.

Example 227. The system of any one of examples 217-226, further comprising a coiling catheter of any one of examples 175-198, and a coil expandable implant device of any one of examples 199-208.

Example 228. The system of example 227, wherein a portion of the elongate body of the coiling catheter passes through the lumen of the guide catheter or guide sheath adjacent to the elongate body of the expandable metallic implant device.

Example 229. The system of any one of examples 227 and 228, wherein the lumen of the guide catheter or guide sheath is configured to allow for the simultaneous passage of the elongate body of the expandable metallic implant device and the elongate body of the coiling catheter through the guide catheter or guide sheath.

Example 230. The system of any one of examples 227 and 228, wherein the difference between the internal diameter of the guide catheter or guide sheath and the outer diameter of the elongate body of the expandable metallic implant device is >0.040 inches.

Example 231. The system of any one of examples 227 and 228, wherein, when the proximal end of the balloon of an expandable metallic implant device is distal to the distal end of the guide catheter or guide sheath, a coiling catheter can be passed through the lumen of the guide catheter or guide sheath.

Example 232. The system of any one of examples 227 and 228, wherein the elongate body of the coiling catheter is at least 1 cm longer than the length of the elongate body of the expandable metallic implant device.

Example 233. The system of example 227 and 228, wherein a Tuohy Borst adaptor is operably and reversibly coupled to the hub of the coiling catheter, and the coil expandable implant device is inserted into the lumen of the coiling catheter through the Tuohy Borst adaptor.

Example 234. The system of example 233, wherein the Tuohy Borst adaptor comprises a side arm for the injection of fluids, including water, saline, radiographic contrast, solutions comprising therapeutic agents or drugs, and mixtures therein.

Example 235. The system of any one of examples 227-234, wherein the coiling catheter can be moved forward or backward while the guide catheter or guide sheath and the expandable metallic implant device remain fixed in position.

Example 236. The system of any one of examples 227-234, wherein the expandable metallic implant device can be moved forward or backward while the guide catheter or guide sheath and the coiling catheter remain fixed in position.

Example 237. The system of any one of examples 227-234, wherein the guide catheter or guide sheath can be moved forward or backward while the expandable metallic implant device and the coiling catheter remain fixed in position.

Example 238. The system of any one of examples 227-234, wherein the coil expandable implant device can be moved forward or backward while the guide catheter or guide sheath, expandable metallic implant device, and the coiling catheter remain fixed in position.

Example 239. A system comprising devices for use in advancing a guide catheter or guide sheath in a human patient, the system comprising a guide catheter or guide sheath of any one of examples 98-124 and a guidewire of any one of examples 209-216.

Example 240. The system of example 239, wherein the outer diameter of the guidewire is at least 0.002 inches smaller than the inner diameter of the guide catheter or guide sheath.

Example 241. The system of any one of examples 239 and 240, wherein a portion of the guidewire passes through the lumen of the guide catheter or guide sheath.

Example 242. The system of any one of examples 239 and 240, wherein the length of the guidewire is at least 1 cm longer than the length of the guide catheter or guide sheath.

Example 243. The system of any one of examples 239 and 240, wherein the lumen of the guide catheter or guide sheath is configured to allow for the passage of the guidewire through the guide catheter or guide sheath.

Example 244. The system of any one of examples 239 and 240, wherein a Tuohy Borst adaptor is operably and reversibly coupled to the hub of the guide catheter or guide sheath, and the guidewire is inserted into the lumen of the guide catheter or guide sheath through the Tuohy Borst adaptor.

Example 245. The system of any one of examples 244, wherein the Tuohy Borst adaptor comprises a side arm for the injection of fluids, including water, saline, radiographic contrast, solutions comprising therapeutic agents or drugs, and mixtures therein.

Example 246. The system of any one of examples 239-245, wherein the guidewire can be moved forward or backward while the guide catheter or guide sheath remains fixed in position.

Example 247. The system of any one of examples 239-245, wherein the guide catheter or guide sheath can be moved forward or backward while the guidewire remains fixed in position.

Example 248. The system of any one of examples 239-247, further comprising an intermediate catheter of any one of examples 125-152.

Example 249. The system of example 248, wherein: (a) the outer diameter of the proximal portion of the intermediate catheter is at least 0.002 inches smaller than the inner diameter of the guide catheter or guide sheath; and (b) the outer diameter of the guidewire is at least 0.002 inches smaller than the inner diameter of the distal portion of the intermediate catheter.

Example 250. The system of any one of examples 248 and 249, wherein a portion of the elongate body of the intermediate catheter passes through the lumen of the guide catheter or guide sheath.

Example 251. The system of any one of examples 248 and 249, wherein a portion of the guidewire passes through the lumen of the intermediate catheter.

Example 252. The system of any one of examples 248 and 249, wherein the intermediate catheter is at least 1 cm longer than the length of the guide catheter or guide sheath.

Example 253. The system of any one of examples 248 and 249, wherein the guidewire is at least 1 cm longer than the length of the intermediate catheter.

Example 254. The system of any one of examples 248 and 249, wherein the lumen of the intermediate catheter is configured to allow for the passage of the guidewire through the intermediate catheter.

Example 255. The system of any one of examples 248 and 249, wherein the hub of the intermediate catheter and the hub of the guide catheter or guide sheath can be reversibly joined or engaged.

Example 256. The system of any one of examples 255, wherein the reversible engagement or joining of the hubs of the intermediate catheter and guide catheter or guide sheath prevents or reduces the leaking of blood from the guide catheter or guide sheath during use.

Example 257. The system of any one of examples 255, wherein, once joined, the hub of the intermediate catheter and guide catheter or guide sheath can be separated, and the intermediate catheter can be removed from the guide catheter or guide sheath.

Example 258. The system of any one of examples 255-257, wherein, the joining or engagement and the separation of the hubs can occur by rotating a lock.

Example 259. The system of any one of examples 248 and 249, wherein a Tuohy Borst adaptor is operably and reversibly coupled to the hub of the intermediate catheter, and the guidewire is inserted into the lumen of the intermediate catheter through the Tuohy Borst adaptor.

Example 260. The system of any one of examples 259, wherein the Tuohy Borst adaptor comprises a side arm for the injection of fluids, including water, saline, radiographic contrast, solutions comprising therapeutic agents or drugs, and mixtures therein.

Example 261. The system of any one of examples 246-262, wherein the guidewire can be moved forward or backward while the guide catheter or guide sheath and intermediate catheter remain fixed in position.

Example 262. The system of any one of examples 246-262, wherein the intermediate catheter can be moved forward or backward while the guide catheter or guide sheath and the guidewire remain fixed in position.

Example 263. The system of any one of examples 246-262, wherein the guide catheter or guide sheath can be moved forward or backward while the intermediate catheter and the guidewire remain fixed in position.

Example 264. The system of any one of examples 246-262, wherein the guide catheter or guide sheath and intermediate catheter can be moved forward or backward while the guidewire remains fixed in position.

Example 265. The system of any one of examples 248-264, further comprising a selective catheter of any one of examples 148-171.

Example 266. The system of example 265, wherein: (a) the inner diameter of the elongate body of the selective catheter is <0.058″ inches; (b) the outer diameter of the selective catheter is at least 0.002 inches smaller than the inner diameter of the distal portion of the intermediate catheter; (c) the outer diameter of the guidewire is at least 0.002 inches smaller than the inner diameter of the selective catheter; and (d) the distal portion of the elongate body of the selective catheter, when in an unconstrained configuration, has a pre-formed shape.

Example 267. The system of any one of examples 265 and 266, wherein a portion of the elongate body of selective catheter passes through the lumen of the intermediate catheter.

Example 268. The system of any one of examples 265 and 266, wherein a portion of the guidewire passes through the lumen of the selective catheter.

Example 269. The system of any one of examples 265 and 266, wherein the selective catheter is at least 1 cm longer than the length of the intermediate catheter.

Example 270. The system of any one of examples 265 and 266, wherein the guidewire is at least 1 cm longer than the length of the selective catheter.

Example 271. The system of any one of examples 265 and 266, wherein the lumen of the intermediate catheter is configured to allow for the passage of the selective catheter through the intermediate catheter.

Example 272. The system of any one of examples 265 and 266, wherein the lumen of the selective catheter is configured to allow for the passage of the guidewire through the selective catheter.

Example 273. The system of any one of examples 265 and 266, wherein a Tuohy Borst adaptor is operably and reversibly coupled to the hub of the selective catheter, and the guidewire is inserted into the lumen of the selective catheter through the Tuohy Borst adaptor.

Example 274. The system of any one of examples 273, wherein the Tuohy Borst adaptor comprises a side arm for the injection of fluids, including water, saline, radiographic contrast, solutions comprising therapeutic agents or drugs, and mixtures therein.

Example 275. The system of any one of examples 265-274, wherein the selective catheter can be moved forward or backward while the guide catheter or guide sheath, intermediate catheter, and guidewire remain fixed in position.

Example 276. The system of any one of examples 265-274, wherein the guide catheter or guide sheath and intermediate catheter can be moved forward or backward while the selective catheter and guidewire remain fixed in position.

Example 277. The system of any one of examples 265-274, wherein the intermediate catheter can be moved forward or backward while the guide catheter or guide sheath, selective catheter, and the guidewire remain fixed in position.

Example 278. The system of any one of examples 265-274, wherein the selective catheter can be moved forward or backward while the guide catheter or guide sheath, intermediate catheter, and the guidewire remain fixed in position.

Example 279. The system of any one of examples 265-274, wherein the guidewire can be moved forward or backward while the guide catheter or guide sheath, intermediate catheter, and selective catheter remain fixed in position.

Example 280. A method for obstructing or reducing the rate of blood flow in an artery or vein of a human patient using a system of devices, the method comprising: (a) providing an expandable metallic implant device of any one of examples 1-97; (b) providing a guide catheter or guide sheath of any one of examples 98-124; (c) providing a guidewire of any one of examples 209-216; (d) inserting the guide catheter or guide sheath into an artery or vein of the human patient and advancing the distal end of the guide catheter or guide sheath to a segment of artery or vein indicated for an obstruction or reduction in the rate of blood flow, optionally using a guidewire, selective catheter, and/or intermediate catheter to assist the advancement; (e) injecting contrast material through the guide catheter or guide sheath and into the segment of artery or vein indicated for obstruction or reduction in the rate of blood flow during angiography; (f) making a measurement of the diameter of the segment of artery or vein indicated for obstruction or reduction in the rate of blood flow using the marker bands of the guide catheter or guide sheath; (g) selecting an expandable metallic implant device with an appropriately sized expandable metallic implant; (h) inserting the distal portion of the expandable metallic implant device through the hub of the guide catheter or guide sheath and into the elongate body of the guide catheter or guide sheath, optionally first inserting the distal portion of the expandable metallic implant device through a Tuohy Borst adapter joined to the hub of the guide catheter or guide sheath; (i) advancing the folded and pleated expandable metallic implant through the guide catheter or guide sheath using the balloon catheter until the folded and pleated expandable metallic implant exits the distal end of the guide catheter or guide sheath and is positioned in the segment of artery or vein indicated for obstruction or reduction in the rate of blood flow; (j) delivering a fluid medium: 1) into the hub of the balloon catheter; 2) through the elongate body of the balloon catheter; and 3) into the interior space of the balloon of the balloon catheter, causing the folded and pleated balloon and expandable metallic implant to expand; (k) removing fluid medium from the interior space of the balloon of the balloon catheter, causing the balloon to deflate or collapse while the expandable metallic implant remains in an expanded configuration; and (1) removing the balloon catheter and the guide catheter or guide sheath from the patient.

Example 281. The method of example 280, wherein the expandable metallic implant of the expandable metallic implant device has an expanded diameter that is 10%-50% larger than the diameter of the lumen of the segment of artery or vein indicated for obstruction or reduction in the rate of blood flow.

Example 282. The method of example 280, wherein a guidewire segment joined or bonded to the neck or neck assembly of the folded and pleated expandable metallic implant is used to guide the folded and pleated expandable metallic implant into and through the guide catheter or guide sheath and into position in the segment of artery or vein indicated for obstruction or reduction in the rate of blood flow.

Example 283. The method of example 280, wherein the balloon is compliant or semi-compliant, and the expandable metallic implant comprises one or more flaps, and the balloon is inflated at a pressure that raises the free end of at least one flap from the surface of the expandable metallic implant during balloon inflation.

Example 284. The method of example 280, wherein, after removing fluid medium from the interior space of the balloon, the distal tip of the guide catheter or guide sheath is advanced over the balloon prior to removing the balloon catheter and the guide catheter or guide sheath from the patient.

Example 285. The method of example 280, further comprising coupling a Tuohy Borst adaptor with a side arm for injection of fluids into the guide catheter or guide sheath and injecting radiographic contrast material through the guide catheter or guide sheath and into the artery or vein indicated for obstruction or reduction in the rate of blood flow using the Tuohy Borst adaptor.

Example 286. The method of example 285, wherein radiographic contrast material is injected into the guide catheter or guide sheath: (a) after positioning the folded and pleated expandable metallic implant in the artery or vein but before the expansion of the folded and pleated expandable metallic implant; (b) after expansion of the expandable metallic implant; or (c) after removal of the balloon catheter from the patient.

Example 287. A method for obstructing or reducing the rate of blood flow in an artery or vein of a human patient using a system of devices, the method comprising: (a) providing an expandable metallic implant device of any one of examples 1-97; (b) providing a guide catheter or guide sheath of any one of examples 98-124; (c) providing a coiling catheter of any one of examples 175-198; (d) providing one more guidewires of any one of examples 209-216; (e) providing one or more coil expandable implant devices of any one of examples 199-208; (f) inserting the guide catheter or guide sheath into an artery or vein of the human patient and advancing the distal end of the guide catheter or guide sheath to a segment of artery or vein indicated for an obstruction or reduction in the rate of blood flow, optionally using a guidewire, selective catheter, and/or intermediate catheter to assist advancement; (g) injecting contrast material through the guide catheter or guide sheath and into the segment of artery or vein indicated for obstruction or reduction in the rate of blood flow during angiography; (h) making a measurement of the diameter of the segment of artery or vein indicated for obstruction or reduction in the rate of blood flow using the marker bands of the guide catheter or guide sheath; (i) selecting an expandable metallic implant device with an appropriately sized expandable metallic implant; (j) inserting the distal portion of the expandable metallic implant device through the hub of the guide catheter or guide sheath and into the elongate body of the guide catheter or guide sheath, optionally first inserting the distal portion of the expandable metallic implant device through a Tuohy Borst adapter joined to the hub of the guide catheter or guide sheath; (k) advancing the folded and pleated expandable metallic implant through the guide catheter or guide sheath using the balloon catheter until the folded and pleated expandable metallic implant exits the distal end of the guide catheter or guide sheath and is positioned in the segment of artery or vein indicated for obstruction or reduction in the rate of blood flow; (1) inserting a guidewire into the lumen of the coiling catheter and inserting the coiling catheter and guidewire through the hub of the guide catheter or guide sheath and advancing the coiling catheter alongside the elongate body of the expandable metallic implant device until the distal end guidewire and coiling catheter exit the distal end of the guide catheter or guide sheath and the distal end of the guide catheter or guide sheath is positioned in artery or vein indicated for obstruction or reduction in the rate of blood flow to a position distal to the folded and pleated expandable metallic implant of the expandable metallic implant device; (m) delivering a fluid medium: 1) into the hub of the balloon catheter; 2) through the elongate body of the expandable metallic implant device; and 3) into the interior space of the balloon of the balloon catheter, causing the balloon and expandable metallic implant to assume a partially expanded configuration; (n) removing the guidewire from the coiling catheter; (o) advancing one or more coil expandable implant devices through the lumen of the coiling catheter and placing the one or more coil expandable implant devices in the segment of artery or vein indicated for obstruction or reduction in the rate of blood flow at a position distal to the partially expanded expandable metallic implant; (p) removing the coiling catheter from the patient; (q) delivering a fluid medium: 1) into the hub of the balloon catheter; 2) through the elongate body of the balloon catheter; and 3) into the interior space of the balloon of the balloon catheter, causing the expandable metallic implant to assume a fully expanded configuration; (r) removing fluid medium from the interior space of the balloon of the balloon catheter, causing the balloon to deflate or collapse while the expandable metallic implant remains in an expanded configuration; and(s) removing the balloon catheter and guide catheter or guide sheath from the patient.

Example 288. The method of example 287, wherein the expandable metallic implant of the expandable metallic implant device has an expanded diameter that is 10%-50% larger than the diameter of the lumen of the segment of artery or vein indicated for obstruction or reduction in the rate of blood flow.

Example 289. The method of example 287, wherein the guidewire segment joined or bonded to the neck or neck assembly of the expandable metallic implant is used to guide the folded and pleated expandable metallic implant into and through the guide catheter or guide sheath and into position in the segment of artery or vein indicated for obstruction or reduction in the rate of blood flow.

Example 290. The method of example 287, wherein the balloon is compliant or semi-compliant, and the expandable metallic implant comprises one or more flaps, and the balloon is inflated at a pressure that raises the free end of at least one flap from the surface of the expandable metallic implant during balloon inflation.

Example 291. The method of example 287, wherein, after removing fluid medium from the interior space of the balloon, the distal tip of the guide catheter or guide sheath is advanced over the balloon prior to removing the balloon catheter from the patient.

Example 292. The method of example 287, further comprising coupling a Tuohy Borst adaptor with a side arm for injection of fluids into the hub of the guide catheter or guide sheath and injecting radiographic contrast material through the guide catheter or guide sheath and into the artery or vein using the Tuohy Borst adaptor.

Example 293. The method of example 287, wherein radiographic contrast material is injected into the guide catheter or guide sheath: (a) after positioning the folded and pleated expandable metallic implant in the artery or vein but before the expansion of the folded and pleated balloon and expandable metallic implant; (b) after expansion of the folded and pleated balloon and expandable metallic implant; (c) after placement of one or more coil expandable implant device in the artery or vein; (d) after additional expansion of the expandable metallic implant; or (e) after removal of the balloon catheter from the patient.

Example 294. A method for obstructing or reducing a flow of blood in an artery or vein of a patient, the method comprising: placing a guidewire at an implantation site in an artery or vein of a patient; advancing a balloon catheter carrying an expandable metallic implant in a collapsed configuration over the guidewire to an implantation site in an artery or vein of a patient wherein the expandable metallic implant in the expanded configuration comprises a wall defining a hollow shape having a proximally facing opening at a proximal end thereof and a closed distal end except for a central opening to allow for the passage of the distal portion of the elongate body; inflating a balloon through an inflation lumen of the balloon catheter to cause the expandable metallic implant to expand to an expanded configuration that provides for an obstruction or reduction of flow of blood at the implantation site; and removing the balloon catheter and the guidewire from the patient while the expandable metallic implant remains at the implantation site in the patient in an expanded configuration wherein at least a portion of the central opening of the expandable metallic implant is covered after separation of the balloon catheter and the expandable metallic implant.

Example 295. The method of example 294, wherein the expandable metallic implant has a pleated and folded configuration when in the collapsed configuration.

Example 296. The method of any one of examples 294-295, wherein the expandable metallic implant in the expanded configuration comprises a wall defining a hollow shape having a proximally facing opening at a proximal end thereof and a closed or partially closed distal end, including wherein the end is closed or partially closed by one or more valves.

Example 297. The method of any one of examples 294-296, wherein the expandable metallic implant that engages with the artery or vein of the patient comprises a layer of gold with a thickness of between 5 microns and 100 microns.

Example 298. The method of any one of examples 294-297, wherein inflating the balloon comprises delivering between about 1 atm and about 10 atm of pressure to the balloon.

Example 299. The method of any one of examples 294-298, further comprising deflating the balloon such that the expandable metallic implant remains in the expanded configuration and engaged with a wall of an artery or vein of the patient.

Example 300. The method of any one of examples 294-299, wherein inflating the balloon causes a flap of the expandable metallic implant to extend radially outward from a surface of the expandable metallic implant.

Example 301. The method of any one of examples 294-300, further comprising delivering the balloon catheter and the expandable metallic implant to the implantation site through a guide catheter or guide sheath.

Example 302. The method of any one of examples 294-301, further comprising selecting the expandable metallic implant from a plurality of expandable metallic implants of different sizes based on a vessel size and shape at the implantation site.

### Additional Considerations and Terminology

Features, materials, characteristics, or groups described in conjunction with a particular aspect, example, or example are to be understood to be applicable to any other aspect, example or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing examples. The protection extends to any novel feature, or any novel combination of features disclosed in this specification (including any accompanying claims, abstract and drawings), or to any novel step, or any novel combination of steps of any method or process so disclosed.

While certain examples have been described, these examples have been presented by way of example only and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some examples, the actual steps taken in the processes illustrated or disclosed may differ from those shown in the figures. Depending on the example, certain of the steps described above may be removed, others may be added. For example, the actual steps or order of steps taken in the disclosed processes may differ from those described or shown in a figure. Depending on the example, certain of the steps described above may be removed, others may be added. Furthermore, the features and attributes of the specific examples disclosed above may be combined in different ways to form additional examples, all of which fall within the scope of the present disclosure.

Although the present disclosure includes certain examples, examples and applications, it will be understood by those skilled in the art that the present disclosure extends beyond the specifically disclosed examples to other alternative examples or uses and obvious modifications and equivalents thereof, including examples which do not provide all of the features and advantages set forth herein. Accordingly, the scope of the present disclosure is not intended to be limited by the described examples and may be defined by claims as presented herein or as presented in the future.

Conditional language, such as “can,” “could,” “might,” or “may,” unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain examples, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The same reference numbers may be used in different figures to denote similar, corresponding, or identical features in different embodiments. Values depicting dimensions may be shown in the figures. These dimensions are shown as non-limiting examples, and the dimensions may be greater or lesser than the dimensions depicted. For example, the dimensions may be lesser or greater than the depicted dimensions by 10%, 20%, 30%, 40%, or 50%. Any use of the phrase “preferred example” does not refer to a limiting example, but rather another non-limiting example.

1-35. (canceled)

36. A system for obstructing or reducing blood flow in an artery or vein in a patient, the system comprising:
a balloon catheter comprising:
an elongate body comprising a proximal portion and a distal portion;
an inflation lumen extending from the proximal portion to the distal portion;
a guidewire lumen extending from the proximal portion to the distal portion; and
a balloon positioned at the distal portion of the elongate body in fluid communication with the inflation lumen; and
an expandable metallic implant configured to be carried by the balloon catheter to an implantation site in an artery or vein in a patient and configured to expand from an unexpanded configuration to an expanded configuration, wherein the expandable metallic implant in the expanded configuration comprises a wall defining a hollow shape having a proximally facing opening at a proximal end thereof and a closed distal end except for a central opening to allow for passage of the distal portion of the elongate body;
wherein the balloon and the expandable metallic implant are folded and pleated together when configured in the unexpanded configuration,
wherein the balloon is configured to be inflated at the implantation site through the inflation lumen to expand the expandable metallic implant to the expanded configuration,
wherein the balloon catheter is configured to be removed from the patient such that the expandable metallic implant remains in the artery or vein in an expanded configuration at the implantation site.

37. The system of claim 36, wherein the expandable metallic implant comprises gold.

38. The system of claim 37, wherein the wall of the expandable metallic implant comprises a layer of gold with a thickness of between 5 microns and 500 microns that is deposited by electroforming.

39. The system of claim 36, wherein at least a portion of the central opening of the expandable metallic implant is covered, obstructed or occluded by a portion of the elongate body and a valve when the balloon catheter and the expandable metallic implant are joined.

40. The system of claim 39, wherein the portion of the central opening of the expandable metallic implant that is covered, obstructed or occluded when the balloon catheter and the expandable metallic implant are joined is covered, obstructed or occluded by a portion of the elongate body and a valve, and wherein a portion of the elongate body passes through the valve when the balloon catheter and the expandable metallic implant are joined.

41. The system of claim 36, wherein at least a portion of the central opening of the expandable metallic implant is covered, obstructed or occluded by a valve when the balloon catheter and the expandable metallic implant are separated.

42. The system of claim 41, wherein the valve is a duckbill valve, and an inflow portion of the duckbill valve faces the proximally facing opening of the expandable metallic implant.

43. The system of claim 36, wherein the distal portion of the expandable metallic implant comprises a wall defining a hollow shape having a distally facing opening at a distal end.

44. The system of claim 43, wherein the proximally facing opening and the distally facing opening are coaxially aligned along a central longitudinal axis.

45. The system of claim 43, wherein the distally facing opening is configured to accept a valve, including a duckbill valve.

46. The system of claim 43, wherein the expandable metallic implant comprises a narrowed region between the proximal opening and the distal opening, wherein a maximum diameter of the narrowed region is smaller than the maximum diameter of the proximal opening and the distally facing opening.

47. The system of claim 46, wherein the proximally facing opening, the narrowed region, and the distally facing opening are coaxially aligned along a central longitudinal axis.

48. The system of claim 36, wherein the expandable metallic implant in the expanded configuration comprises a balloon retention region, wherein, the balloon retention region has a smaller diameter than a portion of the expandable metallic implant distal to the balloon retention region.

49. The system of claim 36, wherein a length of the inflated balloon is greater than a length of the expanded expandable metallic implant.

50. The system of claim 36, wherein the balloon catheter is configured to allow for the passage of a guidewire through the guidewire lumen and, when the guidewire is not present, is configured to allow for the passage of one or more coil expandable implants.

51. The system of claim 36, wherein the elongate body comprises an inner shaft and an outer shaft, a proximal tail of the balloon is joined to the outer shaft, a distal tail of the balloon is joined to the inner shaft, and a fluid path of the inflation lumen passes between an outer surface of the inner shaft and the inner surface of the outer shaft.

52. The system of claim 36, wherein the balloon and the expandable metallic implant are folded together into wings.

53. The system of claim 52, wherein the proximal portion of the wings comprise two layers of balloon and do not comprise layers of expandable metallic implant.

54. The system of claim 53, wherein the two layers of balloon are external layers.

55. The system of claim 52, wherein the distal portion of the wings comprise two layers of expandable metallic implant and two layers of balloon.

56. The system of claim 55, wherein the two layers of balloon are internal layers, and the two layers of expandable metallic implant are outer layers.

57. The system of claim 52, wherein the wings are pleated around a central axis.

58. The system of claim 36, wherein the expandable metallic implant comprises one or more flaps configured to extend radially outward from a surface of the expandable metallic implant during inflation of the balloon.

59. The system of claim 58, wherein the one or more flaps are configured to protrude from a proximal end of the expandable metallic implant and configured such that free ends of the flaps are proximal to fixed ends.

60. The system of claim 58, wherein the one or more flaps are configured to contact the wall of an artery or vein wall in the radially extended configuration.

61. The system of claim 58, wherein the balloon is semi-compliant.

62. A method for obstructing or reducing blood flow in an artery or vein of a patient, the method comprising:
placing a guidewire in a selected segment of artery or vein of a patient;
advancing a system for obstructing or reducing blood flow in an artery or vein of a patient over the guidewire to the selected segment of artery or vein of a patient, the system comprising:
a balloon catheter comprising:
an elongate body comprising a proximal portion and a distal portion;
an inflation lumen extending from the proximal portion to the distal portion;
a guidewire lumen extending from the proximal portion to the distal portion; and
a balloon positioned at the distal portion of the elongate body in fluid communication with the inflation lumen; and
an expandable metallic implant configured to be carried by the balloon catheter to the selected segment of artery or vein in a patient and configured to expand from an unexpanded configuration to an expanded configuration, wherein the expandable metallic implant in the expanded configuration comprises a wall defining a hollow shape having a proximally facing opening at a proximal end thereof and a closed distal end except for a central opening to allow for passage of the distal portion of the elongate body,
wherein the balloon and the expandable metallic implant are folded and pleated together when configured in the unexpanded configuration;
inflating the balloon through the inflation lumen of the balloon catheter to cause the unexpanded expandable metallic implant to expand to an expanded configuration that provides for an obstruction or reduction of blood flow in the artery or vein at an implantation site;
deflating or collapsing the balloon; and
removing the balloon catheter and the guidewire from the patient while the expandable metallic implant remains at the implantation site in the patient in an expanded configuration.

63. The method of claim 62, wherein at least a portion of the central opening of the expandable metallic implant is covered, obstructed, or occluded after separation of the balloon catheter and the expandable metallic implant by a closing of a valve.

64. The method of claim 62, wherein deflating or collapsing the balloon and separating the balloon catheter and the expandable metallic implant results in the expandable metallic implant remaining in the expanded configuration and contacting a wall of an artery or vein segment at the implantation site of the patient after separation from the balloon catheter.

65. The method of claim 62, wherein inflating the balloon causes a flap of the expandable metallic implant to extend radially outward from a surface of the expandable metallic implant.
